(19) United States
(12) Patent Application Publication
(10) Pub. No.: US 2025/0381202 A1
(43) Pub. Date: Dec. 18, 2025

(54) PRODRUG KIT FOR MULTI-PRONGED CHEMOTHERAPY

(71) Applicant: Alex ZOUNEK, Wiesbaden (DE)

(72) Inventor: Alex ZOUNEK, Wiesbaden (DE)

(21) Appl. No.: 18/881,114

(22) PCT Filed: Jul. 5, 2023
(86) PCT No.: PCT/EP2023/068633

§ 371 Date: Jan. 3, 2025

(30) Foreign Application Priority Data
Jul. 5, 2022 (DE) 10 2022 002 443.8
Aug. 27, 2022 (DE) 10 2022 003 131.0
Nov. 16, 2022 (DE) 10 2022 004 229.0
Jan. 30, 2023 (DE) 10 2023 000 257.7
Feb. 10, 2023 (DE) 10 2023 000 423.5
Apr. 12, 2023 (DE) 10 2023 001 426.5

(51) Int. Cl. A61K 31/585; A61K 31/444; A61K 31/454; A61K 31/506; A61K 31/517; A61K 31/519

(52) U.S. Cl. CPC A61K 31/585; A61K 31/444; A61K 31/454; A61K 31/506; A61K 31/517; A61K 31/519

(57) ABSTRACT

A prodrug kit for multifactorial dynamic chemotherapy comprises N different small-molecule-drug-conjugates selected from the group comprising

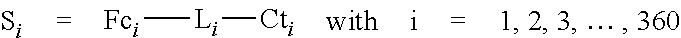

wherein 2≤N≤360, Fcᵢ is a moiety that is cleavable by fibroblast activation protein, Lᵢ is a self-immolative linker and Ctᵢ is a known chemotherapeutic agent.

### PRIOR RELATED APPLICATIONS

The present application is a national entry of PCT Application no. PCT/EP2023/068633, filed on Jul. 5, 2023, which claims priority under the Paris Convention to German Applications DE 10 2022 002 443.8, filed on Jul. 5, 2022; DE 10 2022 003 131.0, filed on Aug. 27, 2022; DE 10 2022 004 229.0, filed on Nov. 16, 2022; DE 10 2023 000 257.7, filed on Jan. 30, 2023; DE 10 2023 000 423.5, filed on Feb. 10, 2023; and DE 10 2023 001 426.5, filed on Apr. 12, 2023. The entire contents of such prior applications are incorporated by reference herein.

### FIELD OF THE DISCLOSURE

The present invention pertains to a prodrug kit for multifactorial dynamic chemotherapy comprising N different small-molecule-drug-conjugates selected from the group comprising

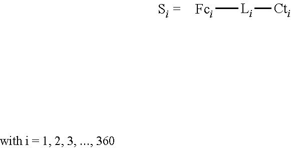

wherein 2≤N≤360, Fcᵢ is a moiety that is cleavable by fibroblast activation protein, Lᵢ is a self-immolative linker and Ctᵢ is a known chemotherapeutic agent.

### BACKGROUND OF THE DISCLOSURE

Cancer still represents a major health problem worldwide. Despite tremendous research effort to understand cancer biology and devise new therapies only limited success has been achieved in the treatment of leukemia and non-solid or soft tissue tumors. According to recent statistics from the International Agency for Research on Cancer (IARC) under the auspices of the World Health Organization (WHO) or the American Cancer Society (ACS), cancer incidence, mortality and financial burden are growing at an alarming pace around the globe. In 2014 the IARC reported that the global war against cancer cannot be won by treatment alone and urged implementation of prevention strategies to mitigate the imminent cancer crisis.

Most cancer tumors are diagnosed at an early stage and can be removed by surgical resection. Treatments for advanced or inoperable malignancies include:
radiation therapy;
systemic or targeted chemotherapy using cytotoxic agents, kinase inhibitors, immune checkpoint inhibitors, antibody-drug-conjugates (ADC) or small-molecule-drug-conjugates (SMDC); and
immunotherapies, particularly, chimeric antigen receptor therapy (CAR-T).

Unfortunately, despite good initial response, gradual resistance to these treatments still constitutes the leading cause of cancer recurrence and mortality. In order to identify novel therapeutic strategies and improve clinical outcomes, a better understanding of the molecular mechanisms underlying cancer progression and acquired drug resistance are urgently needed. In recent years, numerous biological mechanisms leading to therapy resistance have been identified, such as activation of growth factor receptors and their downstream signaling pathways, DNA repair mechanisms, metabolic rewiring, miRNA expression and transfer, ATP-binding cassette transporter-mediated drug extrusion and enrichment of cancer stem cell populations. Another important mechanism implicated in drug and radiation resistance is bidirectional communication between cancer cells and their microenvironment (stromal cells, vascular endothelial cells, immune cells) which appears to be effected by extracellular vesicles and their molecular cargo. More recently, the crucial role of the gut microbiome in tumor progression and patient response to different anticancer agents has been recognized. In this context, naturally occurring phytochemical compounds have also been gaining revived attention.

Increasingly, efforts are directed towards the identification of new biomarkers that allow to predict treatment response and prevent cancer relapse. Circulating cancer cells, as well as circulating tumor DNA, cancer cell secretome and tumor-derived extracellular vesicles and miRNAs can be easily isolated from patient body fluids. Thus, liquid biopsies are currently considered as promising tool for cancer detection and determination of a suitable treatment regime.

The majority of targeted, precision or personalized cancer therapies as well as recent immunotherapies employ drugs that address and modulate specific over- or under-expressed cancer-associated molecules such as hormones, enzymes, epitopes, growth factors, kinases, cytokines, chemokines, cellular receptors or adaptor proteins (e.g. Kras, P-gp, BCR, PI3K, CD11, CD22, CD44, Myc, BRCA2, ALK, IL-10, IL-12, p53, p27, p70, MAPKs, TKIs, VEGF, EGF). These molecular targets derive from modified or mutated genes (e.g. DNA damage, hypo- or hyper-methylated genes and expression products). The targeted molecular entities are part of the highly heterogeneous biochemical landscape of cancer. However, despite yielding promising results in in vitro studies and xenograft tumors in mice, most of the targeted molecular entities-on their own—have limited use for clinical translation. This dichotomy is corroborated by clinical trials where about 97 percent of novel drug candidates do not produce a quantitative improvement.

Further, stage III or IV cancer patients treated according to established chemotherapeutic regimens often develop drug resistance and advance to metastatic stage involving lymph nodes, liver, lung, bone and brain which eventually results in multiple organ failure, vascular damage, induction of a proteolytic cascade and disseminated intravascular coagulation which is most difficult to cure.

In contrast to the substantial body of research on the molecular mechanisms of resistance, understanding of how resistance evolves remains limited. Recent research suggests that resistance may originate from heterogeneous, weakly resistant cell subpopulations with different sensitivity to chemotherapeutic agents. Rather than the commonly assumed stochastic single hit (epi) mutational transition or drug-induced reprogramming, experimental studies point to a hybrid scenario involving gradual multifactorial adaption through various synergistic genetic and epigenetic changes.

Still, the majority of first and second line treatment regimens rely on one or two chemotherapeutic, mostly cytotoxic agents partly complemented by adjuvants that ameliorate side effects. In view of the transient multifactorial adaption capacity of cancer cells clinically established treatment regimens based on one or two chemotherapeutic agents that are repeatedly administered over extended time periods appear inadequate.

Since the 1980s, VAMP and RCHOP achieve better than 90% and 60% cure rates in pediatric ALL (acute lymphocytic leukemia) and DLBCL (diffuse large B cell lymphoma) by combining potent drugs with different mechanisms of action. For solid tumors, which protect cancer cells from the immune system and large xenobiotic molecules (like antibodies), cure rates are much lower. CAF/FAP-targeted prodrugs with reduced systemic toxicity can overcome the stromal barrier.

### SUMMARY OF THE DISCLOSURE

The inventive treatment regimen and FAP-prodrugs are inspired by and harness
(i) the large inventory of vintage and more recent cancer parent drugs with clinically proven potency;
(ii) the ever-growing prevalence of combination therapies; and
(iii) recent advances in cancer research, such as the ones cited beneath:
A. E. Pomeroy, E. V. Schmidt, P. K. Sorger, A. C. Palmer; Drug independence and the curability of cancer by combination chemotherapy; Trends in Cancer, November 2022, Vol. 8, No. 11; https://doi.org/10.1016/j.trecan.2022.06.009:

Concluding remarks: In this article we reviewed three historical principles that describe how combinations of independently active therapies can address the challenge of tumor heterogeneity and kill more cancer cells in more patients. None of these principles requires synergistic drug interaction (meaning supra-additive activity) to improve treatment outcomes, although their substantial clinical benefits are often colloquially called synergistic (meaning good for patients). Thus, the common sentiment that ‘to overcome drug resistance we need synergistic drug combinations’ is false in the quantitative sense. The multiple meanings of ‘synergy’ are a long-recognized source of confusion about mechanisms of combination therapy [38], and have caused tumor heterogeneity and drug cross-resistance to be overlooked as key factors in the efficacy of combination therapy.
A. O. Pisco, A. Brock, J. Zhou, A. Moor, M. Mojtahedi, D. Jackson, S. Huang; Non-Darwinian dynamics in therapy-induced cancer drug resistance; Nat Commun 4, 2467 (2013); https://doi.org/10.1038/ncomms3467:

The development of drug resistance, the prime cause of failure in cancer therapy, is commonly explained by the selection of resistant mutant cancer cells. However, dynamic non-genetic heterogeneity of clonal cell populations continuously produces metastable phenotypic variants (persisters), some of which represent stem-like states that confer resistance . . . . We show by quantitative measurement and modelling that appearance of MDR1-positive cells 1-2 days after treatment with vincristine (VINC) is predominantly mediated by cell-individual induction of MDR1 expression and not by the selection of MDR1-expressing cells.
https://www.sciencedirect.com/science/article/pii/S2589004220308531 Prof. Kornelia Polyak: “Yet, it seems like most cancer therapy research is based on finding a new drug target, ignoring the fact that every cancer drug that has been invented selects for resistance. We need to face this fact head on and figure out how to prevent or control therapeutic resistance.”
J. West, L. You, J. Zhang, R. A. Gatenby, J. S. Brown, P. K. Newton, A. R. A. Anderson; Towards Multidrug Adaptive Therapy; Cancer Res 2020, 80:1578-89; doi: 10.1158/0008-5472.CAN-19-2669.
A. H. Briggs et al.; An Attribution of Value Framework for Combination Therapies; https://assets-dam.takeda.com/raw/upload/v1675187100/legacy-dotcom/siteassets/en-gb/home/what-we-do/combination-treatments/a-value-attribution-framework-for-combination-therapies-takeda-whitepaper.pdf.
https://www.fiercepharma.com/pharma/after-ira-victory-senate-doubles-down-more-initiatives-cut-drug-pices.
AVA6000 clinical results: https://avacta.wistia.com/medias/tc76pkecuy; https://avacta.com/first-patient-dosed-in-fifth-cohort-of-ava6000-phase-ia-dose-escalation-study/.
A. Zana, A. Galbiati, E. Gilardoni, M. Bocci, J. Millul, T. Sturm, R. Stucchi, A. Elsayed, L. Nadal, M. Cirillo, W. Roll, L. Stegger, I. Asmus, P. Backhaus, M. Schaefers, D. Neri, S. Cazzamalli; Fibroblast Activation Protein triggers release of drug payload from non-internalizing small molecule-drug conjugates in solid tumors; Clin Cancer Res CCR-22-1788 Oct. 10 2022; https://doi.org/10.1158/1078-0432.CCR-22-1788.
M. Qi, S. Fan, M. Huang, J. Pan, Y. Li, Q. Miao, W. Lyu, X. Li, L. Deng, S. Qiu, T. Liu, W.-Deng, X. Chu, C. Jiang, W. He, L. Xia, Y. Yang, J. Hong, Q. Qi, W. Yin, X. Liu, C. Shi, M. Chen, W. Ye, D. Zhang; Targeting FAPα-expressing hepatic stellate cells overcomes resistance to antiangiogenics in colorectal cancer liver metastasis models; J Clin Invest. 2022; 132 (19): e157399; doi: 10.1172/JCI157399; https://jci.me/157399/pdf
G. Ye, M. Huang, Y. Li, J. Ouyang, M. Chen, Q. Wen, X. Li, H. Zeng, P. Long, Z. Fan, J. Yin, W. Ye, D. Zhang; The FAPα-activated prodrug Z-GP-DAVLBH inhibits the growth and pulmonary metastasis of osteosarcoma cells by suppressing the AXL pathway; Acta Pharmaceutica Sinica B 2022; 12 (3): 1288e1304; https://doi.org/10.1016/j.apsb.2021.08.015
X. Xu, R. Kumari, J. Zhou, J. Chen, B. Mao, J. Wang, M. Zheng, X. Tu, X. An, X. Chen, L. Zhang, X. Tian, H. Wang, X. Dong, Z. Bao, S. Guo, X. Ouyang, L. Shang, F. Wang, X. Yan, R. Zhang, R. G. J. Vries, H. Clevers, Q.-X. Li; A living biobank of matched pairs of patient-derived xenografts and organoids for cancer pharmacology; PLOS ONE 18 (1): e0279821; https://doi.org/10.1371/journal.pone.0279821.
Y. Kieffer, H. R. Hocine, G. Gentric, F. Pelon, C. Bernard, B. Bourachot, S. Lameiras, L. Albergante, C. Bonneau, A. Guyard, K. Tarte, A. Zinovyev, S. Baulande, G. Zalcman, A. Vincent-Salomon, F. Mechta-Grigoriou; Single-Cell Analysis Reveals Fibroblast Clusters Linked to Immunotherapy Resistance in Cancer; Cancer Discov 2020, 10:1330-51; doi: 10.1158/2159-8290.CD-19-1384.
N. Ortiz-Otero, J. R. Marshall, B. Lash, M. R. King; Chemotherapy-induced release of circulating-tumor cells into the bloodstream in collective migration units with cancer-associated fibroblasts in metastatic cancer patients; BMC Cancer (2020) 20:873; https://doi.org/10.1186/s12885-020-07376-1.
J.-W. Seo, K. Fu, S. Correa, M. Eisenstein, E. A. Appel, H. T. Soh; Real-time monitoring of drug pharmacokinetics within tumor tissue in live animals; Sci. Adv. 8, eabk2901 (2022); https://www.science.org/doi/epdf/10.1126/sciadv.abk2901

Cancer treatment and medication often follow the “magic bullet” and fractionated, i.e. protracted “maximum tolerated dose” paradigm. Cancer, though, involves heterogeneous cell populations that adapt metabolically, transcriptionally, epigenetically and evolutionarily to immunological, chemical, or radiological stresses within hours, days, weeks and months. To combat cancer variability, the present invention encompasses:
about 360 different small molecule drug conjugates (SMDC), each comprising one or more FAP-activatable initiators Fc, a self-immolative linker L and a known and proven chemotherapeutic agent Ct;
extracellular prodrug cleavage by fibroblast activation protein (FAP) overexpressed in solid tumors, metastases and aggregated with circulating tumor cells (CTC);
pan-tumor treatment, preferably personalized;
concurrent administration of multiple prodrugs (“total therapy”), preferably without cross-resistance;
high variability, e.g. selection of 2 prodrugs out of 360 affords about 129,000 different combinations;
timewise rapidly varied, non-redundant, non-mutagenic and preferably rational prodrug combinations;
ablation of heterogeneous cancer cell populations;
circumvention of neoplastic adaptation and resistance;
collateral attrition of cancer associated fibroblasts (CAF) and tumor micro environment (TME) which enhances immune system access to cancer cells;
more than 10-fold reduction of systemic toxicity;
low-risk/benign clinical trial and facilitated approval due to low toxicity and use of known chemotherapeutic agents Ct;
economic and affordable prodrug kits;
improved PK/PD versus prior art FAP-prodrugs.

Generally, a small prodrug kit of about four of the inventive CAF/FAP-targeted prodrugs should suffice to achieve total therapy (i.e. >10¹² cancer cell kills) and incidentally reinstate immune response.

Using a novel sensor Seo et al. measured pharmacokinetic parameters for cancer drug doxorubicin in an in vivo tumor model:

| PK parameter* | In blood | Tumor Mean PK |
|---|---|---|
| AUC₀₋ₜ (min · μg/ml) | 80.9 | 33.4 |
| t_{1/2} (min) | 3.4 | 89 |

*AUC₀₋ₜ is the area under the curve until the drug concentration reaches zero

Pisco et al., Seo et al. and a vast body of scientific literature show that:
cancer cells—like most cells—are resilient and utilize a variety of evolutionary defense mechanisms that allow them to adapt quickly and flexibly to therapeutic attack;
intratumoral drug exposure is anisotropic, which promotes cancer cell resistance in low dose regions;
systemic and tumoral drug clearance is rapid and requires fast and efficient intratumoral drug delivery.

Hence, in order to overcome refractory cancer the present invention proposes a multi-pronged, temporally rapidly varying treatment regimen comprising two, three, four, five or more stages, wherein
each stage extends over a time period of 48 hours to several weeks;
each stage comprises one or repeated administration of a set of two, three, four, five or more different tumor-targeted prodrugs simultaneously; and
a set of prodrugs administered in one stage differs from each set administered in a preceding or subsequent stage.

The proposed treatment regimen is advantageous in that it
enables high tumor-targeted dosing of chemotherapeutic agents with minimal side effects;
exposes cancer cells to a multitude of different chemotherapeutic agents in rapidly varying sequence;
addresses heterogeneous cancer cell populations;
counters cancer cell adaption and resistance; and
increases the chance for complete eradication of cancer stem cells.

### Dra

The above outlined treatment regimen is implemented through use of a chemotherapeutic drug kit comprising N different ready-made small-molecule-drug-conjugates selected from the group comprising

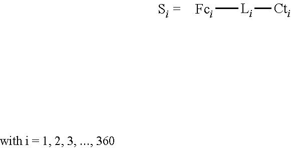

wherein

$$2 \leq N \leq 360 ,$$

each Ctᵢ is a residue of a known chemotherapeutic compound,
Ctᵢ≠Ctⱼ for i≠j,
each Fcᵢ is a residue of a fibroblast activation protein (FAP) cleavable moiety,
each Lᵢ is a residue of a self-immolative linker, and
Fcᵢ and Ctᵢ are covalently coupled to Lᵢ.

In a preferred embodiment of the chemotherapeutic drug kit each Sᵢ is provided in a separate container (e.g. medical vial or ampoule).

According to the present invention each Ctᵢ is a residue of one of the known chemotherapeutic agents depicted beneath in Table 1 and Table 2. Many of the chemotherapeutic agents listed in Table 1 and Table 2 have been used in clinical practice for years and in some cases for decades.

In the chemical structures shown in Table 1 and Table 2 groups suitable for covalent coupling with self-immolative linker Lᵢ are indicated by circles circumscribed with a dashed line. Generally, hydroxy (OH—), primary amine (NH₂—) or secondary amine (R—NH—R′) groups are suitable for conjugation via substitution of hydrogen (H) with self-immolative linker Lᵢ.

TABLE 1
Mainly inhibitory or modulating chemotherapeutic compounds (Ctᵢ)

| Name | CAS-No. | Structure and IUPAC Name |
|---|---|---|
| 1,2,3,4- Tetrahydrogen- staurosporine | 220038-19-7 | 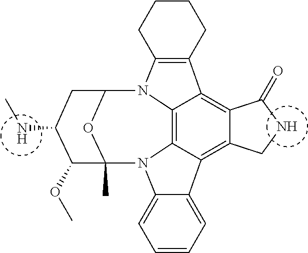 [SMILES: CN[C@@H]1CC2O[C@@](C)([C@@H]1OC)n1c3ccccc3c3c4c(c5c6c(n2c5c31)CCCC6)C(=O)NC4] |
| | | (2S,3R,4R,6R)-3-methoxy-2-methyl-4-(methyl- |
| | | amino)-29-oxa-1,7,17-triazaoctacyclo |
| | | [12.12.2.1²,⁶.0^{7,28}.0^{8,13}.0^{15,19}.0^{20,27}.0^{21,26}]nonacosa- |
| | | 8(13),14,19,21,23,25,27-heptaen-16-one |
| 17-Dmag | 467214-20-6 | 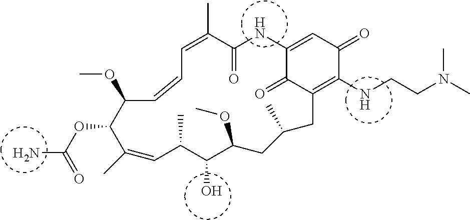 [SMILES: CO[C@H]1/C=C\C=C(\C)C(=O)NC2=CC(=O)C(NCCN(C)C)=C(C[C@@H](C)C[C@H](OC)[C@H](O)[C@@H](C)/C=C(/C)[C@@H]1OC(N)=O)C2=O] |
| | | [(4Z,6Z,8S,9S,10Z,12S,13R,14S,16R)-19-[2-(dimethylamino)ethylamino]- |
| | | 13-hydroxy-8,14-dimethoxy-4,10,12,16-tetramethyl-3,20,22-trioxo-2- |
| | | azabicyclo[16.3.1]docosa-1(21),4,6,10,18-pentaen-9-yl]carbamate |
| 2-Amino- pronanenitrile | 2134-48-7 | 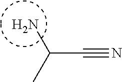 |
| | | 2-aminopropane- |
| | | nitrile |
| 4SC202 | 1186222-89-8 | 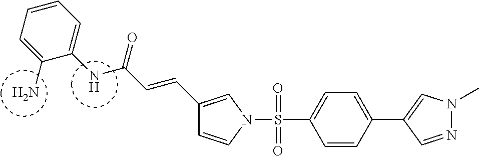 [SMILES: Cn1cc(-c2ccc(S(=O)(=O)n3ccc(/C=C/C(=O)Nc4ccccc4N)c3)cc2)cn1] |
| | | (E)-N-(2-aminophenyl)-3-[1-[4-(1-methylpyrazol-4-yl)phenyl]sulfonylpyrrol- |
| | | 3-yl]prop-2-enamide |
| ABBV-CLS-484 | 2489404-97-7 | 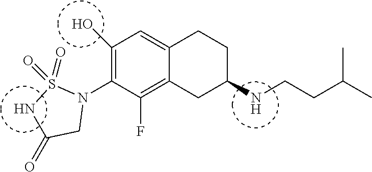 [SMILES: CC(C)CCN[C@@H]1CCc2cc(O)c(N3CC(=O)NS3(=O)=O)c(F)c2C1] |
| | | 5-[(7R)-1-fluoro-3-hydroxy-7-(3-methylbutylamino)-5,6,7,8- |
| | | tetrahydronaphthalen-2-yl]-1,1-dioxo-1,2,5-thiadiazolidin-3-one |
| Abemaciclib | 1231929-97-7 | 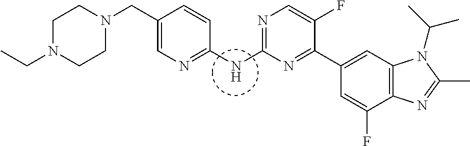 [SMILES: CCN1CCN(Cc2ccc(Nc3ncc(F)c(-c4cc(F)c5nc(C)n(C(C)C)c5c4)n3)nc2)CC1] |
| | | N-[5-[(4-ethylpiperazin-1-yl)methyl]pyridin-2-yl]-5-fluoro-4-(7-fluoro-2- |
| | | methyl-3-propan-2-ylbenzimidazol-5-yl)pyrimidin-2-amine |
| Abexinostat | 783355-60-2 | 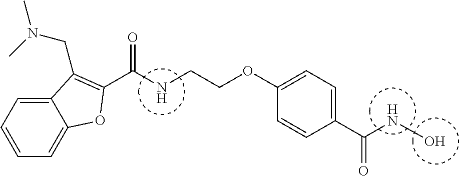 [SMILES: CN(C)Cc1c(C(=O)NCCOc2ccc(C(=O)NO)cc2)oc2ccccc12] |
| | | 3-[(dimethylamino)methyl]-N-[2-[4-(hydroxycarbamoyl)phenoxy]ethyl]- |
| | | 1-benzofuran-2-carboxamide |
| Acalabrutinib | 1420477-60-6 | 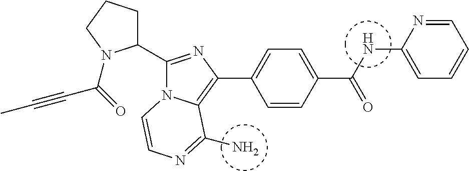 [SMILES: CC#CC(=O)N1CCCC1c1nc(-c2ccc(C(=O)Nc3ccccn3)cc2)c2c(N)nccn12] |
| | | 4-[8-amino-3-[(2S)-1-but-2-ynoylpyrrolidin-2-yl]imidazo[1,5-a]pyrazin-1- |
| | | yl]-N-pyridin-2-ylbenzamide |
| Acetylbufalin | 4029-66-7 | 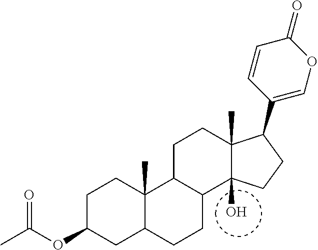 |
| | | [14-hydroxy-10,13-dimethyl-17-(6-oxopyran-3-yl)- |
| | | 1,2,3,4,5,6,7,8,9,11,12,15,16,17-tetradecahydro- |
| | | cyclopenta[a]phenanthren-3-yl]acetate |
| Aderbasib | 791828-58-5 | 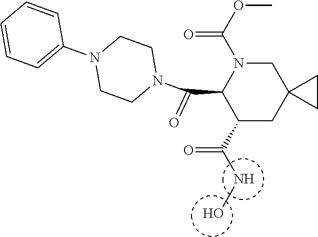 [SMILES: COC(=O)N1CC2(CC2)C[C@H](C(=O)NO)[C@H]1C(=O)N1CCN(c2ccccc2)CC1] |
| | | methyl(6S,7S)-7-(hydroxycarbamoyl)-6-(4-phenyl- |
| | | piperazine-1-carbonyl)-5-azaspiro[2.5]octane-5- |
| | | carboxylate |
| Afatinib | 850140-72-6 | 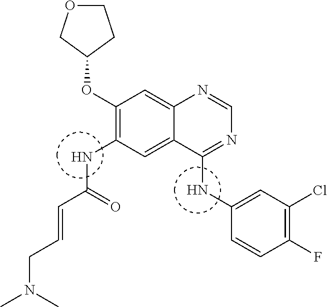 [SMILES: CN(C)C/C=C/C(=O)Nc1cc2c(Nc3ccc(F)c(Cl)c3)ncnc2cc1O[C@H]1CCOC1] |
| | | (E)-N-[4-(3-chloro-4-fluoroanilino)-7-[(3S)-oxolan- |
| | | 3-yl]oxyquinazolin-6-yl]-4-(dimethylamino)but- |
| | | 2-enamide |
| Afuresertib | 1047634-63-8 | 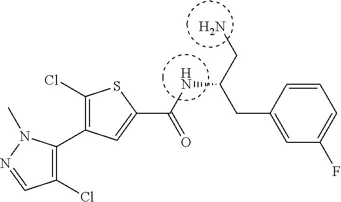 [SMILES: Cn1ncc(Cl)c1-c1cc(C(=O)N[C@H](CN)Cc2cccc(F)c2)sc1Cl] |
| | | N-[(2S)-1-amino-3-(3-fluorophenyl)propan-2-yl]-5- |
| | | chloro-4-(4-chloro-2-methylpyrazol-3-yl)thiophene- |
| | | 2-carboxamide |
| Alectinib | 1256580-46-7 | 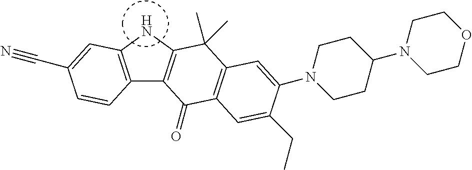 [SMILES: CCc1cc2c(cc1N1CCC(N3CCOCC3)CC1)C(C)(C)c1[nH]c3cc(C#N)ccc3c1C2=O] |
| | | 9-ethyl-6,6-dimethyl-8-(4-morpholin-4-ylpiperidin-1-yl)-11-oxo-5H-benzo |
| | | [b]carbazole-3-carbonitrile |
| Alisertib | 1028486-01-2 | 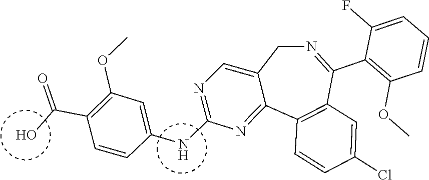 [SMILES: COc1cc(Nc2ncc3c(n2)-c2ccc(Cl)cc2C(c2c(F)cccc2OC)=NC3)ccc1C(=O)O] |
| | | 4-[[9-chloro-7-(2-fluoro-6-methoxyphenyl)-5H-pyrimido[5,4-d][2] |
| | | benzazepin-2-yl]amino]-2-methoxybenzoic acid |
| Alpelisib | 1217486-61-7 | 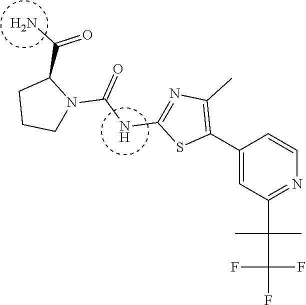 [SMILES: Cc1nc(NC(=O)N2CCC[C@H]2C(N)=O)sc1-c1ccnc(C(C)(C)C(F)(F)F)c1] |
| | | (2S)-1-N-[4-methyl-5-[2-(1,1,1-trifluoro-2-meth- |
| | | ylpropan-2-yl)pyridin-4-yl]-1,3-thiazol-2-yl] |
| | | pyrrolidine-1,2-dicarboxamide |
| Alvocidib | 146426-40-6 | 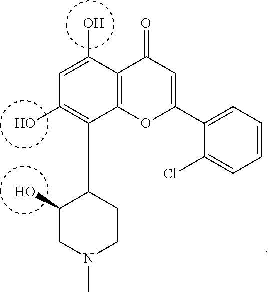 [SMILES: CN1CCC(c2c(O)cc(O)c3c(=O)cc(-c4ccccc4Cl)oc23)[C@H](O)C1] |
| | | 2-(2-chlorophenyl)-5,7-dihydroxy-8- |
| | | [(3S,4R)-3-hydroxy-1-methylpiperidin- |
| | | 4-yl]chromen-4-one |
| AMD3465 | 185991-24-6 | 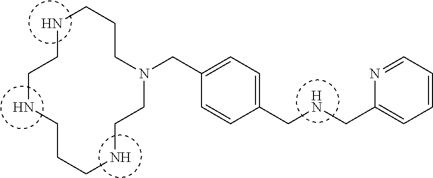 [SMILES: c1ccc(CNCc2ccc(CN3CCCNCCNCCCNCC3)cc2)nc1] |
| | | N-(pyridin-2-ylmethyl)-1-[4-(1,4,8,11-tetrazacyclotetradec-1-ylmeth- |
| | | yl)phenyl]methanamine |
| Anlotinib | 1058156-90-3 | 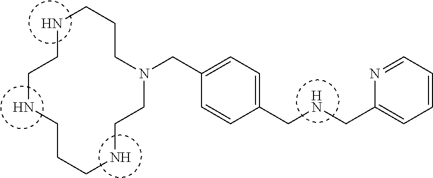 [SMILES: c1ccc(CNCc2ccc(CN3CCCNCCNCCCNCC3)cc2)nc1] |
| | | 1-[[4-[(4-fluoro-2-methyl-1H-indol-5-yl)oxy]-6-methoxyquinolin-7- |
| | | yl]oxymethyl]cyclopropan-1-amine |
| Apalutamide | 956104-40-8 | 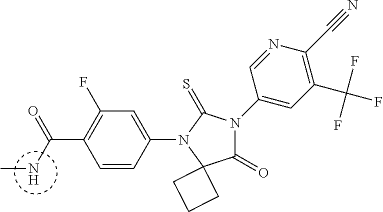 [SMILES: CNC(=O)c1ccc(N2C(=S)N(c3cnc(C#N)c(C(F)(F)F)c3)C(=O)C23CCC3)cc1F] |
| | | 4-[7-[6-cyano-5-(trifluoromethyl)pyridin-3-yl]-8-oxo-6- |
| | | sulfanylidene-5,7-diazaspiro[3.4]octan-5-yl]-2-fluoro-N- |
| | | methylbenzamide |
| AR-42 | 935881-37-1 | 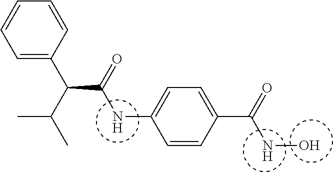 [SMILES: CC(C)[C@H](C(=O)Nc1ccc(C(=O)NO)cc1)c1ccccc1] |
| | | N-hydroxy-4-[[(2S)-3-methyl-2-phenylbutanoyl] |
| | | amino]benzamide |
| Asciminib | 1492952-76-7 | 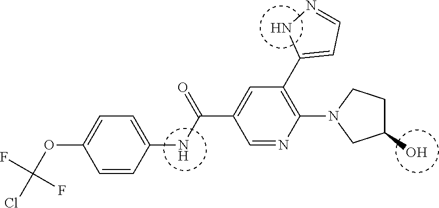 |
| | | N-[4-[chloro(difluoro)methoxy]phenyl]-6-[(3R)-3-hydroxypyrrolidin- |
| | | 1-yl]-5-(1H-pyrazol-5-yl)pyridine-3-carboxamide |
| Atuveciclib | 1414943-94-4 | 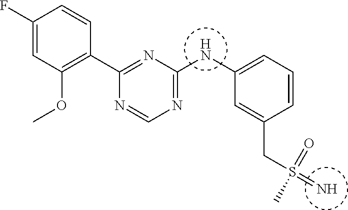 [SMILES: COc1cc(F)ccc1-c1ncnc(Nc2cccc(C[S@](C)(=N)=O)c2)n1] |
| | | 4-(4-fluoro-2-methoxyphenyl)-N-[3-[(methylsulfonimido- |
| | | yl)methyl]phenyl]-1,3,5-triazin-2-amine |
| Avapritinib | 1703793-34-3 | 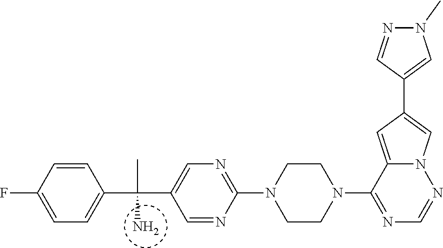 |
| | | (1S)-1-(4-fluorophenyl)-1-[2-[4-[6-(1-methylpyrazol-4-yl)pyrrolo[2,1-f] |
| | | [1,2,4]triazin-4-yl]piperazin-1-yl]pyrimidin-5-yl]ethanamine |
| Axitinib | 319460-85-0 | 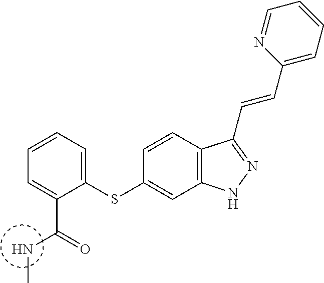 [SMILES: CNC(=O)c1ccccc1Sc1ccc2c(/C=C/c3ccccn3)n[nH]c2c1] |
| | | N-methyl-2-[[3-[(E)-2-pyridin-2-ylethenyl]-1H- |
| | | indazol-6-yl]sulfanyl]benzamide |
| AZD7762 | 860352-01-8 | 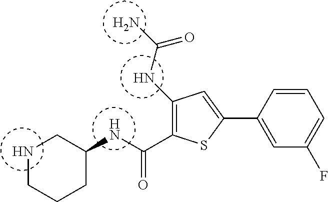 [SMILES: NC(=O)Nc1cc(-c2cccc(F)c2)sc1C(=O)N[C@H]1CCCNC1] |
| | | 3-(carbamoylamino)-5-(3-fluorophenyl)-N-[(3S)- |
| | | piperidin-3-yl]thiophene-2-carboxamide |
| BAYL125976 | 1402608-02-9 | 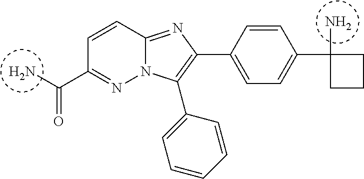 [SMILES: NC(=O)c1ccc2nc(-c3ccc(C4(N)CCC4)cc3)c(-c3ccccc3)n2n1] |
| | | 2-[4-(1-aminocyclobutyl)phenyl]-3-phenylimidazo[1,2-b] |
| | | pyridazine-6-carboxamide |
| Belinostat | 866323-14-0 | 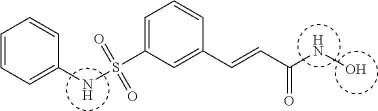 [SMILES: O=C(/C=C/c1cccc(S(=O)(=O)Nc2ccccc2)c1)NO] |
| | | (E)-N-hydroxy-3-[3-(phenylsulfamoyl)phenyl]prop-2-enamide |
| β-Hydroxy- isovaleric acid | 625-08-1 | 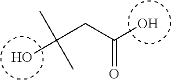 |
| | | 3-hydroxy-3-methylbutanoic |
| | | acid |
| BF211 | — | 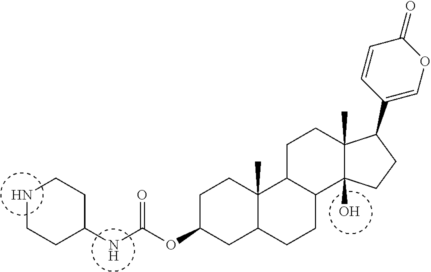 |
| | | [14-hydroxy-10,13-dimethyl-17-(6-oxopyran-3-yl)-1,2,3,4,5,6,7,8,9,11, |
| | | 12,15,16,17-tetradecahydrocyclopenta[a]phenanthren-3-yl]piperidin- |
| | | 4-ylcarbamate |
| Bicalutamide | 90357-06-5 | 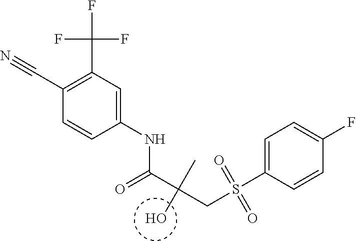 [SMILES: CC(O)(CS(=O)(=O)c1ccc(F)cc1)C(=O)Nc1ccc(C#N)c(C(F)(F)F)c1] |
| | | N-[4-cyano-3-(trifluoromethyl)phenyl]-3-(4-fluorophenyl)- |
| | | sulfonyl-2-hydroxy-2-methylpropanamide |
| Binimetinib | 606143-89-9 | 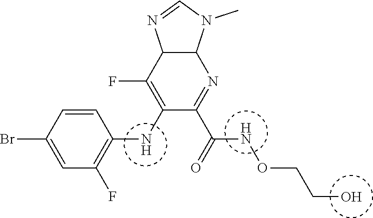 [SMILES: CN1C=NC2C(F)=C(Nc3ccc(Br)cc3F)C(C(=O)NOCCO)=NC21] |
| | | 6-(4-bromo-2-fluoroanilino)-7-fluoro-N-(2-hydroxyethoxy)- |
| | | 3-methylbenzimidazole-5-carboxamide |
| Bortezomib | 179324-69-7 | 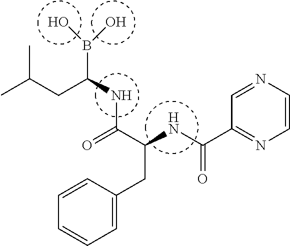 [SMILES: CC(C)C[C@H](NC(=O)[C@H](Cc1ccccc1)NC(=O)c1cnccn1)B(O)O] |
| | | [(1R)-3-methyl-1-[[(2S)-3-phenyl-2-(pyrazine- |
| | | 2-carbonylamino)propanoyl]amino]butyl] |
| | | boronic acid |
| Bosutinib | 380843-75-4 | 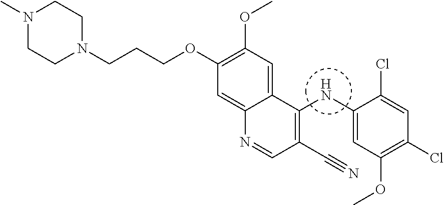 [SMILES: COc1cc(Nc2c(C#N)cnc3cc(OCCCN4CCN(C)CC4)c(OC)cc23)c(Cl)cc1Cl] |
| | | 4-(2,4-dichloro-5-methoxyanilino)-6-methoxy-7-[3-(4-methylpiperazin- |
| | | 1-yl)propoxy]quinoline-3-carbonitrile |
| Brigatinib | 1197953-54-0 | 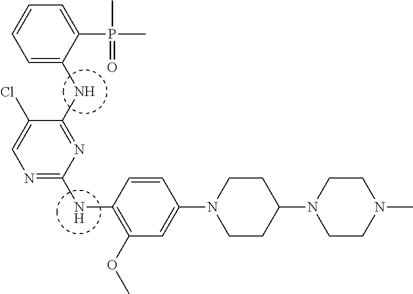 [SMILES: COc1cc(N2CCC(N3CCN(C)CC3)CC2)ccc1Nc1ncc(Cl)c(Nc2ccccc2P(C)(C)=O)n1] |
| | | 5-chloro-4-N-(2-dimethylphosphorylphenyl)-2-N-[2-methoxy-4- |
| | | [4-(4-methylpiperazin-1-yl)piperidin-1-yl]phenyl]pyrimidine- |
| | | 2,4-diamine |
| Bufalin | 465-21-4 | 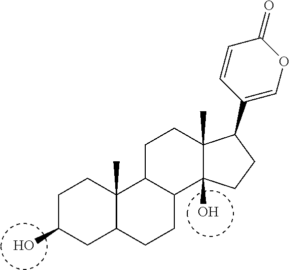 |
| | | 5-[(3S,5R,8R,9S,10S,13R,14S,17R)-3,14- |
| | | dihydroxy-10,13-dimethyl-1,2,3,4,5,6,7,8, |
| | | 9,11,12,15,16,17-tetradecahydrocyclopenta |
| | | [a]phenanthren-17-yl]pyran-2-one |
| Buparlisib | 944396-07-0 | 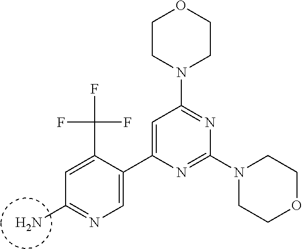 [SMILES: Nc1cc(C(F)(F)F)c(-c2cc(N3CCOCC3)nc(N3CCOCC3)n2)cn1] |
| | | 5-(2,6-dimorpholin-4-ylpyrimidin-4-yl)-4- |
| | | (trifluoromethyl)pyridin-2-amine |
| Buthionine sulfoximine | 5072-26-4 | 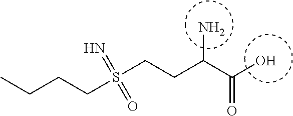 |
| | | 2-amino-4-(butylsulfonimidoyl)butanoic acid |
| Cabozantinib | 849217-68-1 | 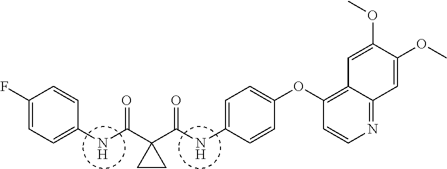 [SMILES: COc1cc2nccc(Oc3ccc(NC(=O)C4(C(=O)Nc5ccc(F)cc5)CC4)cc3)c2cc1OC] |
| | | 1-N-[4-(6,7-dimethoxyquinolin-4-yl)oxyphenyl]-1-N′-(4-fluorophenyl)- |
| | | cyclopropane-1,1-dicarboxamide |
| Capivasertib | 1143532-39-1 | 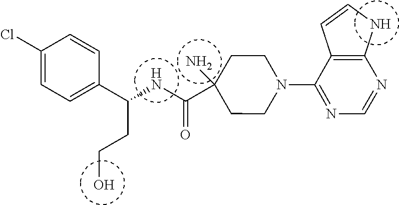 |
| | | 4-amino-N-[(1S)-1-(4-chlorophenyl)-3-hydroxypropyl]-1-(7H- |
| | | pyrrolo[2,3-d]pyrimidin-4-yl)piperidine-4-carboxamide |
| Capmatinib | 1029712-80-8 | 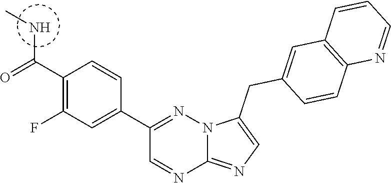 [SMILES: CNC(=O)c1ccc(-c2cnc3ncc(Cc4ccc5ncccc5c4)n3n2)cc1F] |
| | | 2-fluoro-N-methyl-4-[7-(quinolin-6-ylmethyl)imidazo[1,2-b] |
| | | [1,2,4]triazin-2-yl]benzamide |
| Carfilzomib | 868540-17-4 | 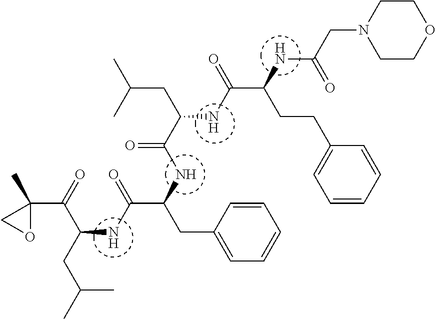 [SMILES: CC(C)C[C@H](NC(=O)[C@H](CCc1ccccc1)NC(=O)CN1CCOCC1)C(=O)N[C@@H](Cc1ccccc1)C(=O)N[C@@H](CC(C)C)C(=O)[C@@]1(C)CO1] |
| | | (2S)-4-methyl-N-[(2S)-1-[[(2S)-4-methyl-1-[(2R)-2-methyloxiran-2- |
| | | yl]-1-oxopentan-2-yl]amino]-1-oxo-3-phenylpropan-2-yl]-2-[[(2S)-2- |
| | | [(2-morpholin-4-ylacetyl)amino]-4-phenylbutanoyl]amino]pentanamide |
| CEP-9722 | 916574-83-9 | 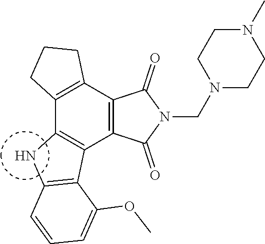 [SMILES: COc1cccc2[nH]c3c4c(c5c(c3c12)C(=O)N(CN1CCN(C)CC1)C5=O)CCC4] |
| | | 14-methoxy-9-[(4-methylpiperazin-1-yl) |
| | | methyl]-9,19-diazapentacyclo[10.7.0.0^{2,6}. |
| | | 0^{7,11}.0^{13,18}]nonadeca-1(12),2(6),7(11), |
| | | 13(18),14,16-hexaene-8,10-dione |
| Ceralasertib | 1352226-88-0 | 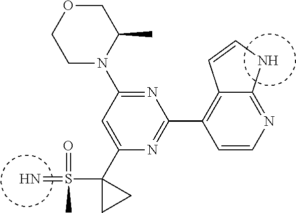 [SMILES: C[C@@H]1COCCN1c1cc(C2([S@](C)(=N)=O)CC2)nc(-c2ccnc3[nH]ccc23)n1] |
| | | imino-methyl-[1-[6-[(3R)-3-methylmorpholin- |
| | | 4-yl]-2-(1H-pyrrolo[2,3-b]pyridin-4-yl) |
| | | pyrimidin-4-yl]cyclopropyl]-oxo-λ⁶-sulfane |
| Ceritinib | 1032900-25-6 | 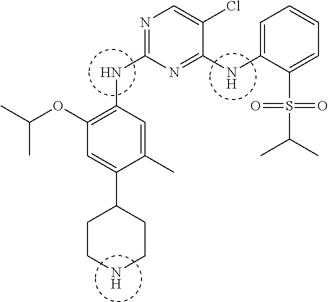 [SMILES: Cc1cc(Nc2ncc(Cl)c(Nc3ccccc3S(=O)(=O)C(C)C)n2)c(OC(C)C)cc1C1CCNCC1] |
| | | 5-chloro-2-N-(5-methyl-4-piperidin-4-yl-2-propan- |
| | | 2-yloxyphenyl)-4-N-(2-propan-2-ylsulfonylphenyl)- |
| | | pyrimidine-2,4-diamine |
| Chidamide | 1883690-47-8 | 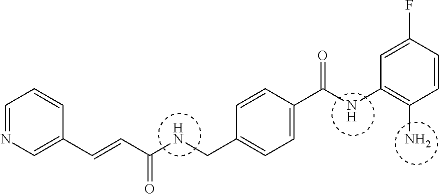 [SMILES: Nc1ccc(F)cc1NC(=O)c1ccc(CNC(=O)/C=C/c2cccnc2)cc1] |
| | | N-(2-amino-5-fluorophenyl)-4-[[[(E)-3-pyridin-3-ylprop-2-enoyl]amino] |
| | | methyl]benzamide |
| CHR-3996 | 1256448-47-1 | 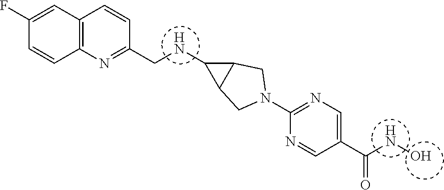 [SMILES: O=C(NO)c1cnc(N2CC3C(C2)C3NCc2ccc3cc(F)ccc3n2)nc1] |
| | | 2-[(1S,5R)-6-[(6-fluoroquinolin-2-yl)methylamino]-3-azabicyclo[3.1.0] |
| | | hexan-3-yl]-N-hydroxypyrimidine-5-carboxamide |
| Citarinostat | 1316215-12-9 | 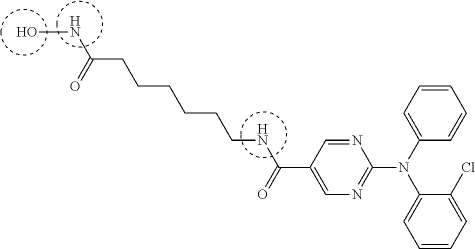 [SMILES: O=C(CCCCCCNC(=O)c1cnc(N(c2ccccc2)c2ccccc2Cl)nc1)NO] |
| | | 2-(N-(2-chlorophenyl)anilino)-N-[7-(hydroxyamino)-7-oxoheptyl]pyrimidine- |
| | | 5-carboxamide |
| Cobimetinib | 934660-93-2 | 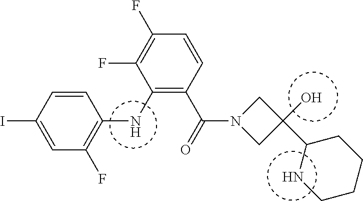 |
| | | [3,4-difluoro-2-(2-fluoro-4-iodoanilino)phenyl]-[3-hydroxy- |
| | | 3-[(2S)-piperidin-2-yl]azetidin-1-yl]methanone |
| CompK | 2320462-65-3 | 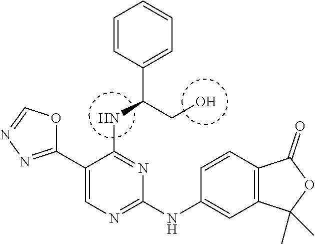 [SMILES: CC1(C)OC(=O)c2ccc(Nc3ncc(-c4nnco4)c(N[C@H](CO)c4ccccc4)n3)cc21] |
| | | (S)-5-((4-((2-Hydroxy-1-phenylethyl)amino)-5- |
| | | (1,3,4-oxadiazol-2-yl)pyrimidin-2-yl)amino)-3,3- |
| | | dimethylisobenzofuran-1(3H)-one |
| Copanlisib | 1032568-63-0 | 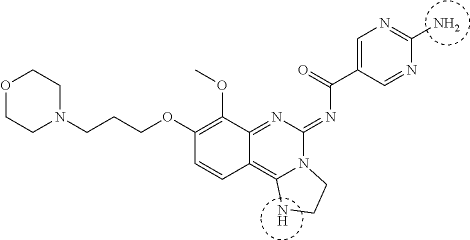 [SMILES: COc1c(OCCCN2CCOCC2)ccc2c3n(/c(=N/C(=O)c4cnc(N)nc4)nc12)CCN3] |
| | | 2-amino-N-[7-methoxy-8-(3-morpholin-4-ylpropoxy)-2,3-dihydro-1H- |
| | | imidazo[1,2-c]quinazolin-5-ylidene]pyrimidine-5-carboxamide |
| Crenolanib | 670220-88-9 | 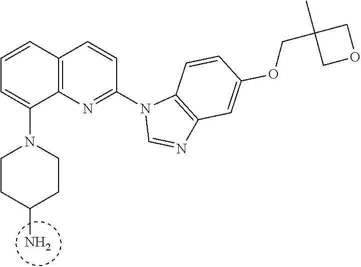 [SMILES: CC1(COc2ccc3c(c2)ncn3-c2ccc3cccc(N4CCC(N)CC4)c3n2)COC1] |
| | | 1-[2-[5-[(3-methyloxetan-3-yl)methoxy]benzimidazol-1- |
| | | yl]quinolin-8-yl]piperidin-4-amine |
| Crizotinib | 877399-52-5 | 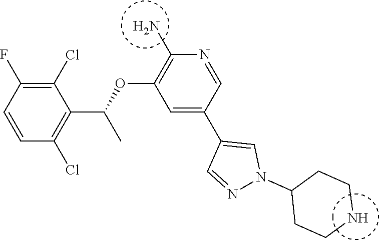 [SMILES: C[C@@H](Oc1cc(-c2cnn(C3CCNCC3)c2)cnc1N)c1c(Cl)ccc(F)c1Cl] |
| | | 3-[(1R)-1-(2,6-dichloro-3-fluorophenyl)ethoxy]-5-(1- |
| | | piperidin-4-ylpyrazol-4-yl)pyridin-2-amine |
| CUDC-101 | 1012054-59-9 | 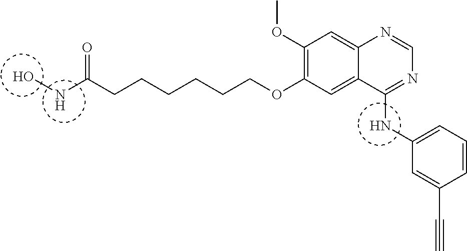 [SMILES: C#Cc1cccc(Nc2ncnc3cc(OC)c(OCCCCCCC(=O)NO)cc23)c1] |
| | | 7-[4-(3-ethynylanilino)-7-methoxyquinazolin-6-yl]oxy-N-hydroxyheptanamide |
| Dabrafenib | 1195765-45-7 | 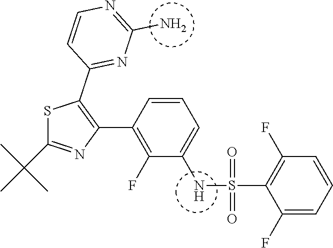 [SMILES: CC(C)(C)c1nc(-c2cccc(NS(=O)(=O)c3c(F)cccc3F)c2F)c(-c2ccnc(N)n2)s1] |
| | | N-[3-[5-(2-aminopyrimidin-4-yl)-2-tert-butyl-1,3- |
| | | thiazol-4-yl]-2-fluorophenyl]-2,6-difluorobenzene- |
| | | sulfonamide |
| Daclatasvir | 1009119-64-5 | 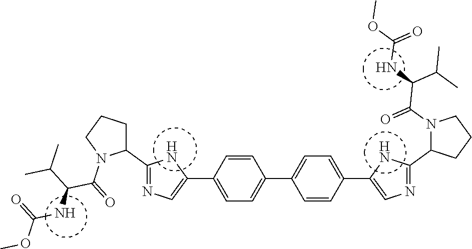 [SMILES: COC(=O)N[C@H](C(=O)N1CCCC1c1ncc(-c2ccc(-c3ccc(-c4cnc(C5CCCN5C(=O)[C@@H](NC(=O)OC)C(C)C)[nH]4)cc3)cc2)[nH]1)C(C)C] |
| | | methyl N-[(2S)-1-[(2S)-2-[5-[4-[4-[2-[(2S)-1-[(2S)-2-(methoxycarbonyl- |
| | | amino)-3-methylbutanoyl]pyrrolidin-2-yl]-1H-imidazol-5-yl]phenyl]phenyl]- |
| | | 1H-imidazol-2-yl]pyrrolidin-1-yl]-3-methyl-1-oxobutan-2-yl]carbamate |
| Dacomitinib | 1110813-31-4 | 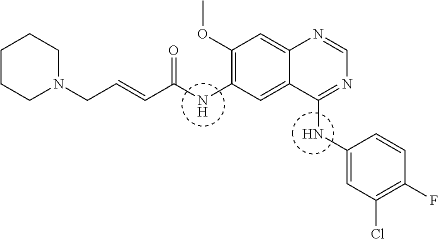 [SMILES: COc1cc2ncnc(Nc3ccc(F)c(Cl)c3)c2cc1NC(=O)/C=C/CN1CCCCC1] |
| | | (E)-N-[4-(3-chloro-4-fluoroanilino)-7-methoxyquinazolin-6-yl]-4- |
| | | piperidin-1-ylbut-2-enamide |
| Darolutamide | 1297538-32-9 | 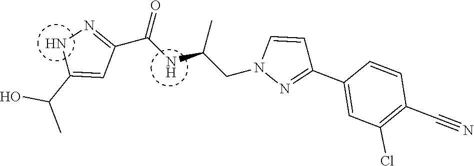 [SMILES: CC(O)c1cc(C(=O)N[C@@H](C)Cn2ccc(-c3ccc(C#N)c(Cl)c3)n2)n[nH]1] |
| | | N-[(2S)-1-[3-(3-chloro-4-cyanophenyl)pyrazol-1-yl]propan-2-yl]-5-(1-hydroxy- |
| | | ethyl)-1H-pyrazole-3-carboxamide |
| Dasatinib | 302962-49-8 | 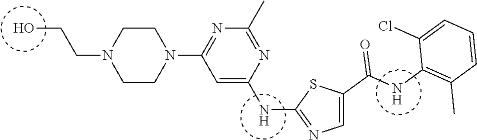 [SMILES: Cc1nc(Nc2ncc(C(=O)Nc3c(C)cccc3Cl)s2)cc(N2CCN(CCO)CC2)n1] |
| | | N-(2-chloro-6-methylphenyl)-2-[[6-[4-(2-hydroxyethyl)piperazin-1-yl]-2- |
| | | methylpyrimidin-4-yl]amino]-1,3-thiazole-5-carboxamide |
| Dasatinib D1 | — | 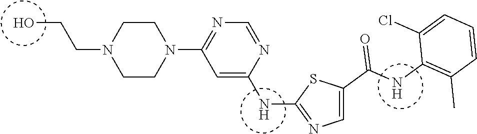 [SMILES: Cc1cccc(Cl)c1NC(=O)c1cnc(Nc2cc(N3CCN(CCO)CC3)ncn2)s1] |
| Dasatinib D2 | — | 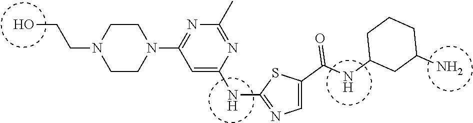 [SMILES: Cc1nc(Nc2ncc(C(=O)NC3CCCC(N)C3)s2)cc(N2CCN(CCO)CC2)n1] |
| Dasatinib D3 | — | 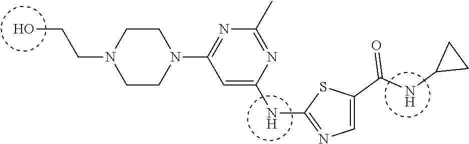 [SMILES: Cc1nc(Nc2ncc(C(=O)NC3CC3)s2)cc(N2CCN(CCO)CC2)n1] |
| Dasatinib D4 | — | 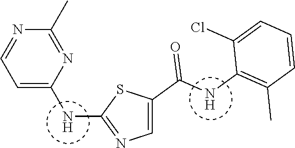 [SMILES: Cc1nccc(Nc2ncc(C(=O)Nc3c(C)cccc3Cl)s2)n1] |
| Decitabine | 2353-33-5 | 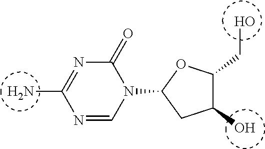 [SMILES: Nc1ncn([C@H]2C[C@H](O)[C@@H](CO)O2)c(=O)n1] |
| | | 4-amino-1-[(2R,4S,5R)-4-hydroxy-5- |
| | | (hydroxymethyl)oxolan-2-yl]-1,3,5- |
| | | triazin-2-one |
| Defactinib | 1073154-85-4 | 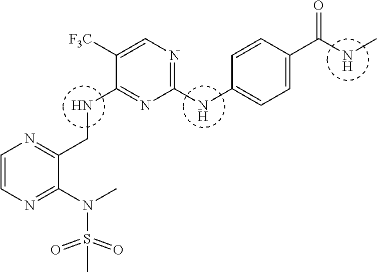 [SMILES: CNC(=O)c1ccc(Nc2ncc(C(F)(F)F)c(NCc3nccnc3N(C)S(C)(=O)=O)n2)cc1] |
| | | N-methyl-4-[[4-[[3-[methyl(methylsulfonyl)amino]pyrazin- |
| | | 2-yl]methylamino]-5-(trifluoromethyl)pyrimidin-2-yl] |
| | | amino]benzamide |
| Degarelix | 214766-78-6 | 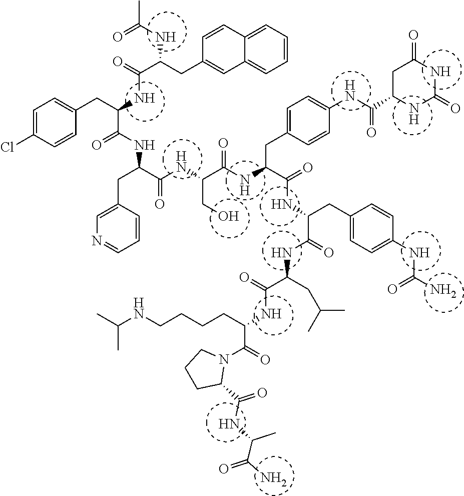 [SMILES: CC(=O)N[C@H](Cc1ccc2ccccc2c1)C(=O)N[C@H](Cc1ccc(Cl)cc1)C(=O)N[C@H](Cc1cccnc1)C(=O)N[C@@H](CO)C(=O)N[C@@H](Cc1ccc(NC(=O)[C@@H]2CC(=O)NC(=O)N2)cc1)C(=O)N[C@H](Cc1ccc(NC(N)=O)cc1)C(=O)N[C@@H](CC(C)C)C(=O)N[C@@H](CCCCNC(C)C)C(=O)N1CCC[C@H]1C(=O)N[C@H](C)C(N)=O] |
| | | (4S)-N-[4-[(2S)-2-[(2S)-2-[(2R)-2-[[(2R)-2-[[(2R)-2-acetamido-3-naphthalen- |
| | | 2-ylpropanoyl]amino]-3-(4-chlorophenyl)propanoyl]amino]-3-pyridin-3- |
| | | ylpropanoyl]amino]-3-hydroxypropanoyl]amino]-3-[(2R)-1-[(2S)-1-[(2S)-1- |
| | | [(2S)-2-[[(2R)-1-amino-1-oxopropan-2-yl]carbamoyl]pyrrolidin-1-yl]-1-oxo- |
| | | 6-(propan-2-ylamino)hexan-2-yl]amino]-4-methyl-1-oxopentan-2-yl]amino]- |
| | | 3-[4-(carbamoylamino)phenyl]-1-oxopropan-2-yl]amino]-3-oxopropyl]phen- |
| | | yl]-2,6-dioxo-1,3-diazinane-4-carboxamide |
| Diethylstilbestrol | 56-53-1 | 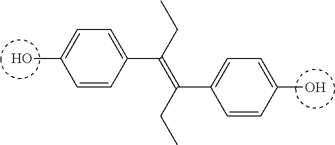 [SMILES: CC/C(=C(/CC)c1ccc(O)cc1)c1ccc(O)cc1] |
| | | 4-[(E)-4-(4-hydroxyphenyl)hex-3-en-3-yl]phenol |
| Dinaciclib | 779353-01-4 | 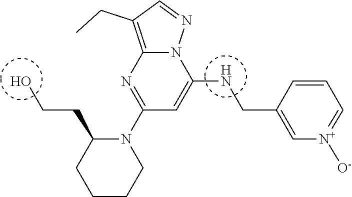 [SMILES: CCc1cnn2c(NCc3ccc[n+]([O-])c3)cc(N3CCCC[C@H]3CCO)nc12] |
| | | 2-[(2S)-1-[3-ethyl-7-[(1-oxidopyridin-1-ium-3-yl)methyl- |
| | | amino]pyrazolo[1,5-a]pyrimidin-5-yl]piperidin-2-yl] |
| | | ethanol |
| Dp44mT | 152095-12-0 | 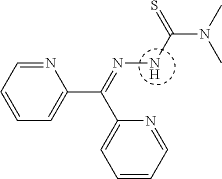 [SMILES: CN(C)C(=S)NN=C(c1ccccn1)c1ccccn1] |
| | | 3-(dipyridin-2-ylmethylideneamino)- |
| | | 1,1-dimethylthiourea |
| DpC | 1382469-39-7 | 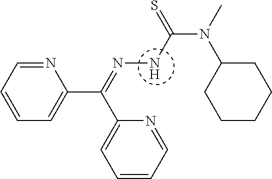 [SMILES: CN(C(=S)NN=C(c1ccccn1)c1ccccn1)C1CCCCC1] |
| | | 1-cyclohexyl-3-(dipyridin-2-ylmethylidene- |
| | | amino)-1-methylthiourea |
| DUPA | — | 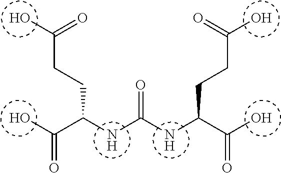 [SMILES: O=C(O)CC[C@H](NC(=O)N[C@@H](CCC(=O)O)C(=O)O)C(=O)O] |
| | | (2S)-2-[(1S)-1,3-dicarboxypropyl]carbamoyl- |
| | | amino]pentanedioic acid |
| Duvelisib | 1201438-56-3 | 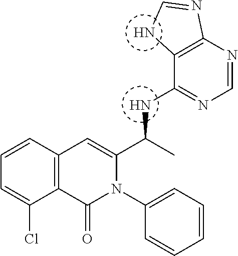 [SMILES: C[C@H](Nc1ncnc2nc[nH]c12)c1cc2cccc(Cl)c2c(=O)n1-c1ccccc1] |
| | | 8-chloro-2-phenyl-3-[(1S)-1-(7H-purin- |
| | | 6-ylamino)ethyl]isoquinolin-1-one |
| E7016 | 902128-92-1 | 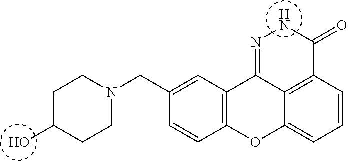 [SMILES: O=c1[nH]nc2c3c(cccc13)Oc1ccc(CN3CCC(O)CC3)cc1-2] |
| | | 4-[(4-hydroxypiperidin-1-yl)methyl]-8-oxa-15,16-diaza- |
| | | tetracyclo[7.7.1.0^{2,7}.0^{13,17}]heptadeca-1(16),2(7),3,5,9,11, |
| | | 13(17)-heptaen-14-one |
| Ebvaciclib | 2185857-97-8 | 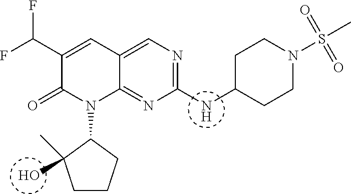 [SMILES: C[C@@]1(O)CCC[C@H]1n1c(=O)c(C(F)F)cc2cnc(NC3CCN(S(C)(=O)=O)CC3)nc21] |
| | | 6-(difluoromethyl)-8-[(1R,2R)-2-hydroxy-2-methylcyclo- |
| | | pentyl]-2-[(1-methylsulfonylpiperidin-4-yl)amino]pyrido |
| | | [2,3-d]pyrimidin-7-one |
| Eganelisib | 1693758-51-8 | 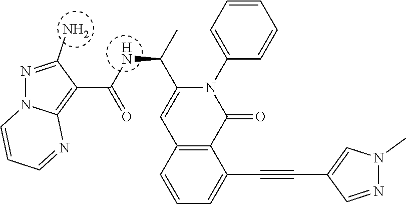 [SMILES: C[C@H](NC(=O)c1c(N)nn2cccnc12)c1cc2cccc(C#Cc3cnn(C)c3)c2c(=O)n1-c1ccccc1] |
| | | 2-amino-N-[(1S)-1-[8-[2-(1-methylpyrazol-4-yl)ethynyl]-1-oxo- |
| | | 2-phenylisoquinolin-3-yl]ethyl]pyrazolo[1,5-a]pyrimidine-3- |
| | | carboxamide |
| Elimusertib | 1876467-74-1 | 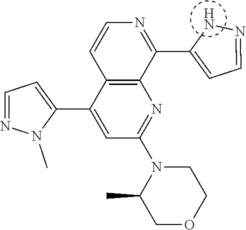 [SMILES: C[C@@H]1COCCN1c1cc(-c2ccnn2C)c2ccnc(-c3ccn[nH]3)c2n1] |
| | | (3R)-3-methyl-4-[4-(2-methylpyrazol-3- |
| | | yl)-8-(1H-pyrazol-5-yl)-1,7-naphthyr- |
| | | idin-2-yl]morpholine |
| Emavusertib | 1801344-14-8 | 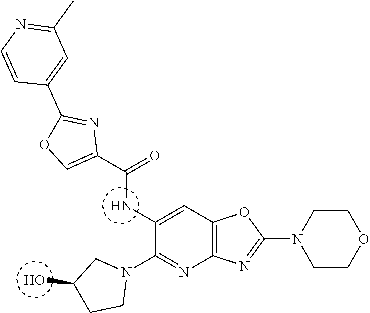 [SMILES: Cc1cc(-c2nc(C(=O)Nc3cc4oc(N5CCOCC5)nc4nc3N3CC[C@@H](O)C3)co2)ccn1] |
| | | N-[5-[(3R)-3-hydroxypyrrolidin-1-yl]-2-morpholin-4-yl-[1,3] |
| | | oxazolo[4,5-b]pyridin-6-yl]-2-(2-methylpyridin-4-yl)-1,3- |
| | | oxazole-4-carboxamide |
| Enasidenib | 1446502-11-9 | 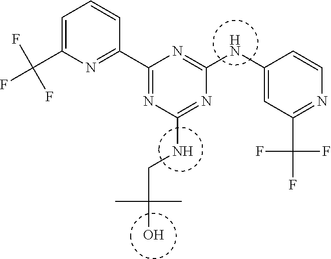 [SMILES: CC(C)(O)CNc1nc(Nc2ccnc(C(F)(F)F)c2)nc(-c2cccc(C(F)(F)F)n2)n1] |
| | | 2-methyl-1-[[4-[6-(trifluoromethyl)pyridin-2-yl]-6-[2- |
| | | (trifluoromethyl)pyridin-4-yl]amino]-1,3,5-triazin-2- |
| | | yl]amino]propan-2-ol |
| Encorafenib | 1269440-17-6 | 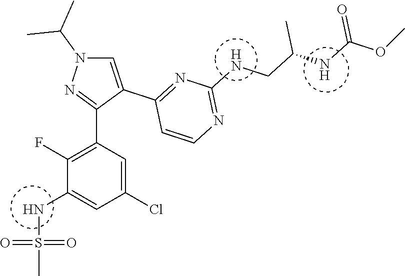 [SMILES: COC(=O)N[C@@H](C)CNc1nccc(-c2cn(C(C)C)nc2-c2cc(Cl)cc(NS(C)(=O)=O)c2F)n1] |
| | | methyl N-[(2S)-1-[[4-[3-[5-chloro-2-fluoro-3-(methanesulfon- |
| | | amido)phenyl]-1-propan-2-ylpyrazol-4-yl]pyrimidin-2-yl]amino] |
| | | propan-2-yl]carbamate |
| Enitociclib | 1610408-97-3 | 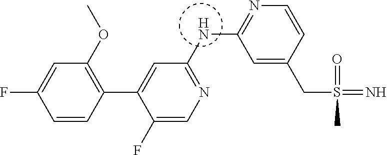 [SMILES: COc1cc(F)ccc1-c1cc(Nc2cc(C[S@@](C)(=N)=O)ccn2)ncc1F] |
| | | 5-fluoro-4-(4-fluoro-2-methoxyphenyl)-N-[4-[(methylsulfon- |
| | | imidoyl)methyl]pyridin-2-yl]pyridin-2-amine |
| Entinostat | 209783-80-2 | 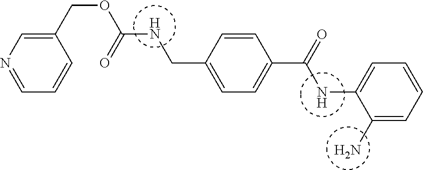 [SMILES: Nc1ccccc1NC(=O)c1ccc(CNC(=O)OCc2cccnc2)cc1] |
| | | pyridin-3-ylmethyl N-[[4-[(2-aminophenyl)carbamoyl]phenyl]methyl] |
| | | carbamate |
| Entrectinib | 1108743-60-7 | 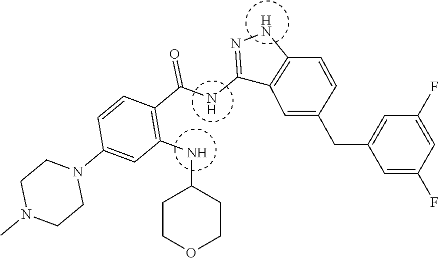 [SMILES: CN1CCN(c2ccc(C(=O)Nc3n[nH]c4ccc(Cc5cc(F)cc(F)c5)cc34)c(NC3CCOCC3)c2)CC1] |
| | | N-[5-[(3,5-difluorophenyl)methyl]-1H-indazol-3-yl]-4-(4-methylpiper- |
| | | azin-1-yl)-2-(oxan-4-ylamino)benzamide |
| Enzalutamide | 915087-33-1 | 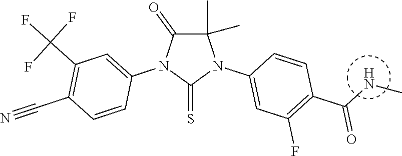 [SMILES: CNC(=O)c1ccc(N2C(=S)N(c3ccc(C#N)c(C(F)(F)F)c3)C(=O)C2(C)C)cc1F] |
| | | 4-[3-[4-cyano-3-(trifluoromethyl)phenyl]-5,5-dimethyl-4-oxo-2- |
| | | sulfanylideneimidazolidin-1-yl]-2-fluoro-N-methylbenzamide |
| Epacadostat | 1204669-58-8 | 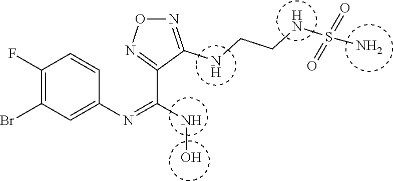 |
| | | N′-(3-bromo-4-fluorophenyl)-N-hydroxy-4-[2-(sulfamoylamino)- |
| | | ethylamino]-1,2,5-oxadiazole-3-carboximidamide |
| Epigallocatechin | 989-51-5 | 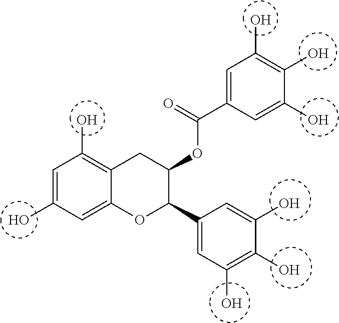 [SMILES: O=C(O[C@@H]1Cc2c(O)cc(O)cc2O[C@@H]1c1cc(O)c(O)c(O)c1)c1cc(O)c(O)c(O)c1] |
| | | [(2R,3R)-5,7-dihydroxy-2-(3,4,5-trihydroxyphenyl)-3,4- |
| | | dihydro-2H-chromen-3-yl]3,4,5-trihydroxybenzoate |
| Epoxomicin | 134381-21-8 | 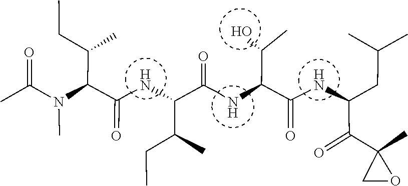 [SMILES: CC[C@H](C)[C@H](NCC(=O)[C@H]([C@@H](C)CC)N(C)C(C)=O)C(=O)CN[C@H](C(=O)CN[C@@H](CC(C)C)C(=O)[C@@]1(C)CO1)[C@@H](C)O] |
| | | (2S,3S)-2-[(2S,3S)-2-[acetyl(methyl)amino]-3-methylpentanoyl] |
| | | amino]-N-[(2S,3R)-3-hydroxy-1-[[(2S)-4-methyl-1-[(2R)-2- |
| | | methyloxiran-2-yl]-1-oxopentan-2-yl]amino]-1-oxobutan-2-yl]- |
| | | 3-methylpentanamide |
| Erdafitinib | 1346242-81-6 | 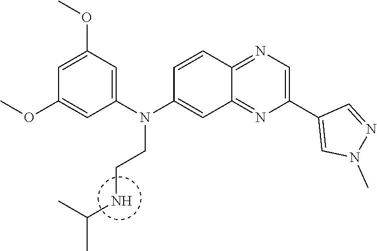 [SMILES: COc1cc(OC)cc(N(CCNC(C)C)c2ccc3ncc(-c4cnn(C)c4)nc3c2)c1] |
| | | N′-(3,5-dimethoxyphenyl)-N′-[3-(1-methylpyrazol-4-yl)- |
| | | quinoxalin-6-yl]-N-propan-2-ylethane-1,2-diamine |
| Erismodegib | — | 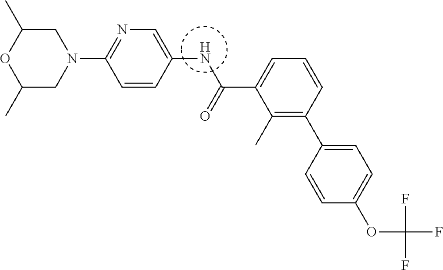 [SMILES: Cc1c(C(=O)Nc2ccc(N3CC(C)OC(C)C3)nc2)cccc1-c1ccc(OC(F)(F)F)cc1] |
| | | N-[6-(2,6-dimethylmorpholin-4-yl)pyridin-3-yl]-2-methyl-3-[4-(trifluoro- |
| | | methoxy)phenyl]benzamide |
| Erlotinib | 183321-74-6 | 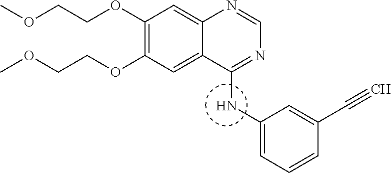 [SMILES: C#Cc1cccc(Nc2ncnc3cc(OCCOC)c(OCCOC)cc23)c1] |
| | | N-(3-ethynylphenyl)-6,7-bis(2-methoxyethoxy)quinazolin- |
| | | 4-amine |
| Everolimus | 159351-69-6 | 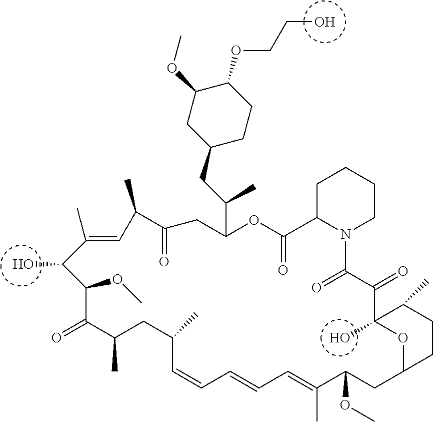 [SMILES: CO[C@H]1CC2CC[C@@H](C)[C@@](O)(O2)C(=O)C(=O)N2CCCCC2C(=O)OC([C@H](C)C[C@@H]2CC[C@@H](OCCO)[C@H](OC)C2)CC(=O)[C@H](C)/C=C(\C)[C@@H](O)[C@@H](OC)C(=O)[C@H](C)C[C@H](C)\C=C/C=C/C=C/1C] |
| | | (1R,9S,12S,15R,16E,18R,19R,21R,23S,24E,26E,28E,30S,32S,35R)- |
| | | 1,18-dihydroxy-12-[(2R)-1-[(1S,3R,4R)-4-(2-hydroxyethoxy)-3- |
| | | methoxycyclohexyl]propan-2-yl]-19,30-dimethoxy-15,17,21,23,29,35- |
| | | hexamethyl-11,36-dioxa-4-azatricyclo[30.3.1.0^{4,9}]hexatriaconta- |
| | | 16,24,26,28-tetraene-2,3,10,14,20-pentone |
| Fasudil | 103745-39-7 | 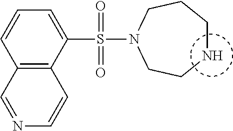 [SMILES: O=S(=O)(c1cccc2cnccc12)N1CCCNCC1] |
| | | 5-(1,4-diazepan-1-ylsulfonyl)- |
| | | isoquinoline |
| Fedratinib | 936091-26-8 | 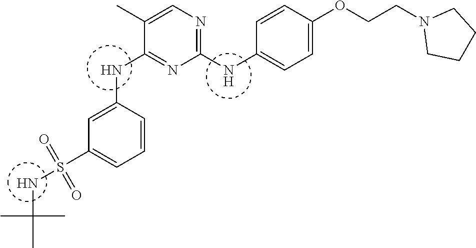 [SMILES: Cc1cnc(Nc2ccc(OCCN3CCCC3)cc2)nc1Nc1cccc(S(=O)(=O)NC(C)(C)C)c1] |
| | | N-tert-butyl-3-[[5-methyl-2-[4-(2-pyrrolidin-1-ylethoxy)anilino]pyrimidin-4- |
| | | yl]amino]benzenesulfonamide |
| Filgotinib | 1206161-97-8 | 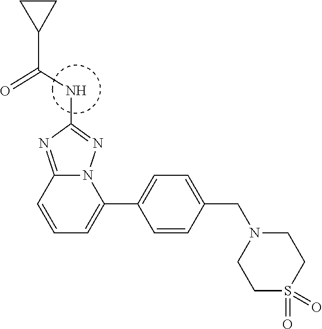 [SMILES: O=C(CNc1nc2cccc(-c3ccc(CN4CCS(=O)(=O)CC4)cc3)n2n1)C1CC1] |
| | | N-[5-[4-[(1,1-dioxo-1,4-thiazinan-4-yl)methyl]phen- |
| | | yl]-[1,2,4]triazolo[1,5-a]pyridin-2-yl]cyclopropane- |
| | | carboxamide |
| Foslinanib | 1256037-60-1 | 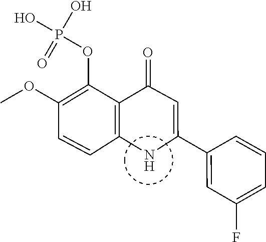 [SMILES: COc1ccc2c(c1OP(=O)(O)O)C(=O)C=C(c1cccc(F)c1)NC2] |
| | | [2-(3-fluorophenyl)-6-methoxy-4-oxo-1H- |
| | | quinolin-5-yl]dihydrogen phosphate |
| Fostamatinib | 901119-35-5 | 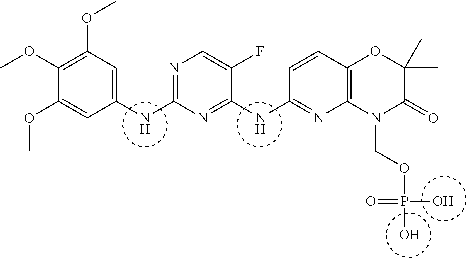 [SMILES: COc1cc(Nc2ncc(F)c(Nc3ccc4c(n3)N(COP(=O)(O)O)C(=O)C(C)(C)O4)n2)cc(OC)c1OC] |
| | | [6-[[5-fluoro-2-(3,4,5-trimethoxyanilino)pyrimidin-4-yl]amino]-2,2-dimethyl- |
| | | 3-oxopyrido[3,2-b][1,4]oxazin-4-yl]methyl dihydrogen phosphate |
| Fruquintinib | 1194506-26-7 | 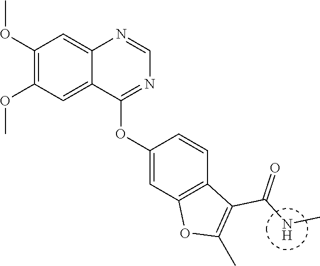 |
| | | 6-(6,7-dimethoxyquinazolin-4-yl)oxy-N,2-dimethyl- |
| | | 1-benzofuran-3-carboxamide |
| Galunisertib | 700874-72-2 | 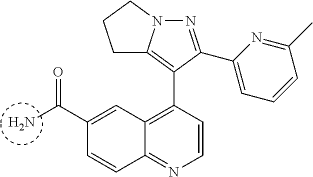 [SMILES: Cc1cccc(-c2nn3c(c2-c2ccnc4ccc(C(N)=O)cc24)CCC3)n1] |
| | | 4-[2-(6-methylpyridin-2-yl)-5,6-dihydro-4H-pyrrolo |
| | | [1,2-b]pyrazol-3-yl]quinoline-6-carboxamide |
| Ganetespib | 888216-25-9 | 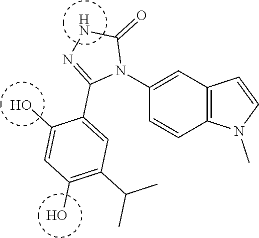 |
| | | 3-(2,4-dihydroxy-5-propan-2-ylphenyl)- |
| | | 4-(1-methylindol-5-yl)-1H-1,2,4-triazol- |
| | | 5-one |
| Gedatolisib | 1197160-78-3 | 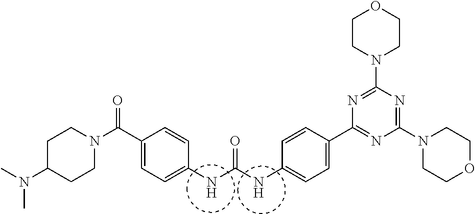 [SMILES: CN(C)C1CCN(C(=O)c2ccc(NC(=O)Nc3ccc(-c4nc(N5CCOCC5)nc(N5CCOCC5)n4)cc3)cc2)CC1] |
| | | 1-[4-[4-(dimethylamino)piperidine-1-carbonyl]phenyl]-3-[4-(4,6-dimorpholin- |
| | | 4-yl-1,3,5-triazin-2-yl)phenyl]urea |
| Gefitinib | 184475-35-2 | 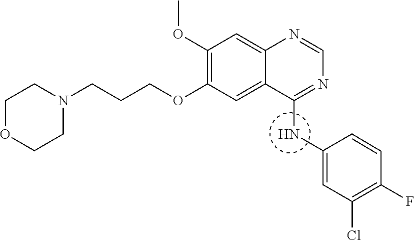 [SMILES: COc1cc2ncnc(Nc3ccc(F)c(Cl)c3)c2cc1OCCCN1CCOCC1] |
| | | N-(3-chloro-4-fluorophenyl)-7-methoxy-6-(3-morpholin-4-yl- |
| | | propoxy)quinazolin-4-amine |
| GFH018 | 2169299-67-4 | 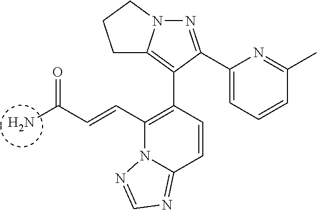 [SMILES: Cc1cccc(-c2nn3c(c2-c2ccc4ncnn4c2/C=C/C(N)=O)CCC3)n1] |
| | | (E)-3-[6-[2-(6-methylpyridin-2-yl)-5,6-dihydro-4H- |
| | | pyrrolo[1,2-b]pyrazol-3-yl]-[1,2,4]triazolo[1,5-a] |
| | | pyridin-5-yl]prop-2-enamide |
| Gilteritinib | 1254053-43-4 | 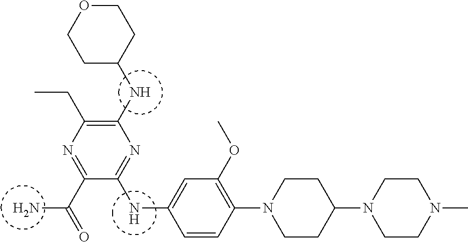 [SMILES: CCc1nc(C(N)=O)c(Nc2ccc(N3CCC(N4CCN(C)CC4)CC3)c(OC)c2)nc1NC1CCOCC1] |
| | | 6-ethyl-3-[3-methoxy-4-[4-(4-methylpiperazin-1-yl)piperidin-1-yl]anilino]- |
| | | 5-(oxan-4-ylamino)pyrazine-2-carboxamide |
| Givinostat | 497833-27-9 | 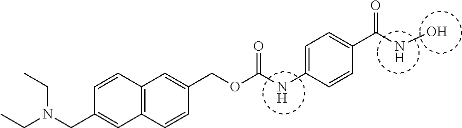 [SMILES: CCN(CC)Cc1ccc2cc(COC(=O)Nc3ccc(C(=O)NO)cc3)ccc2c1] |
| | | [6-(diethylaminomethyl)naphthalen-2-yl]methyl N-[4-(hydroxycarbamoyl)- |
| | | phenyl]carbamate |
| Glasdegib | 1095173-27-5 | 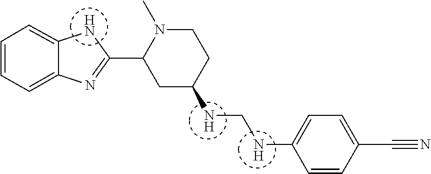 [SMILES: CN1CC[C@@H](NCNc2ccc(C#N)cc2)CC1c1nc2ccccc2[nH]1] |
| | | 1-[(2R,4R)-2-(1H-benzimidazol-2-yl)-1-methylpiperidin-4-yl]-3-(4- |
| | | cyanophenyl)urea |
| Goserelin | 65807-02-5 | 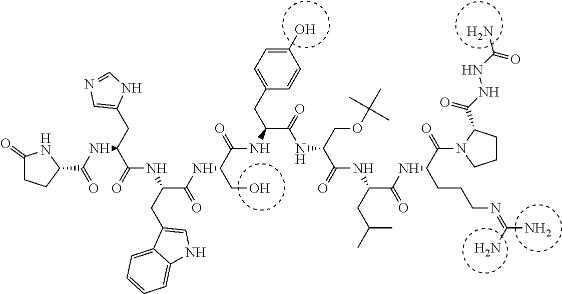 |
| | | (2S)-N-[(2S)-1-[(2S)-1-[(2S)-1-[(2S)-1-[(2R)-1-[[(2S)-1-[(2S)-1-[(2S)-2-[(carbamoylamino)- |
| | | carbamoyl]pyrrolidin-1-yl]-5-(diaminomethylideneamino)-1-oxopentan-2-yl]amino]-4- |
| | | methyl-1-oxopentan-2-yl]amino]-3-[(2-methylpropan-2-yl)oxy]-1-oxopropan-2-yl]amino]- |
| | | 3-(4-hydroxyphenyl)-1-oxopropan-2-yl]amino]-3-hydroxy-1-oxopropan-2-yl]amino]-3- |
| | | (1H-indol-3-yl)-1-oxopropan-2-yl]amino]-3-(1H-imidazol-5-yl)-1-oxopropan-2-yl]-5- |
| | | oxopyrrolidine-2-carboxamide |
| GSK2256098 | 1224887-10-8 | 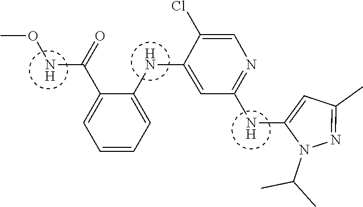 |
| | | 2-[[5-chloro-2-[(5-methyl-2-propan-2-ylpyrazol-3-yl)amino] |
| | | pyridin-4-yl]amino]-N-methoxybenzamide |
| GSK269962A | 850664-21-0 | 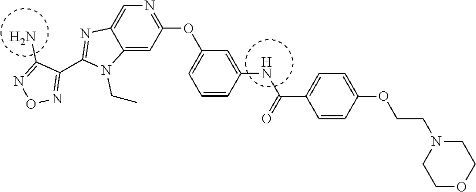 [SMILES: CCn1c(-c2nonc2N)nc2cnc(Oc3cccc(NC(=O)c4ccc(OCCN5CCOCC5)cc4)c3)cc21] |
| | | N-[3-[2-(4-amino-1,2,5-oxadiazol-3-yl)-1-ethylimidazo[4,5-c]pyridin-6-yl] |
| | | oxyphenyl]-4-(2-morpholin-4-ylethoxy)benzamide |
| GSK690693 | 937174-76-0 | 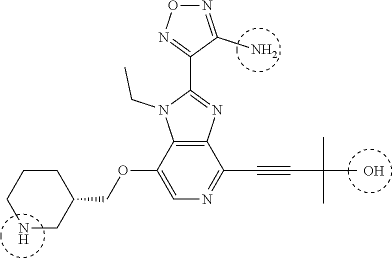 |
| | | 4-[2-(4-amino-1,2,5-oxadiazol-3-yl)-1-ethyl-7-[(3S)-piperidin-3- |
| | | yl]methoxy]imidazo[4,5-c]pyridin-4-yl]-2-methylbut-3-yn-2-ol |
| GUL | 1025796-32-0 | 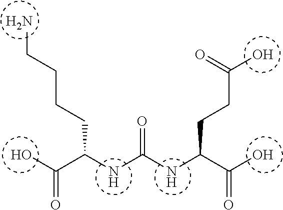 [SMILES: NCCCC[C@H](NC(=O)N[C@@H](CCC(=O)O)C(=O)O)C(=O)O] |
| | | (2S)-2-[[(1S)-5-amino-1-carboxypentyl]car- |
| | | bamoylamino]pentanedioic acid |
| Halofuginone | 55837-20-2 | 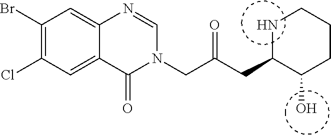 [SMILES: O=C(C[C@H]1NCCC[C@@H]1O)Cn1cnc2cc(Br)c(Cl)cc2c1=O] |
| | | 7-bromo-6-chloro-3-[3-[(2R,3S)-3-hydroxypiperidin- |
| | | 2-yl]-2-oxopropyl]quinazolin-4-one |
| Hymecromone | 90-33-5 | 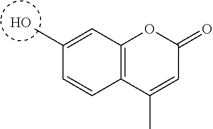 |
| | | 7-hydroxy-4-methylchromen-2-one |
| Ibrutinib | 936563-96-1 | 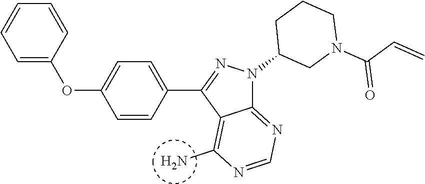 [SMILES: C=CC(=O)N1CCC[C@@H](n2nc(-c3ccc(Oc4ccccc4)cc3)c3c(N)ncnc32)C1] |
| | | 1-[(3R)-3-[4-amino-3-(4-phenoxyphenyl)pyrazolo[3,4-d]pyrimidin-1- |
| | | yl]piperidin-1-yl]prop-2-en-1-one |
| Icotinib | 610798-31-7 | 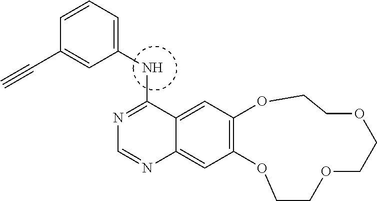 [SMILES: C#Cc1cccc(Nc2ncnc3cc4c(cc23)OCCOCCOCCO4)c1] |
| | | N-(3-ethynylphenyl)-2,5,8,11-tetraoxa-15,17-diazatricyclo |
| | | [10.8.0.014,19]icosa-1(12),13,15,17,19-pentaen-18-amine |
| Idelalisib | 870281-82-6 | 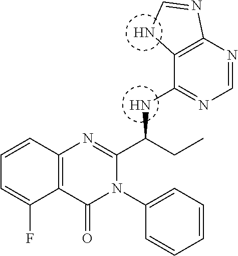 [SMILES: CC[C@H](Nc1ncnc2nc[nH]c12)c1nc2cccc(F)c2c(=O)n1-c1ccccc1] |
| | | 5-fluoro-3-phenyl-2-[(1S)-1-(7H-purin- |
| | | 6-ylamino)propyl]quinazolin-4-one |
| Imatinib | 152459-95-5 | 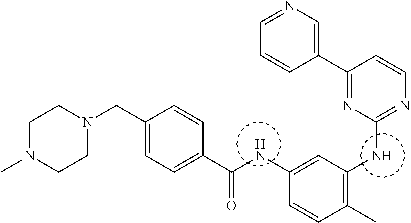 [SMILES: Cc1ccc(NC(=O)c2ccc(CN3CCN(C)CC3)cc2)cc1Nc1nccc(-c2cccnc2)n1] |
| | | 4-[(4-methylpiperazin-1-yl)methyl]-N-[4-methyl-3-[(4-pyridin-3- |
| | | ylpyrimidin-2-yl)amino]phenyl]benzamide |
| Imiquimod | 99011-02-6 | 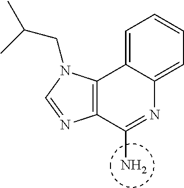 [SMILES: CC(C)Cn1cnc2c(N)nc3ccccc3c21] |
| | | 1-(2-methylpropyl)imidazo |
| | | [4,5-c]quinolin-4-amine |
| Infigratinib | 872511-34-7 | 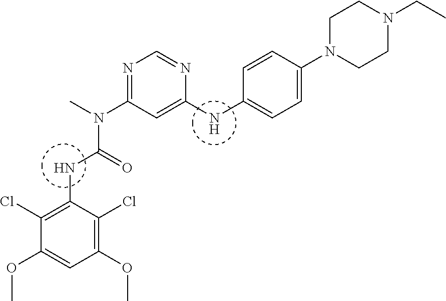 [SMILES: CCN1CCN(c2ccc(Nc3cc(N(C)C(=O)Nc4c(Cl)c(OC)cc(OC)c4Cl)ncn3)cc2)CC1] |
| | | 3-(2,6-dichloro-3,5-dimethoxyphenyl)-1-[6-[4-(4-ethylpiperazin-1-yl)- |
| | | anilino]pyrimidin-4-yl]-1-methylurea |
| Iniparib | 160003-66-7 | 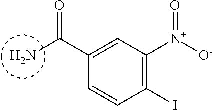 [SMILES: NC(=O)c1ccc(I)c([N+](=O)[O-])c1] |
| | | 4-iodo-3-nitrobenzamide |
| Ipatasertib | 1001264-89-6 | 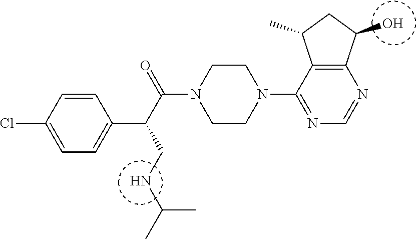 [SMILES: CC(C)NC[C@@H](C(=O)N1CCN(c2ncnc3c2[C@H](C)C[C@H]3O)CC1)c1ccc(Cl)cc1] |
| | | (2S)-2-(4-chlorophenyl)-1-[4-[(5R,7R)-7-hydroxy-5-methyl-6,7- |
| | | dihydro-5H-cyclopenta[d]pyrimidin-4-yl]piperazin-1-yl]-3- |
| | | (propan-2-ylamino)propan-1-one |
| Itacitinib | 1334298-90-6 | 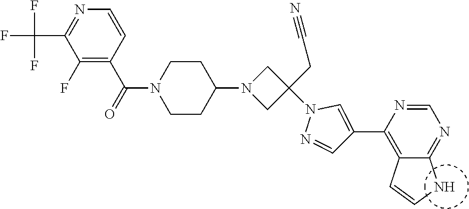 [SMILES: N#CCC1(n2cc(-c3ncnc4[nH]ccc34)cn2)CN(C2CCN(C(=O)c3ccnc(C(F)(F)F)c3F)CC2)C1] |
| | | 2-[1-[1-[3-fluoro-2-(trifluoromethyl)pyridine-4-carbonyl]piperidin-4-yl]-3- |
| | | [4-(7H-pyrrolo[2,3-d]pyrimidin-4-yl)pyrazol-1-yl]azetidin-3-yl]acetonitrile |
| Ivaltinostat | 936221-33-9 | 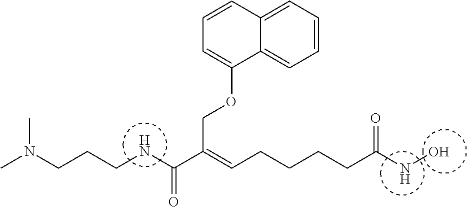 [SMILES: CN(C)CCCNC(=O)/C(=C/CCCCC(=O)NO)COc1cccc2ccccc12] |
| | | (E)-N-[3-(dimethylamino)propyl]-N′-hydroxy-2-(naphthalen-1-yloxymethyl)- |
| | | oct-2-enediamide |
| Ivosidenib | 1448347-49-6 | 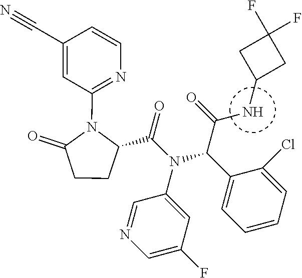 [SMILES: N#Cc1ccnc(N2C(=O)CC[C@H]2C(=O)N(c2cncc(F)c2)[C@H](C(=O)NC2CC(F)(F)C2)c2ccccc2Cl)c1] |
| | | (2S)-N-[(1S)-1-(2-chlorophenyl)-2-[(3,3-difluoro- |
| | | cyclobutyl)amino]-2-oxoethyl]-1-(4-cyanopyridin- |
| | | 2-yl)-N-(5-fluoropyridin-3-yl)-5-oxopyrrolidine- |
| | | 2-carboxamide |
| Ixazomib | 1072833-77-2 | 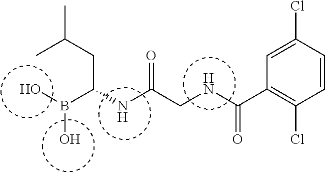 [SMILES: CC(C)C[C@H](NC(=O)CNC(=O)c1cc(Cl)ccc1Cl)B(O)O] |
| | | [(1R)-1-[[2-[(2,5-dichlorobenzoyl)amino]acetyl] |
| | | amino]-3-methylbutyl]boronic acid |
| Kevetrin | 66592-89-0 | 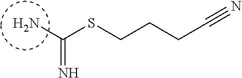 |
| | | 3-cyanopropyl carbamimidothioate |
| Lapatinib | 231277-92-2 | 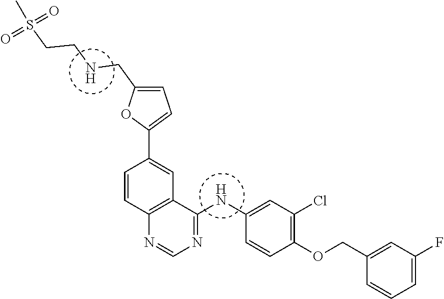 [SMILES: CS(=O)(=O)CCNCc1ccc(-c2ccc3ncnc(Nc4ccc(OCc5cccc(F)c5)c(Cl)c4)c3c2)o1] |
| | | N-[3-chloro-4-[(3-fluorophenyl)methoxy]phenyl]-6-[5-[(2-methylsulfon- |
| | | ylethylamino)methyl]furan-2-yl]quinazolin-4-amine |
| Larotrectinib | 1223403-58-4 | 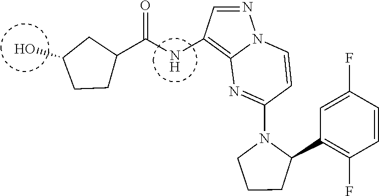 [SMILES: O=C(Nc1cnn2ccc(N3CCC[C@@H]3c3cc(F)ccc3F)nc12)C1CC[C@H](O)C1] |
| | | (3S)-N-[5-[(2R)-2-(2,5-difluorophenyl)pyrrolidin-1-yl]pyrazolo |
| | | [1,5-a]pyrimidin-3-yl]-3-hydroxypyrrolidine-1-carboxamide |
| Lenalidomide | 191732-72-6 | 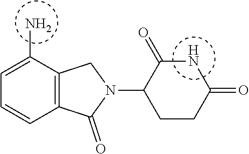 [SMILES: Nc1cccc2c1CN(C1CCC(=O)NC1=O)C2=O] |
| | | 3-(7-amino-3-oxo-1H-isoindol-2-yl)- |
| | | piperidine-2,6-dione |
| Leniolisib | 1354690-24-6 | 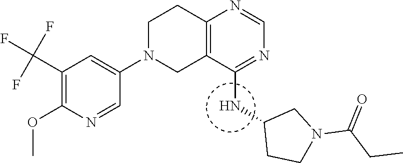 [SMILES: CCC(=O)N1CC[C@H](Nc2ncnc3c2CN(c2cnc(OC)c(C(F)(F)F)c2)CC3)C1] |
| | | 1-[(3S)-3-[6-[6-methoxy-5-(trifluoromethyl)pyridin-3-yl]-7,8- |
| | | dihydro-5H-pyrido[4,3-d]pyrimidin-4-yl]amino]pyrrolidin-1- |
| | | yl]propan-1-one |
| Lenvatinib | 417716-92-8 | 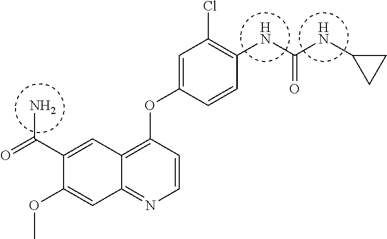 [SMILES: COc1cc2nccc(Oc3ccc(NC(=O)NC4CC4)c(Cl)c3)c2cc1C(N)=O] |
| | | 4-[3-chloro-4-(cyclopropylcarbamoylamino)phenoxy]-7-meth- |
| | | oxyquinoline-6-carboxamide |
| Leuprolide | 35925-21-4 | 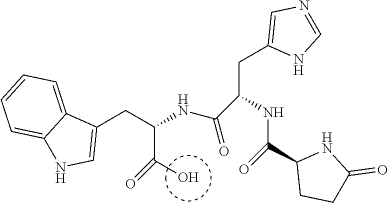 [SMILES: O=C1CC[C@@H](C(=O)N[C@@H](Cc2cnc[nH]2)C(=O)N[C@@H](Cc2c[nH]c3ccccc23)C(=O)O)N1] |
| | | (2S)-2-[[(2S)-3-(1H-imidazol-5-yl)-2-[[(2S)-5-oxopyrrolidine- |
| | | 2-carbonyl]amino]propanoyl]amino]-3-(1H-indol-3-yl)- |
| | | propanoic acid |
| Linsitinib | 867160-71-2 | 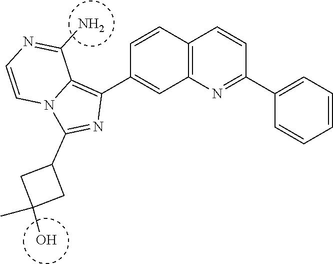 [SMILES: CC1(O)CC(c2nc(-c3ccc4ccc(-c5ccccc5)nc4c3)c3c(N)nccn23)C1] |
| | | 3-[8-amino-1-(2-phenylquinolin-7-yl)imidazo[1,5-a] |
| | | pyrazin-3-yl]-1-methylcyclobutan-1-ol |
| Lonafarnib | 193275-84-2 | 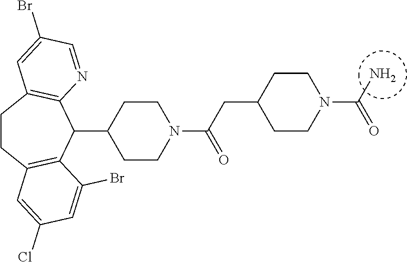 [SMILES: NC(=O)N1CCC(CC(=O)N2CCC(C3c4ncc(Br)cc4CCc4cc(Cl)cc(Br)c43)CC2)CC1] |
| | | 4-[2-[4-[(2R)-6,15-dibromo-13-chloro-4-azatricyclo[9.4.0.0^{3,8}] |
| | | pentadeca-1(11),3(8),4,6,12,14-hexaen-2-yl]piperidin-1-yl]-2- |
| | | oxoethyl]piperidine-1-carboxamide |
| Lorlatinib | 1454846-35-5 | 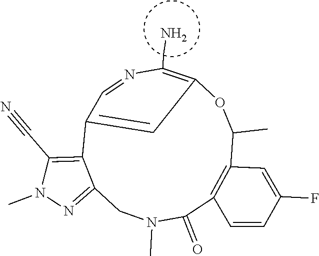 [SMILES: CC1Oc2cc(cnc2CN)-c2c(nn(C)c2C#N)CN(C)C(=O)c2ccc(F)cc21] |
| | | (16R)-19-amino-13-fluoro-4,8,16-trimethyl-9-oxo- |
| | | 17-oxa-4,5,8,20-tetrazatetracyclo[16.3.1.0^{2,6}.0^{10,15}] |
| | | docosa-1(22),2,5,10(15),11,13,18,20-octaene- |
| | | 3-carbonitrile |
| Losartan | 114798-26-4 | 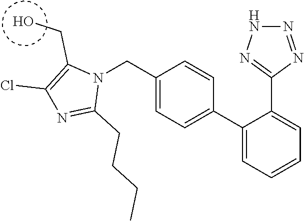 [SMILES: CCCCc1nc(Cl)c(CO)n1Cc1ccc(-c2ccccc2-c2nn[nH]n2)cc1] |
| | | [2-butyl-5-chloro-3-[[4-[2-(2H-tetrazol-5-yl) |
| | | phenyl]phenyl]methyl]imidazol-4-yl]methanol |
| Lucitanib | 1058137-23-7 | 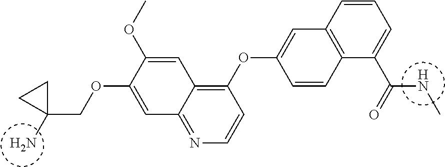 [SMILES: CNC(=O)c1cccc2cc(Oc3ccnc4cc(OCC5(N)CC5)c(OC)cc34)ccc12] |
| | | 6-[7-[(1-aminocyclopropyl)methoxy]-6-methoxyquinolin-4-yl]oxy-N- |
| | | methylnaphthalene-1-carboxamide |
| Luminespib | 747412-49-3 | 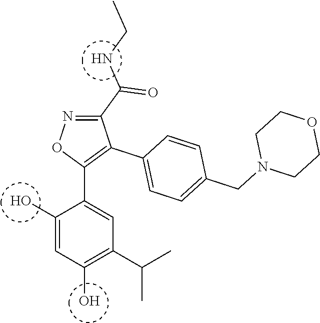 [SMILES: CCNC(=O)c1noc(-c2cc(C(C)C)c(O)cc2O)c1-c1ccc(CN2CCOCC2)cc1] |
| | | 5-(2,4-dihydroxy-5-propan-2-ylphenyl)-N-ethyl- |
| | | 4-[4-(morpholin-4-ylmethyl)phenyl]-1,2-oxazole- |
| | | 3-carboxamide |
| M1096 | 172478-00-1 | 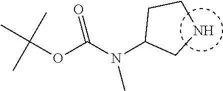 |
| | | tert-butyl N-methyl-N-pyrrolidin- |
| | | 3-ylcarbamate |
| Marizomib | 437742-34-2 | 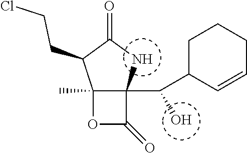 |
| | | (1R,4R,5S)-4-(2-chloroethyl)-1-[(S)- |
| | | [(1S)-cyclohex-2-en-1-yl]-hydroxymeth- |
| | | yl]-5-methyl-6-oxa-2-azabicyclo[3.2.0] |
| | | heptane-3,7-dione |
| ME-344 | 1374524-68-1 | 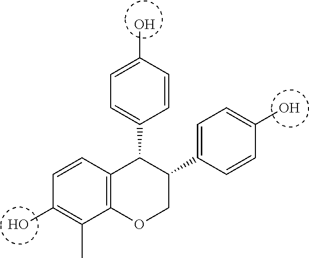 [SMILES: Cc1c(O)ccc2c1OC[C@@H](c1ccc(O)cc1)[C@H]2c1ccc(O)cc1] |
| | | (3R,4S)-3,4-bis(4-hydroxyphenyl)-8-methyl-3,4- |
| | | dihydro-2H-chromen-7-ol |
| Merestinib | 1206799-15-6 | 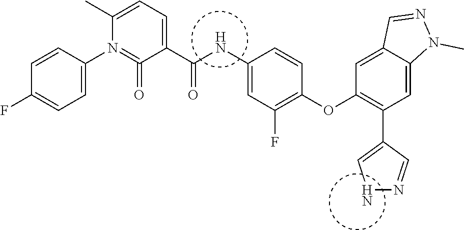 |
| | | N-[3-fluoro-4-[1-methyl-6-(1H-pyrazol-4-yl)indazol-5-yl]oxyphenyl]-1-(4- |
| | | fluorophenyl)-6-methyl-2-oxopyridine-3-carboxamide |
| Metformin | 657-24-9 | 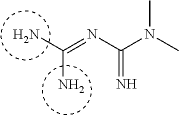 |
| | | 3-(diaminomethylidene)-1,1- |
| | | dimethylguanidine |
| MG132 | 133407-82-6 | 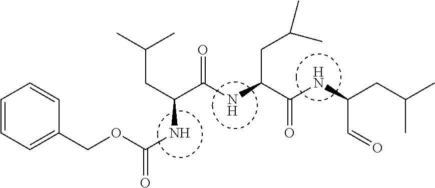 [SMILES: CC(C)C[C@@H](C=O)NC(=O)[C@H](CC(C)C)NC(=O)[C@H](CC(C)C)NC(=O)OCc1ccccc1] |
| | | benzyl N-[(2S)-4-methyl-1-[[(2S)-4-methyl-1-[[(2S)-4-methyl-1- |
| | | oxopentan-2-yl]amino]-1-oxopentan-2-yl]amino]-1-oxopentan-2- |
| | | yl]carbamate |
| Midostaurin | 120685-11-2 | 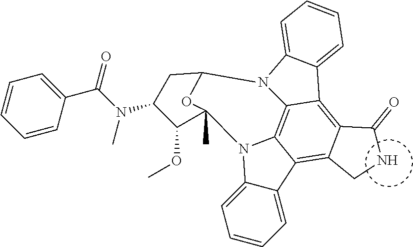 [SMILES: CO[C@@H]1[C@H](N(C)C(=O)c2ccccc2)CC2O[C@]1(C)n1c3ccccc3c3c4c(c5c6ccccc6n2c5c31)C(=O)NC4] |
| | | N-[(2S,3R,4R,6R)-3-methoxy-2-methyl-16-oxo-29-oxa-1,7,17-triaza- |
| | | octacyclo[12.12.2.12,6.07,28.08,13.015,19.020,27.021,26]nonacosa- |
| | | 8,10,12,14,19,21,23,25,27-nonaen-4-yl]-N-methylbenzamide |
| Miransertib | 1313881-70-7 | 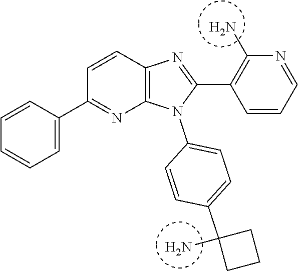 |
| | | 3-[3-[4-(1-aminocyclobutyl)phenyl]-5-phenyl- |
| | | imidazo[4,5-b]pyridin-2-yl]pyridin-2-amine |
| Mivavotinib | 1312691-33-0 | 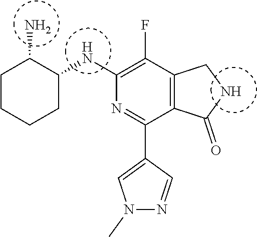 [SMILES: Cn1cc(-c2nc(N[C@@H]3CCCC[C@@H]3N)c(F)c3c2C(=O)NC3)cn1] |
| | | 6-[[(1R,2S)-2-aminocyclohexyl]amino]-7- |
| | | fluoro-4-(1-methylpyrazol-4-yl)-1,2- |
| | | dihydropyrrolo[3,4-c]pyridin-3-one |
| MK2206 | 1032349-93-1 | 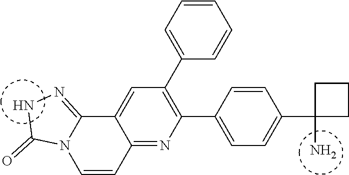 [SMILES: NC1(c2ccc(-c3nc4ccn5c(=O)[nH]nc5c4cc3-c3ccccc3)cc2)CCC1] |
| | | 8-[4-(1-aminocyclobutyl)phenyl]-9-phenyl-2H-[1,2,4] |
| | | triazolo[3,4-f][1,6]naphthyridin-3-one |
| MMP-9 | — | 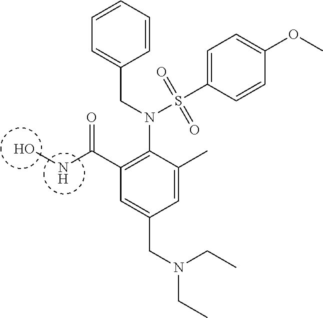 [SMILES: CCN(CC)Cc1cc(C)c(N(Cc2ccccc2)S(=O)(=O)c2ccc(OC)cc2)c(C(=O)NO)c1] |
| | | 2-[benzyl-(4-methoxyphenyl)sulfonylamino]-5-(dieth- |
| | | ylaminomethyl)-N-hydroxy-3-methylbenzamide |
| Mobocertinib | 1847461-43-1 | 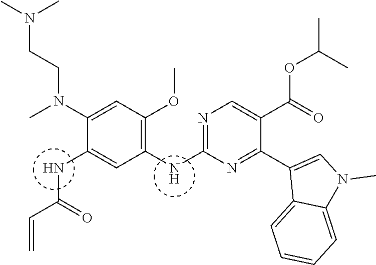 |
| | | propan-2-yl 2-[4-[2-(dimethylamino)ethyl-methylamino]-2- |
| | | methoxy-5-(prop-2-enoylamino)anilino]-4-(1-methylindol-3- |
| | | yl)pyrimidine-5-carboxylate |
| Mocetinostat | 726169-73-9 | 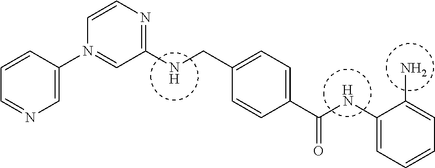 |
| | | N-(2-aminophenyl)-4-[[(4-pyridin-3-ylpyrimidin-2-yl)amino]methyl] |
| | | benzamide |
| Motesanib | 453562-69-1 | 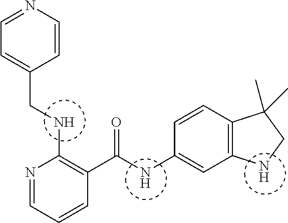 [SMILES: CC1(C)CNc2cc(NC(=O)c3cccnc3NCc3ccncc3)ccc21] |
| | | N-(3,3-dimethyl-1,2-dihydroindol-6-yl)-2- |
| | | (pyridin-4-ylmethylamino)pyridine-3- |
| | | carboxamide |
| MRTX1133 | 2621928-55-8 | 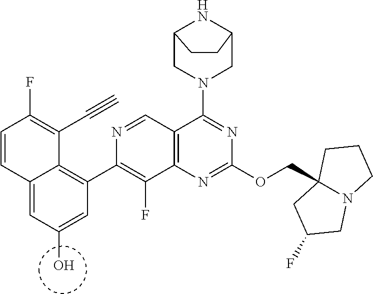 [SMILES: C#Cc1c(F)ccc2cc(O)cc(-c3ncc4c(N5CC6CCC(C5)N6)nc(OC[C@@]56CCCN5C[C@H](F)C6)nc4c3F)c12] |
| | | 4-(4-((1R,5S)-3,8-Diazabicyclo[3.2.1]octan-3-yl)-8-fluoro-2- |
| | | (((2R,7aS)-2-fluorohexahydro-1H-pyrrolizin-7a-yl)methoxy)- |
| | | pyrido[4,3-d]pyrimidin-7-yl)-5-ethynyl-6-fluoronaphthalen- |
| | | 2-ol |
| Navitoclax | 923564-51-6 | 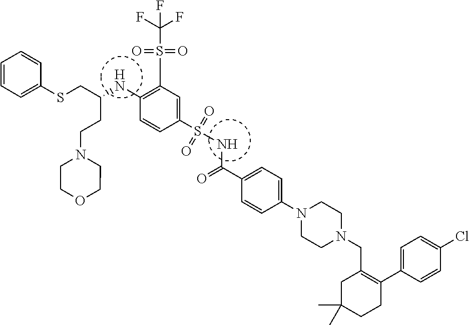 |
| | | 4-[4-[2-(4-chlorophenyl)-5,5-dimethylcyclohexen-1-yl]methyl]piperazin-1- |
| | | yl]-N-[4-[[(2R)-4-morpholin-4-yl-1-phenylsulfanylbutan-2-yl]amino]-3- |
| | | (trifluoromethylsulfonyl)phenyl]sulfonylbenzamide |
| Nazartinib | 1508250-71-2 | 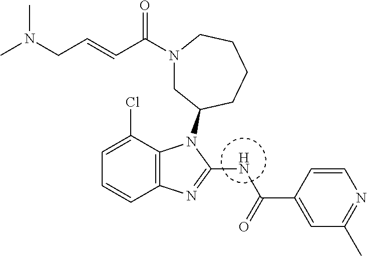 |
| | | N-[7-chloro-1-[(3R)-1-[(E)-4-(dimethylamino)but-2- |
| | | enoyl]azepan-3-yl]benzimidazol-2-yl]-2-methylpyr- |
| | | idine-4-carboxamide |
| Nedisertib | 1637542-33-6 | 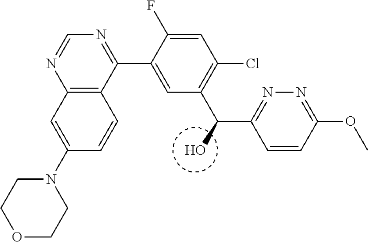 [SMILES: COc1ccc([C@@H](O)c2cc(-c3ncnc4cc(N5CCOCC5)ccc34)c(F)cc2Cl)nn1] |
| | | (S)-[2-chloro-4-fluoro-5-(7-morpholin-4-ylquinazolin-4-yl)- |
| | | phenyl]-(6-methoxypyridazin-3-yl)methanol |
| Neratinib | 698387-09-6 | 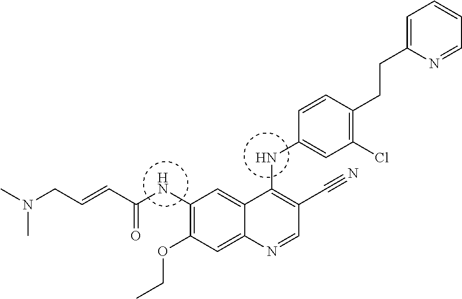 [SMILES: CCOc1cc2ncc(C#N)c(Nc3ccc(CCc4ccccn4)c(Cl)c3)c2cc1NC(=O)/C=C/CN(C)C] |
| | | (E)-N-[4-[3-chloro-4-(pyridin-2-ylmethoxy)anilino]-3-cyano-7-ethoxyquin- |
| | | olin-6-yl]-4-(dimethylamino)but-2-enamide |
| Nilotinib | 641571-10-0 | 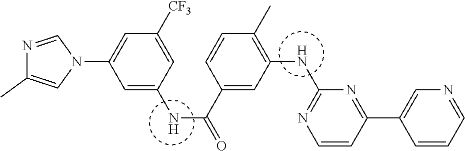 [SMILES: Cc1cn(-c2cc(NC(=O)c3ccc(C)c(Nc4nccc(-c5cccnc5)n4)c3)cc(C(F)(F)F)c2)cn1] |
| | | 4-methyl-N-[3-(4-methylimidazol-1-yl)-5-(trifluoromethyl)phenyl]-3-[(4- |
| | | pyridin-3-ylpyrimidin-2-yl)amino]benzamide |
| Nilutamide | 63612-50-0 | 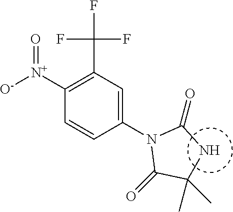 [SMILES: CC1(C)NC(=O)N(c2ccc([N+](=O)[O-])c(C(F)(F)F)c2)C1=O] |
| | | 5,5-dimethyl-3-[4-nitro-3-(trifluoro- |
| | | methyl)phenyl]imidazolidine-2,4-dione |
| Nintedanib | 656247-17-5 | 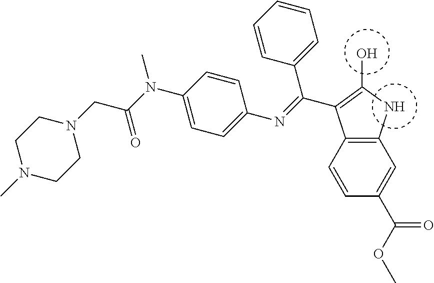 [SMILES: COC(=O)c1ccc2c(/C(=N/c3ccc(N(C)C(=O)CN4CCN(C)CC4)cc3)c3ccccc3)c(O)[nH]c2c1] |
| | | methyl 2-hydroxy-3-[N-[4-[methyl-[2-(4-methylpiperazin-1-yl)acetyl] |
| | | amino]phenyl]-C-phenylcarbonimidoyl]-1H-indole-6-carboxylate |
| Niraparib | 1038915-60-4 | 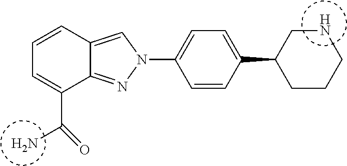 [SMILES: NC(=O)c1cccc2cn(-c3ccc([C@@H]4CCCNC4)cc3)nc12] |
| | | 2-[4-[(3S)-piperidin-3-yl]phenyl]indazole-7-carboxamide |
| NMS-P118 | 1262417-51-5 | 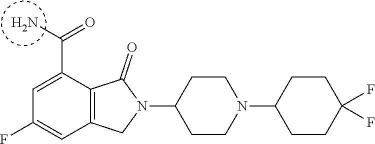 [SMILES: NC(=O)c1cc(F)cc2c1C(=O)N(C1CCN(C3CCC(F)(F)CC3)CC1)C2] |
| | | 2-[1-(4,4-difluorocyclohexyl)piperidin-4-yl]-6-fluoro-3-oxo- |
| | | 1H-isoindole-4-carboxamide |
| NMS-P515 | 1262395-13-0 | 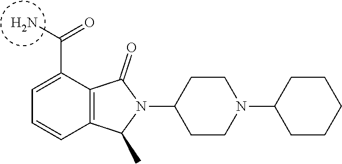 [SMILES: C[C@H]1c2cccc(C(N)=O)c2C(=O)N1C1CCN(C2CCCCC2)CC1] |
| | | (1S)-2-(1-cyclohexylpiperidin-4-yl)-1-methyl-3-oxo-1H- |
| | | isoindole-4-carboxamide |
| NSC668394 | — | 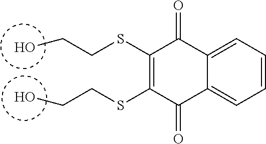 [SMILES: O=C1C(SCCO)=C(SCCO)C(=O)c2ccccc21] |
| | | 7-[2-(3,5-dibromo-4-hydroxyphenyl)ethyl- |
| | | amino]quinoline-5,8-dione |
| NSC95397 | 93718-83-3 | 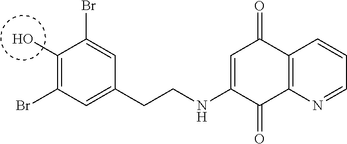 [SMILES: O=C1C=C(NCCc2cc(Br)c(O)c(Br)c2)C(=O)c2ncccc21] |
| | | 2,3-bis(2-hydroxyethylsulfanyl)naphthalene-1,4-dione |
| Numidargistat | 2095732-06-0 | 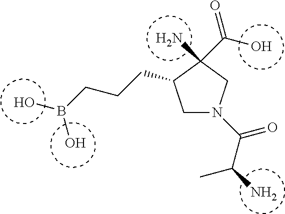 |
| | | (3R,4S)-3-amino-1-[(2S)-2-aminopropanoyl]-4- |
| | | (3-boronopropyl)pyrrolidine-3-carboxylic acid |
| NVP-2 | — | 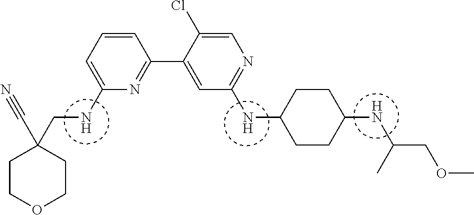 [SMILES: COCC(C)NC1CCC(Nc2cc(-c3cccc(NCC4(C#N)CCOCC4)n3)c(Cl)cn2)CC1] |
| | | 4-[[[6-[5-chloro-2-[[4-(1-methoxypropan-2-ylamino)cyclohexyl]amino]pyridin- |
| | | 4-yl]pyridin-2-yl]amino methyl]oxane-4-carbonitrile |
| Olaparib | 763113-22-0 | 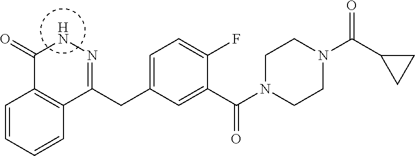 [SMILES: O=C(c1cc(Cc2n[nH]c(=O)c3ccccc23)ccc1F)N1CCN(C(=O)C2CC2)CC1] |
| | | 4-(3-{[4-(Cyclopropylcarbonyl)-1-piperazinyl]carbonyl}-4-fluoro- |
| | | benzyl)-1(2H)-phthalazinone |
| Olmutinib | 1353550-13-6 | 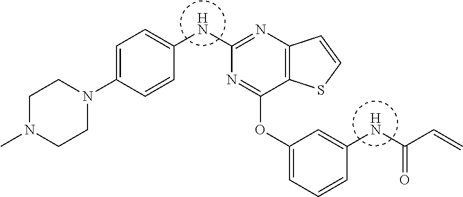 [SMILES: C=CC(=O)Nc1cccc(Oc2nc(Nc3ccc(N4CCN(C)CC4)cc3)nc3ccsc23)c1] |
| | | N-[3-[2-[4-(4-methylpiperazin-1-yl)anilino]thieno[3,2-d]pyrimidin-4-yloxy- |
| | | phenyl]prop-2-enamide |
| Omipalisib | 1086062-66-9 | 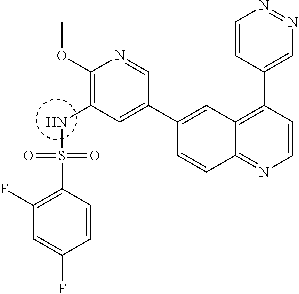 [SMILES: COc1ncc(-c2ccc3nccc(-c4ccnnc4)c3c2)cc1NS(=O)(=O)c1ccc(F)cc1F] |
| | | 2,4-difluoro-N-[2-methoxy-5-(4-pyridazin-4-yl |
| | | quinolin-6-yl)pyridin-3-yl]benzenesulfonamide |
| Oprozomib | 935888-69-0 | 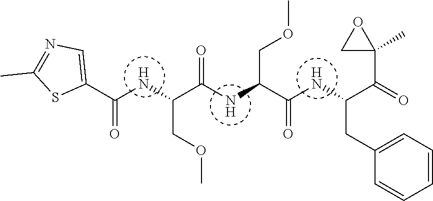 [SMILES: COC[C@H](NC(=O)c1cnc(C)s1)C(=O)N[C@@H](COC)C(=O)N[C@@H](Cc1ccccc1)C(=O)[C@@]1(C)CO1] |
| | | N-[(2S)-3-methoxy-1-[(2S)-3-methoxy-1-[(2S)-1-[(2R)-2-methyloxiran- |
| | | 2-yl]-1-oxo-3-phenylpropan-2-yl]amino]-1-oxopropan-2-yl]amino]-1- |
| | | oxopropan-2-yl]-2-methyl-1,3-thiazole-5-carboxamide |
| Osimertinib | 1421373-65-0 | 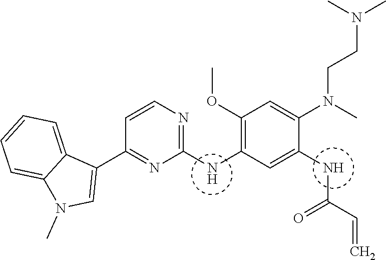 [SMILES: C=CC(=O)Nc1cc(Nc2nccc(-c3cn(C)c4ccccc34)n2)c(OC)cc1N(C)CCN(C)C] |
| | | N-[2-[2-(dimethylamino)ethyl-methylamino]-4-methoxy-5- |
| | | [[4-(1-methylindol-3-yl)pyrimidin-2-yl]amino]phenyl]prop- |
| | | 2-enamide |
| OTS-964 | — | 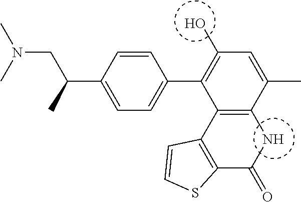 |
| | | 9-[4-[(2R)-1-(dimethylamino)propan-2-yl]phen- |
| | | yl]-8-hydroxy-6-methyl-5H-thieno[2,3-c]quinolin- |
| | | 4-one |
| Palbociclib | 571190-30-2 | 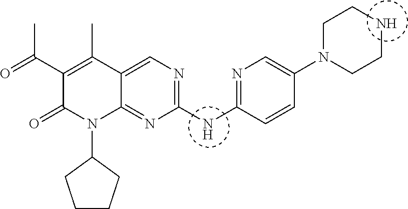 [SMILES: CC(=O)c1c(C)c2cnc(Nc3ccc(N4CCNCC4)cn3)nc2n(C2CCCC2)c1=O] |
| | | 6-acetyl-8-cyclopentyl-5-methyl-2-[(5-piperazin-1-ylpyridin-2-yl)- |
| | | amino]pyrido[2,3-d]pyrimidin-7-one |
| Pamiparib | 1446261-44-4 | 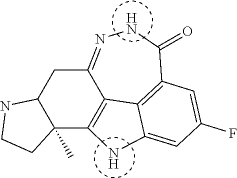 [SMILES: C[C@]12CCNC1CC1=NNC(=O)c3cc(F)cc4[nH]c2c1c34] |
| | | (2R)-14-fluoro-2-methyl-6,9,10,19- |
| | | tetrazapentacyclo[14.2.1.0^{2,6}.0^{8,18}.0^{12,17}] |
| | | nonadeca-1(18),8,12(17),13,15-pentaen- |
| | | 11-one |
| Panobinostat | 404950-80-7 | 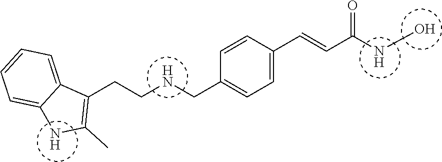 [SMILES: Cc1[nH]c2ccccc2c1CCNCc1ccc(/C=C/C(=O)NO)cc1] |
| | | (E)-N-hydroxy-3-[4-[2-(2-methyl-1H-indol-3-yl)ethylamino]methyl]phen- |
| | | yl]prop-2-enamide |
| Paricalcitol | 131918-61-1 | 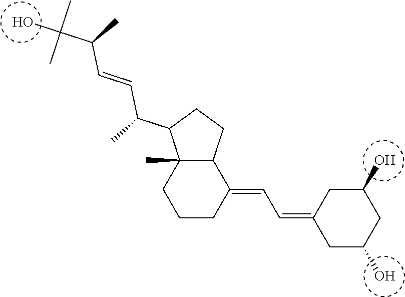 [SMILES: C[C@H](/C=C/[C@H](C)C(C)(C)O)C1CCC2/C(=C/C=C3C[C@@H](O)C[C@H](O)C3)CCC[C@@]21C] |
| | | (1R,3R)-5-[(2E)-2-[(1R,3aS,7aR)-1-[(E,2R,5S)-6-hydroxy-5,6- |
| | | dimethylhept-3-en-2-yl]-7a-methyl-2,3,3a,5,6,7-hexahydro-1H- |
| | | inden-4-ylidene]ethylidene]cyclohexane-1,3-diol |
| Parsaclisib | 1426698-88-5 | 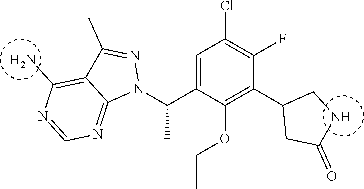 [SMILES: CCOc1c([C@H](C)n2nc(C)c3c(N)ncnc32)cc(Cl)c(F)c1C1CNC(=O)C1] |
| | | (4R)-4-[3-[(1S)-1-(4-amino-3-methylpyrazolo[3,4-d]pyr- |
| | | imidin-1-yl)ethyl]-5-chloro-2-ethoxy-6-fluorophenyl] |
| | | pyrrolidin-2-one |
| Pazopanib | 444731-52-6 | 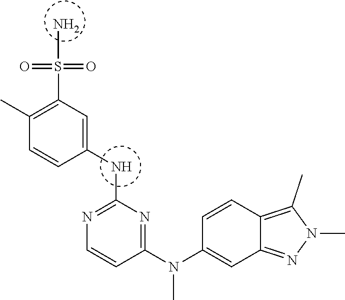 |
| | | 5-[[4-[(2,3-dimethylindazol-6-yl)-methylamino]pyrim- |
| | | idin-2-yl]amino]-2-methylbenzenesulfonamide |
| Pemetrexed | 137281-23-3 | 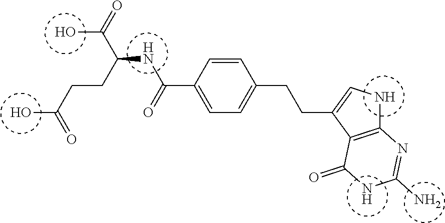 [SMILES: Nc1nc2[nH]cc(CCc3ccc(C(=O)N[C@@H](CCC(=O)O)C(=O)O)cc3)c2c(=O)[nH]1] |
| | | (2S)-2-[[4-[2-(2-amino-4-oxo-3,7-dihydropyrrolo[2,3-d]pyrimidin-5- |
| | | yl)ethyl]benzoyl]amino]pentanedioic acid |
| Pemigatinib | 1513857-77-6 | 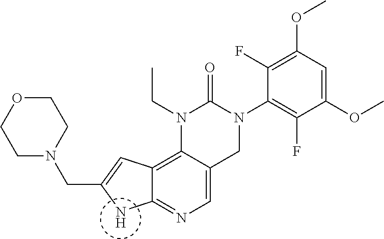 [SMILES: CCN1C(=O)N(c2c(F)c(OC)cc(OC)c2F)Cc2cnc3[nH]c(CN4CCOCC4)cc3c21] |
| | | 11-(2,6-difluoro-3,5-dimethoxyphenyl)-13-ethyl-4-(morpholin- |
| | | 4-ylmethyl)-5,7,11,13-tetrazatricyclo[7.4.0.02,6]trideca-1,3,6,8- |
| | | tetraen-12-one |
| Pevonedistat | 905579-51-3 | 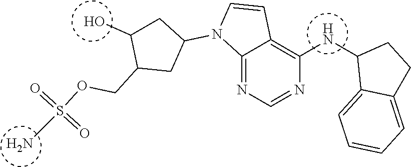 [SMILES: NS(=O)(=O)OCC1CC(n2ccc3c(NC4CCc5ccccc54)ncnc32)CC1O] |
| | | [(1S,2S,4R)-4-[4-[[(1S)-2,3-dihydro-1H-inden-1-yl]amino]pyrrolo |
| | | [2,3-d]pyrimidin-7-yl]-2-hydroxycyclopentyl]methyl sulfamate |
| Pexidartinib | 1029044-16-3 | 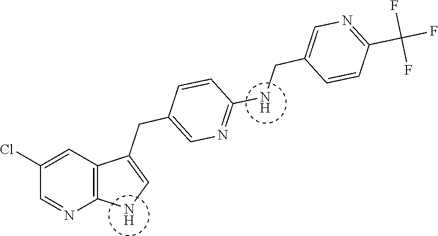 [SMILES: FC(F)(F)c1ccc(CNc2ccc(Cc3c[nH]c4ncc(Cl)cc34)cn2)cn1] |
| | | 5-[(5-chloro-1H-pyrrolo[2,3-b]pyridin-3-yl)methyl]-N-[[6-(trifluoro- |
| | | methyl)pyridin-3-yl]methyl]pyridin-2-amine |
| Pifusertib | 1402602-94-1 | 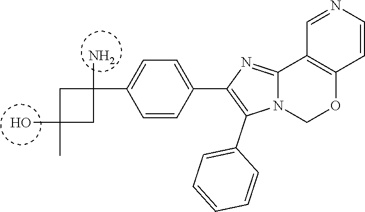 |
| | | 3-amino-1-methyl-3-[4-(5-phenyl-8-oxa-3,6,12-triazatri- |
| | | cyclo[7.4.0.0^{2,6}]trideca-1(9),2,4,10,12-pentaen-4-yl)phen- |
| | | yl]cyclobutan-1-ol |
| Plerixafor | 110078-46-1 | 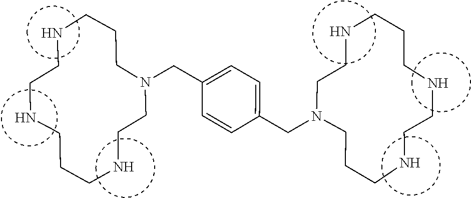 [SMILES: c1cc(CN2CCCNCCNCCCNCC2)ccc1CN1CCCNCCCNCCCCNCC1] |
| | | 1-[4-(1,4,8,11-tetrazacyclotetradec-1-ylmethyl)phenyl]methyl]-1,4,8,11-tetraza- |
| | | cyclotetradecane |
| PMPA | 173039-10-6 | 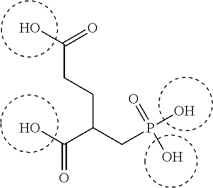 [SMILES: O=C(O)CCC(CP(=O)(O)O)C(=O)O] |
| | | 2-(phosphonomethyl)pentanedioic |
| | | acid |
| Ponatinib | 943319-70-8 | 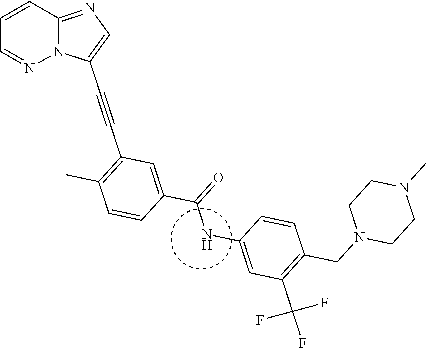 [SMILES: Cc1ccc(C(=O)Nc2ccc(CN3CCN(C)CC3)c(C(F)(F)F)c2)cc1C#Cc1cnc2cccnn12] |
| | | 3-(2-imidazo[1,2-b]pyridazin-3-ylethynyl)-4-methyl-N-[4-[(4-methyl- |
| | | piperazin-1-yl)methyl]-3-(trifluoromethyl)phenyl]benzamide |
| Practinostat | 929016-96-6 | 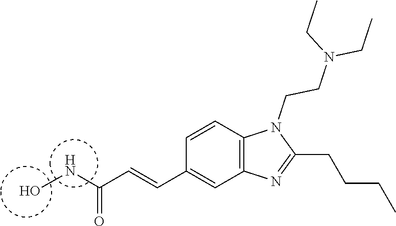 [SMILES: CCCCc1nc2cc(/C=C/C(=O)NO)ccc2n1CCN(CC)CC] |
| | | (E)-3-[2-butyl-1-[2-(diethylamino)ethyl]benzimidazol-5-yl]-N- |
| | | hydroxyprop-2-enamide |
| Pralsetinib | 2097132-94-8 | 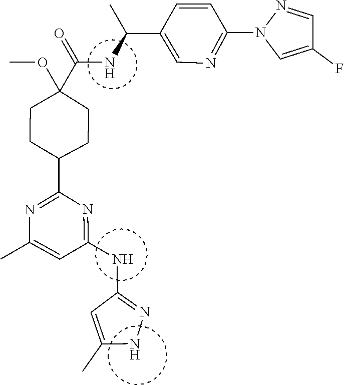 [SMILES: COC1(C(=O)N[C@@H](C)c2ccc(-n3cc(F)cn3)nc2)CCC(c2nc(C)cc(Nc3cc(C)[nH]n3)n2)CC1] |
| | | N-[(1S)-1-[6-(4-fluoropyrazol-1-yl)pyridin-3-yl]ethyl]- |
| | | 1-methoxy-4-[4-methyl-6-[(5-methyl-1H-pyrazol-3-yl)- |
| | | amino]pyrimidin-2-yl]cyclohexane-1-carboxamide |
| Prednisone | 53-03-2 | 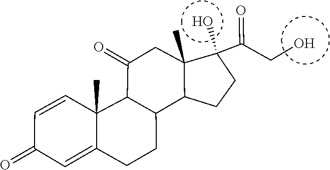 |
| | | (8S,9S,10R,13S,14S,17R)-17-hydroxy-17-(2-hydroxy- |
| | | acetyl)-10,13-dimethyl-6,7,8,9,12,14,15,16-octahydro- |
| | | cyclopenta[a]phenanthrene-3,11-dione |
| Prexasertib | 1234015-52-1 | 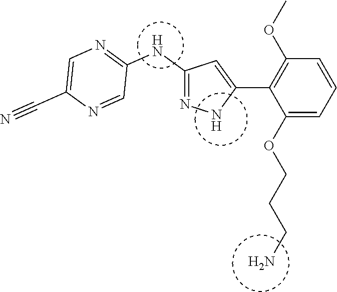 [SMILES: COc1cccc(OCCCN)c1-c1cc(Nc2cnc(C#N)cn2)n[nH]1] |
| | | 5-[[5-[2-(3-aminopropoxy)-6-methoxyphenyl]-1H- |
| | | pyrazol-3-yl]amino]pyrazine-2-carbonitrile |
| Prinomastat | 192329-42-3 | 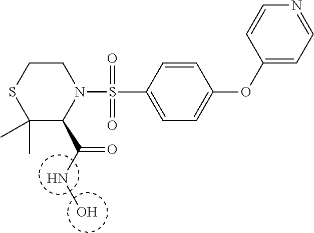 [SMILES: CC1(C)SCCN(S(=O)(=O)c2ccc(Oc3ccncc3)cc2)[C@H]1C(=O)NO] |
| | | (3S)-N-hydroxy-2,2-dimethyl-4-(4-pyridin-4-yloxy- |
| | | phenyl)sulfonylthiomorpholine-3-carboxamide |
| Propranolol | 525-66-6 | 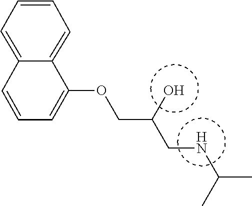 [SMILES: CC(C)NCC(O)COc1cccc2ccccc12] |
| | | 1-naphthalen-1-yloxy-3-(propan-2-yl- |
| | | amino)propan-2-ol |
| Quisinostat | 875320-29-9 | 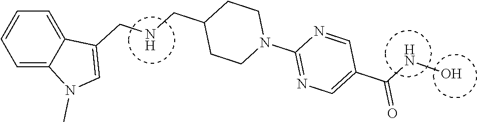 [SMILES: Cn1cc(CNCC2CCN(c3ncc(C(=O)NO)cn3)CC2)c2ccccc21] |
| | | N-hydroxy-2-[4-[[(1-methylindol-3-yl)methylamino]methyl]piperidin-1-yl] |
| | | pyrimidine-5-carboxamide |
| Quizartinib | 950769-58-1 | 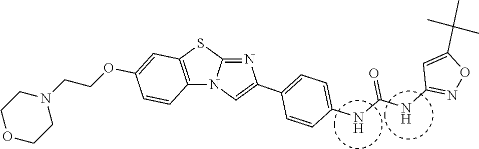 [SMILES: CC(C)(C)c1cc(NC(=O)Nc2ccc(-c3cn4c(n3)sc3cc(OCCN5CCOCC5)ccc34)cc2)no1] |
| | | 1-(5-tert-butyl-1,2-oxazol-3-yl)-3-[4-[6-(2-morpholin-4-ylethoxy)imidazo[2,1- |
| | | b][1,3]benzothiazol-2-yl]phenyl]urea |
| Ralimetinib | 862505-00-8 | 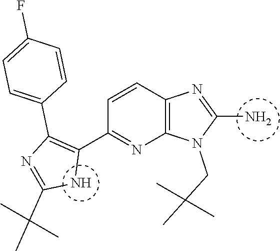 [SMILES: CC(C)(C)Cn1c(N)nc2ccc(-c3[nH]c(C(C)(C)C)nc3-c3ccc(F)cc3)nc21] |
| | | 5-[2-tert-butyl-4-(4-fluorophenyl)-1H-imid- |
| | | azol-5-yl]-3-(2,2-dimethylpropyl)imidazo |
| | | [4,5-b]pyridin-2-amine |
| Ravoxertinib | 1453848-26-4 | 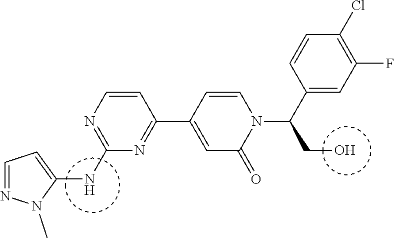 [SMILES: Cn1nccc1Nc1nccc(-c2ccn([C@H](CO)c3ccc(Cl)c(F)c3)c(=O)c2)n1] |
| | | 1-[(1S)-1-(4-chloro-3-fluorophenyl)-2-hydroxyethyl]-4-[2-[(2- |
| | | methylpyrazol-3-yl)amino]pyrimidin-4-yl]pyridin-2-one |
| Regorafenib | 755037-03-7 | 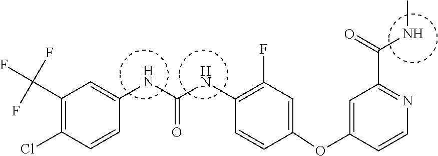 [SMILES: CNC(=O)c1cc(Oc2ccc(NC(=O)Nc3ccc(Cl)c(C(F)(F)F)c3)c(F)c2)ccn1] |
| | | 4-[4-[[4-chloro-3-(trifluoromethyl)phenyl]carbamoylamino]-3-fluoro- |
| | | phenoxy]-N-methylpyridine-2-carboxamide |
| Relugolix | 737789-87-6 | 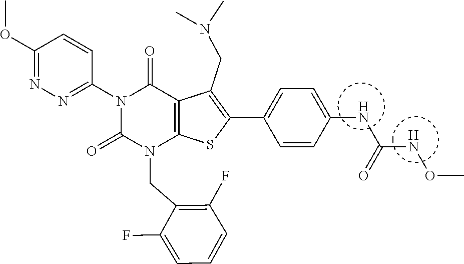 [SMILES: CONC(=O)Nc1ccc(-c2sc3c(c2CN(C)C)c(=O)n(-c2ccc(OC)nn2)c(=O)n3Cc2c(F)cccc2F)cc1] |
| | | 1-[4-[1-[(2,6-difluorophenyl)methyl]-5-[(dimethylamino)methyl]-3-(6- |
| | | methoxypyridazin-3-yl)-2,4-dioxothieno[2,3-d]pyrimidin-6-yl]phenyl]- |
| | | 3-methoxyurea |
| Resminostat | 864814-88-0 | 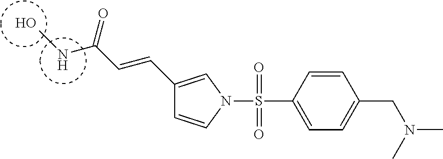 |
| | | (E)-3-[1-[4-[(dimethylamino)methyl]phenyl]sulfonylpyrrol-3-yl]-N- |
| | | hydroxyprop-2-enamide |
| Resveratrol | 501-36-0 | 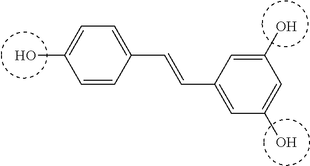 [SMILES: Oc1ccc(/C=C/c2cc(O)cc(O)c2)cc1] |
| | | 5-[(E)-2-(4-hydroxyphenyl)ethenyl]benzene-1,3-diol |
| Retaspimycin | 857402-23-4 | 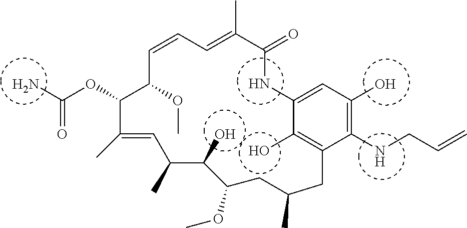 [SMILES: C=CCNc1c(O)cc2c(O)c1C[C@@H](C)C[C@H](OC)[C@H](O)[C@@H](C)/C=C(\C)[C@H](OC(N)=O)[C@@H](OC)/C=C\C=C(/C)C(=O)N2] |
| | | [(4E,6Z,8S,9S,10E,12S,13R,14S,16R)-13,20,22-trihydroxy-8,14-dimethoxy- |
| | | 4,10,12,16-tetramethyl-3-oxo-19-(prop-2-enylamino)-2-azabicyclo[16.3.1] |
| | | docosa-1(21),4,6,10,18(22),19-hexaen-9-yl]carbamate |
| Retinoic acid | 302-79-4 | 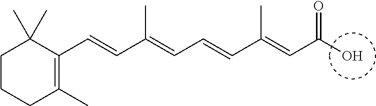 [SMILES: CC1=C(/C=C/C(C)=C/C=C/C(C)=C/C(=O)O)C(C)(C)CCC1] |
| | | (2E,4E,6E,8E)-3,7-dimethyl-9-(2,6,6-trimethylcyclohexen- |
| | | 1-yl)nona-2,4,6,8-tetraenoic acid |
| Ribociclib | 1211441-98-3 | 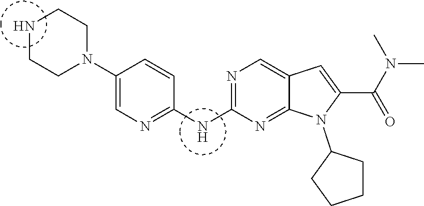 [SMILES: CN(C)C(=O)c1cc2cnc(Nc3ccc(N4CCNCC4)cn3)nc2n1C1CCCC1] |
| | | 7-cyclopentyl-N,N-dimethyl-2-[(5-piperazin-1-ylpyridin-2-yl)amino] |
| | | pyrrolo[2,3-d]pyrimidine-6-carboxamide |
| Ricolinostat | 1316214-52-4 | 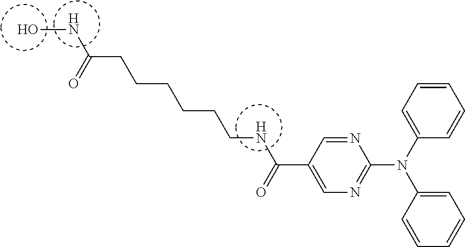 [SMILES: O=C(CCCCCCNC(=O)c1cnc(N(c2ccccc2)c2ccccc2)nc1)NO] |
| | | N-[7-(hydroxyamino)-7-oxoheptyl]-2-(N-phenylanilino)pyrimidine- |
| | | 5-carboxamide |
| Rigosertib | 592542-59-1 | 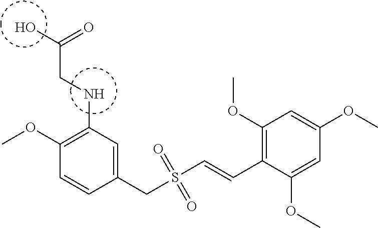 |
| | | 2-[2-methoxy-5-[[(E)-2-(2,4,6-trimethoxyphenyl)ethenyl] |
| | | sulfonylmethyl]anilino]acetic acid |
| Ripretinib | 1442472-39-0 | 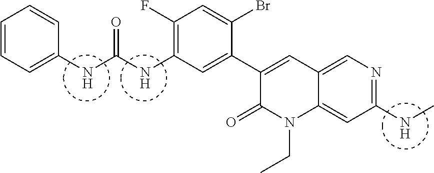 [SMILES: CCn1c(=O)c(-c2cc(NC(=O)Nc3ccccc3)c(F)cc2Br)cc2cnc(NC)cc21] |
| | | 1-[4-bromo-5-[1-ethyl-7-(methylamino)-2-oxo-1,6-naphthyridin-3-yl]- |
| | | 2-fluorophenyl]-3-phenylurea |
| RO-3306 | 872573-93-8 | 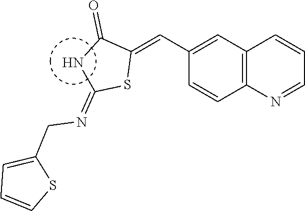 [SMILES: O=C1N/C(=N\Cc2cccs2)S/C1=C\c1ccc2ncccc2c1] |
| | | (5Z)-5-(quinolin-6-ylmethylidene)-2-(thiophen- |
| | | 2-ylmethylimino)-1,3-thiazolidin-4-one |
| Rocilinostat | 1316214-52-4 | 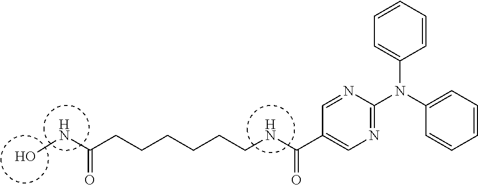 [SMILES: O=C(CCCCCCNC(=O)c1cnc(N(c2ccccc2)c2ccccc2)nc1)NO] |
| | | N-[7-(hydroxyamino)-7-oxoheptyl]-2-(N-phenylanilino)pyrimidine- |
| | | 5-carboxamide |
| Rogaratinib | 1443530-05-9 | 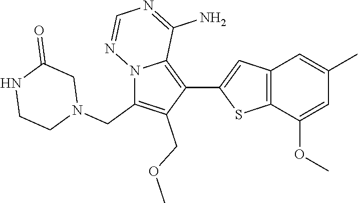 [SMILES: COCc1c(-c2cc3cc(C)cc(OC)c3s2)c2c(N)ncnn2c1CN1CCNC(=O)C1] |
| | | 4-[[4-amino-6-(methoxymethyl)-5-(7-methoxy-5-methyl- |
| | | 1-benzothiophen-2-yl)pyrrolo[2,1-f][1,2,4]triazin-7-yl] |
| | | methyl]piperazin-2-one |
| Romidepsin | 128517-07-7 | 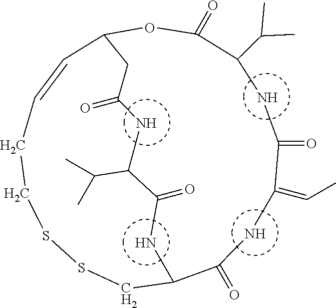 |
| | | 7-ethylidene-4,21-di(propan-2-yl)-2-oxa-12,13-dithia- |
| | | 5,8,20,23-tetrazabicyclo[8.7.6]tricos-16-ene-3,6,9, |
| | | 19,22-pentone |
| Rucaparib | 283173-50-2 | 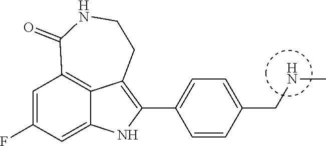 [SMILES: CNCc1ccc(-c2[nH]c3cc(F)cc4c3c2CCNC4=O)cc1] |
| | | 6-fluoro-2-[4-(methylaminomethyl)phenyl]-3,10-diaza- |
| | | tricyclo[6.4.1.04,13]trideca-1,4,6,8(13)-tetraen-9-one |
| Ruxolitinib | 941678-49-5 | 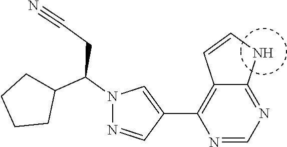 [SMILES: N#CC[C@H](C1CCCC1)n1cc(-c2ncnc3[nH]ccc23)cn1] |
| | | (3R)-3-cyclopentyl-3-[4-(7H-pyrrolo[2,3-d] |
| | | pyrimidin-4-yl)pyrazol-1-yl]propanenitrile |
| S2 | 108900-05-6 | 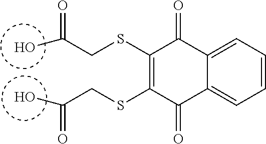 [SMILES: O=C(O)CSC1=C(SCC(=O)O)C(=O)c2ccccc2C1=O] |
| | | 2-[3-(carboxymethylsulfanyl)-1,4-dioxo- |
| | | naphthalen-2-yl]sulfanylacetic acid |
| S5 | 57414-03-6 | 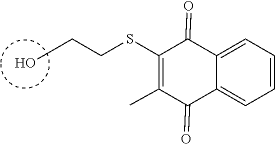 [SMILES: CC1=C(SCCCO)C(=O)c2ccccc2C1=O] |
| | | 2-(1,4-dioxonaphthalen-2-yl)sulfanylacetic |
| | | acid |
| Saridegib | 1037210-93-7 | 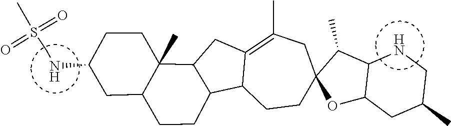 |
| | | N-[(3R,3′R,3′aS,4aR,6′S,6aR,6bS,T′aR,9S,12aS,12bS)-3′,6′,11,12b-tetra- |
| | | methylspiro[1,2,3,4,4a,5,6,6a,6b,7,8,10,12,12a-tetradecahydronaphtho |
| | | [2,1-a]azulene-9,2′-3a,4,5,6,7,7a-hexahydro-3H-furo[3,2-b]pyridine]-3- |
| | | yl]methanesulfonamide |
| SBI-0654454 | — | 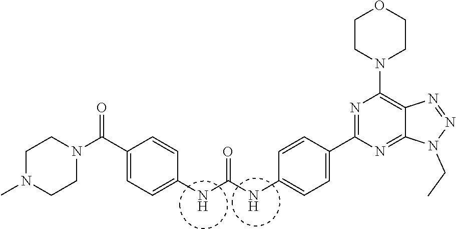 [SMILES: CCn1nnc2c(N3CCOCC3)nc(-c3ccc(NC(=O)Nc4ccc(C(=O)N5CCN(C)CC5)cc4)cc3)nc21] |
| SCH772984 | 942183-80-4 | 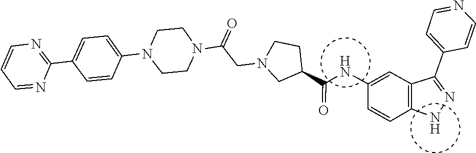 |
| | | (3R)-1-[2-oxo-2-[4-(4-pyrimidin-2-ylphenyl)piperazin-1-yl]ethyl]-N-(3-pyridin- |
| | | 4-yl-1H-indazol-5-yl)pyrrolidine-3-carboxamide |
| Seliciclib | 186692-46-6 | 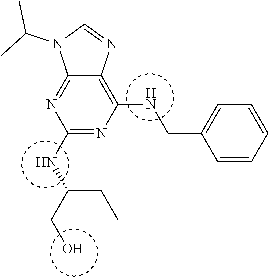 [SMILES: CC[C@H](CO)Nc1nc(NCc2ccccc2)c2ncn(C(C)C)c2n1] |
| | | (2R)-2-[[6-(benzylamino)-9-propan-2-yl- |
| | | purin-2-yl]amino]butan-1-ol |
| Selitrectinib | 2097002-61-2 | 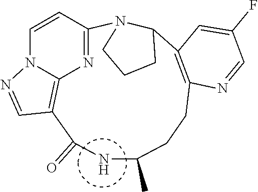 [SMILES: C[C@@H]1CCc2ncc(F)cc2C2CCCN2c2ccn3ncc(c3n2)C(=O)N1] |
| | | (6R,15R)-9-fluoro-15-methyl-2,11,16,20, |
| | | 21,24-hexazapentacyclo[16.5.2.0^{2,6}.0^{7,12}. |
| | | 0^{21,25}]pentacosa-1(24),7(12),8,10,18(25), |
| | | 19,22-heptaen-17-one |
| Selpercatinib | 2152628-33-4 | 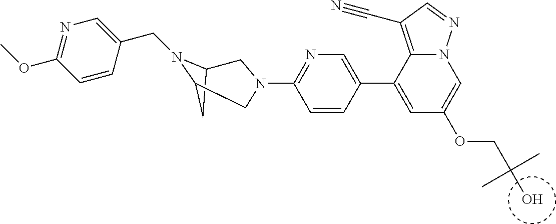 [SMILES: COc1ccc(CN2C3CC2CN(c2ccc(-c4cc(OCC(C)(C)O)cn5ncc(C#N)c45)cn2)C3)cn1] |
| | | 6-(2-hydroxy-2-methylpropoxy)-4-[6-[6-[(6-methoxypyridin-3-yl)methyl]-3,6-diazabicyclo |
| | | [3.1.1]heptan-3-yl]pyridin-3-yl]pyrazolo[1,5-a]pyridine-3-carbonitrile |
| Selumetinib | 606143-52-6 | 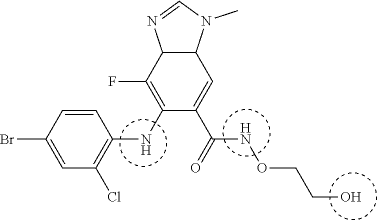 [SMILES: CN1C=NC2C(F)=C(Nc3ccc(Br)cc3Cl)C(C(=O)NOCCO)=CC21] |
| | | 6-(4-bromo-2-chloroanilino)-7-fluoro-N-(2-hydroxyethoxy)- |
| | | 3-methylbenzimidazole-5-carboxamide |
| SGN-2FF | 2089647-47-0 | 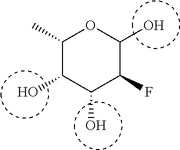 |
| | | (3S,4R,5S,6S)-3-fluoro-6- |
| | | methyloxane-2,4,5-triol |
| SGX393 | 1030610-86-6 | 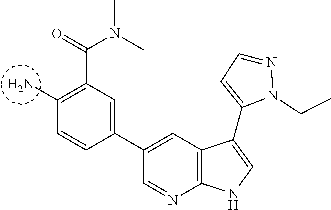 [SMILES: CCn1nccc1-c1c[nH]c2ncc(-c3ccc(N)c(C(=O)N(C)C)c3)cc12] |
| | | 2-amino-5-[3-(2-ethylpyrazol-3-yl)-1H-pyrrolo[2,3-b] |
| | | pyridin-5-yl]-N,N-dimethylbenzamide |
| Shikonin | 517-89-5 | 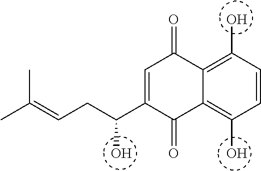 [SMILES: CC(C)=CC[C@@H](O)C1=CC(=O)c2c(O)ccc(O)c2C1=O] |
| | | 5,8-dihydroxy-2-[(1R)-1-hydroxy-4-meth- |
| | | ylpent-3-enyl]naphthalene-1,4-dione |
| Silibinin | 22888-70-6 | 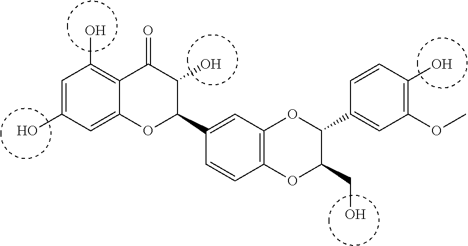 [SMILES: COc1cc([C@H]2Oc3cc([C@H]4Oc5cc(O)cc(O)c5C(=O)[C@@H]4O)ccc3O[C@@H]2CO)ccc1O] |
| | | (2R,3R)-3,5,7-trihydroxy-2-[(2R,3R)-3-(4-hydroxy-3-methoxyphenyl)-2- |
| | | (hydroxymethyl)-2,3-dihydro-1,4-benzodioxin-6-yl]-2,3-dihydrochromen- |
| | | 4-one |
| Sitravatinib | 1123837-84-2 | 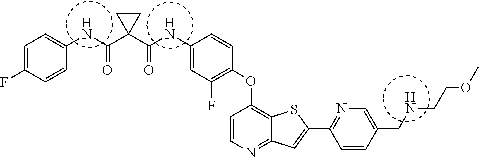 |
| | | 1-N-[3-fluoro-4-[2-[5-[(2-methoxyethylamino)methyl]pyridin-2-yl]thieno |
| | | [3,2-b]pyridin-7-ylJoxyphenyl]-1-N-(4-fluorophenyl)cyclopropane-1,1- |
| | | dicarboxamide |
| Sonidegib | 956697-53-3 | 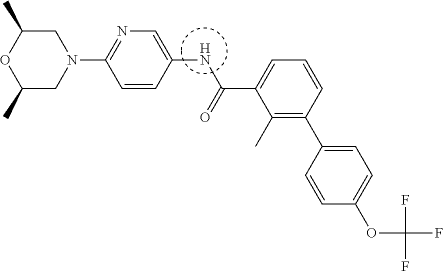 [SMILES: Cc1c(C(=O)Nc2ccc(N3C[C@@H](C)O[C@@H](C)C3)nc2)cccc1-c1ccc(OC(F)(F)F)cc1] |
| | | N-[6-[(2S,6R)-2,6-dimethylmorpholin-4-yl]pyridin-3-yl]-2-methyl-3-[4- |
| | | (trifluoromethoxy)phenyl]benzamide |
| Sorafenib | 284461-73-0 | 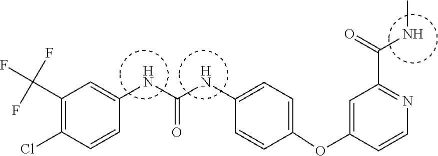 [SMILES: CNC(=O)c1cc(Oc2ccc(NC(=O)Nc3ccc(Cl)c(C(F)(F)F)c3)cc2)ccn1] |
| | | 4-[4-[[4-chloro-3-(trifluoromethyl)phenyl]carbamoylamino]phenoxy]-N- |
| | | methylpyridine-2-carboxamide |
| Sotorasib | 2252403-56-6 | 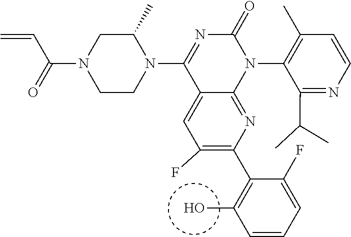 [SMILES: C=CC(=O)N1CCN(c2nc(=O)n(-c3c(C)ccnc3C(C)C)c3nc(-c4c(O)cccc4F)c(F)cc23)[C@@H](C)C1] |
| | | 6-fluoro-7-(2-fluoro-6-hydroxyphenyl)-1-(4-methyl-2- |
| | | propan-2-ylpyridin-3-yl)-4-[(2S)-2-methyl-4-prop-2- |
| | | enoylpiperazin-1-yl]pyrido[2,3-d]pyrimidin-2-one |
| Staurosporine | 62996-74-1 | 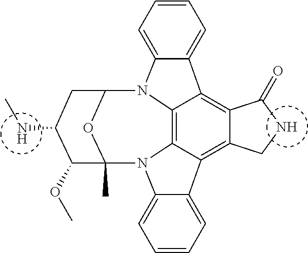 [SMILES: CN[C@@H]1CC2O[C@@](C)([C@@H]1OC)n1c3ccccc3c3c4c(c5c6ccccc6n2c5c31)C(=O)NC4] |
| | | (2S,3R,4R,6R)-3-methoxy-2-methyl-4-(methyl- |
| | | amino)-29-oxa-1,7,17-triazaoctacyclo[12.12.2.1^{2,6}. |
| | | 0^{7,28}.0^{8,13}.0^{15,19}.0^{20,27}.0^{21,26}]nonacosa-8,10,12,14,19, |
| | | 21,23,25,27-nonaen-16-one |
| SU11274 | 658084-23-2 | 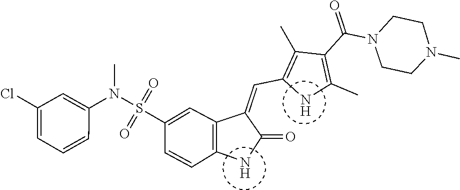 [SMILES: Cc1[nH]c(/C=C2\C(=O)NCc3ccc(S(=O)(=O)N(C)c4cccc(Cl)c4)cc32)c(C)c1C(=O)N1CCN(C)CC1] |
| | | (3Z)-N-(3-chlorophenyl)-3-[[3,5-dimethyl-4-(4-methylpiperazine-1-carbon- |
| | | yl)-1H-pyrrol-2-yl]methylidene]-N-methyl-2-oxo-1H-indole-5-sulfonamide |
| Sunitinib | 557795-19-4 | 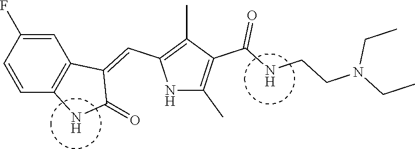 [SMILES: CCN(CC)CCNC(=O)c1c(C)[nH]c(/C=C2\C(=O)Nc3ccc(F)cc32)c1C] |
| | | N-[2-(diethylamino)ethyl]-5-[(Z)-(5-fluoro-2-oxo-1H-indol-3-yl- |
| | | idene)methyl]-2,4-dimethyl-1H-pyrrole-3-carboxamide |
| Surufatinib | 1308672-74-3 | 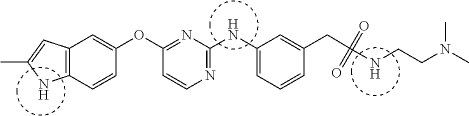 |
| | | N-[2-(dimethylamino)ethyl]-1-[3-[[4-[(2-methyl-1H-indol-5-yl)oxy]pyrim- |
| | | idin-2-yl]amino]phenyl]methanesulfonamide |
| Tacedinaline | 112522-64-2 | 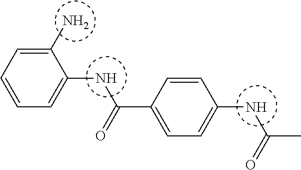 [SMILES: CC(=O)Nc1ccc(C(=O)Nc2ccccc2N)cc1] |
| | | 4-acetamido-N-(2-aminophenyl)benzamide |
| Tadalafil | 171596-29-5 | 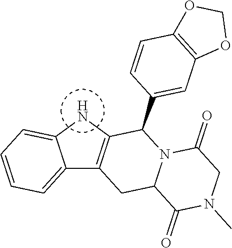 [SMILES: CN1CC(=O)N2C(Cc3c([nH]c4ccccc34)[C@H]2c2ccc3c(c2)OCO3)C1=O] |
| | | (2R,8R)-2-(1,3-benzodioxol-5-yl)-6- |
| | | methyl-3,6,17-triazatetracyclo[8.7.0.03, |
| | | 8.011,16]heptadeca-1(10),11,13,15- |
| | | tetraene-4,7-dione |
| Talazoparib | 1207456-01-6 | 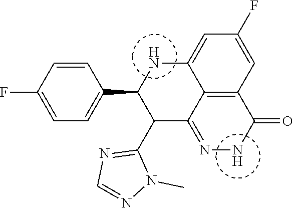 [SMILES: Cn1ncnc1C1c2n[nH]c(=O)c3cc(F)cc(c23)N[C@@H]1c1ccc(F)cc1] |
| | | (11S,12R)-7-fluoro-11-(4-fluorophenyl)-12-(2- |
| | | methyl-1,2,4-triazol-3-yl)-2,3,10-triazatricyclo |
| | | [7.3.1.05,13]trideca-1,5(13),6,8-tetraen-4-one |
| Taletrectinib | 1505515-69-4 | 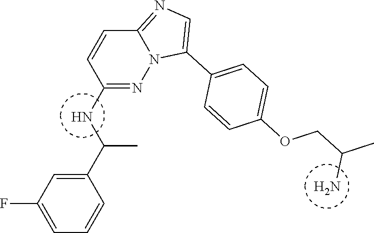 [SMILES: CC(N)COc1ccc(-c2cnc3ccc(NC(C)c4cccc(F)c4)nn23)cc1] |
| | | 3-[4-[(2R)-2-aminopropoxyphenyl]-N-[(1R)-1-(3-fluorophen- |
| | | yl)ethyl]imidazo[1,2-b]pyridazin-6-amine;hexanedioic acid |
| Tarloxotinib | 1636938-13-0 | 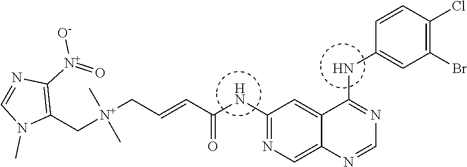 [SMILES: Cn1cnc([N+](=O)[O-])c1C[N+](C)(C)C/C=C/C(=O)Nc1cc2c(Nc3ccc(Cl)c(Br)c3)ncnc2cn1] |
| | | [(E)-4-[[4-(3-bromo-4-chloroanilino)pyrido[3,4-d]pyrimidin-6-yl]amino]-4- |
| | | oxobut-2-enyl]-dimethyl-[(3-methyl-5-nitroimidazol-4-yl)methyl]azanium |
| Taselisib | 1282512-48-4 | 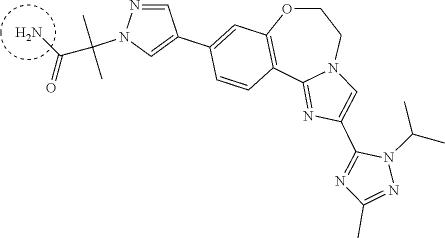 [SMILES: Cc1nc(-c2cn3c(n2)-c2ccc(-c4cnn(C(C)(C)C(N)=O)c4)cc2OCC3)n(C(C)C)n1] |
| | | 2-methyl-2-[4-[2-(5-methyl-2-propan-2-yl-1,2,4-triazol-3-yl)-5,6-dihydro- |
| | | imidazo[1,2-d][1,4]benzoxazepin-9-yl]pyrazol-1-yl]propanamide |
| Tazemetostat | 1403254-99-8 | 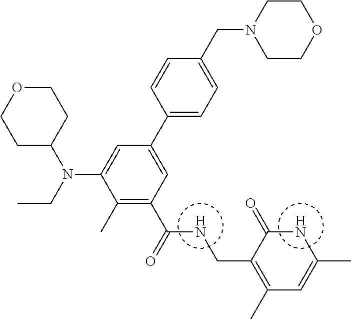 [SMILES: CCN(c1cc(-c2ccc(CN3CCOCC3)cc2)cc(C(=O)NCc2c(C)cc(C)[nH]c2=O)c1C)C1CCOCC1] |
| | | N-[(4,6-dimethyl-2-oxo-1H-pyridin-3-yl)methyl]-3-[ethyl- |
| | | (oxan-4-yl)amino]-2-methyl-5-[4-(morpholin-4-ylmethyl)- |
| | | phenyl]benzamide |
| Tefinostat | 914382-60-8 | 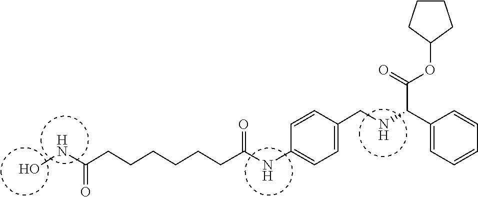 |
| | | cyclopentyl (2S)-2-[[4-[[8-(hydroxyamino)-8-oxooctanoyl]amino]phenyl]meth- |
| | | ylamino]-2-phenylacetate |
| Temsirolimus | 162635-04-3 | 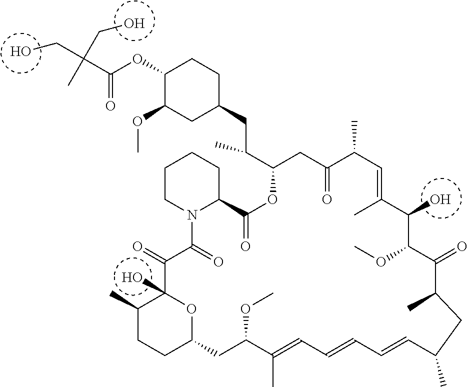 |
| | | [(1R,2R,4S)-4-[(2R)-2-[(1R,9S,12S,15R,16E,18R,19R,21R,23S,24E,26E, |
| | | 28E,30S,32S,35R)-1,18-dihydroxy-19,30-dimethoxy-15,17,21,23,29,35- |
| | | hexamethyl-2,3,10,14,20-pentaoxo-11,36-dioxa-4-azatricyclo[30.3.1.0^{4,9}] |
| | | hexatriaconta-16,24,26,28-tetraen-12-yl]propyl]-2-methoxycyclohexyl]3- |
| | | hydroxy-2-(hydroxymethyl)-2-methylpropanoate |
| Tetrazole | 288-94-8 | 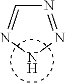 |
| | | 2H- |
| | | tetrazole |
| Tivozanib | 475108-18-0 | 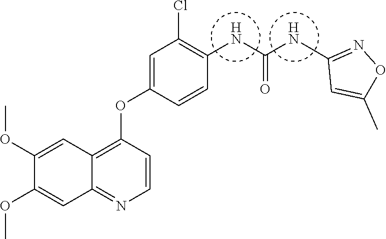 [SMILES: COc1cc2nccc(Oc3ccc(NC(=O)Nc4cc(C)on4)c(Cl)c3)c2cc1OC] |
| | | 1-[2-chloro-4-(6,7-dimethoxyquinolin-4-yl)oxyphenyl]-3-(5- |
| | | methyl-1,2-oxazol-3-yl)urea |
| Tofacitinib | 477600-75-2 | 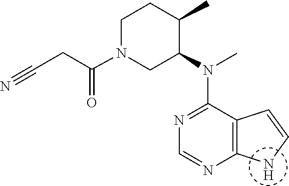 [SMILES: C[C@@H]1CCN(C(=O)CC#N)C[C@@H]1N(C)c1ncnc2[nH]ccc12] |
| | | 3-[(3R,4R)-4-methyl-3-[methyl(7H-pyrrolo[2,3- |
| | | d]pyrimidin-4-yl)amino]piperidin-1-yl]-3-oxo- |
| | | propanenitrile |
| Tozasertib | 639089-54-6 | 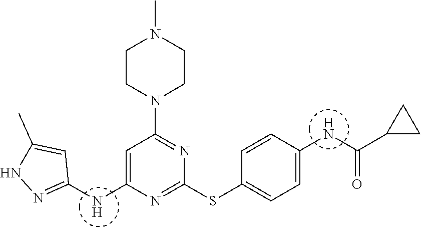 [SMILES: Cc1cc(Nc2cc(N3CCN(C)CC3)nc(Sc3ccc(NC(=O)C4CC4)cc3)n2)n[nH]1] |
| | | N-[4-[4-(4-methylpiperazin-1-yl)-6-[(5-methyl-1H-pyrazol-3-yl)- |
| | | amino]pyrimidin-2-yl]sulfanylphenyl]cyclopropanecarboxamide |
| Trametinib | 871700-17-3 | 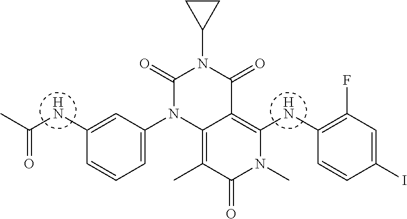 [SMILES: CC(=O)Nc1cccc(-n2c(=O)n(C3CC3)c(=O)c3c(Nc4ccc(I)cc4F)n(C)c(=O)c(C)c32)c1] |
| | | N-[3-[3-cyclopropyl-5-(2-fluoro-4-iodoanilino)-6,8-dimethyl-2,4,7- |
| | | trioxopyrido[4,3-d]pyrimidin-1-yl]phenyl]acetamide |
| Tranilast | 53902-12-8 | 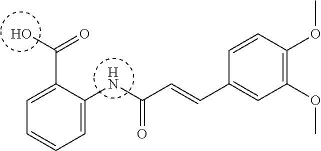 [SMILES: COc1ccc(/C=C/C(=O)Nc2ccccc2C(=O)O)cc1OC] |
| | | 2-[(E)-3-(3,4-dimethoxyphenyl)prop-2-enoyl]amino] |
| | | benzoic acid |
| Tretinoin | 302-79-4 | 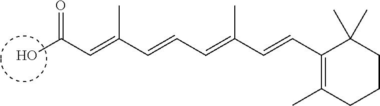 [SMILES: CC1=C(/C=C/C(C)=C/C=C/C(C)=C/C(=O)O)C(C)(C)CCC1] |
| | | (2E,4E,6E,8E)-3,7-dimethyl-9-(2,6,6-trimethylcyclohexen-1- |
| | | yl)nona-2,4,6,8-tetraenoic acid |
| Trichostatin | 58880-19-6 | 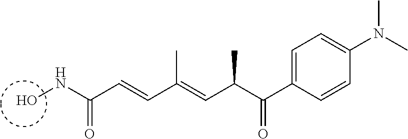 [SMILES: CC(/C=C/C(=O)NO)=C\[C@@H](C)C(=O)c1ccc(N(C)C)cc1] |
| | | (2E,4E,6R)-7-[4-(dimethylamino)phenyl]-N-hydroxy-4,6-dimethyl- |
| | | 7-oxohepta-2,4-dienamide |
| Tucatinib | 937263-43-9 | 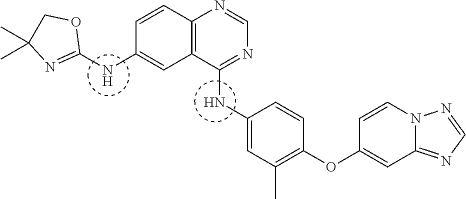 [SMILES: Cc1cc(Nc2ncnc3ccc(NC4=NC(C)(C)CO4)cc23)ccc1Oc1ccn2ncnc2c1] |
| | | 6-N-(4,4-dimethyl-5H-1,3-oxazol-2-yl)-4-N-[3-methyl-4-([1,2,4]triazolo |
| | | [1,5-a]pyridin-7-yloxy)phenyl]quinazoline-4,6-diamine |
| Tucidinostat | 1616493-44-7 | 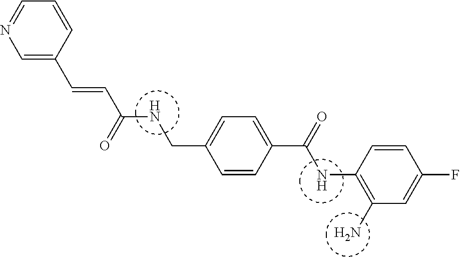 [SMILES: Nc1cc(F)ccc1NC(=O)c1ccc(CNC(=O)/C=C/c2cccnc2)cc1] |
| | | N-(2-amino-4-fluorophenyl)-4-[[[(E)-3-pyridin-3-ylprop-2-enoyl]amino- |
| | | methyl]benzamide |
| Tuvusertib | 1613200-51-3 | 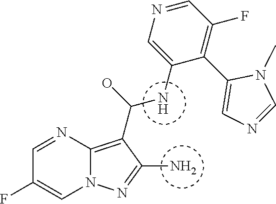 [SMILES: Cn1cncc1-c1c(F)cncc1NC(O)c1c(N)nn2cc(F)cnc12] |
| | | 2-amino-6-fluoro-N-[5-fluoro-4-(3-methyl- |
| | | imidazol-4-yl)pyridin-3-yl]pyrazolo[1,5-a] |
| | | pyrimidine-3-carboxamide |
| Ubenimex | 58970-76-6 | 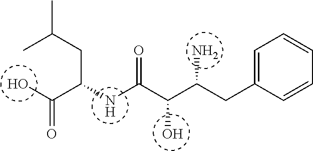 [SMILES: CC(C)C[C@H](NC(=O)[C@@H](O)[C@H](N)Cc1ccccc1)C(=O)O] |
| | | (2S)-2-[[(2S,3R)-3-amino-2-hydroxy-4-phenyl- |
| | | butanoyl]amino]-4-methylpentanoic acid |
| Umbralisib | 1532533-67-7 | 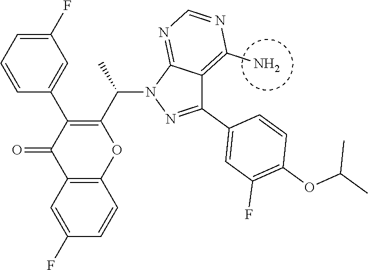 [SMILES: CC(C)Oc1ccc(-c2nn([C@@H](C)c3oc4ccc(F)cc4c(=O)c3-c3cccc(F)c3)c3ncnc(N)c23)cc1F] |
| | | 2-[(1S)-1-[4-amino-3-(3-fluoro-4-propan-2-yloxyphenyl)- |
| | | pyrazolo[3,4-d]pyrimidin-1-yl]ethyl]-6-fluoro-3-(3-fluoro- |
| | | phenyl)chromen-4-one |
| Uprosertib | 1047634-65-0 | 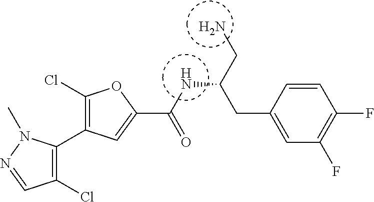 [SMILES: Cn1ncc(Cl)c1-c1cc(C(=O)N[C@H](CN)Cc2ccc(F)c(F)c2)oc1Cl] |
| | | N-[(2S)-1-amino-3-(3,4-difluorophenyl)propan-2-yl]-5-chloro- |
| | | 4-(4-chloro-2-methylpyrazol-3-yl)furan-2-carboxamide |
| USL311 | 1373268-67-7 | 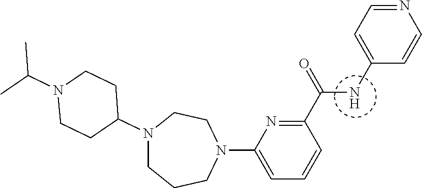 [SMILES: CC(C)N1CCC(N2CCCN(c3cccc(C(=O)Nc4ccncc4)n3)CC2)CC1] |
| | | 6-[4-(1-propan-2-ylpiperidin-4-yl)-1,4-diazepan-1-yl]-N-pyridin-4- |
| | | ylpyridine-2-carboxamide |
| Vactosertib | 1352608-82-2 | 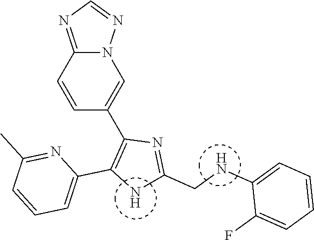 [SMILES: Cc1cccc(-c2[nH]c(CNc3ccccc3F)nc2-c2ccc3ncnn3c2)n1] |
| | | 2-fluoro-N-[[5-(6-methylpyridin-2-yl)-4-([1,2,4] |
| | | triazolo[1,5-a]pyridin-6-yl)-1H-imidazol-2-yl] |
| | | methyl]aniline |
| Valproic acid | 99-66-1 | 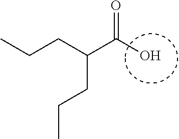 |
| | | 2-propylpentanoic acid |
| Valsartan | 137862-53-4 | 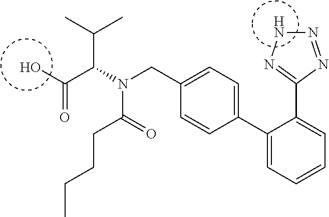 [SMILES: CCCCC(=O)N(Cc1ccc(-c2ccccc2-c2nn[nH]n2)cc1)[C@H](C(=O)O)C(C)C] |
| | | (2S)-3-methyl-2-[pentanoy]-[[4-[2-(2H-tetrazol-5-yl)- |
| | | phenyl]phenyl]methyl]amino]butanoic acid |
| Vandetanib | 443913-73-3 | 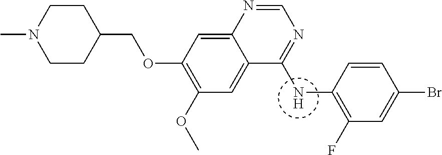 [SMILES: COc1cc2c(Nc3ccc(Br)cc3F)ncnc2cc1OCC1CCN(C)CC1] |
| | | N-(4-bromo-2-fluorophenyl)-6-methoxy-7-[(1-methylpiperidin-4-yl)- |
| | | methoxy]quinazolin-4-amine |
| Veliparib | 912444-00-9 | 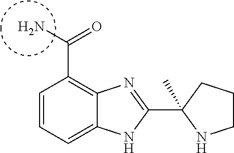 [SMILES: C[C@]1(c2nc3c(C(N)=O)cccc3[nH]2)CCCN1] |
| | | 2-[(2R)-2-methylpyrrolidin-2-yl]- |
| | | 1H-benzimidazole-4-carboxamide |
| Vemurafenib | 918504-65-1 | 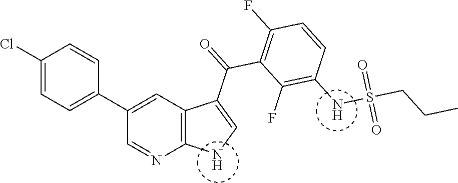 [SMILES: CCCS(=O)(=O)Nc1ccc(F)c(C(=O)c2c[nH]c3ncc(-c4ccc(Cl)cc4)cc23)c1F] |
| | | N-[3-[5-(4-chlorophenyl)-1H-pyrrolo[2,3-b]pyridine-3-carbonyl]-2,4- |
| | | difluorophenyl]propane-1-sulfonamide |
| Venetoclax | 1257044-40-8 | 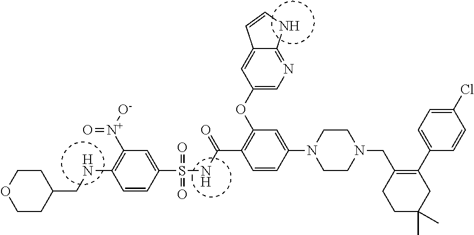 |
| | | 4-[4-[2-(4-chlorophenyl)-4,4-dimethylcyclohexen-1-yl]methyl]piperazin-1-yl]- |
| | | N-[3-nitro-4-(oxan-4-ylmethylamino)phenyl]sulfonyl-2-(1H-pyrrolo[2,3-b] |
| | | pyridin-5-yloxy)benzamide |
| Verteporfin | — | 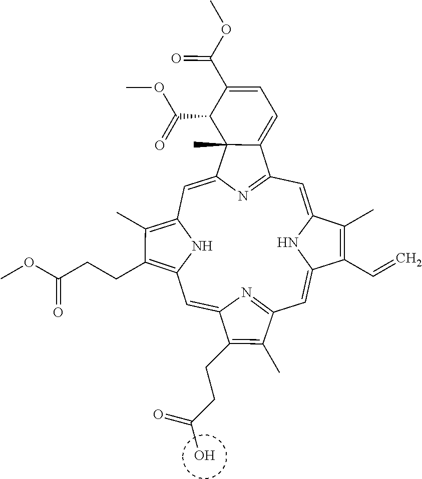 [SMILES: C=Cc1c(C)c2cc3nc(cc4[nH]c(cc5nc(cc1[nH]2)C(C)=C5CCC(=O)O)c(CCC(=O)OC)c4C)[C@@]1(C)C3=CC=C(C(=O)OC)[C@H]1C(=O)OC] |
| | | 3-[(23S,24R)-14-ethenyl-22,23-bis(methoxycarbonyl)-5-(3-methoxy- |
| | | 3-oxopropyl)-4,10,15,24-tetramethyl-25,26,27,28-tetrazahexacyclo |
| | | [16.6.1.1^{3,6}.1^{8,11}.11^{3,16}.0^{19,24}]octacosa-1,3,5,7,9,11(27),12,14,16,18(25), |
| | | 19,21-dodecaen-9-yl]propanoic acid; 3-[(23S,24R)-14-ethenyl-22,23- |
| | | bis(methoxycarbonyl)-9-(3-methoxy-3-oxopropyl)-4,10,15,24- |
| | | tetramethyl-25,26,27,28-tetrazahexacyclo[16.6.1.1^{3,6}.1^{8,11}.1^{13,16}.0^{19,24}] |
| | | octacosa-1,3,5,7,9,11(27),12,14,16,18(25),19,21-dodecaen-5-yl] |
| | | propanoic acid |
| Vismodegib | 879085-55-9 | 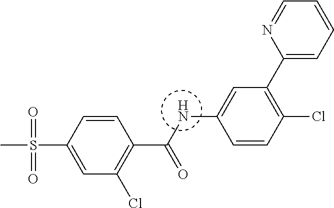 [SMILES: CS(=O)(=O)c1ccc(C(=O)Nc2ccc(Cl)c(-c3ccccn3)c2)c(Cl)c1] |
| | | 2-chloro-N-(4-chloro-3-pyridin-2-ylphenyl)-4-methyl- |
| | | sulfonylbenzamide |
| Vorinostat | 149647-78-9 | 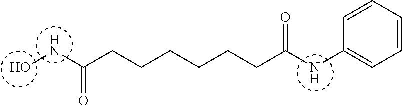 [SMILES: O=C(CCCCCCC(=O)Nc1ccccc1)NO] |
| | | N′-hydroxy-N-phenyloctanediamide |
| WRG-28 | 1913291-02-7 | 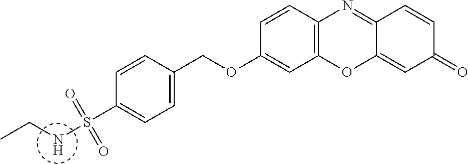 [SMILES: CCNS(=O)(=O)c1ccc(COc2ccc3nc4ccc(=O)cc-4oc3c2)cc1] |
| | | N-Ethyl-4-(((3-oxo-3H-phenoxazin-7-yl)oxy)methyl)benzenesulfonamide |
| WZ811 | 55778-02-4 | 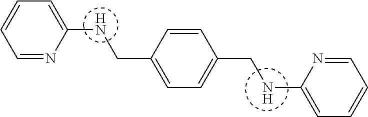 [SMILES: c1ccc(NCc2ccc(CNc3ccccn3)cc2)nc1] |
| | | N-[[4-[(pyridin-2-ylamino)methyl]phenyl]methyl]pyridin- |
| | | 2-amine |
| Xevinapant | 1071992-99-8 | 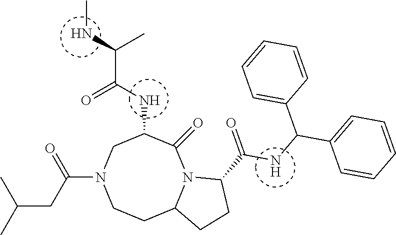 [SMILES: CN[C@@H](C)C(=O)N[C@H]1CN(C(=O)CC(C)C)CCC2CC[C@@H](C(=O)NC(c3ccccc3)c3ccccc3)N2C1=O] |
| | | (5S,8S,10aR)-N-benzhydryl-5-[[(2S)-2-(methylamino)propanoyl] |
| | | amino]-3-(3-methylbutanoyl)-6-oxo-1,2,4,5,8,9,10,10a-octahydro- |
| | | pyrrolo[1,2-a][1,5]diazocine-8-carboxamide |
| Zandelisib | 1401436-95-0 | 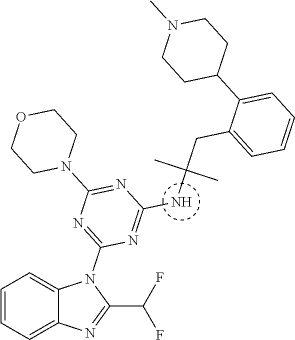 [SMILES: CN1CCC(c2ccccc2CC(C)(C)Nc2nc(N3CCOCC3)nc(-n3c(C(F)F)nc4ccccc43)n2)CC1] |
| | | 4-[2-(difluoromethyl)benzimidazol-1-yl]-N-[2- |
| | | methyl-1-[2-(1-methylpiperidin-4-yl)phenyl]pro- |
| | | pan-2-yl]-6-morpholin-4-yl-1,3,5-triazin-2-amine |
| Zanubrutinib | 1691249-45-2 | 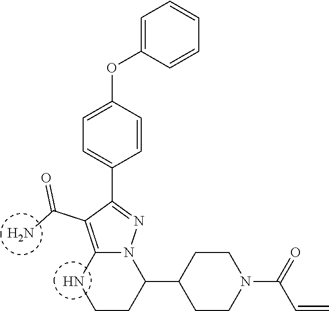 [SMILES: C=CC(=O)N1CCC(C2CCNc3c(C(N)=O)c(-c4ccc(Oc5ccccc5)cc4)nn32)CC1] |
| | | (7S)-2-(4-phenoxyphenyl)-7-(1-prop-2-enoylpiperidin- |
| | | 4-yl)-4,5,6,7-tetrahydropyrazolo[1,5-a]pyrimidine- |
| | | 3-carboxamide |
| ZM447439 | 331771-20-1 | 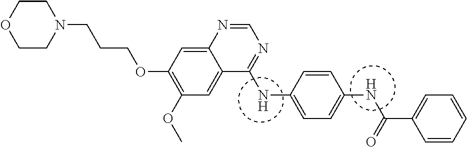 [SMILES: COc1cc2c(Nc3ccc(NC(=O)c4ccccc4)cc3)ncnc2cc1OCCCN1CCOCC1] |
| | | N-[4-[[6-methoxy-7-(3-morpholin-4-ylpropoxy)quinazolin-4-yl]amino]phen- |
| | | yl]benzamide |

TABLE 2
Mainly cytotoxic chemotherapeutic compounds (Ctᵢ)

| Name | CAS-No. | Structure and IUPAC Name |
|---|---|---|
| Abiraterone | 154229-19-3 | 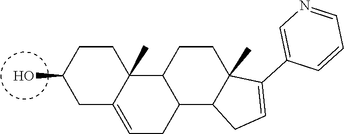 [SMILES: C[C@]12CC[C@H](O)CC1=CCC1C2CC[C@]2(C)C(c3cccnc3)=CCC12] |
| | | (3S,8R,9S,10R,13S,14S)-10,13-dimethyl-17-pyridin-3-yl-2,3,4,7,8,9,11,12,14,15- |
| | | decahydro-1H-cyclopenta[a]phenanthren-3-ol |
| Aclarubicin | 57576-44-0 | 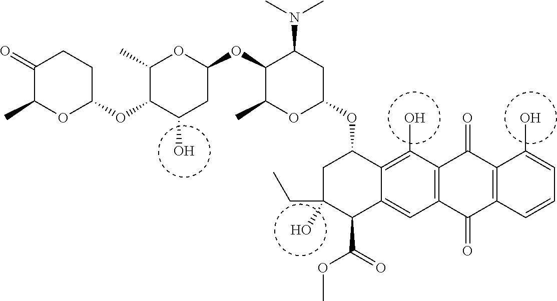 |
| | | methyl (1R,2R,4S)-4-[(2R,4S,5S,6S)-4-(dimethylamino)-5-[(2S,4S,5S,6S)-4-hydroxy- |
| | | 6-methyl-5-[(2R,6S)-6-methyl-5-oxooxan-2-yl]oxyoxan-2-yl]oxy-6-methyloxan-2- |
| | | yl]oxy-2-ethyl-2,5,7-trihydroxy-6,11-dioxo-3,4-dihydro-1H-tetracene-1-carboxylate |
| Adozelesin | 110314-48-2 | 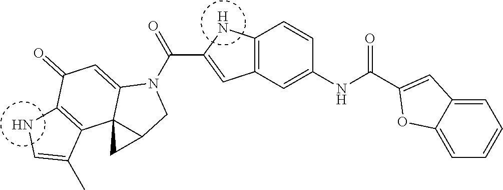 |
| | | N-[2-[(1R,12S)-3-methyl-7-oxo-5,10-diazatetracyclo[7.4.0.0^{1,12}.0^{2,6}]trideca-2(6),3,8- |
| | | triene-10-carbonyl]-1H-indol-5-yl]-1-benzofuran-2-carboxamide |
| Alrestatin | 51411-04-2 | 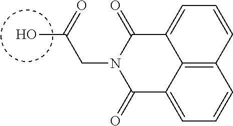 [SMILES: O=C(O)CN1C(=O)c2cccc3cccc(c23)C1=O] |
| | | 2-(1,3-dioxobenzo[de]isoquinolin-2-yl)acetic acid |
| Amanitin | 23109-05-9 | 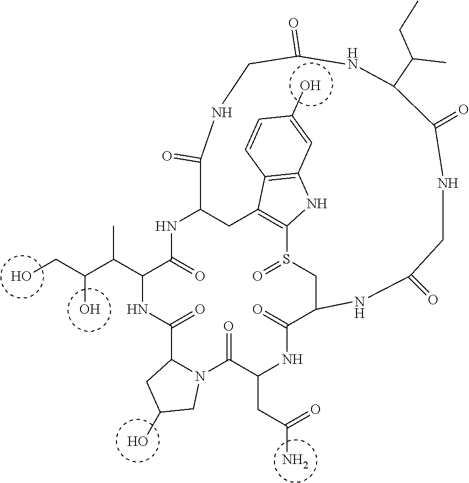 |
| | | 2-[34-butan-2-yl-13-(3,4-dihydroxybutan-2-yl)-8,22-dihydroxy- |
| | | 2,5,11,14,27,30,33,36,39-nonaoxo-27λ⁴-thia-3,6,12,15,25,29,32,35,38- |
| | | nonazapentacyclo[14.12.11.0^{6,10}.0^{18,26}.0^{19,24}]nonatriaconta-18(26),19(24),20,22- |
| | | tetraen-4-yl]acetamide |
| Amrubicin | 110267-81-7 | 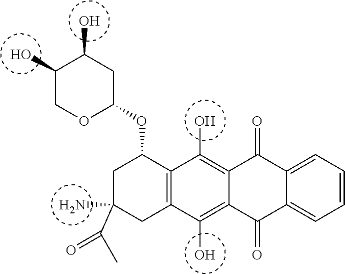 |
| | | (7S,9S)-9-acetyl-9-amino-7-[(2S,4S,5R)-4,5-dihydroxyoxan-2-yl]oxy-6,11- |
| | | dihydroxy-8,10-dihydro-7H-tetracene-5,12-dione |
| Anthramycin | 4803-27-4 | 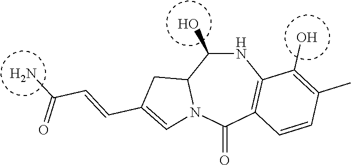 [SMILES: Cc1ccc2c(c1O)N[C@H](O)C1CC(/C=C/C(N)=O)=CN1C2=O] |
| | | (E)-3-[(6R,6aS)-4,6-dihydroxy-3-methyl-11-oxo-5,6,6a,7-tetrahydropyrrolo[2,1- |
| | | c][1,4]benzodiazepin-8-yl]prop-2-enamide |
| Arenastatin | — | 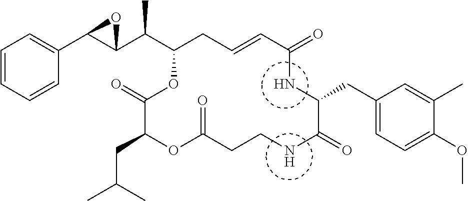 [SMILES: COc1ccc(C[C@H]2NC(=O)/C=C/C[C@@H]([C@H](C)[C@H]3O[C@@H]3c3ccccc3)OC(=O)[C@H](CC(C)C)OC(=O)CCNC2=O)cc1C] |
| | | (3S,10R,13E,16S)-10-[(4-methoxyphenyl)methyl]-3-(2-methylpropyl)-16-[(1S)-1- |
| | | [[(2R,3R)-3-phenyloxiran-2-yl]ethyl]-1,4-dioxa-8,11-diazacyclohexadec-13-ene- |
| | | 2,5,9,12-tetrone |
| Bizelesin | 129655-21-6 | 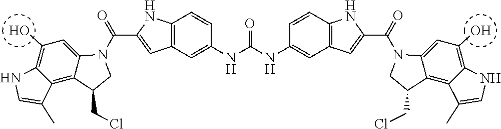 [SMILES: Cc1c[nH]c2c(O)cc3c(c12)[C@H](CCl)CN3C(=O)c1cc2cc(NC(=O)Nc3ccc4[nH]c(C(=O)N5C[C@@H](CCl)c6c5cc(O)c5[nH]cc(C)c65)cc4c3)ccc2[nH]1] |
| | | 1,3-bis[2-[(8S)-8-(chloromethyl)-4-hydroxy-1-methyl-7,8-dihydro-3H-pyrrolo[3,2- |
| | | e]indole-6-carbonyl]-1H-indol-5-yl]urea |
| Bleomycin | 11056-06-7 | 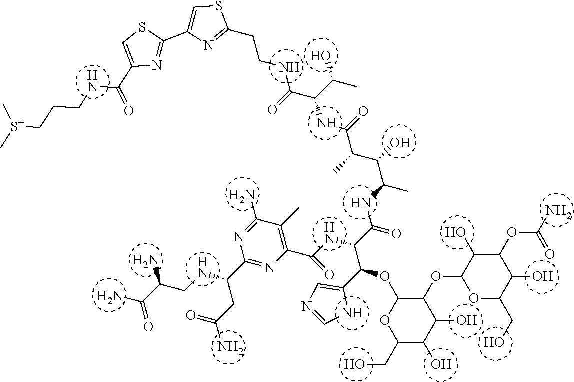 [SMILES: Cc1c(N)nc([C@H](CC(N)=O)NC[C@H](N)C(N)=O)nc1C(=O)N[C@H](C(=O)N[C@H](C)[C@@H](O)[C@H](C)C(=O)N[C@H](C(=O)NCCc1nc(-c2nc(C(=O)NCCC[S+](C)C)cs2)cs1)[C@@H](C)O)[C@@H](OC1OC(CO)C(O)C(O)C1OC1OC(CO)C(O)C(OC(N)=O)C1O)c1cnc[nH]1] |
| | | 3-[[2-[2-[2-[[(2S,3R)-2-[[(2S,3S,4R)-4-[[(2S,3R)-2-[[6-amino-2-[(1S)-3-amino-1- |
| | | [[[(2S)-2,3-diamino-3-oxopropyl]amino]-3-oxopropyl]-5-methylpyrimidine-4- |
| | | carbonyl]amino]-3-[3-[4-carbamoyloxy-3,5-dihydroxy-6-(hydroxymethyl)oxan-2- |
| | | yl]oxy-4,5-dihydroxy-6-(hydroxymethyl)oxan-2-yl]oxy-3-(1H-imidazol-5- |
| | | yl)propanoyl]amino]-3-hydroxy-2-methylpentanoyl]amino]-3- |
| | | hydroxybutanoyl]amino]ethyl]-1,3-thiazol-4-yl]-1,3-thiazole-4- |
| | | carbonyl]amino]propyl-dimethylsulfanium |
| Camptothecin | 7689-03-4 | 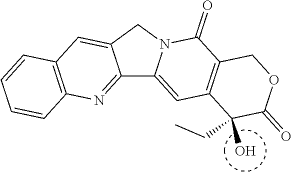 |
| | | (19S)-19-ethyl-19-hydroxy-17-oxa-3,13- |
| | | diazapentacyclo[11.8.0.0^{2,11}.0^{4,9}.0^{15,20}]henicosa-1(21),2,4,6,8,10,15(20)-heptaene- |
| | | 14,18-dione |
| Capecitabine | 154361-50-9 | 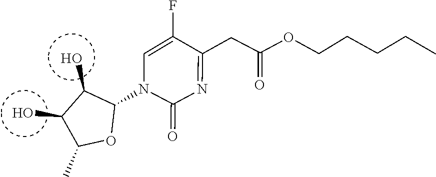 [SMILES: CCCCCOC(=O)Cc1nc(=O)n([C@@H]2O[C@H](C)[C@@H](O)[C@H]2O)cc1F] |
| | | pentyl N-[1-[(2R,3R,4S,5R)-3,4-dihydroxy-5-methyloxolan-2-yl]-5-fluoro-2- |
| | | oxopyrimidin-4-yl]carbamate |
| Carzelesin | 119813-10-4 | 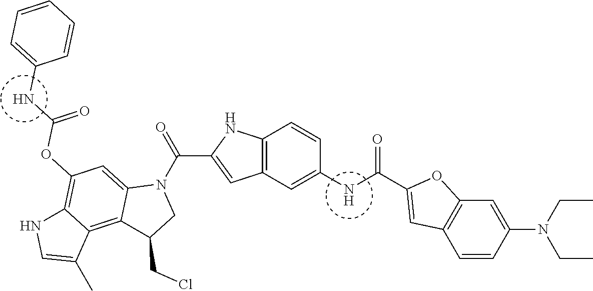 [SMILES: CCN(CC)c1ccc2cc(C(=O)Nc3ccc4[nH]c(C(=O)N5C[C@@H](CCl)c6c5cc(OC(=O)Nc5ccccc5)c5[nH]cc(C)c65)cc4c3)oc2c1] |
| | | [(8S)-8-(chloromethyl)-6-[5-[[6-(diethylamino)-1-benzofuran-2-carbonyl]amino]-1H- |
| | | indole-2-carbonyl]-1-methyl-7,8-dihydro-3H-pyrrolo[3,2-e]indol-4-yl] N- |
| | | phenylcarbamate |
| CC-1065 | 69866-21-3 | 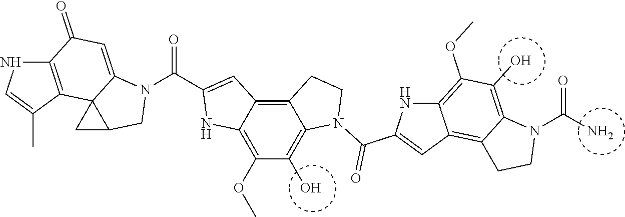 [SMILES: COc1c(O)c2c(c3cc(C(=O)N4CCc5c4c(O)c(OC)c4[nH]c(C(=O)N6CC7CC78C6=CC(=O)c6[nH]cc(C)c68)cc54)[nH]c13)CCN2C(N)=O] |
| | | 5-hydroxy-2-[5-hydroxy-4-methoxy-2-(3-methyl-7-oxo-5,10- |
| | | diazatetracyclo[7.4.0.0^{1,12}.0^{2,6}]trideca-2(6),3,8-triene-10-carbonyl)-7,8-dihydro-3H- |
| | | pyrrolo[3,2-e]indole-6-carbonyl]-4-methoxy-7,8-dihydro-3H-pyrrolo[3,2-e]indole-6- |
| | | carboxamide |
| Chaconine | 20562-03-2 | 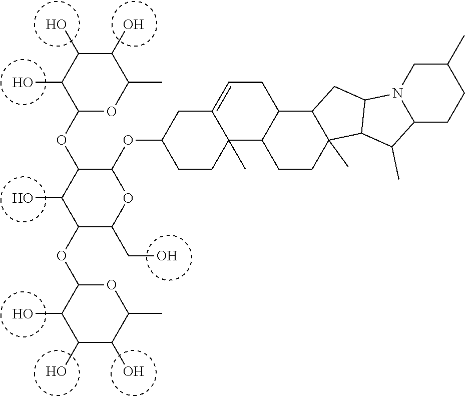 [SMILES: CC1CCC2C(C)C3C(CC4C5CC=C6CC(OC7OC(CO)C(OC8OC(C)C(O)C(O)C8O)C(O)C7OC7OC(C)C(O)C(O)C7O)CCC6(C)C5CCC43C)N2C1] |
| | | 2-[4-hydroxy-2-(hydroxymethyl)-6-[(10,14,16,20-tetramethyl-22- |
| | | azahexacyclo[12.10.0.0^{2,11}.0^{5,10}.0^{15,23}.0^{17,22}]tetracos-4-en-7-yl)oxy]-5-(3,4,5- |
| | | trihydroxy-6-methyloxan-2-yl)oxyoxan-3-ylloxy-6-methyloxane-3,4,5-triol |
| Chlorambucil | 305-03-3 | 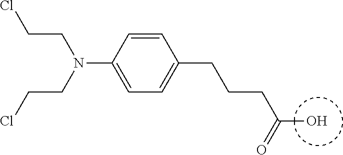 [SMILES: O=C(O)CCCc1ccc(N(CCCl)CCCl)cc1] |
| | | 4-[4-[bis(2-chloroethyl)amino]phenyl]butanoic acid |
| Cryptophycin-24 | — | 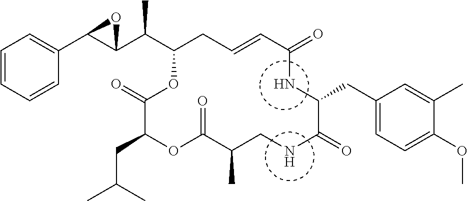 [SMILES: COc1ccc(C[C@H]2NC(=O)/C=C/C[C@@H]([C@H](C)[C@H]3O[C@@H]3c3ccccc3)OC(=O)[C@H](CC(C)C)OC(=O)[C@H](C)CNC2=O)cc1C] |
| | | 10-[(3-chloro-4-methoxyphenyl)methyl]-6-methyl-3-(2-methylpropyl)-16-[1-(3- |
| | | phenyloxiran-2-yl)ethyl]-1,4-dioxa-8,11-diazacyclohexadec-13-ene-2,5,9,12-tetrone |
| Cyclophosphamide | 50-18-0 | 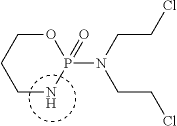 |
| | | N,N-bis(2-chloroethyl)-2-oxo-1,3,2λ⁵-oxazaphosphinan-2-amine |
| Cytarabine | 147-94-4 | 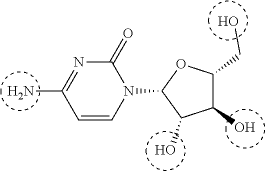 [SMILES: Nc1ccn([C@@H]2O[C@H](CO)[C@@H](O)[C@@H]2O)c(=O)n1] |
| | | 4-amino-1-[(2R,3S,4S,5R)-3,4-dihydroxy-5-(hydroxymethyl)oxolan-2-yl]pyrimidin- |
| | | 2-one |
| Dacarbazine | 4342-03-4 | 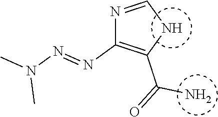 |
| | | 4-[(E)-dimethylaminodiazenyl]-1H-imidazole-5-carboxamide |
| Dactinomycin | 50-76-0 | 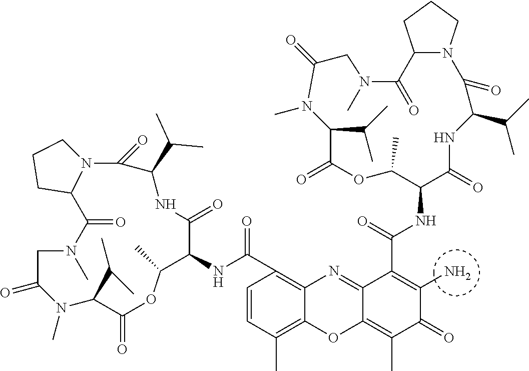 [SMILES: Cc1c2oc3c(C)ccc(C(=O)N[C@@H]4C(=O)N[C@H](C(C)C)C(=O)N5CCCC5C(=O)N(C)CC(=O)N(C)[C@@H](C(C)C)C(=O)O[C@@H]4C)c3nc-2c(C(=O)N[C@@H]2C(=O)N[C@H](C(C)C)C(=O)N3CCCC3C(=O)N(C)CC(=O)N(C)[C@@H](C(C)C)C(=O)O[C@@H]2C)c(N)c1=O] |
| | | 2-amino-4,6-dimethyl-3-oxo-1-N,9-N-bis[(3R,6S,7R,10S,16S)-7,11,14-trimethyl- |
| | | 2,5,9,12,15-pentaoxo-3,10-di(propan-2-yl)-8-oxa-1,4,11,14- |
| | | tetrazabicyclo[14.3.0]nonadecan-6-yl]phenoxazine-1,9-dicarboxamide |
| Daunorubicin | 20830-81-3 | 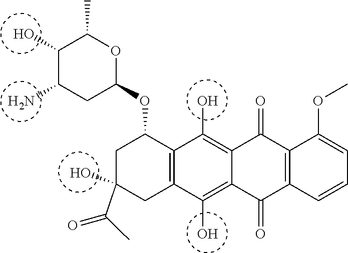 |
| | | (7S,9S)-9-acetyl-7-[(2R,4S,5S,6S)-4-amino-5-hydroxy-6-methyloxan-2-yl]oxy- |
| | | 6,9,11-trihydroxy-4-methoxy-8,10-dihydro-7H-tetracene-5,12-dione |
| DAVLBH | 55383-37-4 | 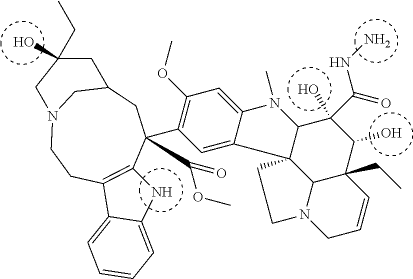 |
| | | methyl (13S,15R,17S)-17-ethyl-13-[(1R,9R,10S,11R,12R,19R)-12-ethyl-10- |
| | | (hydrazinecarbonyl)-10,11-dihydroxy-5-methoxy-8-methyl-8,16-diazapenta- |
| | | cyclo[10.6.1.0^{1,9}.0^{2,7}.0^{16,19}]nonadeca-2,4,6,13-tetraen-4-yl]-17-hydroxy-1,11- |
| | | diazatetracyclo[13.3.1.0^{4,12}.0^{5,10}]nonadeca-4(12),5,7,9-tetraene-13-carboxylate |
| Deruxtecan | 1599440-13-7 | 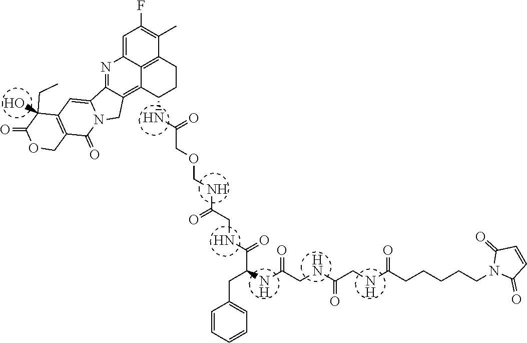 |
| | | 6-(2,5-dioxopyrrol-1-yl)-N-[2-[2-[(2S)-1-[2-[2-[(10S,23S)-10-ethyl-18-fluoro-10- |
| | | hydroxy-19-methyl-5,9-dioxo-8-oxa-4,15- |
| | | diazahexacyclo[14.7.1.0^{2,14}.0^{4,13}.0^{6,11}.0^{20,24}]tetracosa-1,6(11),12,14,16,18,20(24)- |
| | | heptaen-23-yl]amino]-2-oxoethoxy]methylamino]-2-oxoethyl]amino]-1-oxo-3- |
| | | phenylpropan-2-yl]amino]-2-oxoethyl]amino]-2-oxoethyl]hexanamide |
| Dexamethasone | 50-02-2 | 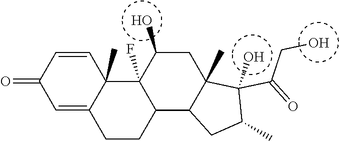 |
| | | (8S,9R,10S,11S,13S,14S,16R,17R)-9-fluoro-11,17-dihydroxy-17-(2-hydroxyacetyl)- |
| | | 10,13,16-trimethyl-6,7,8,11,12,14,15,16-octahydrocyclopenta[a]phenanthren-3-one |
| Dichloroacetic acid | 79-43-6 | 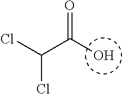 |
| | | 2,2-dichloroacetic acid |
| Dimethyl- SGD-1882 | — | 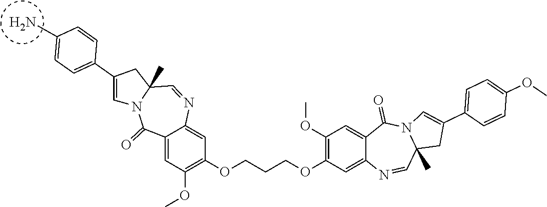 [SMILES: COc1ccc(C2=CN3C(=O)c4cc(OC)c(OCCCOc5cc6c(cc5OC)C(=O)N5C=C(c7ccc(N)cc7)C[C@]5(C)C=N6)cc4N=C[C@@]3(C)C2)cc1] |
| | | (6aR)-3-[3-[[(6aR)-2-methoxy-8-(4-methoxyphenyl)-6a-methyl-11-oxo-7H- |
| | | pyrrolo[2,1-c][1,4]benzodiazepin-3-yl]oxy]propoxy]-8-(4-aminophenyl)-2-methoxy- |
| | | 6a-methyl-7H-pyrrolo[2,1-c][1,4]benzodiazepin-11-one |
| Docetaxel | 114977-28-5 | 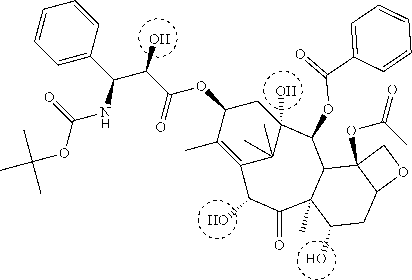 |
| | | [(1S,2S,3R,4S,7R,9S,10S,12R,15S)-4-acetyloxy-1,9,12-trihydroxy-15-[(2R,3S)-2- |
| | | hydroxy-3-[(2-methylpropan-2-yl)oxycarbonylamino]-3-phenylpropanoyl]oxy- |
| | | 10,14,17,17-tetramethyl-11-oxo-6-oxatetracyclo[11.3.1.0^{3,10}.0^{4,7}]heptadec-13-en-2- |
| | | yl]benzoate |
| Dolastatin-10 | 110417-88-4 | 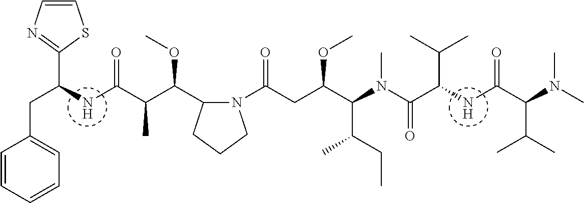 [SMILES: CC[C@H](C)[C@@H]([C@@H](CC(=O)N1CCCC1[C@H](OC)[C@@H](C)C(=O)N[C@@H](Cc1ccccc1)c1nccs1)OC)N(C)C(=O)[C@@H](NC(=O)[C@H](C(C)C)N(C)C)C(C)C] |
| | | (2S)-2-[[(2S)-2-(dimethylamino)-3-methylbutanoyl]amino]-N-[(3R,4S,5S)-3- |
| | | methoxy-1-[(2S)-2-[(1R,2R)-1-methoxy-2-methyl-3-oxo-3-[(1S)-2-phenyl-1-(1,3- |
| | | thiazol-2-yl)ethyl]amino]propyl]pyrrolidin-1-yl]-5-methyl-1-oxoheptan-4-yl]-N,3- |
| | | dimethylbutanamide |
| Doxorubicin | 23214-92-8 | 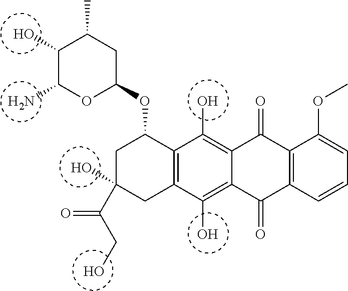 |
| | | (7S,9S)-7-[(2R,4S,5S,6S)-4-amino-5-hydroxy-6-methyloxan-2-yl]oxy-6,9,11- |
| | | trihydroxy-9-(2-hydroxyacetyl)-4-methoxy-8,10-dihydro-7H-tetracene-5,12-dione |
| Duocarmycin A | 118292-34-5 | 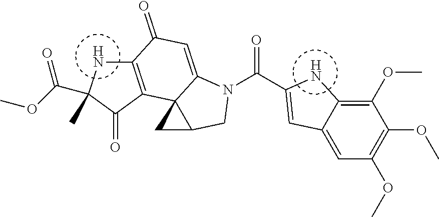 |
| | | methyl (1R,4R,12S)-4-methyl-3,7-dioxo-10-(5,6,7-trimethoxy-1H-indole-2- |
| | | carbonyl)-5,10-diazatetracyclo[7.4.0.0^{1,12}.0^{2,6}]trideca-2(6),8-diene-4-carboxylate |
| Duocarmycin B1 | 124325-93-5 | 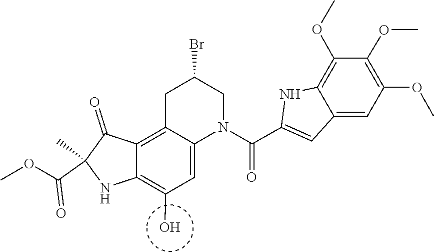 |
| | | methyl (2R,8S)-8-bromo-4-hydroxy-2-methyl-1-oxo-6-(5,6,7-trimethoxy-1H-indole- |
| | | 2-carbonyl)-3,7,8,9-tetrahydropyrrolo[3,2-f]quinoline-2-carboxylate |
| Duocarmycin B2 | 124325-94-6 | 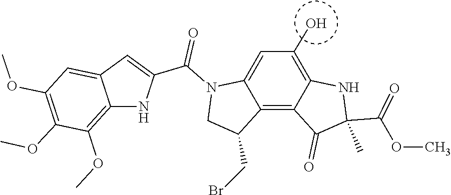 |
| | | methyl (2R,8S)-8-(bromomethyl)-4-hydroxy-2-methyl-1-oxo-6-(5,6,7-trimethoxy- |
| | | 1H-indole-2-carbonyl)-7,8-dihydro-3H-pyrrolo[3,2-e]indole-2-carboxylate |
| Duocarmycin C1 | — | 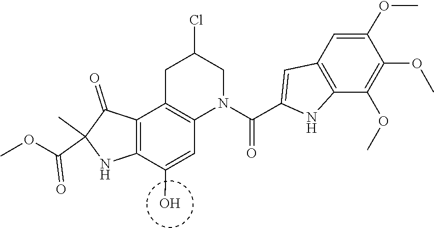 |
| Duocarmycin C2 | — | 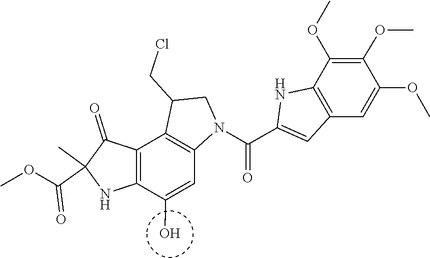 |
| Duocarmycin D | — | 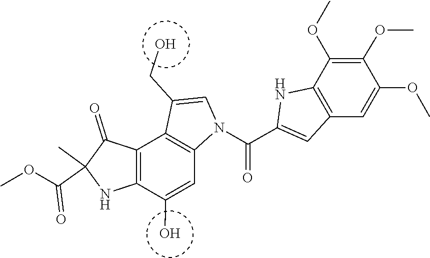 |
| Duocarmycin GA | — | 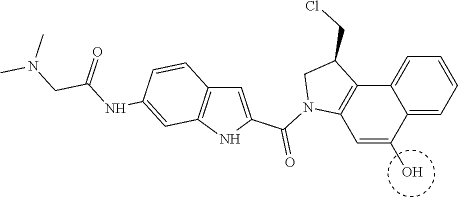 [SMILES: CN(C)CC(=O)Nc1ccc2cc(C(=O)N3C[C@@H](CCl)c4c3cc(O)c3ccccc43)[nH]c2c1] |
| | | N-[2-[(1S)-1-(chloromethyl)-5-hydroxy-1,2-dihydrobenzo[e]indole-3-carbonyl]-1H- |
| | | indol-6-yl]-2-(dimethylamino)acetamide |
| Duocarmycin SA | 130288-24-3 | 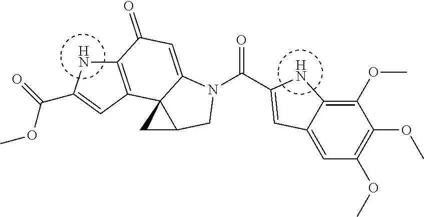 |
| | | methyl (1R,12S)-7-oxo-10-(5,6,7-trimethoxy-1H-indole-2-carbonyl)-5,10- |
| | | diazatetracyclo[7.4.0.0^{1,12}.0^{2,6}]trideca-2(6),3,8-triene-4-carboxylate |
| Emetine | 483-18-1 | 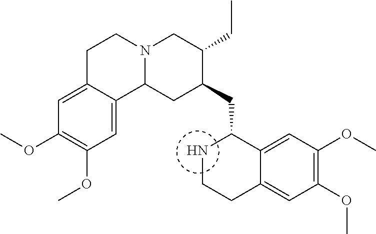 |
| | | (2S,3R,11bS)-2-[(1R)-6,7-dimethoxy-1,2,3,4-tetrahydroisoquinolin-1-yl]methyl]-3- |
| | | ethyl-9,10-dimethoxy-2,3,4,6,7,11b-hexahydro-1H-benzo[a]quinolizine |
| Epirubicin | 56420-45-2 | 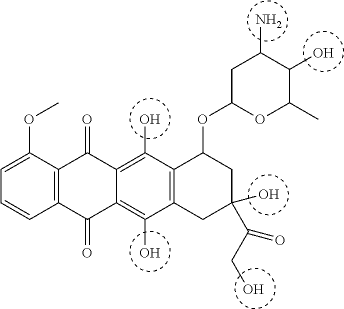 |
| | | (7S,9S)-7-[(2R,4S,5R,6S)-4-amino-5-hydroxy-6-methyloxan-2-yl]oxy-6,9,11- |
| | | trihydroxy-9-(2-hydroxyacetyl)-4-methoxy-8,10-dihydro-7H-tetracene-5,12-dione |
| Eribulin | 253128-41-5 | 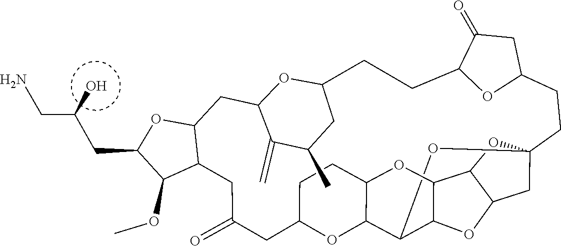 [SMILES: C=C1C2CC3O[C@H](C[C@H](O)CN)[C@H](OC)C3CC(=O)CC3CCC4OC5C6O[C@]7(CCC8CC(=O)C(CCC(C[C@H]1C)O2)O8)CC6OC5C(O7)C4O3] |
| | | (1S,3S,6S,9S,12S,14R,16R,18S,20R,21R,22S,26R,29S,31R,32S,33R,35R,36S)-20- |
| | | [(2S)-3-amino-2-hydroxypropyl]-21-methoxy-14-methyl-8,15-dimethylidene- |
| | | 2,19,30,34,37,39,40,41- |
| | | octaoxanonacyclo[24.9.2.1^{3,32}.1^{3,33}.1^{6,9}.1^{12,16}.0^{18,22}.0^{29,36}.0^{31,35}]hentetracontan-24-one |
| Etoposide | 33419-42-0 | 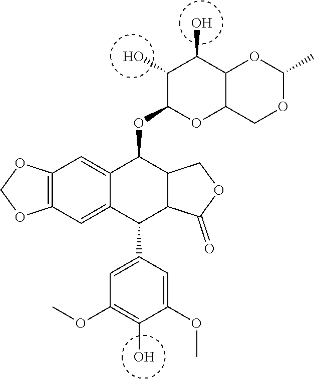 [SMILES: COc1cc([C@@H]2c3cc4c(cc3[C@@H](O[C@@H]3OC5CO[C@@H](C)OC5[C@H](O)[C@H]3O)C3COC(=O)C32)OCO4)cc(OC)c1O] |
| | | (5S,5aR,8aR,9R)-5-[[(2R,4aR,6R,7R,8R,8aS)-7,8-dihydroxy-2-methyl-4,4a,6,7,8,8a- |
| | | hexahydropyrano[3,2-d][1,3]dioxin-6-yl]oxy]-9-(4-hydroxy-3,5-dimethoxyphenyl)- |
| | | 5a,6,8a,9-tetrahydro-5H-[2]benzofuro[6,5-f][1,3]benzodioxol-8-one |
| Floxuridine | 50-91-9 | 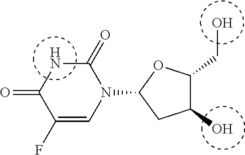 [SMILES: O=c1[nH]c(=O)n([C@H]2C[C@H](O)[C@@H](CO)O2)cc1F] |
| | | 5-fluoro-1-[(2R,4S,5R)-4-hydroxy-5-(hydroxymethyl)oxolan-2-yl]pyrimidine-2,4- |
| | | dione |
| Fludarabine | 21679-14-1 | 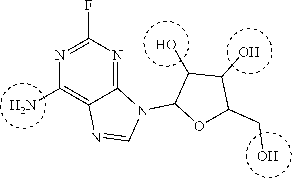 [SMILES: Nc1nc(F)nc2c1ncn2C1OC(CO)C(O)C1O] |
| | | (2R,3S,4S,5R)-2-(6-amino-2-fluoropurin-9-yl)-5-(hydroxymethyl)oxolane-3,4-diol |
| Fluorouracil | 51-21-8 | 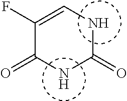 |
| | | 5-fluoro-1H-pyrimidine-2,4-dione |
| Flutamide | 13311-84-7 | 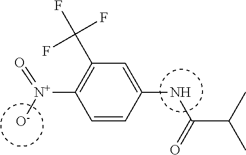 [SMILES: CC(C)C(=O)Nc1ccc([N+](=O)[O-])c(C(F)(F)F)c1] |
| | | 2-methyl-N-[4-nitro-3-(trifluoromethyl)phenyl]propanamide |
| Fulvestrant | 129453-61-8 | 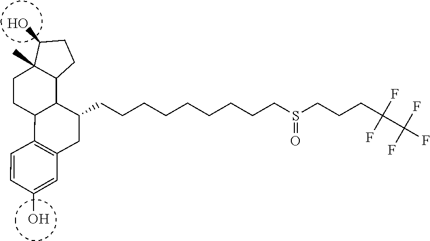 [SMILES: C[C@]12CCC3c4ccc(O)cc4C[C@@H](CCCCCCCCCS(=O)CCCC(F)(F)C(F)(F)F)C3C1CC[C@@H]2O] |
| | | (7R,8R,9S,13S,14S,17S)-13-methyl-7-[9-(4,4,5,5,5-pentafluoropentylsulfinyl)nonyl]- |
| | | 6,7,8,9,11,12,14,15,16, 17-decahydrocyclopenta[a]phenanthrene-3,17-diol |
| Gemcitabine | 95058-81-4 | 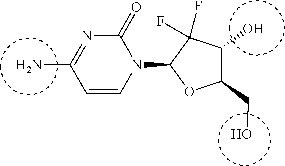 [SMILES: Nc1ccn([C@@H]2O[C@H](CO)[C@@H](O)C2(F)F)c(=O)n1] |
| | | 4-amino-1-[(2R,4R,5R)-3,3-difluoro-4-hydroxy-5-(hydroxymethyl)oxolan-2- |
| | | yllpyrimidin-2-one |
| Idarubicin | 58957-92-9 | 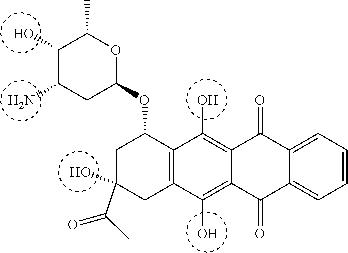 |
| | | (7S,9S)-9-acetyl-7-[(2R,4S,5S,6S)-4-amino-5-hydroxy-6-methyloxan-2-yl]oxy- |
| | | 6,9,11-trihydroxy-8,10-dihydro-7H-tetracene-5,12-dione |
| Ifosfamide | 3778-73-2 | 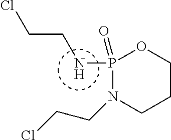 [SMILES: O=P1(NCCCl)OCCCN1CCCl] |
| | | N,3-bis(2-chloroethyl)-2-oxo-1,3,2λ⁵-oxazaphosphinan-2-amine |
| Irinotecan | 97682-44-5 | 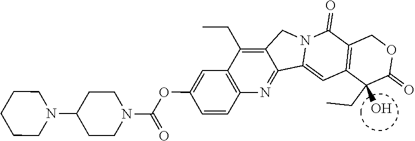 |
| | | [(19S)-10,19-diethyl-19-hydroxy-14,18-dioxo-17-oxa-3,13- |
| | | diazapentacyclo[11.8.0.02,11.04,9.015,20]henicosa-1(21),2,4(9),5,7,10,15(20)-heptaen-7- |
| | | yl] 4-piperidin-1-ylpiperidine-1-carboxylate |
| L-Asparaginase | 9015-68-3 | 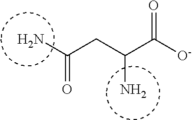 |
| | | 2,4-diamino-4-oxobutanoate |
| Lomustine | 13010-47-4 | 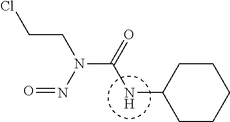 [SMILES: O=NN(CCCl)C(=O)NC1CCCCC1] |
| | | 1-(2-chloroethyl)-3-cyclohexyl-1-nitrosourea |
| Melphalan | 148-82-3 | 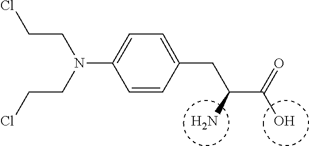 [SMILES: N[C@@H](Cc1ccc(N(CCCl)CCCl)cc1)C(=O)O] |
| | | (2S)-2-amino-3-[4-[bis(2-chloroethyl)amino]phenyl]propanoic acid |
| Mertansine | 139504-50-0 | 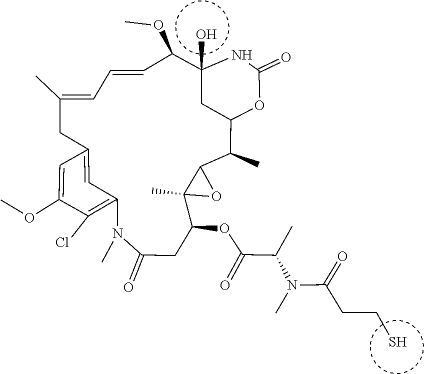 |
| | | [(1S,2R,3S,5S,6S,16E,18E,20R,21S)-11-chloro-21-hydroxy-12,20-dimethoxy- |
| | | 2,5,9,16-tetramethyl-8,23-dioxo-4,24-dioxa-9,22- |
| | | diazatetracyclo[19.3.1.1^{10,14}.0^{3,5}]hexacosa-10,12,14(26),16,18-pentaen-6-yl] (2S)-2- |
| | | [methyl(3-sulfanylpropanoyl)amino]propanoate |
| Methotrexate | 59-05-2 | 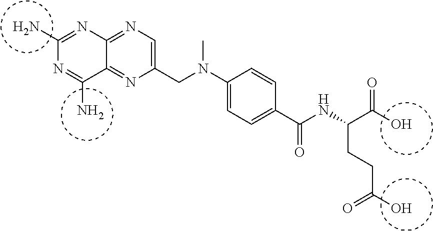 [SMILES: CN(Cc1cnc2nc(N)nc(N)c2n1)c1ccc(C(=O)N[C@@H](CCC(=O)O)C(=O)O)cc1] |
| | | (2S)-2-[4-[(2,4-diaminopteridin-6-yl)methyl- |
| | | methylamino]benzoyl]amino]pentanedioic acid |
| Milataxel | 352425-37-7 | 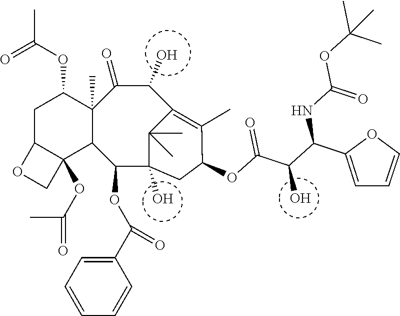 |
| | | [(1S,2S,3R,4S,7R,9S,10S,12R,15S)-4-acetyloxy-15-[(2R,3R)-3-(furan-2-yl)-2- |
| | | hydroxy-3-[(2-methylpropan-2-yl)oxycarbonylamino]propanoyl]oxy-1,12- |
| | | dihydroxy-10,14,17,17-tetramethyl-11-oxo-9-propanoyloxy-6- |
| | | oxatetracyclo[11.3.1.0^{3,10}.0^{4,7}]heptadec-13-en-2-yl] benzoate |
| Mitoxantrone | 65271-80-9 | 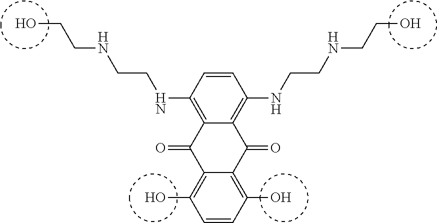 [SMILES: O=C1c2c(O)ccc(O)c2C(=O)c2c(NCCNCCO)ccc(NCCNCCO)c21] |
| | | 1,4-dihydroxy-5,8-bis[2-(2-hydroxyethylamino)ethylamino]anthracene-9,10-dione |
| Monomethyl Auristatin E | 474645-27-7 | 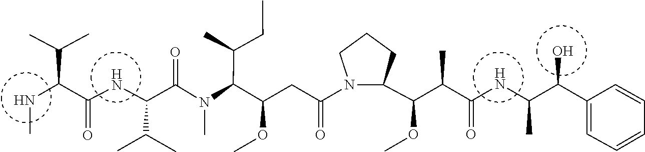 [SMILES: CC[C@H](C)[C@@H]([C@@H](CC(=O)N1CCC[C@H]1[C@H](OC)[C@@H](C)C(=O)N[C@H](C)[C@@H](O)c1ccccc1)OC)N(C)C(=O)[C@@H](NCC(=O)[C@@H](NC)C(C)C)C(C)C] |
| | | (2S)-N-[(2S)-1-[[(3R,4S,5S)-1-[(2S)-2-[(1R,2R)-3-[[(1S,2R)-1-hydroxy-1- |
| | | phenylpropan-2-yl]amino]-1-methoxy-2-methyl-3-oxopropyl]pyrrolidin-1-yl]-3- |
| | | methoxy-5-methyl-1-oxoheptan-4-yl]-methylamino]-3-methyl-1-oxobutan-2-yl]-3- |
| | | methyl-2-(methylamino)butanamide |
| Maytansine | 35846-53-8 | 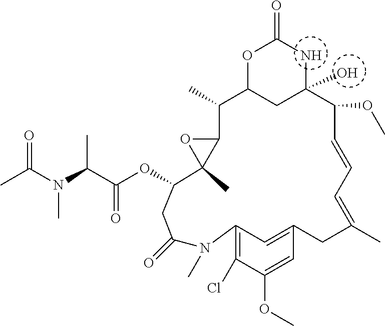 |
| | | [[(1S,2R,3S,5S,6S,16E,18E,20R,21S)-11-chloro-21-hydroxy-12,20-dimethoxy- |
| | | 2,5,9,16-tetramethyl-8,23-dioxo-4,24-dioxa-9,22- |
| | | diazatetracyclo[19.3.1.1^{10,14}.0^{3,5}]hexacosa-10,12,14(26),16,18-pentaen-6-yl] (2S)-2- |
| | | [acetyl(methyl)amino]propanoate |
| Maytansinoid | — | 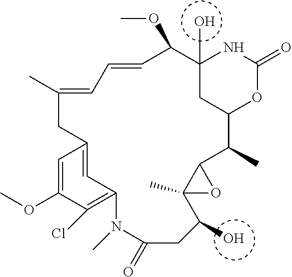 |
| | | (2R,3S,5S,6S,16E,18E,20R)-11-chloro-6,21-dihydroxy-12,20-dimethoxy-2,5,9,16- |
| | | tetramethyl-4,24-dioxa-9,22-diazatetracyclo[19.3.1.1^{10,14}.0^{3,5}]hexacosa- |
| | | 10,12,14(26),16,18-pentaene-8,23-dione |
| Ozogamicin | 400046-53-9 | 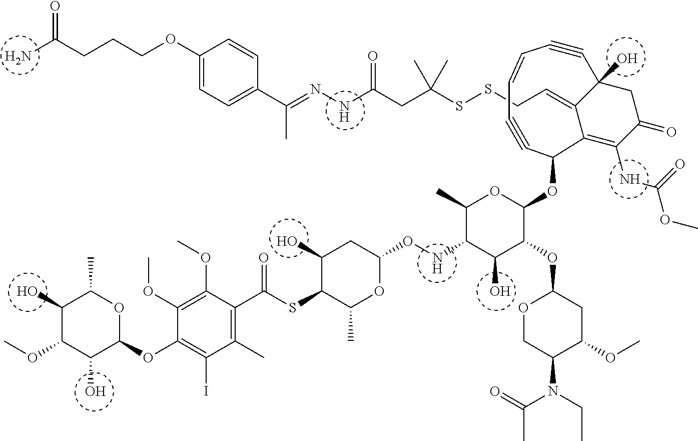 |
| | | S-[(2R,3S,4S,6S)-6-[[(2R,3S,4S,5R,6R)-5-[(2S,4S,5S)-5-[acetyl(ethyl)amino]-4- |
| | | methoxyoxan-2-yl]oxy-6-[[(2S,5Z,9R,13E)-13-[2-[[4-[(2E)-2-[1-[4-(4-amino-4- |
| | | oxobutoxy)phenyl]ethylidene]hydrazinyl]-2-methyl-4-oxobutan-2- |
| | | yl]disulfanyl]ethylidene]-9-hydroxy-12-(methoxycarbonylamino)-11-oxo-2- |
| | | bicyclo[7.3.1]trideca-1(12),5-dien-3,7-diynyl]oxy]-4-hydroxy-2-methyloxan-3- |
| | | yl]amino]oxy-4-hydroxy-2-methyloxan-3-yl] 4-[(2S,3R,4R,5S,6S)-3,5-dihydroxy-4- |
| | | methoxy-6-methyloxan-2-yl]oxy-5-iodo-2,3-dimethoxy-6- |
| | | methylbenzenecarbothioate |
| Paclitaxel | 33069-62-4 | 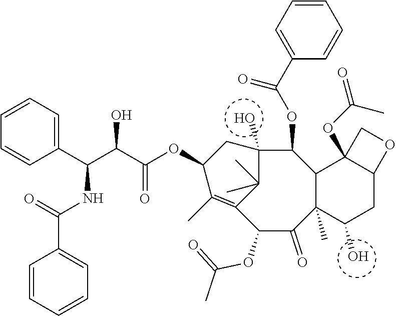 |
| | | [(1S,2S,3R,4S,7R,9S,10S,12R,15S)-4,12-diacetyloxy-15-[(2R,3S)-3-benzamido-2- |
| | | hydroxy-3-phenylpropanoyl]oxy-1,9-dihydroxy-10,14,17,17-tetramethyl-11-oxo-6- |
| | | oxatetracyclo[11.3.1.0^{3,10}.0^{4,7}]heptadec-13-en-2-yl] benzoate |
| Pirarubicin | 72496-41-4 | 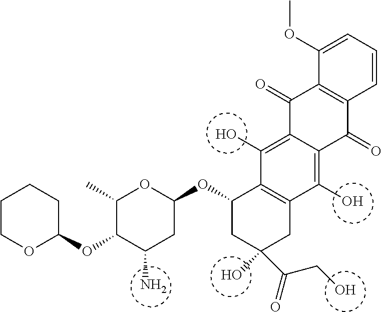 |
| | | (7S,9S)-7-[(2R,4S,5S,6S)-4-amino-6-methyl-5-[(2R)-oxan-2-yl]oxyoxan-2-yl]oxy- |
| | | 6,9,11-trihydroxy-9-(2-hydroxyacetyl)-4-methoxy-8,10-dihydro-7H-tetracene-5,12- |
| | | dione |
| Pixantrone | 144510-96-3 | 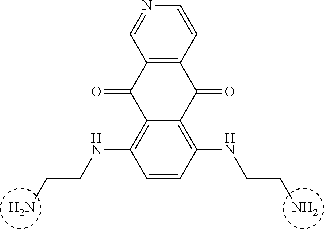 [SMILES: NCCNc1ccc(NCCN)c2c1C(=O)c1ccncc1C2=O] |
| | | 6,9-bis(2-aminoethylamino)benzo[g]isoquinoline-5,10-dione |
| Podophyllotoxin | — | 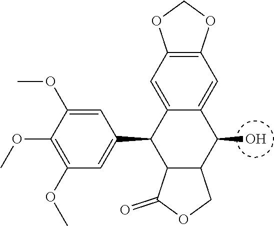 [SMILES: COc1cc([C@H]2c3cc4c(cc3[C@@H](O)C3COC(=O)C32)OCO4)cc(OC)c1OC] |
| | | (5S,5aS,8aS,9S)-5-hydroxy-9-(3,4,5-trimethoxyphenyl)-5a,6,8a,9-tetrahydro-5H- |
| | | [2]benzofuro[5,6-f][1,3]benzodioxol-8-one |
| Procarbazine | 671-16-9 | 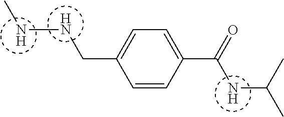 [SMILES: CNNCc1ccc(C(=O)NC(C)C)cc1] |
| | | 4-[(2-methylhydrazinyl)methyl]-N-propan-2-ylbenzamide |
| Rapamycin | — | 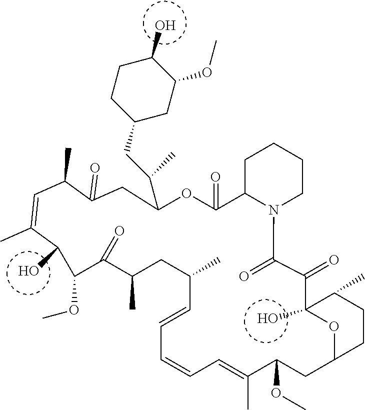 |
| | | (1R,15R,16E,18R,19R,21R,23S,24Z,26E,28E,30S,35R)-1,18-dihydroxy-12-[(2R)-1- |
| | | [(1S,3R,4R)-4-hydroxy-3-methoxycyclohexyl]propan-2-yl]-19,30-dimethoxy- |
| | | 15,17,21,23,29,35-hexamethyl-11,36-dioxa-4-azatricyclo[30.3.1.0^{4,9}]hexatriaconta- |
| | | 16,24,26,28-tetraene-2,3,10,14,20-pentone |
| Rachelmycin | 69866-21-3 | 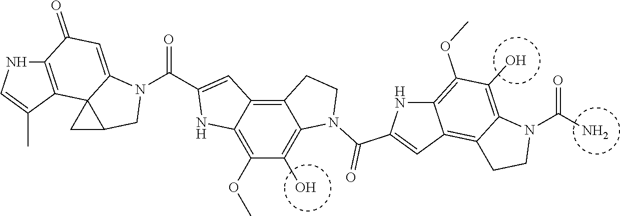 [SMILES: COc1c(O)c2c(c3cc(C(=O)N4CCc5c4c(O)c(OC)c4[nH]c(C(=O)N6CC7CC78C6=CC(=O)c6[nH]cc(C)c68)cc54)[nH]c13)CCN2C(N)=O] |
| | | 5-hydroxy-2-[5-hydroxy-4-methoxy-2-(3-methyl-7-oxo-5,10- |
| | | diazatetracyclo[7.4.0.0^{1,12}.0^{2,6}]trideca-2(6),3,8-triene-10-carbonyl)-7,8-dihydro-3H- |
| | | pyrrolo[3,2-e]indole-6-carbonyl]-4-methoxy-7,8-dihydro-3H-pyrrolo[3,2-e]indole-6- |
| | | carboxamide |
| Salinomycin | 53003-10-4 | 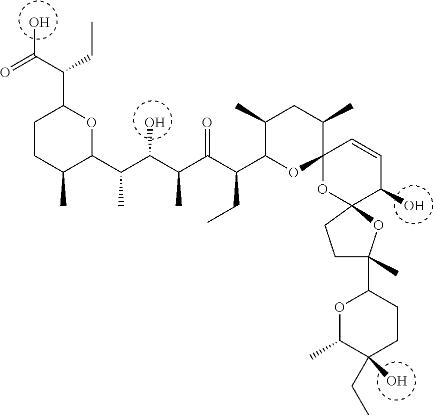 |
| | | (2R)-2-[(2R,5S,6R)-6-[(2S,3S,4S,6R)-6-[(3S,5S,7R,9S,10S,12R,15R)-3-[(2R,5R,6S)-5- |
| | | ethyl-5-hydroxy-6-methyloxan-2-yl]-15-hydroxy-3,10,12-trimethyl-4,6,8- |
| | | trioxadispiro[4.1.5⁷.3⁵]pentadec-13-en-9-yl]-3-hydroxy-4-methyl-5-oxooctan-2-yl]- |
| | | 5-methyloxan-2-yl]butanoic acid |
| SB-T-1214 | 178250-23-2 | 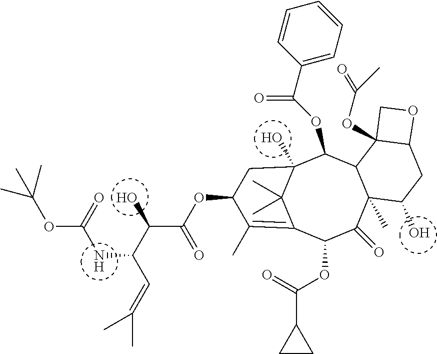 |
| | | [(1S,2S,3R,4S,7R,9S,10S,12R,15S)-4-acetyloxy-12-(cyclopropanecarbonyloxy)-1,9- |
| | | dihydroxy-15-[(2R,3S)-2-hydroxy-5-methyl-3-[(2-methylpropan-2- |
| | | yl)oxycarbonylamino]hex-4-enoyl]oxy-10,14,17,17-tetramethyl-11-oxo-6- |
| | | oxatetracyclo[11.3.1.0^{3,10}.0^{4,7}]heptadec-13-en-2-yl] benzoate |
| Selinexor | 1393477-72-9 | 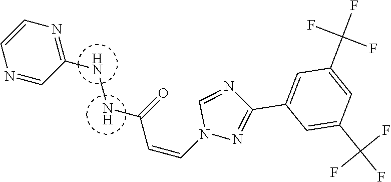 [SMILES: O=C(/C=C\n1cnc(-c2cc(C(F)(F)F)cc(C(F)(F)F)c2)n1)NNc1cnccn1] |
| | | (Z)-3-[3-[3,5-bis(trifluoromethyl)phenyl]-1,2,4-triazol-1-yl]-N′-pyrazin-2-ylprop-2- |
| | | enehydrazide |
| SN-38 | 86639-52-3 | 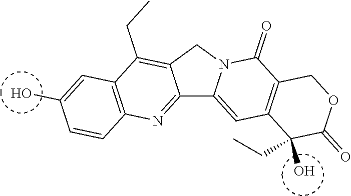 [SMILES: CCc1c2c(nc3ccc(O)cc13)-c1cc3c(c(=O)n1C2)COC(=O)[C@]3(O)CC] |
| | | (19S)-10,19-diethyl-7,19-dihydroxy-17-oxa-3,13- |
| | | diazapentacyclo[11.8.0.0^{2,11}.0^{4,9}.0^{15,20}]henicosa-1(21),2,4(9),5,7,10,15(20)-heptaene- |
| | | 14,18-dione |
| Solamargine | 20311-51-7 | 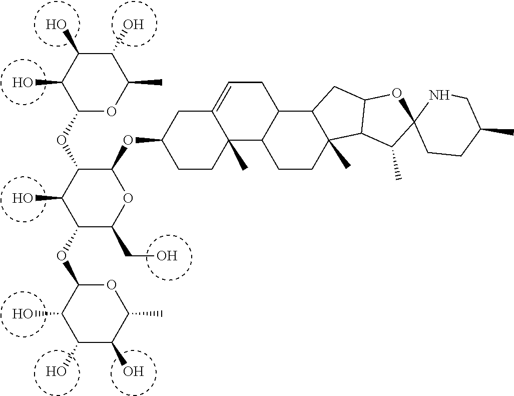 |
| | | (2S,3R,4R,5R,6S)-2-[(2R,3S,4S,5R,6R)-4-hydroxy-2-(hydroxymethyl)-6- |
| | | [[(1S,2S,4S,5′R,6R,7S,8R,9S,12S,13R,16S)-5′,7,9,13-tetramethylspiro[5- |
| | | oxapentacyclo[10.8.0.0^{2,9}.0^{4,8}.0^{13,18}]icos-18-ene-6,2′-piperidine]-16-yl]oxy-5- |
| | | [(2S,3R,4R,5R,6S)-3,4,5-trihydroxy-6-methyloxan-2-yl]oxyoxan-3-yl]oxy-6- |
| | | methyloxane-3,4,5-triol |
| Solanine | 20562-02-1 | 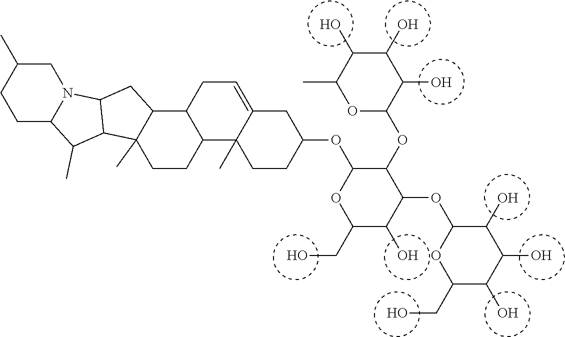 [SMILES: CC1CCC2C(C)C3C(CC4C5CC=C6CC(OC7OC(CO)C(O)C(OC8OC(CO)C(O)C(O)C8O)C7OC7OC(C)C(O)C(O)C7O)CCC6(C)C5CCC43C)N2C1] |
| | | 2-[5-hydroxy-6-(hydroxymethyl)-2-[(10,14,16,20-tetramethyl-22- |
| | | azahexacyclo[12.10.0.0^{2,11}.0^{5,10}.0^{15,23}.0^{17,22}]tetracos-4-en-7-yl)oxy]-4-[3,4,5- |
| | | trihydroxy-6-(hydroxymethyl)oxan-2-yl]oxyoxan-3-yl]oxy-6-methyloxane-3,4,5-triol |
| Talirine | 1342820-51-2 | 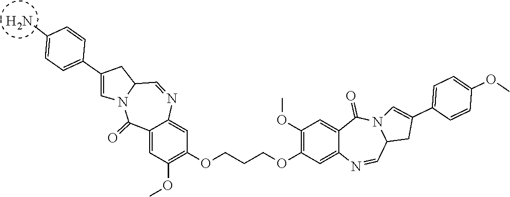 [SMILES: COc1ccc(C2=CN3C(=O)c4cc(OC)c(OCCCOc5cc6c(cc5OC)C(=O)N5C=C(c7ccc(N)cc7)CC5C=N6)cc4N=CC3C2)cc1] |
| | | N-[(2S)-1-[[(2S)-1-[4-[(6aS)-3-[3-[[(6aS)-2-methoxy-8-(4-methoxyphenyl)-11-oxo- |
| | | 6a,7-dihydropyrrolo[2,1-c][1,4]benzodiazepin-3-yl]oxy]propoxy]-2-methoxy-11- |
| | | oxo-6a,7-dihydropyrrolo[2,1-c][1,4]benzodiazepin-8-yl]anilino]-1-oxopropan-2- |
| | | yl]amino]-3-methyl-1-oxobutan-2-yl]-6-(2,5-dioxopyrrol-1-yl)hexanamide |
| Temozolomide | 85622-93-1 | 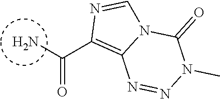 |
| | | 3-methyl-4-oxoimidazo[5,1-d][1,2,3,5]tetrazine-8-carboxamide |
| Tesetaxel | 333754-36-2 | 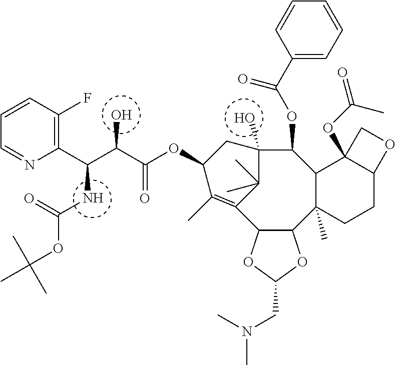 |
| | | [(2R,4S,6S,7R,10R,13S,14R,15S,16S,18S)-13-acetyloxy-4-[(dimethylamino)methyl]- |
| | | 18-[(2R,3S)-3-(3-fluoropyridin-2-yl)-2-hydroxy-3-[(2-methylpropan-2- |
| | | yl)oxycarbonylamino]propanoyl]oxy-16-hydroxy-7,19,20,20-tetramethyl-3,5,11- |
| | | trioxapentacyclo[14.3.1.0^{2,6}.0^{7,14}.0^{10,13}]icos-1(19)-en-15-yl] benzoate |
| SG3199 (Tesirine) | 1595275-71-0 | 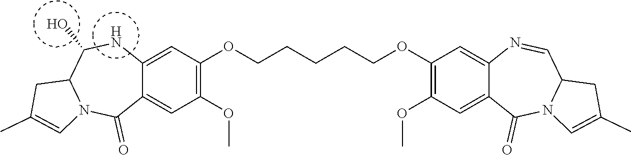 |
| | | 11-(S)-hydroxy-7-methoxy-8-((5-((7-methoxy-2-methyl-5-oxo-5,11a-dihydro-1H- |
| | | benzo[e]pyrrolo[1,2-a][1,4]diazepin-8-yl)oxy)pentyl)oxy)-2-methyl-1,10,11,11a- |
| | | tetrahydro-5H-pyrrolo[2,1-c][1,4]benzodiazepin-5-one |
| Thapsigargin | 67526-95-8 | 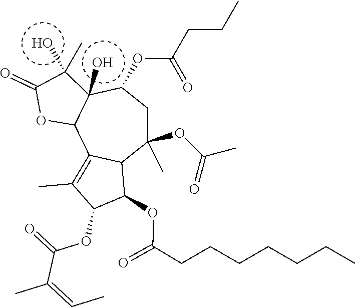 |
| | | [(3S,3aR,4S,6S,6aR,7S,8S,9bS)-6-acetyloxy-4-butanoyloxy-3,3a-dihydroxy-3,6,9- |
| | | trimethyl-8-[(Z)-2-methylbut-2-enoyl]oxy-2-oxo-4,5,6a, 7,8,9b- |
| | | hexahydroazuleno[4,5-b]furan-7-yl] octanoate |
| Tomatine | 17406-45-0 | 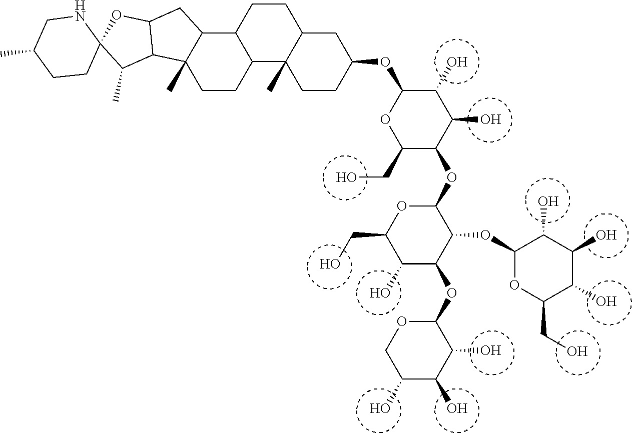 |
| | | (2S,3R,4S,5S,6R)-2-[(2S,3R,4S,5R,6R)-2-[(2R,3R,4R,5R,6R)-4,5-dihydroxy-2- |
| | | (hydroxymethyl)-6-[(1R,2S,4S,5′S,6S,7S,8R,9S,12S,13S,16S,18S)-5′,7,9,13- |
| | | tetramethylspiro[5-oxapentacyclo[10.8.0.0^{2,9}.0^{4,8}.0^{13,18}]icosane-6,2′-piperidine]-16- |
| | | yl]oxyoxan-3-yl]oxy-5-hydroxy-6-(hydroxymethyl)-4-[(2S,3R,4S,5R)-3,4,5- |
| | | trihydroxyoxan-2-yl]oxyoxan-3-yl]oxy-6-(hydroxymethyl)oxane-3,4,5-triol |
| Topotecan | 123948-87-8 | 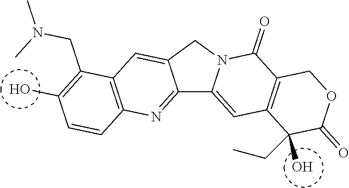 |
| | | (19S)-8-[(dimethylamino)methyl]-19-ethyl-7,19-dihydroxy-17-oxa-3,13- |
| | | diazapentacyclo[11.8.0.0^{2,11}.0^{4,9}.0^{15,20}]henicosa-1(21),2,4(9),5,7,10,15(20)-heptaene- |
| | | 14,18-dione |
| Tubulysin B | 205304-87-6 | 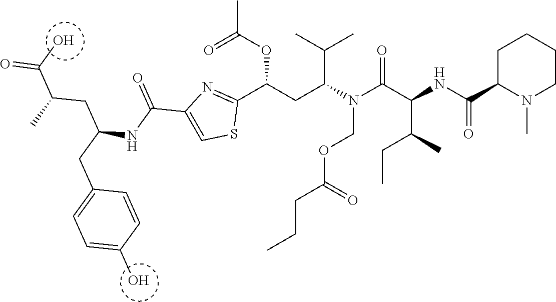 [SMILES: CCCC(=O)OCN(C(=O)[C@@H](NC(=O)[C@H]1CCCCN1C)[C@@H](C)CC)[C@H](C[C@@H](OC(C)=O)c1nc(C(=O)N[C@@H](Cc2ccc(O)cc2)C[C@H](C)C(=O)O)cs1)C(C)C] |
| | | (2S,4R)-4-[[2-[(1R,3R)-1-acetyloxy-3-[butanoyloxymethyl-[(2S,3S)-3-methyl-2- |
| | | [[(2R)-1-methylpiperidine-2-carbonyl]amino]pentanoyl]amino]-4-methylpentyl]-1,3- |
| | | thiazole-4-carbonyl]amino]-5-(4-hydroxyphenyl)-2-methylpentanoic acid |
| Valrubicin | 56124-62-0 | 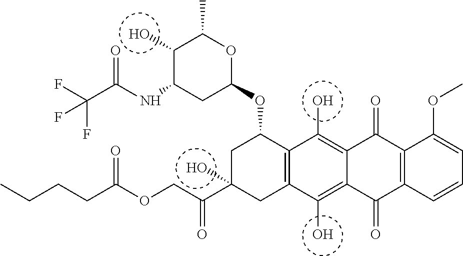 |
| | | [2-oxo-2-[(2S,4S)-2,5,12-trihydroxy-4-[(2R,4S,5S,6S)-5-hydroxy-6-methyl-4-[(2,2,2- |
| | | trifluoroacetyl)amino]oxan-2-yl]oxy-7-methoxy-6,11-dioxo-3,4-dihydro-1H- |
| | | tetracen-2-yl]ethyl] pentanoate |
| Vinblastine | 865-21-4 | 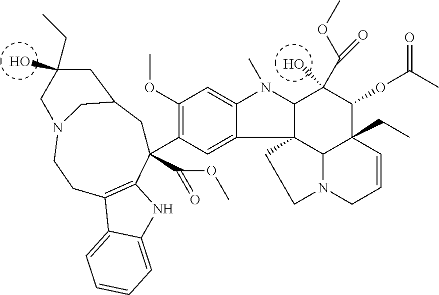 |
| | | methyl (1R,9R,10S,11R,12R,19R)-11-acetyloxy-12-ethyl-4-[(13S,15R,17S)-17-ethyl- |
| | | 17-hydroxy-13-methoxycarbonyl-1,11-diazatetracyclo[13.3.1.0^{4,12}.0^{5,10}]nonadeca- |
| | | 4(12),5,7,9-tetraen-13-yl]-10-hydroxy-5-methoxy-8-methyl-8,16- |
| | | diazapentacyclo[10.6.1.0^{1,9}.0^{2,7}.0^{16,19}]nonadeca-2,4,6,13-tetraene-10-carboxylate |
| Vincristine | 57-22-7 | 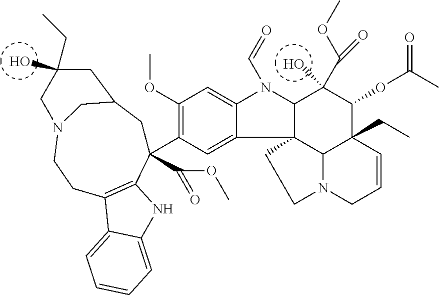 |
| | | methyl (1R,9R,10S,11R,12R,19R)-11-acetyloxy-12-ethyl-4-[(13S,15S,17S)-17-ethyl- |
| | | 17-hydroxy-13-methoxycarbonyl-1,11-diazatetracyclo[13.3.1.0^{4,12}.0^{5,10}]nonadeca- |
| | | 4(12),5,7,9-tetraen-13-yl]-8-formyl-10-hydroxy-5-methoxy-8,16- |
| | | diazapentacyclo[10.6.1.0^{1,9}.0^{2,7}.0^{16,19}]nonadeca-2,4,6,13-tetraene-10-carboxylate |
| Vinorelbine | 71486-22-1 | 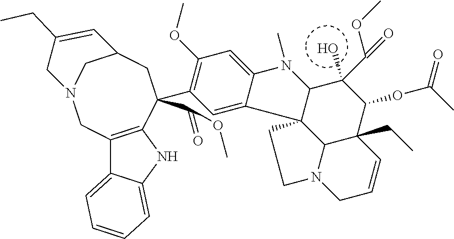 |
| | | methyl (1R,9R,10S,11R,12R,19R)-11-acetyloxy-12-ethyl-4-[(12S,14R)-16-ethyl-12- |
| | | methoxycarbonyl-1,10-diazatetracyclo[12.3.1.0^{3,11}.0^{4,9}]octadeca-3(11),4,6,8,15- |
| | | pentaen-12-yl]-10-hydroxy-5-methoxy-8-methyl-8,16- |
| | | diazapentacyclo[10.6.1.0^{1,9}.0^{2,7}.0^{16,19}]nonadeca-2,4,6,13-tetraene-10-carboxylate |
| VIP126 | — | 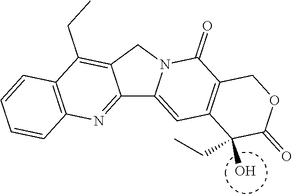 |
| | | (19S)-10,19-diethyl-19-hydroxy-17-oxa-3,13- |
| | | diazapentacyclo[11.8.0.0^{2,11}.0^{4,9}.0^{15,20}]henicosa-1(21),2,4,6,8,10,15(20)-heptaene- |
| | | 14,18-dione |
| Zorubicin | 54083-22-6 | 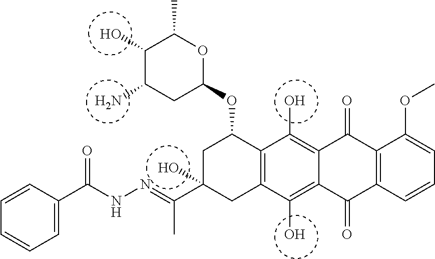 |
| | | N-[1-[(2S,4S)-4-[(2R,4S,5S,6S)-4-amino-5-hydroxy-6-methyloxan-2-yl]oxy-2,5,12- |
| | | trihydroxy-7-methoxy-6,11-dioxo-3,4-dihydro-1H-tetracen-2- |
| | | yllethylideneamino]benzamide |

The vast majority of the chemotherapeutic compounds listed in Table 1 and Table 2 can be readily procured from commercial vendors or prepared from commercial compounds via facile derivatization. The same applies to self-immolative linkers Lᵢ of the present invention (e.g. https://bezwadabiomedical.com/) which can be suitably functionalized and protected for sequential coupling with a hydroxy or amine group of FAP-cleavable moiety Fcᵢ and the chemotherapeutic compounds of Table 1 and Table 2. Strategies and schemes for chemical synthesis and coupling via an amide or ether bond are presented in Examples 1-4.

### DETAILED DESCRIPTION

The invention provides following advantages:
each Ctᵢ is known and has a well characterized pharmaceutical activity;
each Sᵢ is pharmacologically inactive unless ligated to and cleaved by fibroblast activation protein (FAP) expressed primarily by cancer-associated fibroblasts (CAF) in tumor tissue and metastatic lesions;
each Sᵢ is pharmacologically adapted for good solubility and stability in serum and prolonged systemic retention;
each Sᵢ is suitable for large volume synthesis and economic production,
the chemotherapeutic drug kit can be manufactured in an efficient and economic manner, and
the chemotherapeutic drug kit is versatile and facilitates clinical use.

The inventive small-molecule-drug-conjugates (or prodrugs) Sᵢ contain a moiety that is enzymatically cleaved by fibroblast activation protein (FAP). FAP is almost exclusively expressed in somatically healing wounds and in the micro environment (or stroma) of cancer tumors. Many cancer tumors comprise a tumor micro environment (stroma) that surrounds cancer cells (carcinogenic cells). The tumor stroma includes various non-malignant cell types and accounts for up to 90% of the total tumor mass. It plays an important role in the supply of cancer cells as well as in tumor progression and metastasis. Important components of the tumor stroma are the extracellular matrix (ECM), endothelial cells, pericytes, macrophages, immune regulatory cells and activated fibroblasts, commonly referred to as cancer-associated fibroblasts (CAF). During tumor progression, CAF change their morphology and biological function. These changes are induced by intercellular communication between cancer cells and CAF. CAF create an environment that promotes cancer cell growth. It has been shown that therapies that merely target cancer cells are inadequate. Effective therapies must also address the tumor microenvironment and in particular CAF.

In more than 90% of human epithelial tumors CAF overexpress fibroblast activation protein (FAP). Therefore, FAP represents a promising target for cancer medication. The role of FAP in vivo is not fully understood, however, it is known to be a serine protease with unique enzymatic activity. It exhibits both dipeptidyl peptidase (DPP) and prolyl oligopeptidase (PREP) activity. Hence, for CAF targeting, substrates and inhibitors of DPP, PREP and FAP come into consideration as homing ligands. A suitable FAP ligand must possess high selectivity over related enzymes, such as dipeptidyl peptidases DPPII, DPPIV, DPP8, DPP9 and homologous prolyl oligopeptidases that are ubiquitous in healthy tissue.

Small molecule ligands with high affinity and selectivity for FAP are known since 2014 and 2019, respectively (cf. K. Jansen, L. Heirbaut, R. Verkerk, J. D. Cheng, J. Joossens, P. Cos, L. Maes, A.-M. Lambeir, I. De Meester, K. Augustyns, P. Van der Veken; Extended Structure—Activity Relationship and Pharmacokinetic Investigation of (4-Quinolinoyl) glycyl-2-cyanopyrrolidine Inhibitors of Fibroblast Activation Protein (FAP); J. Med. Chem. 2014 Apr. 10; 57 (7): 3053-74, DOI 10.1021/jm500031w; A. De Decker, G. Vliegen, D. Van Rompaey, A. Peeraer, A. Bracke, L. Verckist, K. Jansen, R. Geiss-Friedlander, K. Augustyns, H. De Winter, I. De Meester, A.-M. Lambeir, P. Van der Veken, Novel Small Molecule-Derived, Highly Selective Substrates for Fibroblast Activation Protein (FAP), ACS Med. Chem. Lett. 2019, 10, 8, 1173-1179). These ligands comprise a modified glycine-proline unit and therewith coupled quinoline group.

Regarding circulating tumor cells (CTC) Raskov et al. note: “For instance, CTC have higher viability in the blood stream when accompanied by stroma cells that also provide an advantage with respect to early survival and growth of tumor cells at the metastatic site (31). Traveling in clusters with macrophages, immune cells, and platelets, CAF support, shield, and increase the survival of CTC.” (cf. H. Raskov, A. Orhan, S. Gaggar, I. Gögenur; Cancer-Associated Fibroblasts and Tumor-Associated Macrophages in Cancer and Cancer Immunotherapy; Frontiers in Oncology, May 2021, Volume 11, Article 668731; page 5, left column, line 24-29). Hence, the inventive prodrugs may also be activated by circulating CAF and consequently affect CTC.

Concerning drugs that specifically target CAF Raskov et al. (page 12, left column, 1st paragraph) further remark that: “The regulation/eradication of α-SMA⁺ or FAP⁺ CAF have had variable results and currently, targeting CAF or TAM individually does not seem to be an appropriate approach.”

The present invention, though, utilizes FAP merely as a means for chemotherapeutic drug activation and does not intend to regulate or eradicate CAF. Accordingly, the inventive prodrugs comprise chemotherapeutic compounds that are aimed at oncogenic cells. At the same time CAF constitute bystanders that can be collaterally affected, particularly by cytotoxic agents. In many instances collateral injury to CAF may promote the antitumor effect of the inventive prodrugs.

The inventive chemotherapeutic kit readily provides innumerous possibilities for selection and simultaneous administration of two or more prodrugs. Particularly, in case of cancer relapse distinctly different therapy regimens may be pursued in a flexible and adaptive manner.

In a preferred adaptive mode the inventive therapy is accompanied by frequent quantitative diagnostics, such as liquid biopsy and ultrasound based assessment of tumor size, vasculature and perfusion. If a selected combination of inventive prodrugs does not yield a quantitative improvement within 2-3 weeks a distinctly different prodrug combination can be employed.

As substantiated above the present invention has the object to provide a chemotherapeutic drug kit that
enables facile and cost effective treatment of solid cancer tumors through exposure to a multitude of different chemotherapeutic agents in rapidly varying time sequence;
inhibits cancer proliferation; and
has negligible adverse effects at high tumor-targeted dose.

This object is achieved through a chemotherapeutic drug kit comprising N different small-molecule-drug-conjugates selected from the group comprising

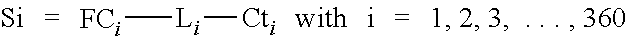

wherein

$$2 \leq N \leq 360 ,$$

each Fcᵢ independently from one another has the structure

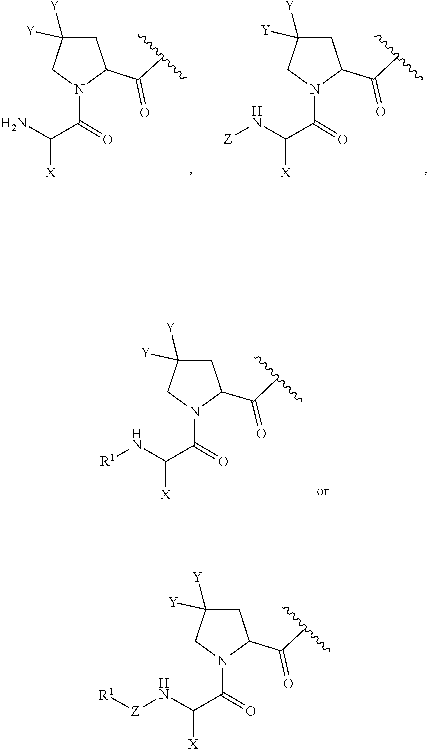

where X=—H or —CH₃, Y=—H or —F, —R¹ is a residue of a first pharmacokinetic modulating moiety and Z is a moiety having a structure selected from the group comprising structures (1), (2), (3), (4), (5), (6), (7), (8), (9), (10), (11), (12), (13), (14) and (15) with

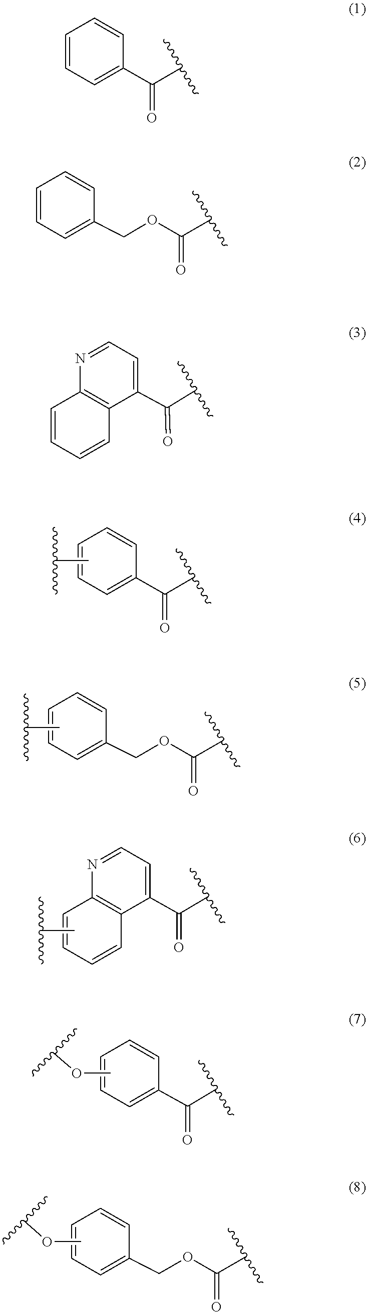

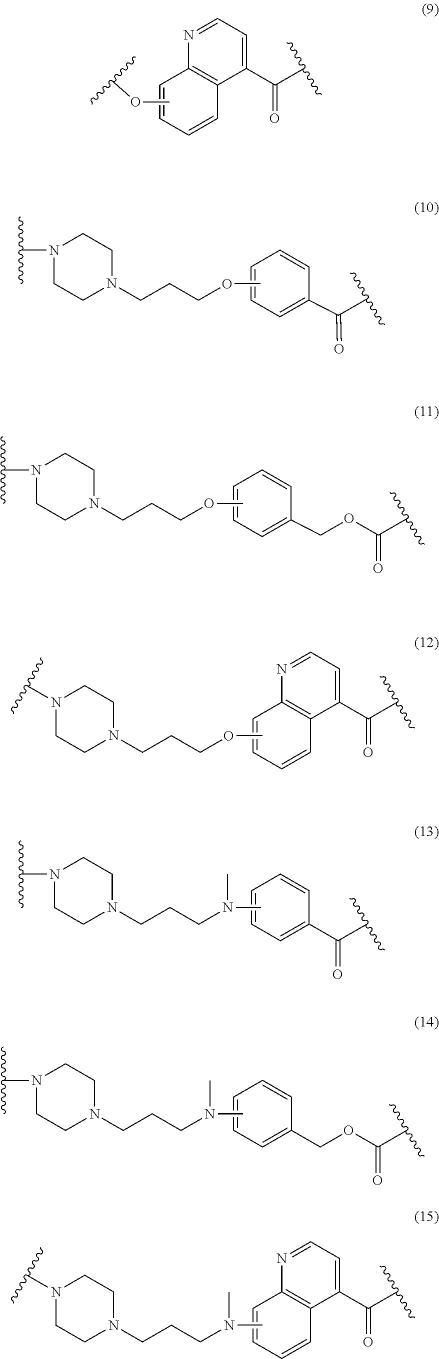

each Lᵢ is a residue of a self-immolative linker;
Ctᵢ and Fcᵢ are covalently bound to Lᵢ;
Ctᵢ #Ctⱼ for i≠j; and

each Ctᵢ is selected from the group comprising a deprotonated residue of 1,2,3,4-Tetrahydrogen-staurosporine, 17-Dmag, 2-Aminopropanenitrile, 4SC202, ABBV-CLS-484, Abemaciclib, Abexinostat, Acalabrutinib, Acetylbufalin, Aderbasib, Afatinib, Afuresertib, Alectinib, Alisertib, Alpelisib, Alvocidib, AMD3465, Anlotinib, Apalutamide, AR-42, Asciminib, Atuveciclib, Avapritinib, Axitinib, AZD7762, BAY1125976, Belinostat, β-Hydroxyisovaleric acid, BF211, Bicalutamide, Binimetinib, Bortezomib, Bosutinib, Brigatinib, Bufalin, Buparlisib, Buthionine sulfoximine, Cabozantinib, Capivasertib, Capmatinib, Carfilzomib, CEP-9722, Ceralasertib, Ceritinib, Chidamide, CHR-3996, Citarinostat, Cobimetinib, CompK, Copanlisib, Crenolanib, Crizotinib, CUDC-101, Dabrafenib, Daclatasvir, Dacomitinib, Darolutamide, Dasatinib, Dasatinib D1, Dasatinib D2, Dasatinib D3, Dasatinib D4, Decitabine, Defactinib, Degarelix, Diethylstilbestrol, Dinaciclib, Dp44mT, DpC, DUPA, Duvelisib, E7016, Ebvaciclib, Eganelisib, Elimusertib, Emavusertib, Enasidenib, Encorafenib, Enitociclib, Entinostat, Entrectinib, Enzalutamide, Epacadostat, Epigallocatechin gallate, Epoxomicin, Erdafitinib, Erismodegib, Erlotinib, Everolimus, Fasudil, Fedratinib, Filgotinib, Foslinanib, Fostamatinib, Fruquintinib, Galunisertib, Ganetespib, Gedatolisib, Gefitinib, GFH018, Gilteritinib, Givinostat, Glasdegib, Goserelin, GSK2256098, GSK269962A, GSK690693, GUL, Halofuginone, Hymecromone, Ibrutinib, Icotinib, Idelalisib, Imatinib, Imiquimod, Infigratinib, Iniparib, Ipatasertib, Itacitinib, Ivaltinostat, Ivosidenib, Ixazomib, Kevetrin, Lapatinib, Larotrectinib, Lenalidomide, Leniolisib, Lenvatinib, Leuprolide, Linsitinib, Lonafarnib, Lorlatinib, Losartan, Lucitanib, Luminespib, M1096, Marizomib, ME-344, Merestinib, Metformin, MG132, Midostaurin, Miransertib, Mivavotinib, MK2206, MMP-9 Inhibitor I, Mobocertinib, Mocetinostat, Motesanib, MRTX1133, Navitoclax, Nazartinib, Nedisertib, Neratinib, Nilotinib, Nilutamide, Nintedanib, Niraparib, NMS-P118, NMS-P515, NSC668394, NSC95397, Numidargistat, NVP-2, Olaparib, Olmutinib, Omipalisib, Oprozomib, Osimertinib, OTS-964, Palbociclib, Pamiparib, Panobinostat, Paricalcitol, Parsaclisib, Pazopanib, Pemetrexed, Pemigatinib, Pevonedistat, Pexidartinib, Pifusertib, Plerixafor, PMPA, Ponatinib, Practinostat, Pralsetinib, Prednisone, Prexasertib, Prinomastat, Propranolol, Quisinostat, Quizartinib, Ralimetinib, Ravoxertinib, Regorafenib, Relugolix, Resminostat, Resveratrol, Retaspimycin, Retinoic acid, Ribociclib, Ricolinostat, Rigosertib, Ripretinib, RO-3306, Rocilinostat, Rogaratinib, Romidepsin, Rucaparib, Ruxolitinib, S2, S5, Saridegib, SBI-0654454, SCH772984, Seliciclib, Selitrectinib, Selpercatinib, Selumetinib, SGN-2FF, SGX393, Shikonin, Silibinin, Sitravatinib, Sonidegib, Sorafenib, Sotorasib, Staurosporine, SU11274, Sunitinib, Surufatinib, Tacedinaline, Tadalafil, Talazoparib, Taletrectinib, Tarloxotinib, Taselisib, Tazemetostat, Tefinostat, Temsirolimus, Tetrazole, Tivozanib, Tofacitinib, Tozasertib, Trametinib, Tranilast, Tretinoin, Trichostatin, Tucatinib, Tucidinostat, Tuvusertib, Ubenimex, Umbralisib, Uprosertib, USL311, Vactosertib, Valproic acid, Valsartan, Vandetanib, Veliparib, Vemurafenib, Venetoclax, Verteporfin, Vismodegib, Vorinostat, WRG-28, WZ811, Xevinapant, Zandelisib, Zanubrutinib, ZM447439, Abiraterone, Aclarubicin, Adozelesin, Alrestatin, Amanitin, Amrubicin, Anthramycin, Arenastatin, Bizelesin, Bleomycin, Camptothecin, Capecitabine, Carzelesin, CC-1065, Chaconine, Chlorambucil, Cryptophycin-24, Cyclophosphamide, Cytarabine, Dacarbazine, Dactinomycin, Daunorubicin, DAVLBH, Deruxtecan, Dexamethasone, Dichloro acetic acid, Dimethyl-SGD-1882, Docetaxel, Dolastatin-10, Doxorubicin, Duocarmycin A, Duocarmycin B1, Duocarmycin B2, Duocarmycin C1, Duocarmycin C2, Duocarmycin D, Duocarmycin GA, Duocarmycin SA, Emetine, Epirubicin, Eribulin, Etoposide, Floxuridine, Fludarabine, Fluorouracil, Flutamide, Fulvestrant, Gemcitabine, Idarubicin, Ifosfamide, Irinotecan, L-Asparaginase, Lomustine, Melphalan, Mertansine, Methotrexate, Milataxel, Mitoxantrone, Monomethyl Auristatin E, Maytansine, Maytansinoid, Ozogamicin, Paclitaxel, Pirarubicin, Pixantrone, Podophyllotoxin, Procarbazine, Rapamycin, Rachelmycin, Salinomycin, SB-T-1214, Selinexor, SN-38, Solamargine, Solanine, Talirine, Temozolomide, Tesetaxel, SG3199 (Tesirine), Thapsigargin, Tomatine, Topotecan, Tubulysin B, Valrubicin, Vinblastine, Vincristine, Vinorelbine, VIP126, Zorubicin.

Expedient embodiments of the inventive prodrug compounds are characterized by one of the following features or a combination of two or more of the following features insofar the combined features are not mutually exclusive or contradictory and according to which:
each Ctᵢ is selected from the group comprising a deprotonated residue of 1,2,3,4-Tetrahydrogen-staurosporine, 17-Dmag, 2-Aminopropanenitrile, 4SC202, ABBV-CLS-484, Abemaciclib, Abexinostat, Acalabrutinib, Acetylbufalin, Aderbasib, Afatinib, Afuresertib, Alectinib, Alisertib, Alpelisib, Alvocidib, AMD3465, Anlotinib, Apalutamide, AR-42, Asciminib, Atuveciclib, Avapritinib, Axitinib, AZD7762, BAY1125976, Belinostat, β-Hydroxyisovaleric acid, BF211, Bicalutamide, Binimetinib, Bortezomib, Bosutinib, Brigatinib, Bufalin, Buparlisib, Buthionine sulfoximine, Cabozantinib, Capivasertib, Capmatinib, Carfilzomib, CEP-9722, Ceralasertib, Ceritinib, Chidamide, CHR-3996, Citarinostat, Cobimetinib, CompK, Copanlisib, Crenolanib, Crizotinib, CUDC-101, Dabrafenib, Daclatasvir, Dacomitinib, Darolutamide, Dasatinib, Dasatinib D1, Dasatinib D2, Dasatinib D3, Dasatinib D4, Decitabine, Defactinib, Degarelix, Diethylstilbestrol, Dinaciclib, Dp44mT, DpC, DUPA, Duvelisib, E7016, Ebvaciclib, Eganelisib, Elimusertib, Emavusertib, Enasidenib, Encorafenib, Enitociclib, Entinostat, Entrectinib, Enzalutamide, Epacadostat, Epigallocatechin gallate, Epoxomicin, Erdafitinib, Erismodegib, Erlotinib, Everolimus, Fasudil, Fedratinib, Filgotinib, Foslinanib, Fostamatinib, Fruquintinib, Galunisertib, Ganetespib, Gedatolisib, Gefitinib, GFH018, Gilteritinib, Givinostat, Glasdegib, Goserelin, GSK2256098, GSK269962A, GSK690693, GUL, Halofuginone, Hymecromone, Ibrutinib, Icotinib, Idelalisib, Imatinib, Imiquimod, Infigratinib, Iniparib, Ipatasertib, Itacitinib, Ivaltinostat, Ivosidenib, Ixazomib, Kevetrin, Lapatinib, Larotrectinib, Lenalidomide, Leniolisib, Lenvatinib, Leuprolide, Linsitinib, Lonafarnib, Lorlatinib, Losartan, Lucitanib, Luminespib, M1096, Marizomib, ME-344, Merestinib, Metformin, MG132, Midostaurin, Miransertib, Mivavotinib, MK2206, MMP-9 Inhibitor I, Mobocertinib, Mocetinostat, Motesanib, MRTX1133, Navitoclax, Nazartinib, Nedisertib, Neratinib, Nilotinib, Nilutamide, Nintedanib, Niraparib, NMS-P118, NMS-P515, NSC668394, NSC95397, Numidargistat, NVP-2, Olaparib, Olmutinib, Omipalisib, Oprozomib, Osimertinib, OTS-964, Palbociclib, Pamiparib, Panobinostat, Paricalcitol, Parsaclisib, Pazopanib, Pemetrexed, Pemigatinib, Pevonedistat, Pexidartinib, Pifusertib, Plerixafor, PMPA, Ponatinib, Practinostat, Pralsetinib, Prednisone, Prexasertib, Prinomastat, Propranolol, Quisinostat, Quizartinib, Ralimetinib, Ravoxertinib, Regorafenib, Relugolix, Resminostat, Resveratrol, Retaspimycin, Retinoic acid, Ribociclib, Ricolinostat, Rigosertib, Ripretinib, RO-3306, Rocilinostat, Rogaratinib, Romidepsin, Rucaparib, Ruxolitinib, S2, S5, Saridegib, SBI-0654454, SCH772984, Seliciclib, Selitrectinib, Selpercatinib, Selumetinib, SGN-2FF, SGX393, Shikonin, Silibinin, Sitravatinib, Sonidegib, Sorafenib, Sotorasib, Staurosporine, SU11274, Sunitinib, Surufatinib, Tacedinaline, Tadalafil, Talazoparib, Taletrectinib, Tarloxotinib, Taselisib, Tazemetostat, Tefinostat, Temsirolimus, Tetrazole, Tivozanib, Tofacitinib, Tozasertib, Trametinib, Tranilast, Tretinoin, Trichostatin, Tucatinib, Tucidinostat, Tuvusertib, Ubenimex, Umbralisib, Uprosertib, USL311, Vactosertib, Valproic acid, Valsartan, Vandetanib, Veliparib, Vemurafenib, Venetoclax, Verteporfin, Vismodegib, Vorinostat, WRG-28, WZ811, Xevinapant, Zandelisib, Zanubrutinib, ZM447439;
each Ctᵢ is selected from the group comprising a deprotonated residue of Abiraterone, Aclarubicin, Adozelesin, Alrestatin, Amanitin, Amrubicin, Anthramycin, Arenastatin, Bizelesin, Bleomycin, Camptothecin, Capecitabine, Carzelesin, CC-1065, Chaconine, Chlorambucil, Cyclophosphamide, Cytarabine, Dacarbazine, Dactinomycin, Daunorubicin, DAVLBH, Deruxtecan, Dexamethasone, Dichloro acetic acid, Dimethyl-SGD-1882, Docetaxel, Dolastatin-10, Doxorubicin, Duocarmycin A, Duocarmycin B1, Duocarmycin B2, Duocarmycin C1, Duocarmycin C2, Duocarmycin D, Duocarmycin GA, Duocarmycin SA, Emetine, Epirubicin, Eribulin, Etoposide, Floxuridine, Fludarabine, Fluorouracil, Flutamide, Fulvestrant, Gemcitabine, Idarubicin, Ifosfamide, Irinotecan, L-Asparaginase, Lomustine, Melphalan, Mertansine, Methotrexate, Milataxel, Mitoxantrone, Monomethyl Auristatin E, Maytansine, Maytansinoid, Ozogamicin, Paclitaxel, Pirarubicin, Pixantrone, Podophyllotoxin, Procarbazine, Rapamycin, Rachelmycin, Salinomycin, SB-T-1214, Selinexor, SN-38, Solamargine, Solanine, Talirine, Temozolomide, Tesetaxel, SG3199 (Tesirine), Thapsigargin, Tomatine, Topotecan, Tubulysin B, Valrubicin, Vinblastine, Vincristine, Vinorelbine, VIP126, Zorubicin;
Z and R¹ form a moiety having a structure selected from the group comprising

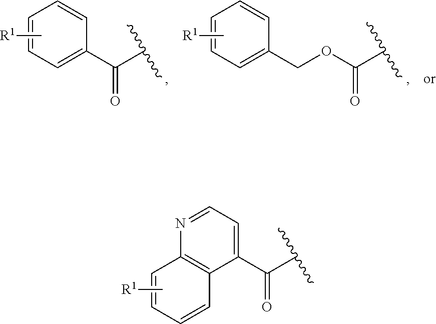

each Fcᵢ independently of each other comprises a moiety selected from the group comprising moieties

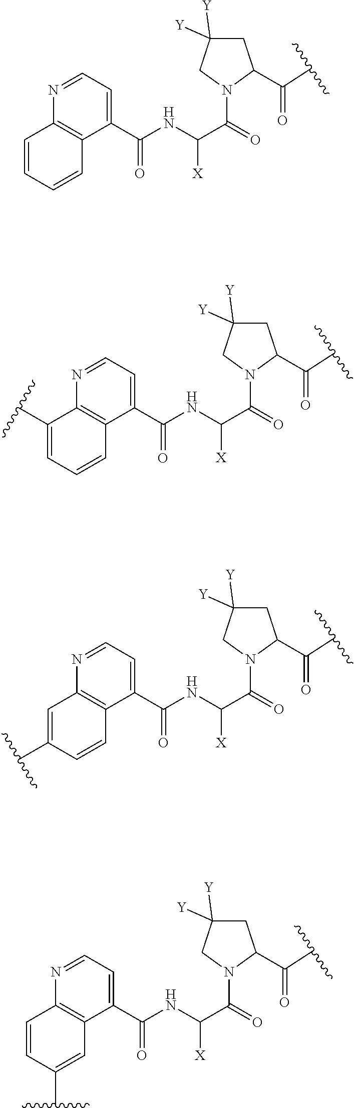

where the pyrrolidine ring is oriented towards the self-immolative linker L, Y=—H or —F and X=—H or —CH₃;
each Fcᵢ independently of each other comprises a moiety selected from the group comprising moieties

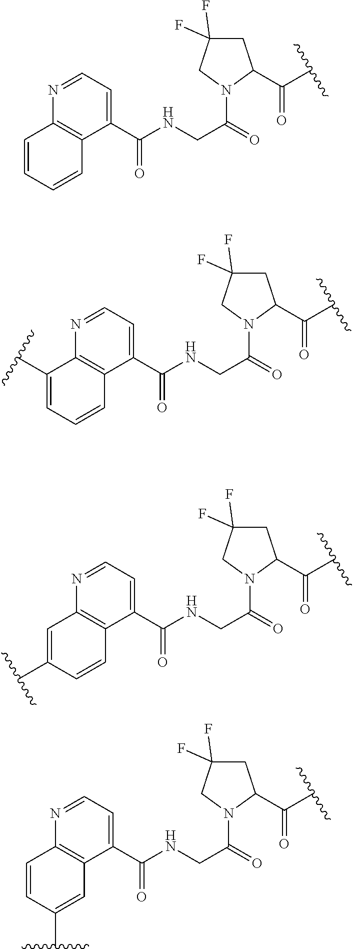 [SMILES: *C(=O)C1CC(F)(F)CN1C(=O)CNC(=O)c1ccnc2ccccc12 ; *C(=O)C1CC(F)(F)CN1C(=O)CNC(=O)c1ccnc2c(*)cccc12 ; *C(=O)C1CC(F)(F)CN1C(=O)CNC(=O)c1ccnc2cc(*)ccc12 ; *C(=O)C1CC(F)(F)CN1C(=O)CNC(=O)c1ccnc2ccc(*)cc12]

where the pyrrolidine ring is oriented towards the self-immolative linker L;
   each Fcᵢ independently from one another has the structure

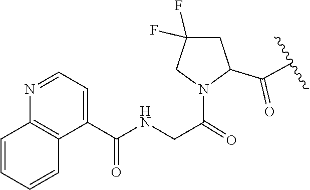 [SMILES: *C(=O)C1CC(F)(F)CN1C(=O)CNC(=O)c1ccnc2ccccc12]

each Fcᵢ independently from one another has the structure

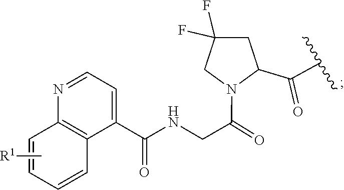

each Fcᵢ independently from one another has the structure

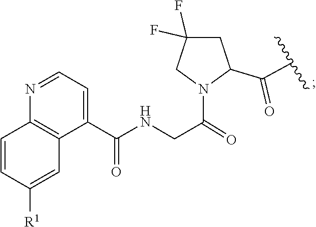

each Fcᵢ independently from one another has the structure

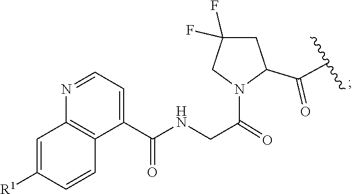

each Fcᵢ independently from one another has the structure

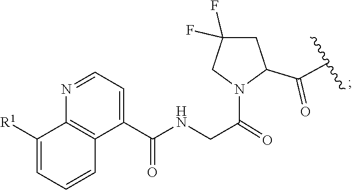

each linker Lᵢ independently of each other comprises a moiety having the structure

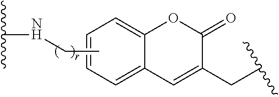

where
   the terminal amine is covalently bound to Fcᵢ; and
   r=0, 1, 2, 3, 4, 5, 6, 7, 8, 9 or 10;
   each Lᵢ independently of one another comprises a moiety having the structure

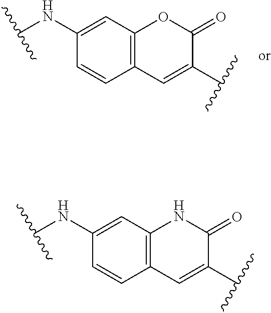 [SMILES: *Nc1ccc2cc(*)c(=O)oc2c1 ; *Nc1ccc2cc(*)c(=O)[nH]c2c1]

where the terminal amine is covalently bound to Fcᵢ;
   each Lᵢ independently of one another comprises a moiety having the structure

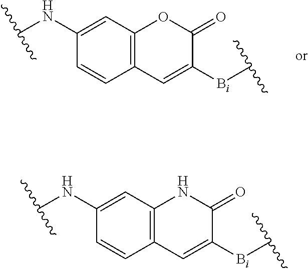

where the terminal amine is covalently bound to Fcᵢ and Bᵢ independently of one another is selected from the group of moieties comprising

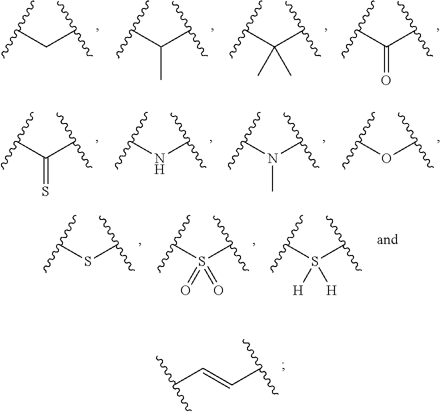

each Lᵢ independently of one another comprises a moiety having a structure selected from the group comprising

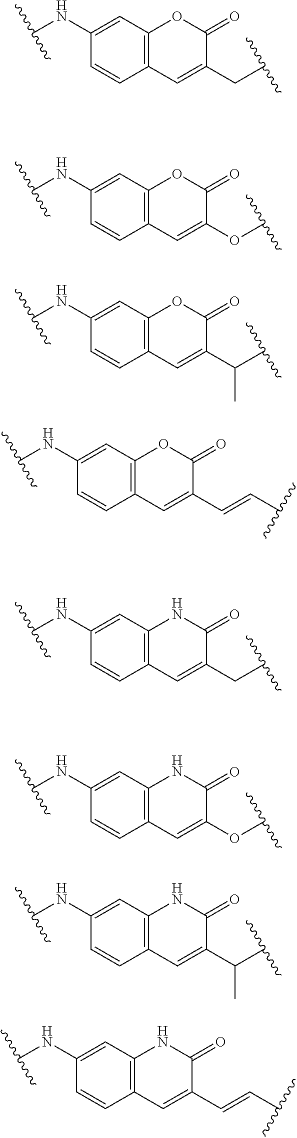

where the terminal amine is covalently bound to Fcᵢ;
   each linker Lᵢ independently of each other comprises a moiety having the structure

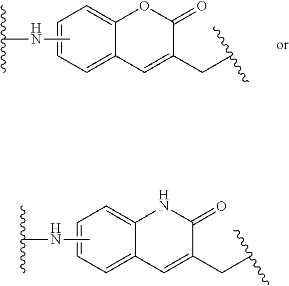

where the terminal amine is covalently bound to Fcᵢ;
   each linker Lᵢ independently of each other comprises a moiety having the structure

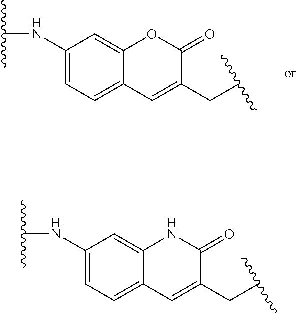 [SMILES: *Cc1cc2ccc(N*)cc2oc1=O ; *Cc1cc2ccc(N*)cc2[nH]c1=O]

where the terminal amine is covalently bound to Fcᵢ;
   each linker Lᵢ independently from one another comprises a second pharmacokinetic modulating moiety R²;
   each linker Lᵢ independently from one another is a linker configured for triggered self-immolation via cyclization;
   each linker Lᵢ independently from one another is a linker configured for triggered self-immolation via 1,4-elimination;
   each linker Lᵢ independently from one another is a linker configured for triggered self-immolation via 1,6-elimination;
   each linker Lᵢ independently from one another is a linker configured for triggered self-immolation via 1,8-elimination;
   each linker Lᵢ independently from one another comprises an amine group covalently bound to Fcᵢ;
   each linker Lᵢ independently from one another has a structure selected from the group comprising structures (a), (b), (c), (d), (e), (f), (g), (h), (i), (j), (k), (l), (m), (n) and (o) with

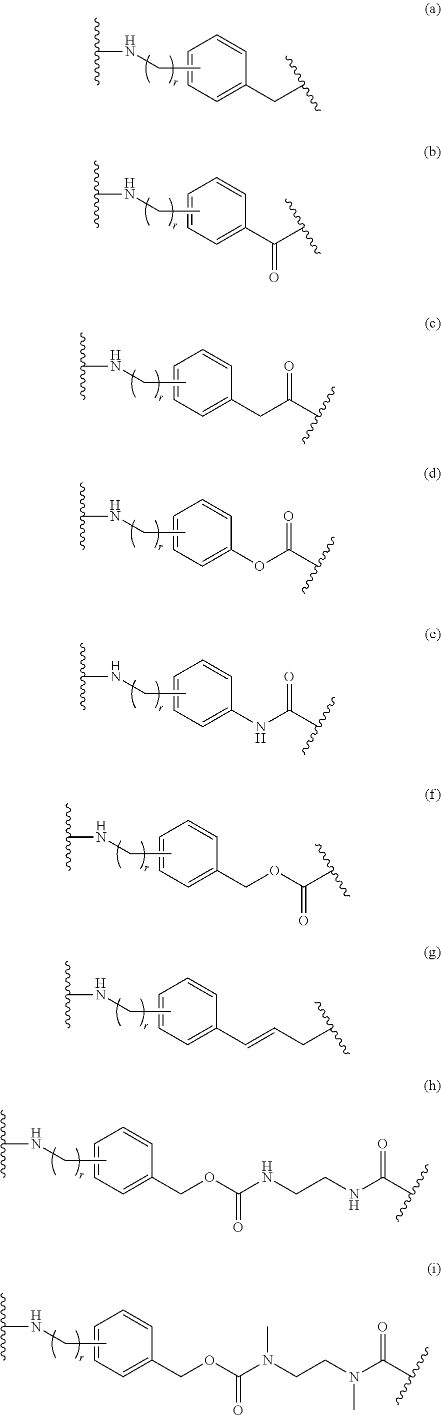

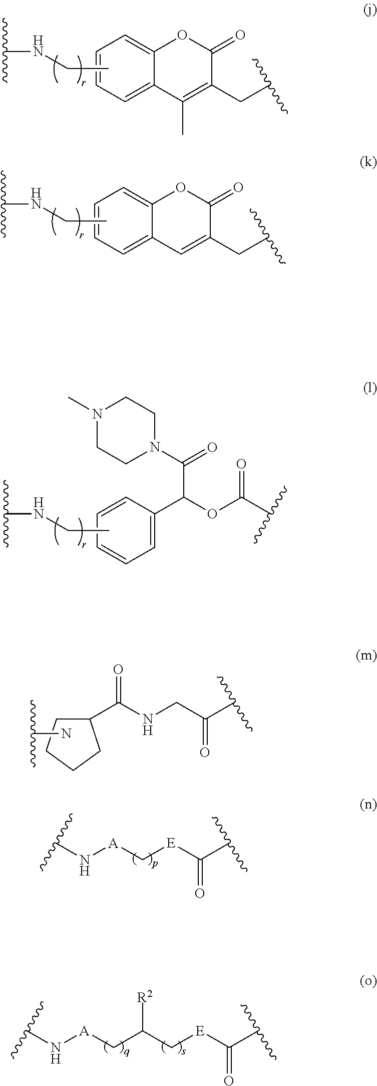

where
   the terminal amine is covalently bound to Fcᵢ;
   r=0, 1, 2, 3, 4, 5, 6, 7, 8, 9 or 10;
   -A- is absent or selected from the group comprising —O—, —NH—, —CH(OH)—, —CO—, —N(CH₃)—, —S— and —SH₂—;
   -E- is selected from the group comprising —CH₂—, —O—, —NH—, —N(CH₃)—, —S— and —SH₂—;
   p=0, 1, 2, 3, 4, 5, 6, 7, 8, 9 or 10;
   q=0, 1, 2, 3, 4, 5, 6, 7, 8, 9 or 10 and s=0, 1, 2, 3, 4, 5, 6, 7, 8, 9 or 10 and q+s=0, 1, 2, 3, 4, 5, 6, 7, 8, 9 or 10;
   —R² is —H, —CH₃ or a second pharmacokinetic modulating moiety;
   each linker Lᵢ independently from one another has the structure

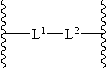

wherein
L¹ is selected from the group comprising structures (a), (b), (c), (d), (e), (f), (g), (h), (i), (j), (k), (l), (m), (n) and (o) with

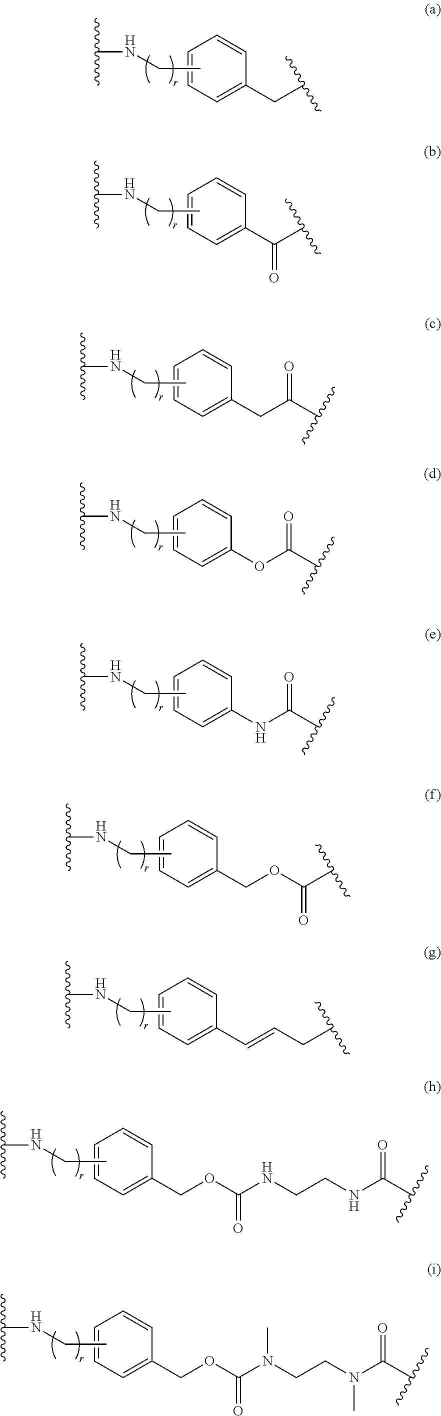

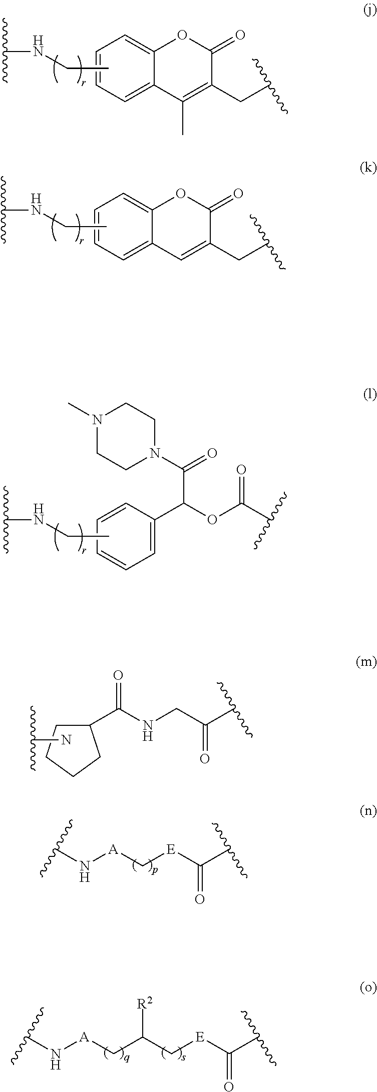

where
   the terminal amine is covalently bound to Fcᵢ;
   r=0, 1, 2, 3, 4, 5, 6, 7, 8, 9 or 10;
   -A- is absent or selected from the group comprising —O—, —NH—, —CH(OH)—, —CO—, —N(CH₃)—, —S— and —SH₂—;
   -E- is selected from the group comprising —CH₂—, —O—, —NH—, —N(CH₃)—, —S— and —SH₂—;
   p=0, 1, 2, 3, 4, 5, 6, 7, 8, 9 or 10;
   q=0, 1, 2, 3, 4, 5, 6, 7, 8, 9 or 10 and s=0, 1, 2, 3, 4, 5, 6, 7, 8, 9 or 10 and q+s=0, 1, 2, 3, 4, 5, 6, 7, 8, 9 or 10;
   —R² is —H, —CH₃ or a second pharmacokinetic modulating moiety; and
L² is selected from the group comprising structures (a′), (b′), (c′), (d′), (e′), (f′), (g′), (h′), (i′), (j′), (k′), (l′), (m′), (n′) and (o′) with

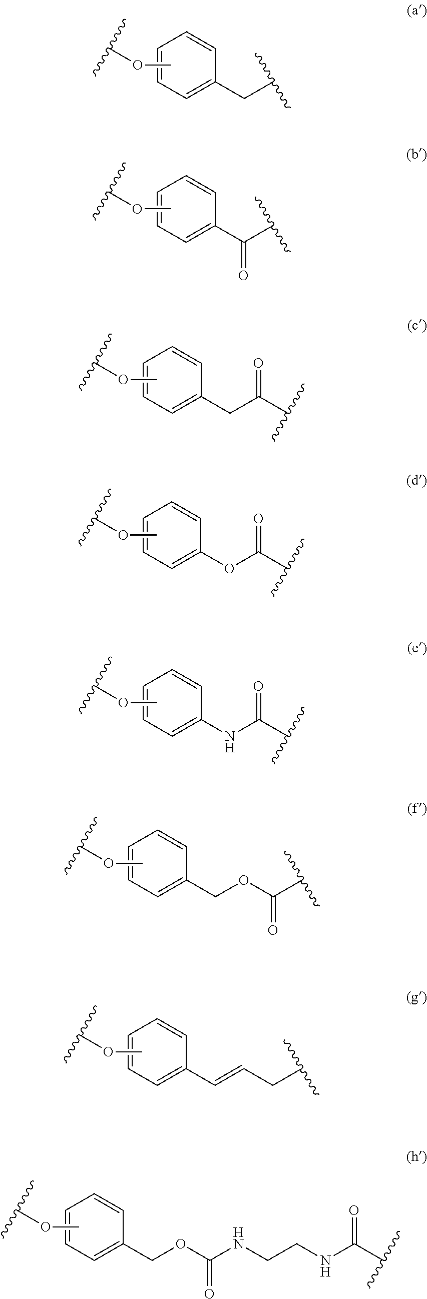

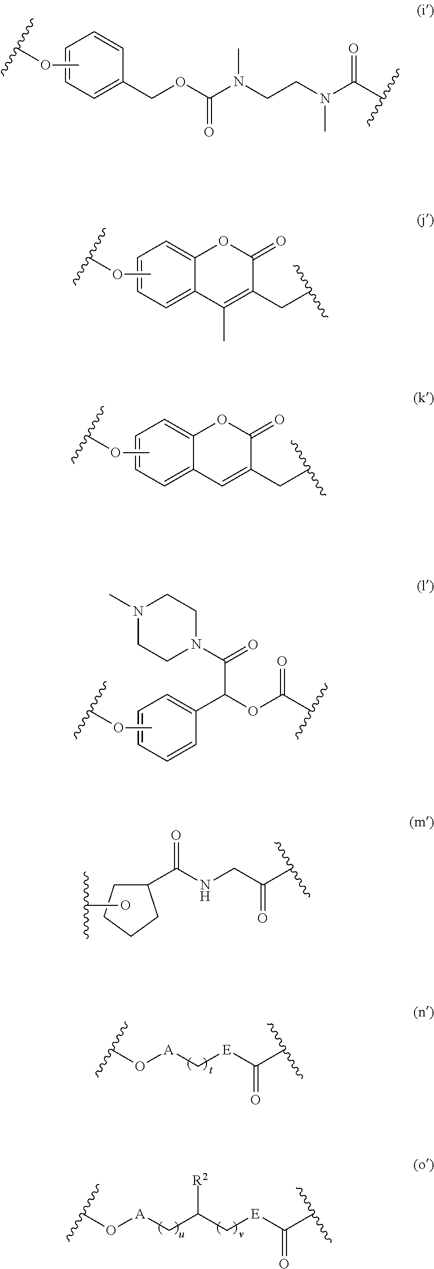

where
   if L¹ is equal to one of structures (a)-(m), then L² is equal to (n′) or (o′); and if L¹ is equal to (n) or (o), then L² is equal to one of structures (a′)-(m′);
   -A- is absent or selected from the group comprising —O—, —NH—, —CH(OH)—, —CO—, —N(CH₃)—, —S— and —SH₂—;
   -E- is selected from the group comprising —CH₂, —O—, —NH—, —N(CH₃)—, —S— and —SH₂—;
   t=0, 1, 2, 3, 4, 5, 6, 7, 8, 9 or 10;
   u=0, 1, 2, 3, 4, 5, 6, 7, 8, 9 or 10 and v=0, 1, 2, 3, 4, 5, 6, 7, 8, 9 or 10 and u+v=0, 1, 2, 3, 4, 5, 6, 7, 8, 9 or 10; and
   —R² is —H, —CH₃ or a second pharmacokinetic modulating moiety;
   each Lᵢ independently from one another has the structure

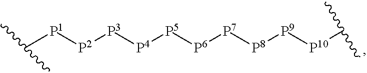

where
P¹ comprises an amine group covalently bound to Fcᵢ, P¹ is selected from the group comprising

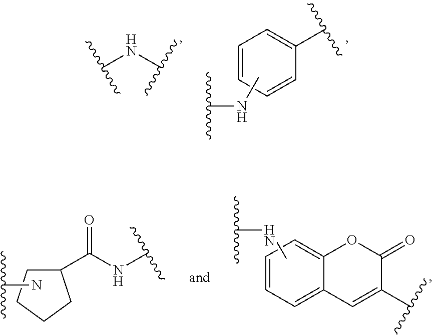

and
moieties P^{j} with 2≤j≤h and 2≤h≤10 independently from one another are selected from the group comprising

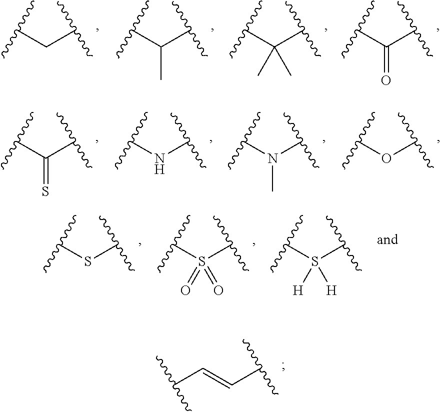

and P^{j} with h<j≤10 are absent;
   each Lᵢ independently from one another has the structure

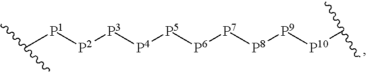

where
P^{l} is selected from the group comprising

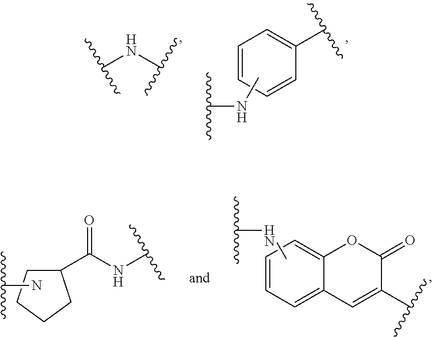

Fcᵢ and P^{l} form a moiety having a structure selected from the group comprising

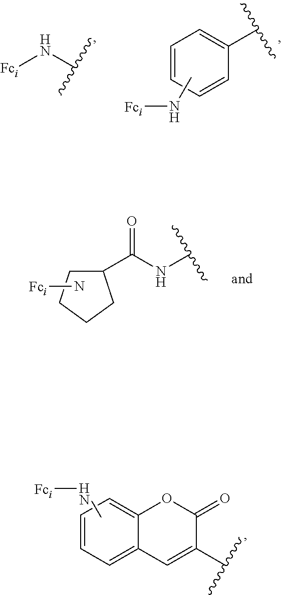

and
moieties P^{j} with 2≤j≤h and 2≤h≤10 independently from one another are selected from the group comprising

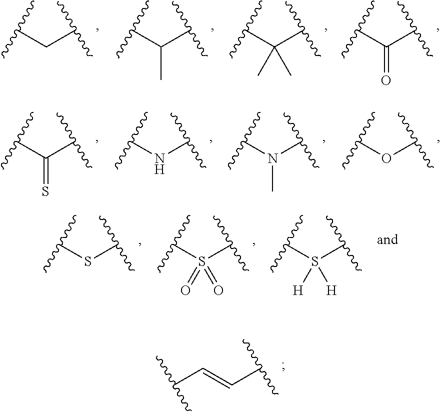

and P^{j} with h<j≤10 are absent;
   each Lᵢ independently from one another has the structure

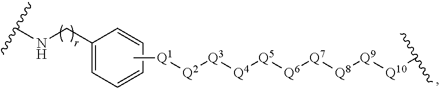

where
the amine group is covalently bound to Fcᵢ, r=0, 1, 2, 3, 4, 5, 6, 7, 8, 9 or 10 and moieties Q^{j} with 1≤j≤h and 1≤h≤10 independently from one another are selected from the group comprising

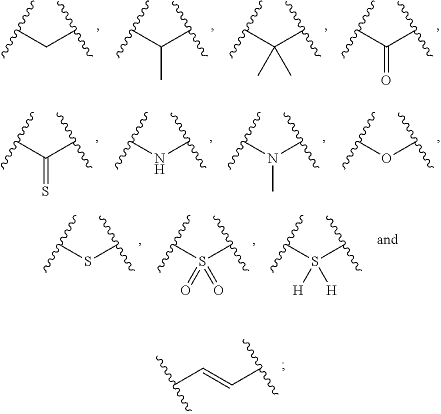

and Q^{j} with h<j≤10 are absent;
   each Lᵢ independently from one another has the structure

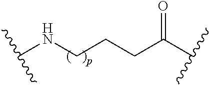

with p=1, 2, 3, 4, 5, 6, 7, 8, 9 or 10;
each Lᵢ independently from one another has the structure

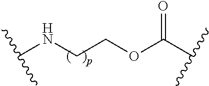

with p=1, 2, 3, 4, 5, 6, 7, 8, 9 or 10;
each Lᵢ independently from one another has the structure

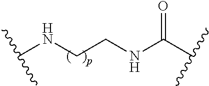

with p=1, 2, 3, 4, 5, 6, 7, 8, 9 or 10;
each Lᵢ independently from one another has the structure

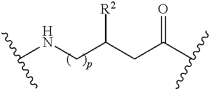

where —R² is a residue of a second pharmacokinetic modulating moiety and p=1, 2, 3, 4, 5, 6, 7, 8, 9 or 10;
each Lᵢ independently from one another has the structure

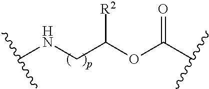

where —R² is a residue of a second pharmacokinetic modulating moiety and p=1, 2, 3, 4, 5, 6, 7, 8, 9 or 10;
each Lᵢ independently from one another has the structure

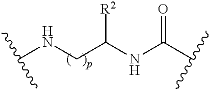

where —R² is a residue of a second pharmacokinetic modulating moiety and p=1, 2, 3, 4, 5, 6, 7, 8, 9 or 10;
each Lᵢ independently from one another has a structure selected from the group comprising structures (a″), (b″), (c″), (d″), (e″), (f″), (g″), (h″), (i″), (j″), (k″), (l″), (m″), (n″), (o″), (p″), (q″), (r″) and (s″) with

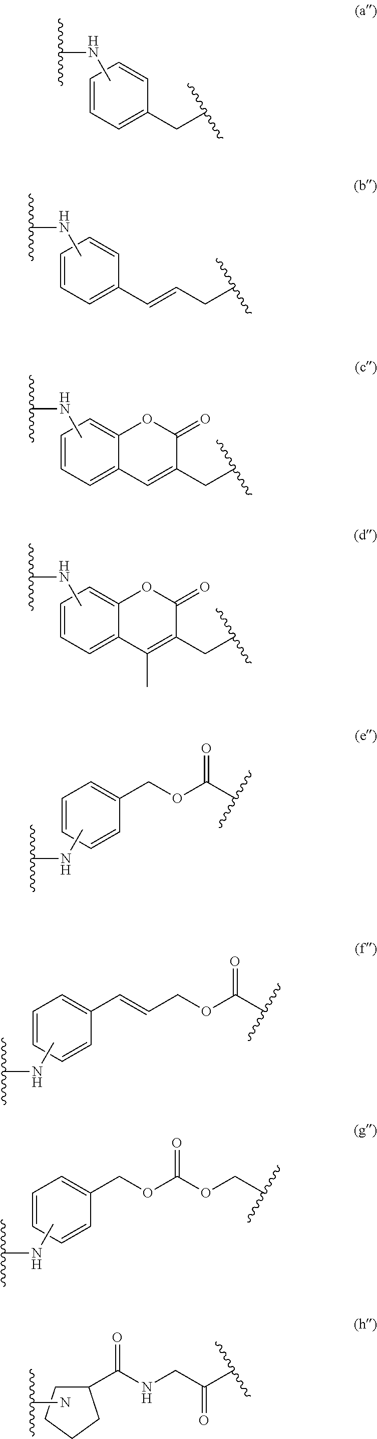

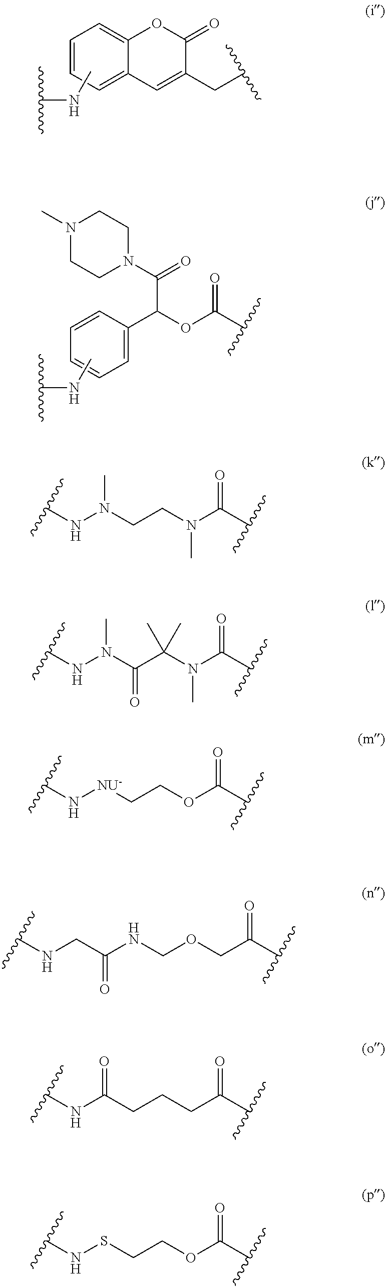

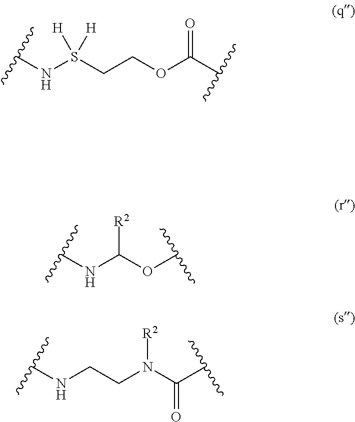

where —R² is —H, —CH₃ or a residue of a second pharmacokinetic modulating moiety and NU⁻ is a nucleophile selected from O, NH or S;
   each Lᵢ independently from one another has a structure selected from the group comprising structures (a″), (b″), (c″), (d″), (e″), (f″), (g″), (h″), (i″), (j″), (k″), (l″), (m″), (n″), (o″), (p″), (q″), (r″), (s″) and Fcᵢ is covalently bound to the amine group of Lᵢ;
   each Lᵢ has structure (a″);
   each Lᵢ has structure (b″);
   each Lᵢ has structure (c″);
   each Lᵢ has structure (d″);
   each Lᵢ has structure (e″);
   each Lᵢ has structure (f″);
   each Lᵢ has structure (g″);
   each Lᵢ has structure (h″);
   each Lᵢ has structure (i″);
   each Lᵢ has structure (j″);
   each Lᵢ has structure (k″);
   each Lᵢ has structure (l″);
   each Lᵢ has structure (m″), wherein NU-designates a nucleophile selected from O, NH and S;
   each Lᵢ has structure (n″);
   each Lᵢ has structure (o″);
   each Lᵢ has structure (p″);
   each Lᵢ has structure (q″);
   each Lᵢ has structure (r″);
   each Lᵢ has structure (s″);

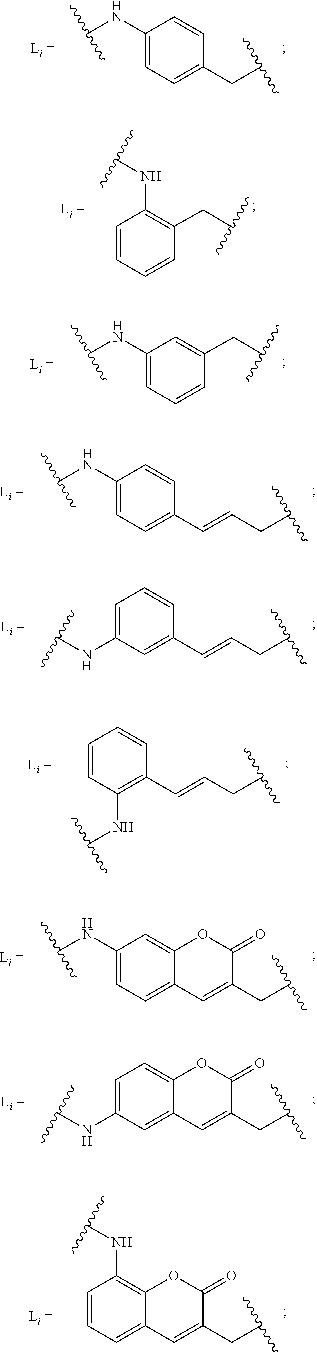

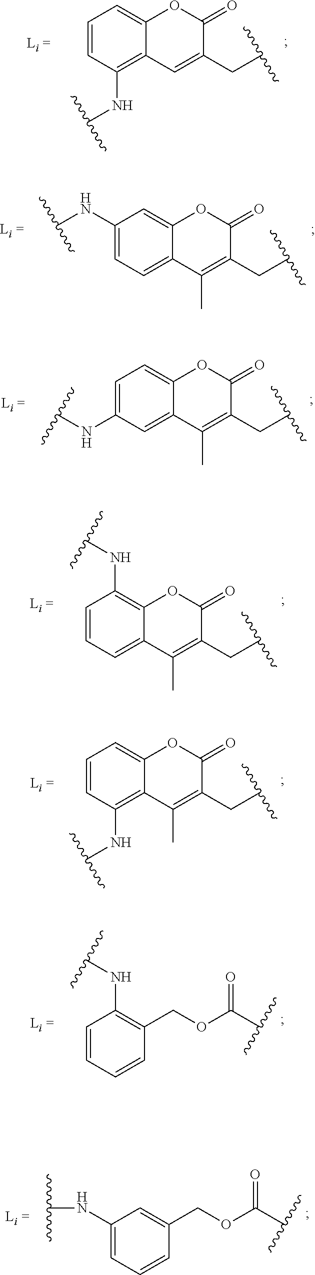

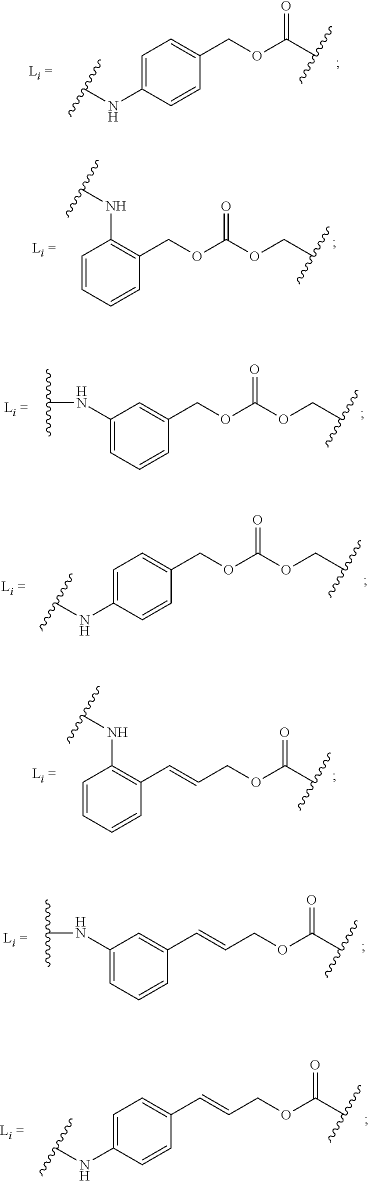

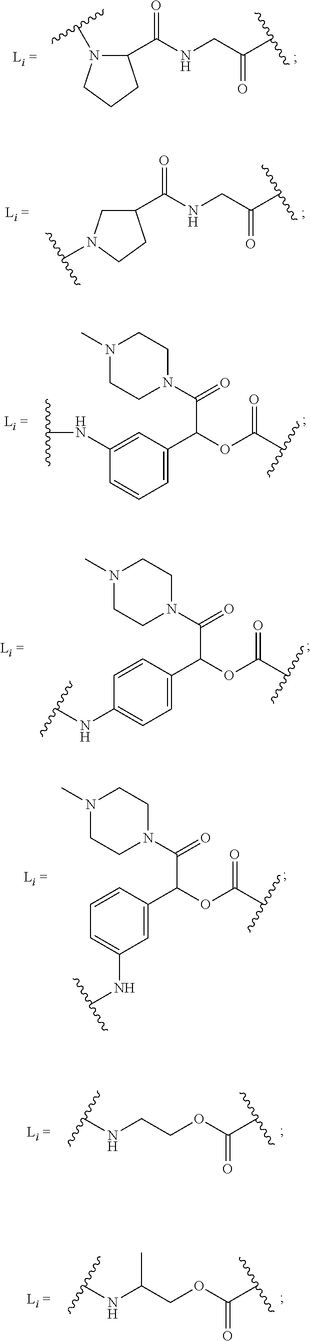

each prodrug Sᵢ independently from one another has a structure of type (x) or (y) with

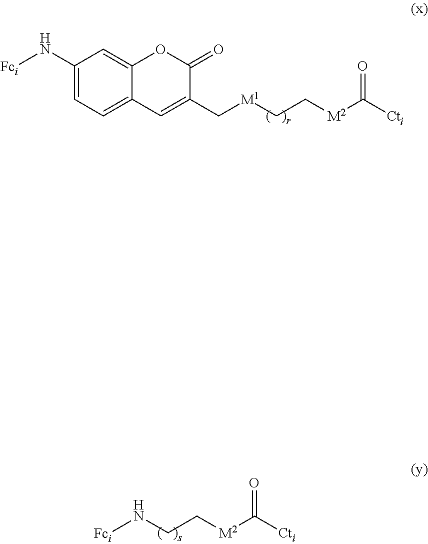

where M¹=—O—, —NH—, —N(CH₃)— or —S—, M²=—CH₂—, —O— or —NH—, r=0, 1, 2, 3, 4, 5, 6, 7, 8, 9 or 10 and s=0, 1, 2, 3, 4, 5, 6, 7, 8, 9 or 10;
   each prodrug Sᵢ has a structure of type (x) with

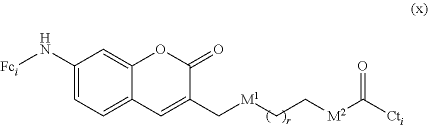

where M¹=—O—, —NH—, —N(CH₃)— or —S—, M²=—CH₂—, —O— or —NH— and r=0, 1, 2, 3, 4, 5, 6, 7, 8, 9 or 10;

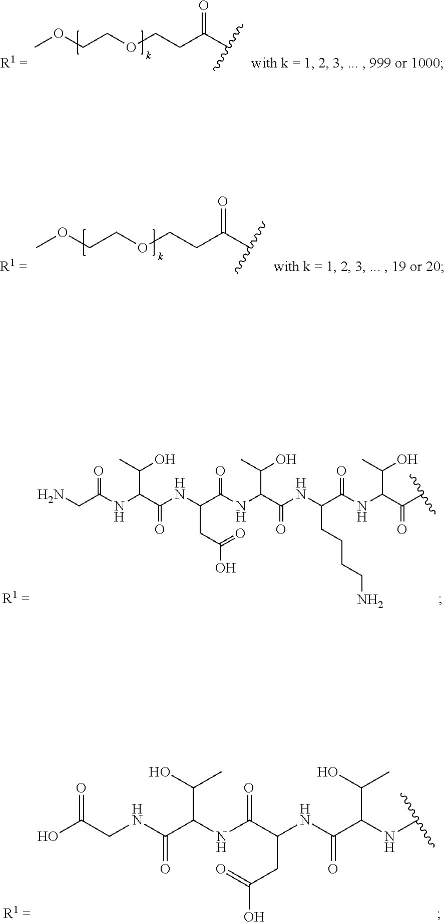

R¹ is a residue of a peptide comprising 1, 2, 3, 4, 5, 6, 7, 8, 9, 10, 11, 12, 13, 14, 15, 16, 17, 18, 19 or 20 amino acids independently selected from the group comprising Ala, Arg, Asn, Asp, Cys, Gln, Glu, Gly, His, Ile, Leu, Lys, Met, Phe, Pro, Ser, Thr, Trp, Tyr, Val, Pyl, Sec, GABA or γ-Aminobutyric acid, Homoserine, DOPA or 3,4-Dihydroxyphenylalanine, Citrulline, β-Alanine and Thyroxine;
R¹ is a residue of a lactide oligomer comprising 4, 5, . . . , 39 or 40 mer units;
R¹ is a residue of a lactide-co-glycolide oligomer comprising 4, 5, . . . , 39 or 40 mer units;
R¹ is a residue of an acrylate oligomer comprising 4, 5, . . . , 39 or 40 mer units;
R¹ is a residue of a methacrylate oligomer comprising 4, 5, . . . , 39 or 40 mer units;

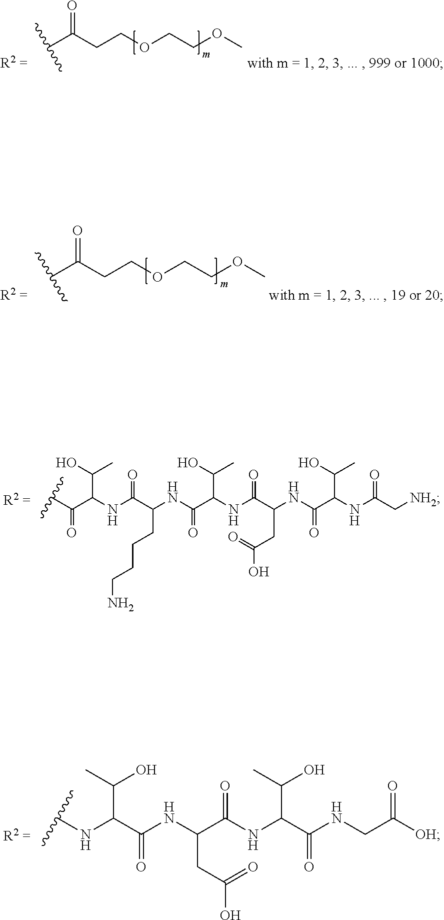

R² is a residue of a peptide comprising 1, 2, 3, 4, 5, 6, 7, 8, 9, 10, 11, 12, 13, 14, 15, 16, 17, 18, 19 or 20 amino acids independently selected from the group comprising Ala, Arg, Asn, Asp, Cys, Gln, Glu, Gly, His, Ile, Leu, Lys, Met, Phe, Pro, Ser, Thr, Trp, Tyr, Val, Pyl, Sec, GABA or γ-Aminobutyric acid, Homoserine, DOPA or 3,4-Dihydroxyphenylalanine, Citrulline, β-Alanine and Thyroxine;
R² is a residue of a lactide oligomer comprising 4, 5, . . . , 39 or 40 mer units;
R² is a residue of a lactide-co-glycolide oligomer comprising 4, 5, . . . , 39 or 40 mer units;
R² is a residue of an acrylate oligomer comprising 4, 5, . . . , 39 or 40 mer units;
R² is a residue of a methacrylate oligomer comprising 4, 5, . . . , 39 or 40 mer units.

A second embodiment of the invention concerns a prodrug having the structure

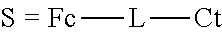

wherein
   Fc has the structure

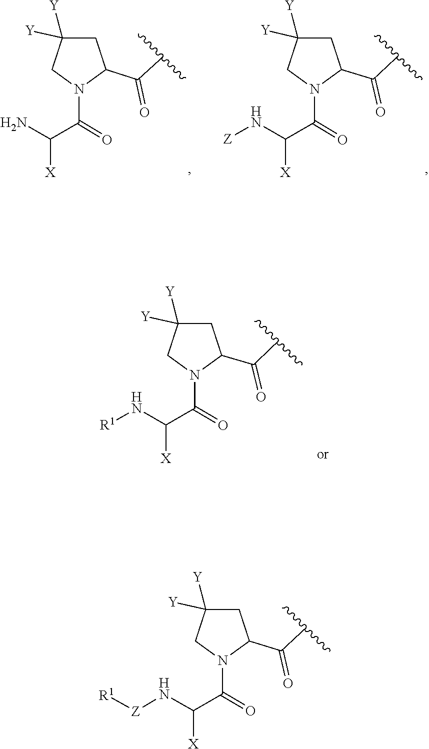

where X=—H or —CH₃, Y=—H or —F, —R¹ is a residue of a first pharmacokinetic modulating moiety and Z is a moiety having a structure selected from the group comprising structures (1), (2), (3), (4), (5), (6), (7), (8), (9), (10), (11), (12), (13), (14) and (15) with

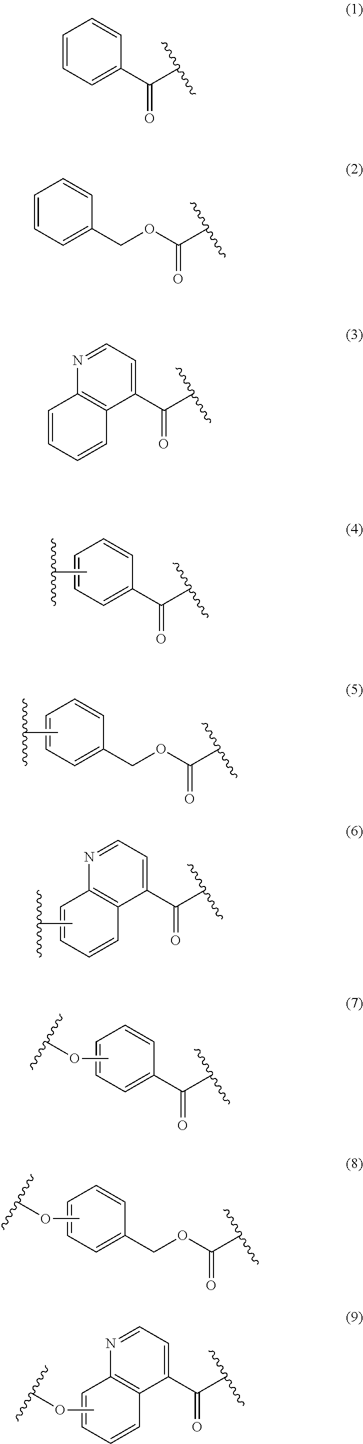

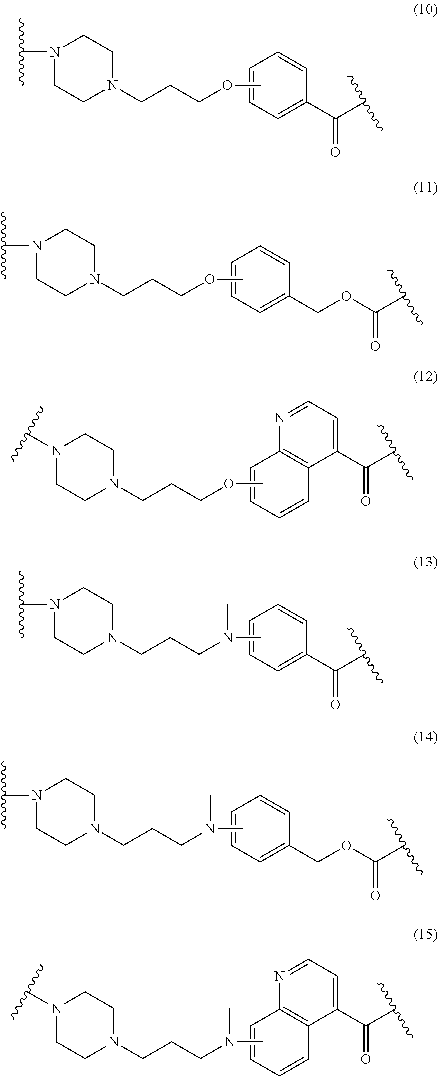

L is a residue of a self-immolative linker;
Ct and Fc are covalently bound to L; and
Ct is equal to a deprotonated residue of 1,2,3,4-Tetrahydrogen-staurosporine, 17-Dmag, 2-Aminopropanenitrile, 4SC202, ABBV-CLS-484, Abemaciclib, Abexinostat, Acalabrutinib, Acetylbufalin, Aderbasib, Afatinib, Afuresertib, Alectinib, Alisertib, Alpelisib, Alvocidib, AMD3465, Anlotinib, Apalutamide, AR-42, Asciminib, Atuveciclib, Avapritinib, Axitinib, AZD7762, BAY1125976, Belinostat, β-Hydroxyisovaleric acid, BF211, Bicalutamide, Binimetinib, Bortezomib, Bosutinib, Brigatinib, Bufalin, Buparlisib, Buthionine sulfoximine, Cabozantinib, Capivasertib, Capmatinib, Carfilzomib, CEP-9722, Ceralasertib, Ceritinib, Chidamide, CHR-3996, Citarinostat, Cobimetinib, CompK, Copanlisib, Crenolanib, Crizotinib, CUDC-101, Dabrafenib, Daclatasvir, Dacomitinib, Darolutamide, Dasatinib, Dasatinib D1, Dasatinib D2, Dasatinib D3, Dasatinib D4, Decitabine, Defactinib, Degarelix, Diethylstilbestrol, Dinaciclib, Dp44mT, DpC, DUPA, Duvelisib, E7016, Ebvaciclib, Eganelisib, Elimusertib, Emavusertib, Enasidenib, Encorafenib, Enitociclib, Entinostat, Entrectinib, Enzalutamide, Epacadostat, Epigallocatechin gallate, Epoxomicin, Erdafitinib, Erismodegib, Erlotinib, Everolimus, Fasudil, Fedratinib, Filgotinib, Foslinanib, Fostamatinib, Fruquintinib, Galunisertib, Ganetespib, Gedatolisib, Gefitinib, GFH018, Gilteritinib, Givinostat, Glasdegib, Goserelin, GSK2256098, GSK269962A, GSK690693, GUL, Halofuginone, Hymecromone, Ibrutinib, Icotinib, Idelalisib, Imatinib, Imiquimod, Infigratinib, Iniparib, Ipatasertib, Itacitinib, Ivaltinostat, Ivosidenib, Ixazomib, Kevetrin, Lapatinib, Larotrectinib, Lenalidomide, Leniolisib, Lenvatinib, Leuprolide, Linsitinib, Lonafarnib, Lorlatinib, Losartan, Lucitanib, Luminespib, M1096, Marizomib, ME-344, Merestinib, Metformin, MG132, Midostaurin, Miransertib, Mivavotinib, MK2206, MMP-9 Inhibitor I, Mobocertinib, Mocetinostat, Motesanib, MRTX1133, Navitoclax, Nazartinib, Nedisertib, Neratinib, Nilotinib, Nilutamide, Nintedanib, Niraparib, NMS-P118, NMS-P515, NSC668394, NSC95397, Numidargistat, NVP-2, Olaparib, Olmutinib, Omipalisib, Oprozomib, Osimertinib, OTS-964, Palbociclib, Pamiparib, Panobinostat, Paricalcitol, Parsaclisib, Pazopanib, Pemetrexed, Pemigatinib, Pevonedistat, Pexidartinib, Pifusertib, Plerixafor, PMPA, Ponatinib, Practinostat, Pralsetinib, Prednisone, Prexasertib, Prinomastat, Propranolol, Quisinostat, Quizartinib, Ralimetinib, Ravoxertinib, Regorafenib, Relugolix, Resminostat, Resveratrol, Retaspimycin, Retinoic acid, Ribociclib, Ricolinostat, Rigosertib, Ripretinib, RO-3306, Rocilinostat, Rogaratinib, Romidepsin, Rucaparib, Ruxolitinib, S2, S5, Saridegib, SBI-0654454, SCH772984, Seliciclib, Selitrectinib, Selpercatinib, Selumetinib, SGN-2FF, SGX393, Shikonin, Silibinin, Sitravatinib, Sonidegib, Sorafenib, Sotorasib, Staurosporine, SU11274, Sunitinib, Surufatinib, Tacedinaline, Tadalafil, Talazoparib, Taletrectinib, Tarloxotinib, Taselisib, Tazemetostat, Tefinostat, Temsirolimus, Tetrazole, Tivozanib, Tofacitinib, Tozasertib, Trametinib, Tranilast, Tretinoin, Trichostatin, Tucatinib, Tucidinostat, Tuvusertib, Ubenimex, Umbralisib, Uprosertib, USL311, Vactosertib, Valproic acid, Valsartan, Vandetanib, Veliparib, Vemurafenib, Venetoclax, Verteporfin, Vismodegib, Vorinostat, WRG-28, WZ811, Xevinapant, Zandelisib, Zanubrutinib, ZM447439, Abiraterone, Aclarubicin, Adozelesin, Alrestatin, Amanitin, Amrubicin, Anthramycin, Arenastatin, Bizelesin, Bleomycin, Camptothecin, Capecitabine, Carzelesin, CC-1065, Chaconine, Chlorambucil, Cryptophycin-24, Cyclophosphamide, Cytarabine, Dacarbazine, Dactinomycin, Daunorubicin, DAVLBH, Deruxtecan, Dexamethasone, Dichloro acetic acid, Dimethyl-SGD-1882, Docetaxel, Dolastatin-10, Doxorubicin, Duocarmycin A, Duocarmycin B1, Duocarmycin B2, Duocarmycin C1, Duocarmycin C2, Duocarmycin D, Duocarmycin GA, Duocarmycin SA, Emetine, Epirubicin, Eribulin, Etoposide, Floxuridine, Fludarabine, Fluorouracil, Flutamide, Fulvestrant, Gemcitabine, Idarubicin, Ifosfamide, Irinotecan, L-Asparaginase, Lomustine, Melphalan, Mertansine, Methotrexate, Milataxel, Mitoxantrone, Monomethyl Auristatin E, Maytansine, Maytansinoid, Ozogamicin, Paclitaxel, Pirarubicin, Pixantrone, Podophyllotoxin, Procarbazine, Rapamycin, Rachelmycin, Salinomycin, SB-T-1214, Selinexor, SN-38, Solamargine, Solanine, Talirine, Temozolomide, Tesetaxel, SG3199 (Tesirine), Thapsigargin, Tomatine, Topotecan, Tubulysin B, Valrubicin, Vinblastine, Vincristine, Vinorelbine, VIP126, Zorubicin.

Expedient embodiments of the inventive prodrug S=Fc-L-Ct are characterized by one of the following features or a combination of two or more of the following features insofar the combined features are not mutually exclusive or contradictory and according to which:
Ct is a radical of Dinaciclib;
Ct is a radical of NVP-2;
Ct is a radical of Erlotinib;
Ct is a radical of Imatinib;
Ct is a radical of Sorafenib;
Ct is a radical of Bufalin;
Ct is a radical of Acetylbufalin;
   Z and R¹ form a moiety having a structure selected from the group comprising

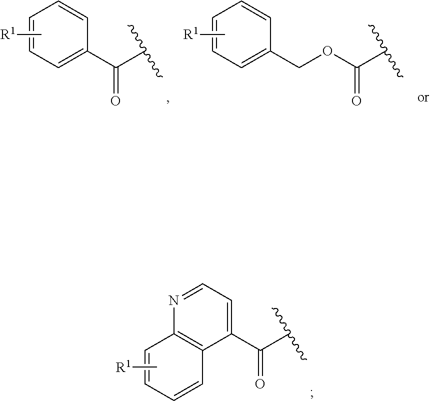

Fc comprises a moiety selected from the group comprising moieties

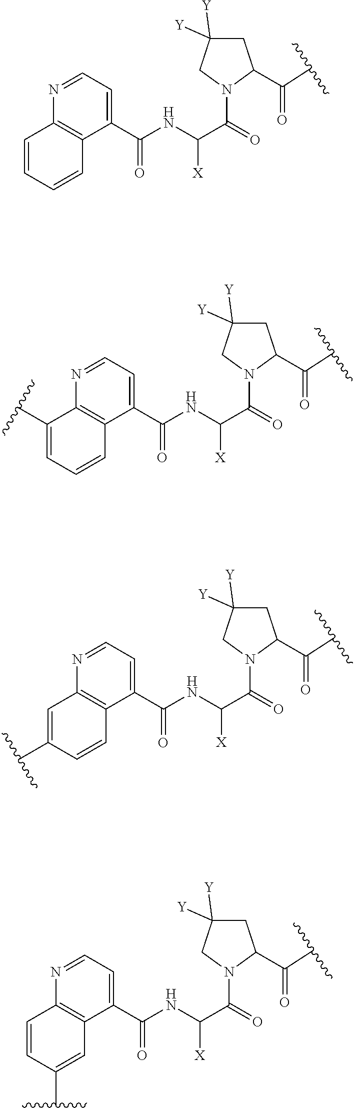

where the pyrrolidine ring is oriented towards the self-immolative linker L, Y=—H or —F and X=—H or —CH₃;
   Fc comprises a moiety selected from the group comprising moieties

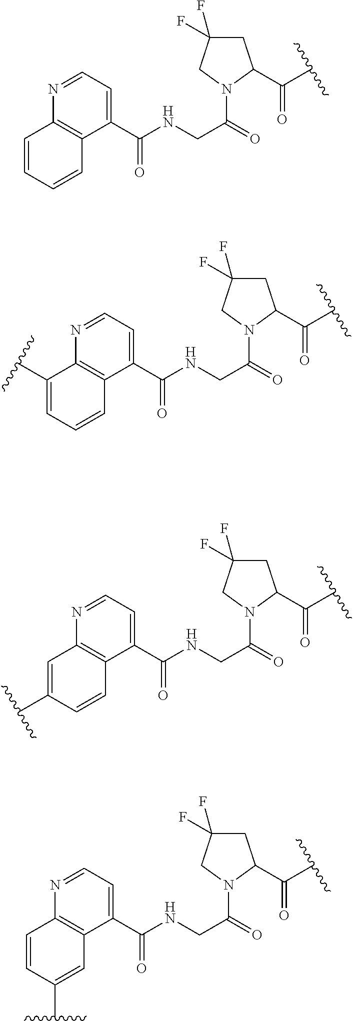 [SMILES: *C(=O)C1CC(F)(F)CN1C(=O)CNC(=O)c1ccnc2ccccc12 ; *C(=O)C1CC(F)(F)CN1C(=O)CNC(=O)c1ccnc2c(*)cccc12 ; *C(=O)C1CC(F)(F)CN1C(=O)CNC(=O)c1ccnc2cc(*)ccc12 ; *C(=O)C1CC(F)(F)CN1C(=O)CNC(=O)c1ccnc2ccc(*)cc12]

where the pyrrolidine ring is oriented towards the self-immolative linker L;
   Fc has the structure

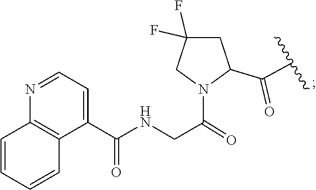 [SMILES: *C(=O)C1CC(F)(F)CN1C(=O)CNC(=O)c1ccnc2ccccc12]

Fc has the structure

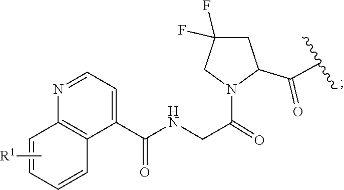

Fc has the structure

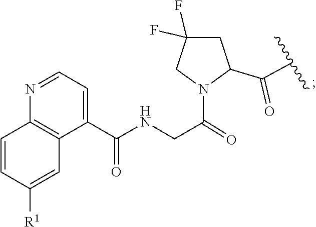

Fc has the structure

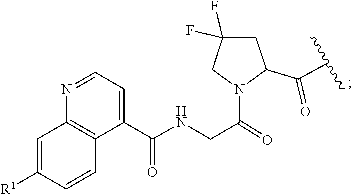

Fc has the structure

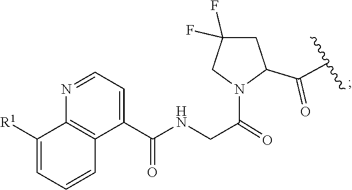

L comprises a moiety having the structure

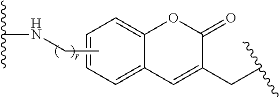

where
   the terminal amine is covalently bound to Fc; and
   r=0, 1, 2, 3, 4, 5, 6, 7, 8, 9 or 10;
   L comprises a moiety having the structure

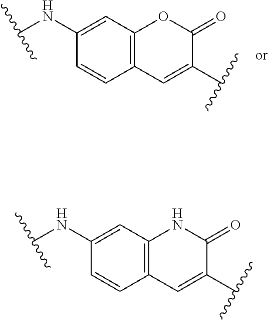 [SMILES: *Nc1ccc2cc(*)c(=O)oc2c1 ; *Nc1ccc2cc(*)c(=O)[nH]c2c1]

where the terminal amine is covalently bound to Fc;
   L comprises a moiety having the structure

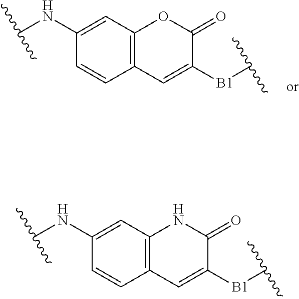

where the terminal amine is covalently bound to Fc and B1 is selected from the group of moieties comprising

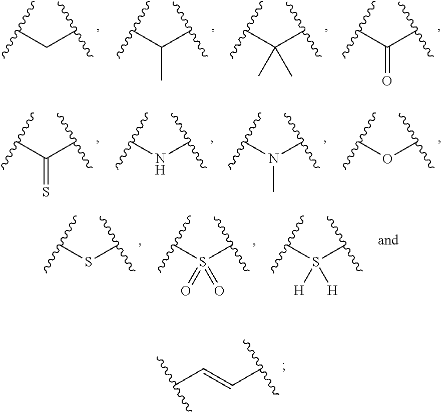

L comprises a moiety having a structure selected from the group comprising

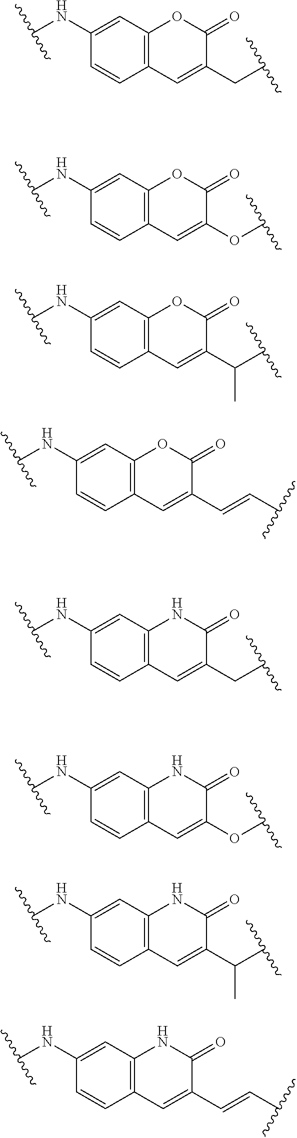

where the terminal amine is covalently bound to Fc;
   L comprises a moiety having the structure

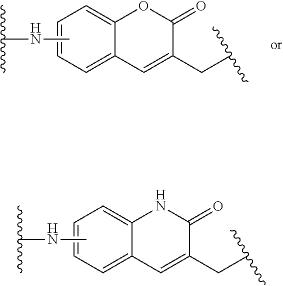

where the terminal amine is covalently bound to Fc;
   L comprises a moiety having the structure

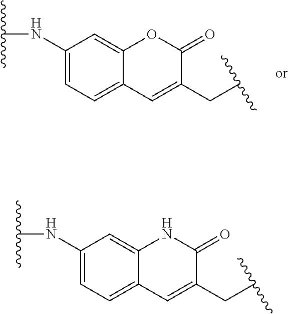 [SMILES: *Cc1cc2ccc(N*)cc2oc1=O ; *Cc1cc2ccc(N*)cc2[nH]c1=O]

where the terminal amine is covalently bound to Fc;
   linker L comprises a second pharmacokinetic moiety R²;
   linker L is configured for triggered self-immolation via cyclization;
   linker L is configured for triggered self-immolation via 1,4-elimination;
   linker L is configured for triggered self-immolation via 1,6-elimination;
   linker L is configured for triggered self-immolation via 1,8-elimination;
   linker L comprises an amine group covalently bound to Fc;
   linker L has a structure selected from the group comprising structures (a), (b), (c), (d), (e), (f), (g), (h), (i), (j), (k), (l), (m), (n) and (o) with

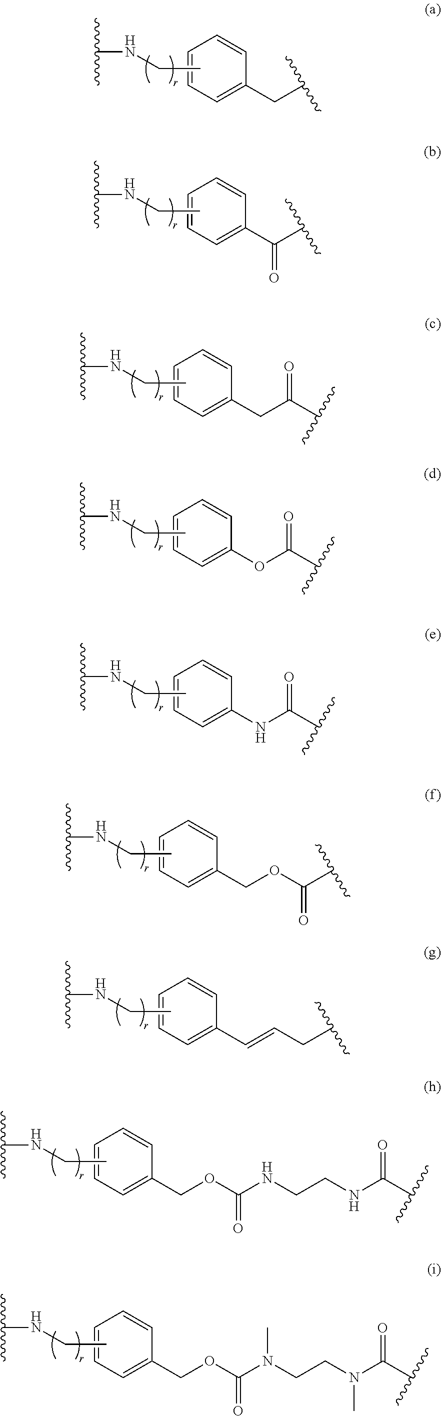

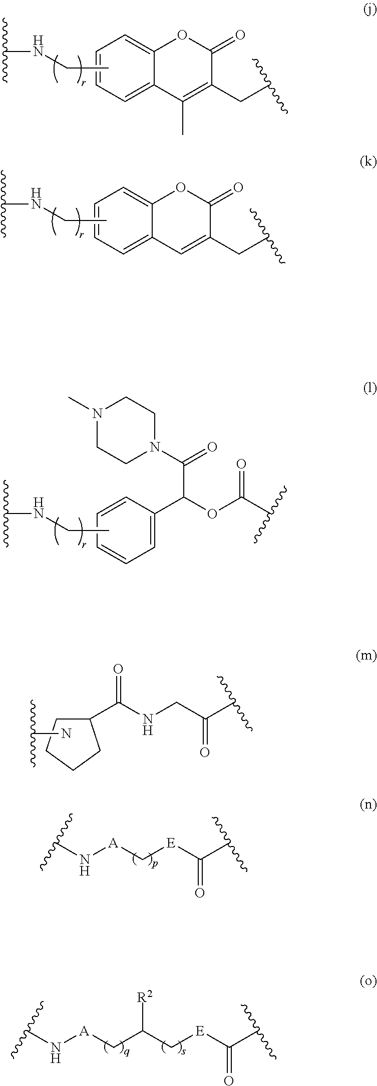

where
   the terminal amine is covalently bound to Fc;
   r=0, 1, 2, 3, 4, 5, 6, 7, 8, 9 or 10;
   -A- is absent or selected from the group comprising —O—, —NH—, —CH(OH)—, —CO—, —N(CH₃)—, —S— and —SH₂—;
   -E- is selected from the group comprising —CH₂—, —O—, —NH—, —N(CH₃)—, —S— and —SH₂—;
   p=0, 1, 2, 3, 4, 5, 6, 7, 8, 9 or 10;
   q=0, 1, 2, 3, 4, 5, 6, 7, 8, 9 or 10 and s=0, 1, 2, 3, 4, 5, 6, 7, 8, 9 or 10 and q+s=0, 1, 2, 3, 4, 5, 6, 7, 8, 9 or 10;
   —R² is —H, —CH₃ or a second pharmacokinetic modulating moiety;
   linker L has the structure

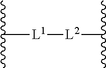

wherein
   L¹ is selected from the group comprising structures (a), (b), (c), (d), (e), (f), (g), (h), (i), (j), (k), (l), (m), (n) and (o) with

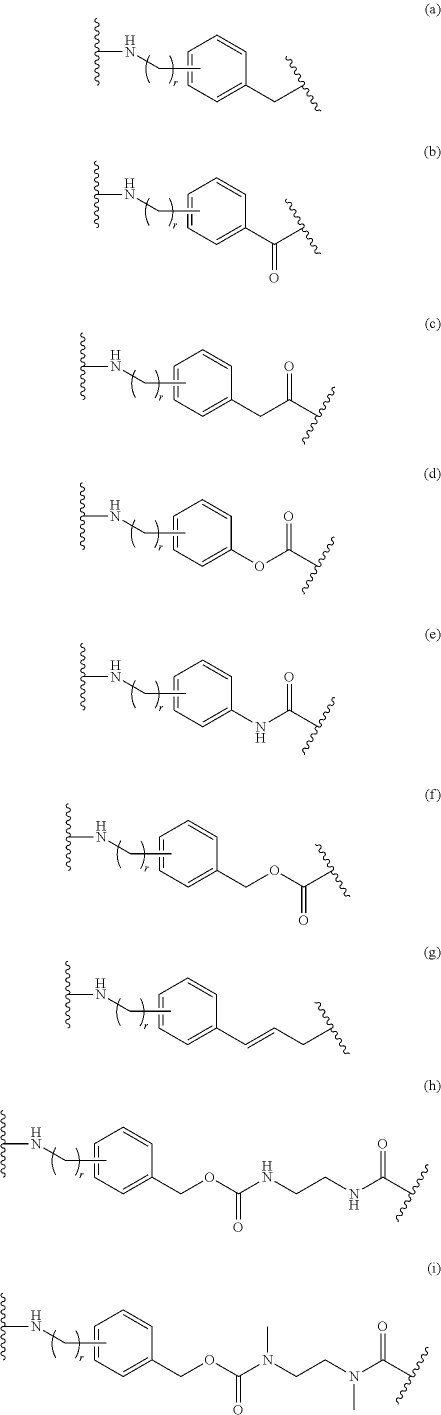

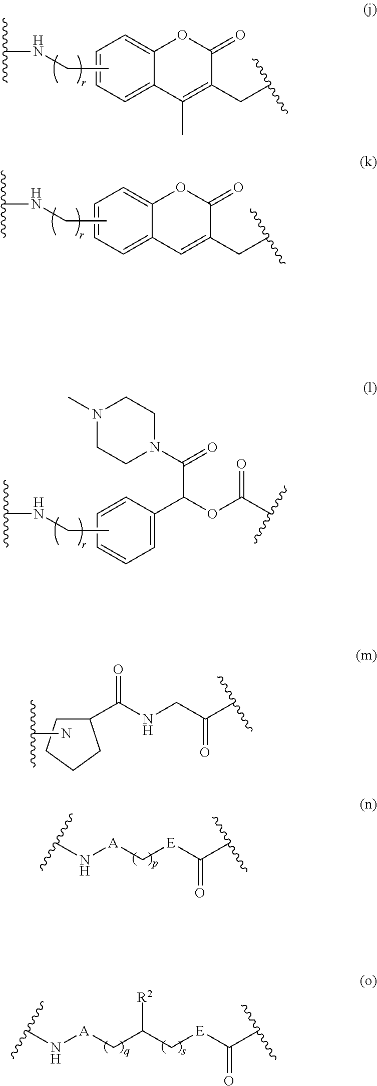

where
   the terminal amine is covalently bound to Fc;
   r=0, 1, 2, 3, 4, 5, 6, 7, 8, 9 or 10;
   -A- is absent or selected from the group comprising —O—, —NH—, —CH(OH)—, —CO—, —N(CH₃)—, —S— and —SH₂—;
   -E- is selected from the group comprising —CH₂—, —O—, —NH—, —N(CH₃)—, —S— and —SH₂—;
   p=0, 1, 2, 3, 4, 5, 6, 7, 8, 9 or 10;
   q=0, 1, 2, 3, 4, 5, 6, 7, 8, 9 or 10 and s=0, 1, 2, 3, 4, 5, 6, 7, 8, 9 or 10 and q+s=0, 1, 2, 3, 4, 5, 6, 7, 8, 9 or 10;
   —R² is —H, —CH₃ or a second pharmacokinetic modulating moiety; and
L² is selected from the group comprising structures (a′), (b′), (c′), (d′), (e′), (f), (g′), (h′), (i′), (j′), (k′), (l′), (m′), (n′) and (o′) with

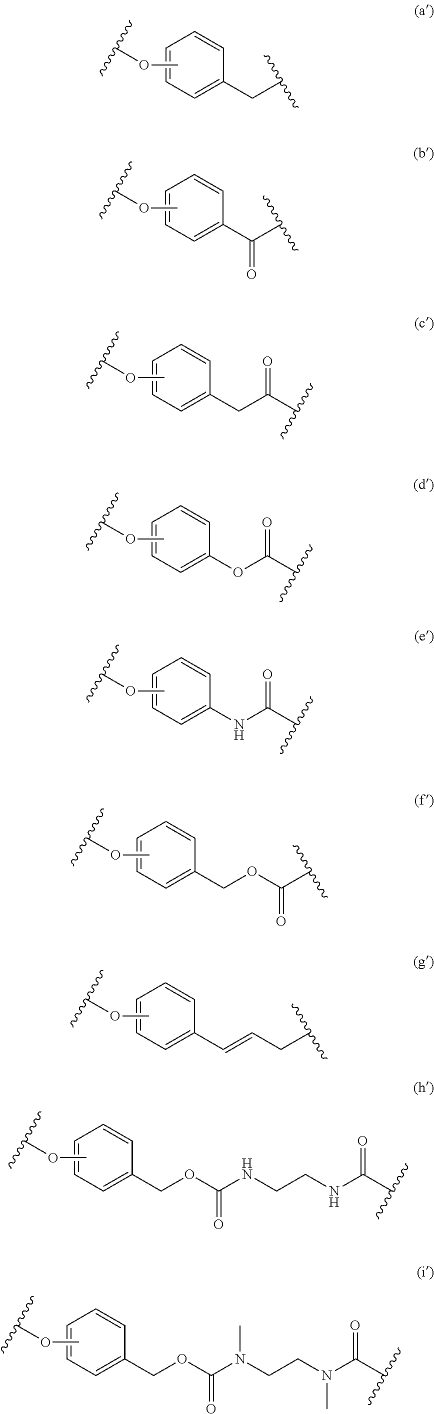

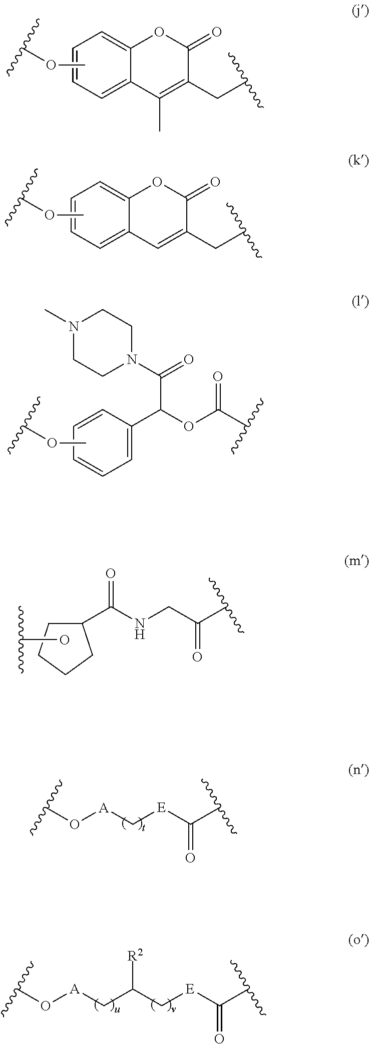

where
   if L¹ is equal to one of structures (a)-(m), then L² is equal to (n′) or (o′); and if L¹ is equal to (n) or (o), then L² is equal to one of structures (a′)-(m′);
   -A- is absent or selected from the group comprising —O—, —NH—, —CH(OH)—, —CO—, —N(CH₃)—, —S— and —SH₂—;
   -E- is selected from the group comprising —CH₂—, —O—, —NH—, —N(CH₃)—, —S— and —SH₂—;
   t=0, 1, 2, 3, 4, 5, 6, 7, 8, 9 or 10;
   u=0, 1, 2, 3, 4, 5, 6, 7, 8, 9 or 10 and v=0, 1, 2, 3, 4, 5, 6, 7, 8, 9 or 10 and u+v=0, 1, 2, 3, 4, 5, 6, 7, 8, 9 or 10; and
   —R² is —H, —CH₃ or a second pharmacokinetic modulating moiety;
   Linker L has the structure

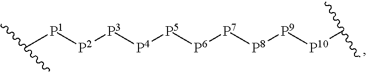

where
P¹ comprises an amine group covalently bound to Fc, P¹ is selected from the group comprising

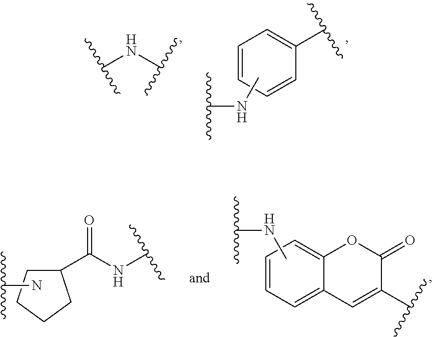

and
moieties P^{j} with 2≤j≤h and 2≤h≤10 independently from one another are selected from the group comprising

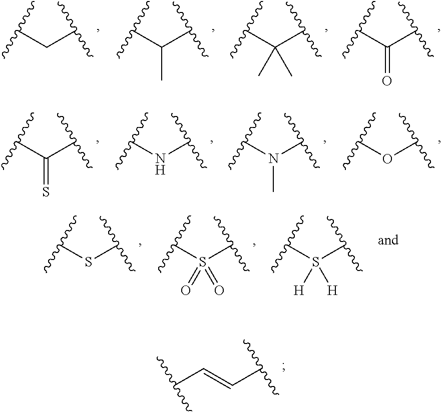

and P^{j} with h<j≤10 are absent;
   L has the structure

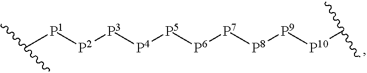

where
P^{l} is selected from the group comprising

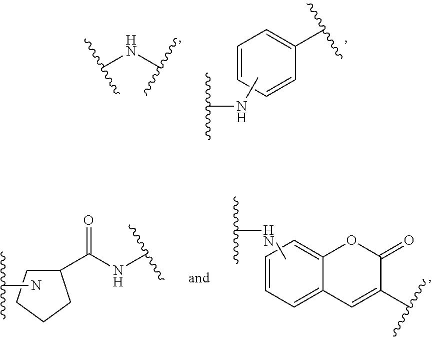

Fc and P^{l} form a moiety having a structure selected from the group comprising

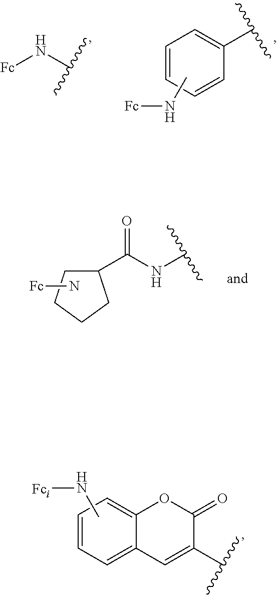

and
moieties P^{j} with 2≤j≤h and 2≤h≤10 independently from one another are selected from the group comprising

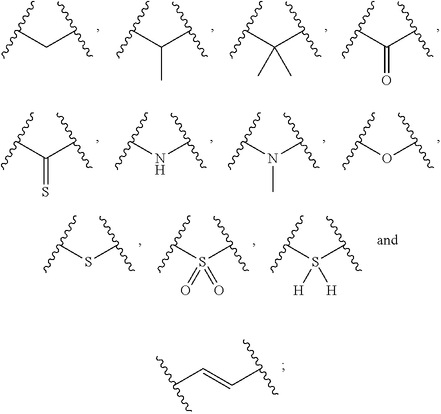

and P^{j} with h<j≤10 are absent;
   linker L has the structure

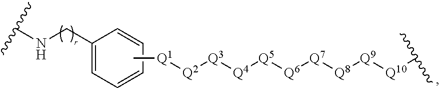

where
the amine group is covalently bound to Fc, r=0, 1, 2, 3, 4, 5, 6, 7, 8, 9 or 10 and moieties Q^{j} with 1≤j≤h and 1≤h≤10 independently from one another are selected from the group comprising

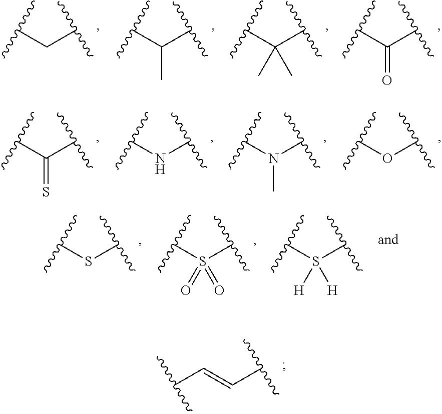

and Q^{j} with h<j≤10 are absent;
   linker L has the structure

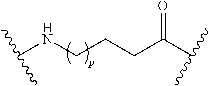

with p=1, 2, 3, 4, 5, 6, 7, 8, 9 or 10;
linker L has the structure

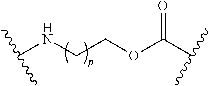

with p=1, 2, 3, 4, 5, 6, 7, 8, 9 or 10;
linker L has the structure

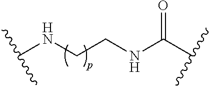

with p=1, 2, 3, 4, 5, 6, 7, 8, 9 or 10;
linker L has the structure

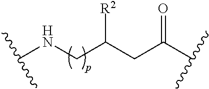

where —R² is a residue of a second pharmacokinetic modulating moiety and p=1, 2, 3, 4, 5, 6, 7, 8, 9 or 10;
linker L has the structure

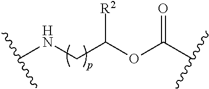

where —R² is a residue of a second pharmacokinetic modulating moiety and p=1, 2, 3, 4, 5, 6, 7, 8, 9 or 10;
linker L has the structure

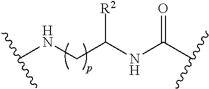

where —R² is a residue of a second pharmacokinetic modulating moiety and p=1, 2, 3, 4, 5, 6, 7, 8, 9 or 10;
linker L has a structure selected from the group comprising structures (a″), (b″), (c″), (d″), (e″), (f″), (g″), (h″), (i″), (j″), (k″), (l″), (m″), (n″), (o″), (p″), (q″), (r″) and (s″) with

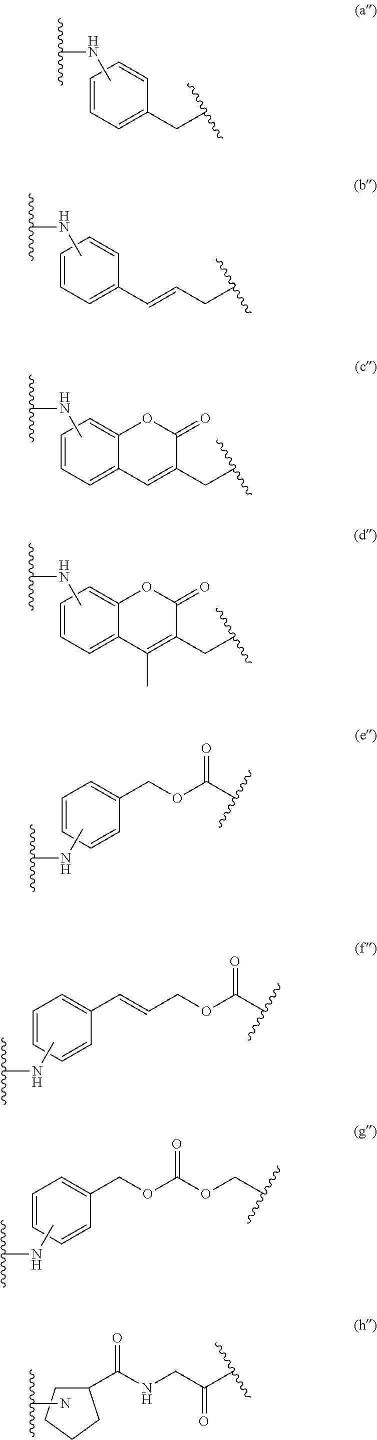

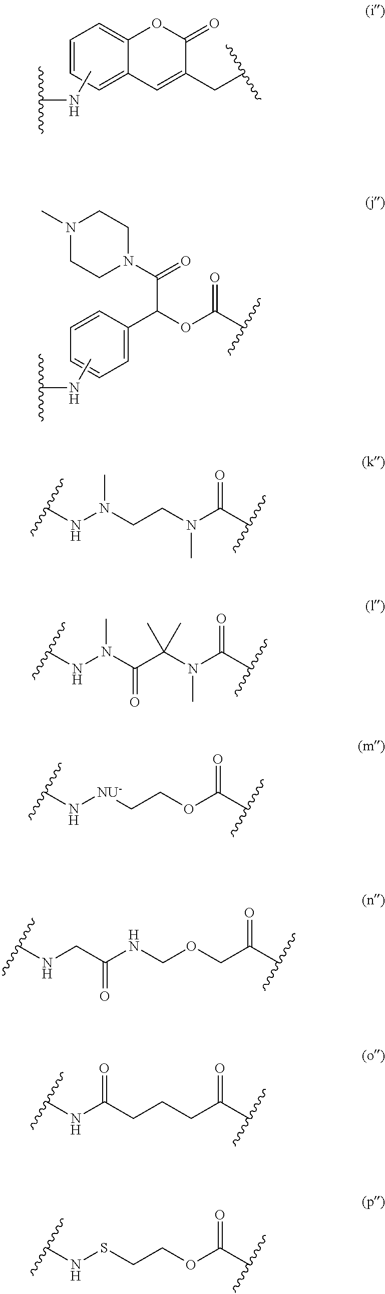

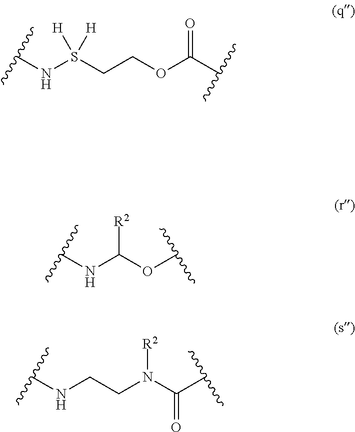

where —R² is —H, —CH₃ or a residue of a second pharmacokinetic modulating moiety and NU⁻ is a nucleophile selected from O, NH or S;
   linker L has a structure selected from the group comprising structures (a″), (b″), (c″), (d″), (e″), (f″), (g″), (h″), (i″), (j″), (k″), (l″), (m″), (n″), (o″), (p″), (q″), (r″), (s″) and Fc is covalently bound to the amine group of L;
   linker L has structure (a″);
   linker L has structure (b″);
   linker L has structure (c″);
   linker L has structure (d″);
   linker L has structure (e″);
   linker L has structure (f″);
   linker L has structure (g″);
   linker L has structure (h″);
   linker L has structure (i″);
   linker L has structure (j″);
   linker L has structure (k″);
   linker L has structure (l″);
   linker L has structure (m″), wherein NU-designates a nucleophile selected from O, NH and S;
   linker L has structure (n″);
   linker L has structure (o″);
   linker L has structure (p″);
   linker L has structure (q″);
   linker L has structure (r″);
   linker L has structure (s″);

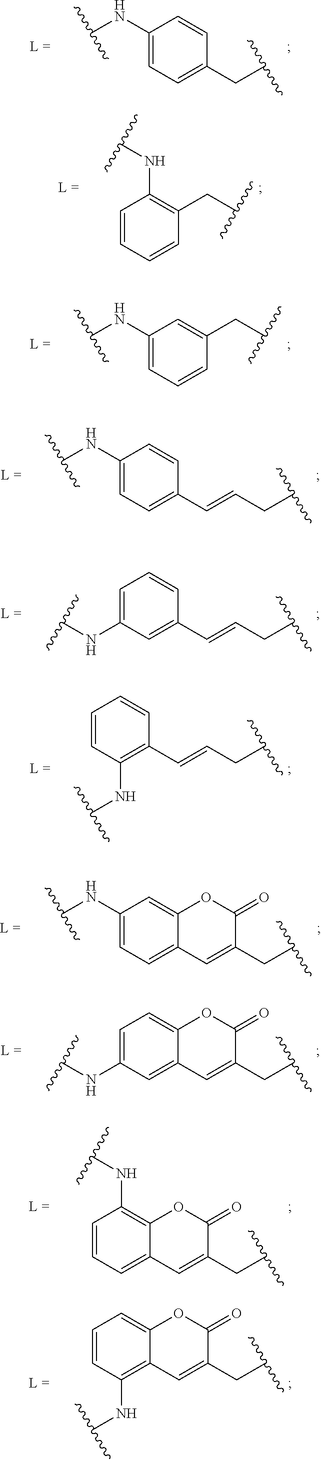

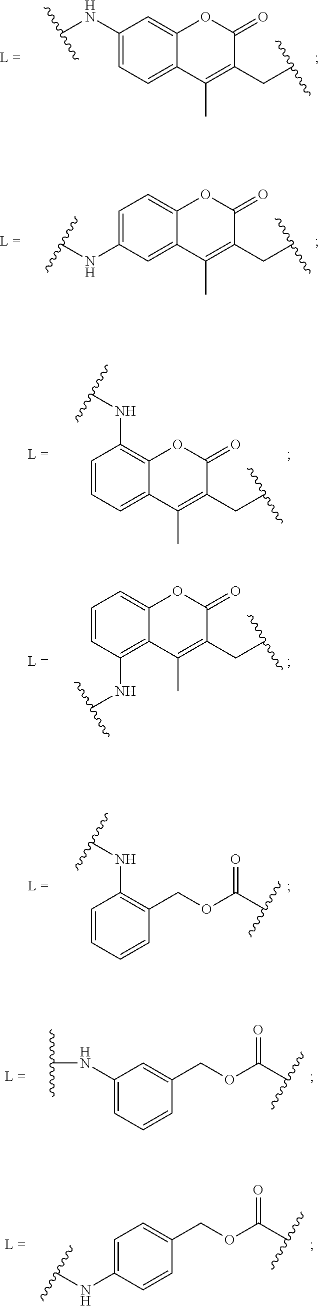

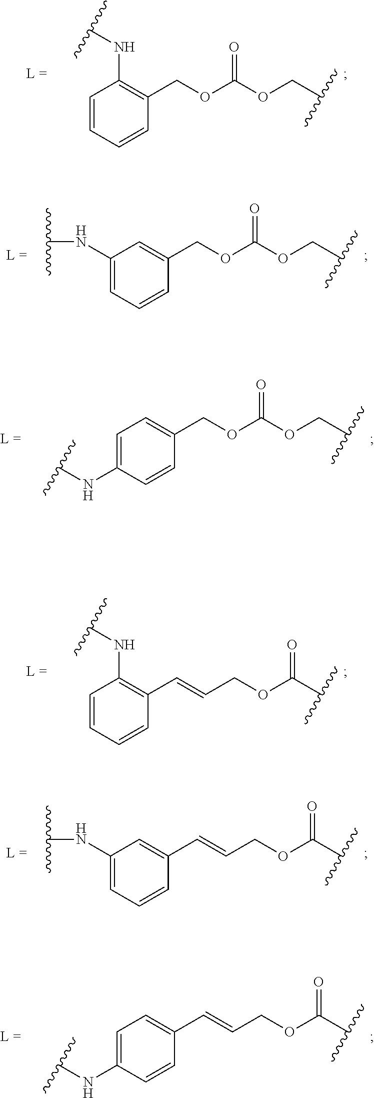 [SMILES: *COC(=O)OCc1ccccc1N* ; *COC(=O)OCc1cccc(N*)c1 ; *COC(=O)OCc1ccc(N*)cc1 ; *Nc1ccccc1/C=C/COC(*)=O ; *Nc1cccc(/C=C/COC(*)=O)c1 ; *Nc1ccc(/C=C/COC(*)=O)cc1]

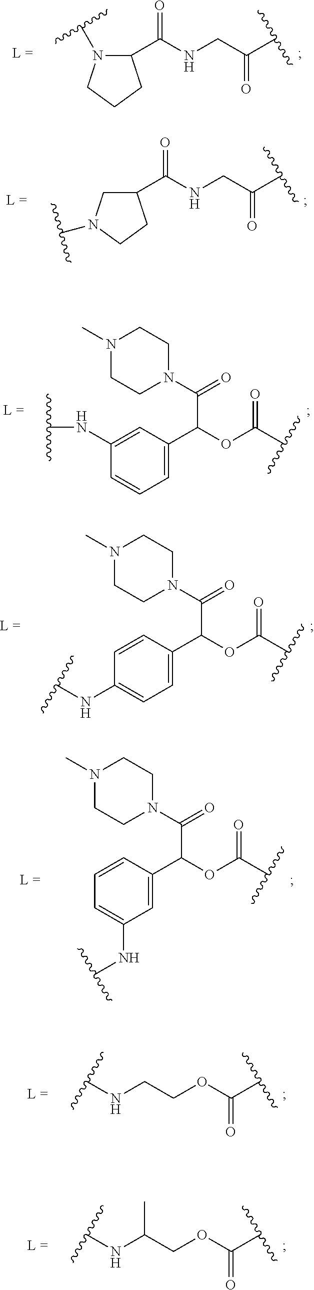

the prodrug S has a structure of type (x) or (y) with

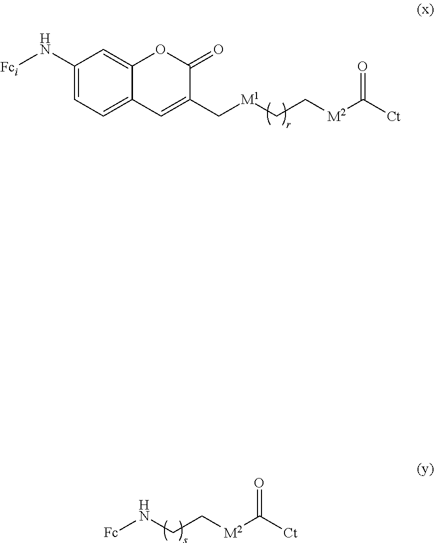

where M¹=—O—, —NH—, —N(CH₃)— or —S—, M²=—CH₂—, —O— or —NH—, r=0, 1, 2, 3, 4, 5, 6, 7, 8, 9 or 10 and s=0, 1, 2, 3, 4, 5, 6, 7, 8, 9 or 10;
   the prodrug S has a structure of type (x) with

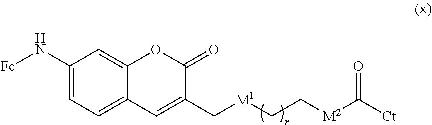

where M¹=—O—, —NH—, —N(CH₃)— or —S—, M²=—CH₂—, —O— or —NH— and r=0, 1, 2, 3, 4, 5, 6, 7, 8, 9 or 10;

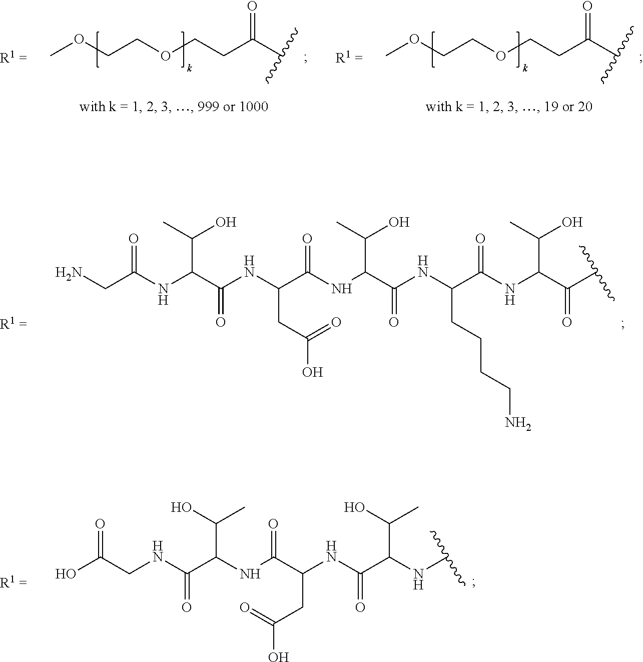

R¹ is a residue of a peptide comprising 1, 2, 3, 4, 5, 6, 7, 8, 9, 10, 11, 12, 13, 14, 15, 16, 17, 18, 19 or 20 amino acids independently selected from the group comprising Ala, Arg, Asn, Asp, Cys, Gln, Glu, Gly, His, Ile, Leu, Lys, Met, Phe, Pro, Ser, Thr, Trp, Tyr, Val, Pyl, Sec, GABA or γ-Aminobutyric acid, Homoserine, DOPA or 3,4-Dihydroxyphenylalanine, Citrulline, β-Alanine and Thyroxine;
R¹ is a residue of a lactide oligomer comprising 4, 5, . . . , 39 or 40 mer units;
R¹ is a residue of a lactide-co-glycolide oligomer comprising 4, 5, . . . , 39 or 40 mer units;
R¹ is a residue of an acrylate oligomer comprising 4, 5, . . . , 39 or 40 mer units;
R¹ is a residue of a methacrylate oligomer comprising 4, 5, . . . , 39 or 40 mer units;

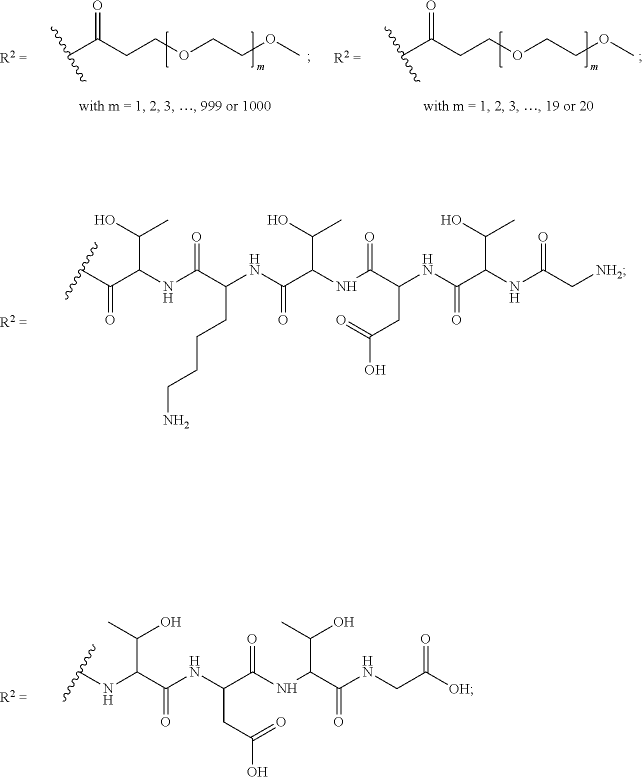

R² is a residue of a peptide comprising 1, 2, 3, 4, 5, 6, 7, 8, 9, 10, 11, 12, 13, 14, 15, 16, 17, 18, 19 or 20 amino acids independently selected from the group comprising Ala, Arg, Asn, Asp, Cys, Gln, Glu, Gly, His, Ile, Leu, Lys, Met, Phe, Pro, Ser, Thr, Trp, Tyr, Val, Pyl, Sec, GABA or γ-Aminobutyric acid, Homoserine, DOPA or 3,4-Dihydroxyphenylalanine, Citrulline, β-Alanine and Thyroxine;
R² is a residue of a lactide oligomer comprising 4, 5, . . . , 39 or 40 mer units;
R² is a residue of a lactide-co-glycolide oligomer comprising 4, 5, . . . , 39 or 40 mer units;
R² is a residue of an acrylate oligomer comprising 4, 5, . . . , 39 or 40 mer units;
R² is a residue of a methacrylate oligomer comprising 4, 5, . . . , 39 or 40 mer units.

The present invention further proposes multivalent prodrugs with two or more FAP-activatable initiators. Multivalent prodrugs exhibit increased tumor uptake and release of the respective parent drug. Compared to monovalent prodrugs with one FAP-activatable initiator multivalent prodrugs have a higher docking and activation probability or—in physics terminology—a larger effective cross section. Improved tumor uptake and parent drug release affords reduction of the administered dose and further mitigation of adverse side effects.

In the inventive prodrugs or SMDC each of the one or more FAP-activatable initiator or trigger moieties is covalently bound to a linear or branched self-immolative linker which in turn is covalently bound to a radical or residue of a chemotherapeutic compound (the parent drug). The inventive prodrugs or SMDC are configured for extracellular activation by FAP, which is overexpressed in various solid tumors. FAP-catalyzed cleavage of any one of the initiator or trigger moieties from the linear or branched self-immolative linker causes dissociation of the latter from and subsequent protonation of the chemotherapeutic compound radical. The chemotherapeutic compound (parent drug) is, hence, released into the extracellular compartment of the tumor.

Accordingly, the invention also pertains to a small-molecule-drug-conjugate (SMDC) comprising a chemotherapeutic compound radical Ct, a linear or branched self-immolative linker L and one, two, three, four or more initiators (F1, F2, F3, F4), wherein
L is covalently coupled to a nitrogen, amine or oxygen radical of Ct;
L comprises one, two, three, four or more amine radicals;
each of initiators (F1, F2, F3, F4) is covalently coupled to an amine radical of L;
each of initiators (F1, F2, F3, F4) is configured for enzymatic cleavage from L by fibroblast activation protein (FAP);
L is configured for release of Ct upon cleavage of any one of initiators (F1, F2, F3, F4);
initiators (F1, F2, F3, F4) independently of each other comprise or have a structure selected from the group of structures comprising

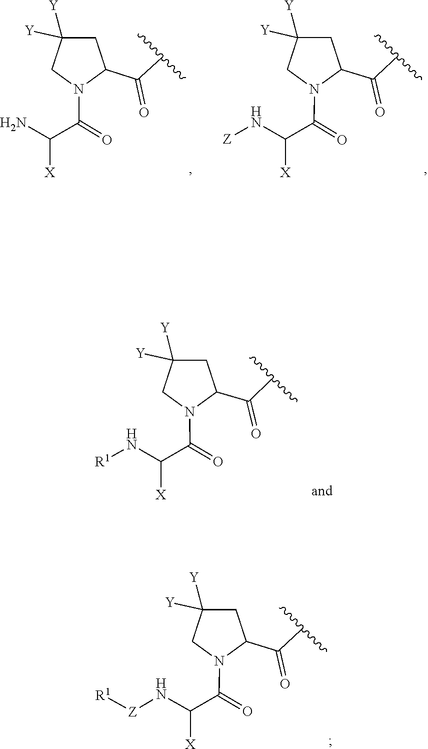

where the pyrrolidine ring is oriented towards the self-immolative linker L, X=—H or —CH₃, Y=—H or —F, —R¹ is a radical of a first pharmacokinetic modulating moiety and Z is a moiety having a structure selected from the group comprising structures (1), (2), (3), (4), (5), (6), (7), (8), (9), (10), (11), (12), (13), (14) and (15) with

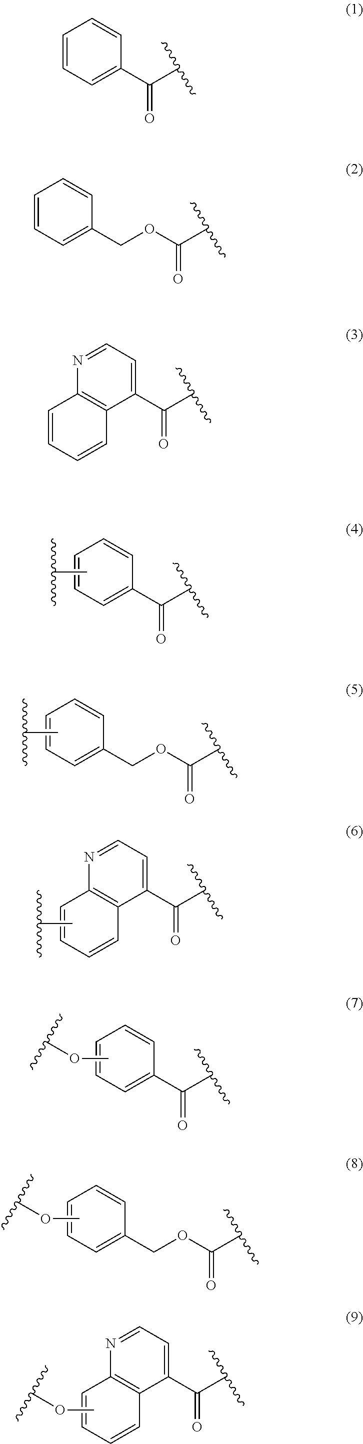

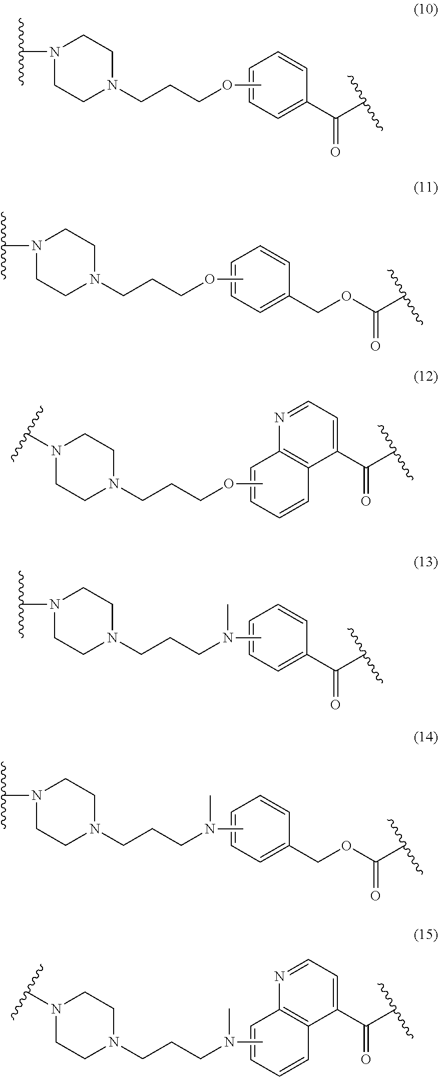

and

Ct is a radical of a chemotherapeutic compound selected from the group comprising 1,2,3,4-Tetrahydrogen-staurosporine, 17-Dmag, 2-Aminopropanenitrile, 4SC202, ABBV-CLS-484, Abemaciclib, Abexinostat, Acalabrutinib, Acetylbufalin, Aderbasib, Afatinib, Afuresertib, Alectinib, Alisertib, Alpelisib, Alvocidib, AMD3465, Anlotinib, Apalutamide, AR-42, Asciminib, Atuveciclib, Avapritinib, Axitinib, AZD7762, BAY1125976, Belinostat, β-Hydroxyisovaleric acid, BF211, Bicalutamide, Binimetinib, Bortezomib, Bosutinib, Brigatinib, Bufalin, Buparlisib, Buthionine sulfoximine, Cabozantinib, Capivasertib, Capmatinib, Carfilzomib, CEP-9722, Ceralasertib, Ceritinib, Chidamide, CHR-3996, Citarinostat, Cobimetinib, CompK, Copanlisib, Crenolanib, Crizotinib, CUDC-101, Dabrafenib, Daclatasvir, Dacomitinib, Darolutamide, Dasatinib, Dasatinib D1, Dasatinib D2, Dasatinib D3, Dasatinib D4, Decitabine, Defactinib, Degarelix, Diethylstilbestrol, Dinaciclib, Dp44mT, DpC, DUPA, Duvelisib, E7016, Ebvaciclib, Eganelisib, Elimusertib, Emavusertib, Enasidenib, Encorafenib, Enitociclib, Entinostat, Entrectinib, Enzalutamide, Epacadostat, Epigallocatechin gallate, Epoxomicin, Erdafitinib, Erismodegib, Erlotinib, Everolimus, Fasudil, Fedratinib, Filgotinib, Foslinanib, Fostamatinib, Fruquintinib, Galunisertib, Ganetespib, Gedatolisib, Gefitinib, GFH018, Gilteritinib, Givinostat, Glasdegib, Goserelin, GSK2256098, GSK269962A, GSK690693, GUL, Halofuginone, Hymecromone, Ibrutinib, Icotinib, Idelalisib, Imatinib, Imiquimod, Infigratinib, Iniparib, Ipatasertib, Itacitinib, Ivaltinostat, Ivosidenib, Ixazomib, Kevetrin, Lapatinib, Larotrectinib, Lenalidomide, Leniolisib, Lenvatinib, Leuprolide, Linsitinib, Lonafarnib, Lorlatinib, Losartan, Lucitanib, Luminespib, M1096, Marizomib, ME-344, Merestinib, Metformin, MG132, Midostaurin, Miransertib, Mivavotinib, MK2206, MMP-9 Inhibitor I, Mobocertinib, Mocetinostat, Motesanib, MRTX1133, Navitoclax, Nazartinib, Nedisertib, Neratinib, Nilotinib, Nilutamide, Nintedanib, Niraparib, NMS-P118, NMS-P515, NSC668394, NSC95397, Numidargistat, NVP-2, Olaparib, Olmutinib, Omipalisib, Oprozomib, Osimertinib, OTS-964, Palbociclib, Pamiparib, Panobinostat, Paricalcitol, Parsaclisib, Pazopanib, Pemetrexed, Pemigatinib, Pevonedistat, Pexidartinib, Pifusertib, I PMPA, Ponatinib, Practinostat, Pralsetinib, Prednisone, Prexasertib, Prinomastat, Propranolol, Quisinostat, Quizartinib, Ralimetinib, Ravoxertinib, Regorafenib, Relugolix, Resminostat, Resveratrol, Retaspimycin, Retinoic acid, Ribociclib, Ricolinostat, Rigosertib, Ripretinib, RO-3306, Rocilinostat, Rogaratinib, Romidepsin, Rucaparib, Ruxolitinib, S2, S5, Saridegib, SBI-0654454, SCH772984, Seliciclib, Selitrectinib, Selpercatinib, Selumetinib, SGN-2FF, SGX393, Shikonin, Silibinin, Sitravatinib, Sonidegib, Sorafenib, Sotorasib, Staurosporine, SU11274, Sunitinib, Surufatinib, Tacedinaline, Tadalafil, Talazoparib, Taletrectinib, Tarloxotinib, Taselisib, Tazemetostat, Tefinostat, Temsirolimus, Tetrazole, Tivozanib, Tofacitinib, Tozasertib, Trametinib, Tranilast, Tretinoin, Trichostatin, Tucatinib, Tucidinostat, Tuvusertib, Ubenimex, Umbralisib, Uprosertib, USL311, Vactosertib, Valproic acid, Valsartan, Vandetanib, Veliparib, Vemurafenib, Venetoclax, Verteporfin, Vismodegib, Vorinostat, WRG-28, WZ811, Xevinapant, Zandelisib, Zanubrutinib, ZM447439, Abiraterone, Aclarubicin, Adozelesin, Alrestatin, Amanitin, Amrubicin, Anthramycin, Arenastatin, Bizelesin, Bleomycin, Camptothecin, Capecitabine, Carzelesin, CC-1065, Chaconine, Chlorambucil, Cryptophycin-24, Cyclophosphamide, Cytarabine, Dacarbazine, Dactinomycin, Daunorubicin, DAVLBH, Deruxtecan, Dexamethasone, Dichloro acetic acid, Dimethyl-SGD-1882, Docetaxel, Dolastatin-10, Doxorubicin, Duocarmycin A, Duocarmycin B1, Duocarmycin B2, Duocarmycin C1, Duocarmycin C2, Duocarmycin D, Duocarmycin GA, Duocarmycin SA, Emetine, Epirubicin, Eribulin, Etoposide, Floxuridine, Fludarabine, Fluorouracil, Flutamide, Fulvestrant, Gemcitabine, Idarubicin, Ifosfamide, Irinotecan, L-Asparaginase, Lomustine, Melphalan, Mertansine, Methotrexate, Milataxel, Mitoxantrone, Monomethyl Auristatin E, Maytansine, Maytansinoid, Ozogamicin, Paclitaxel, Pirarubicin, Pixantrone, Podophyllotoxin, Procarbazine, Rapamycin, Rachelmycin, Salinomycin, SB-T-1214, Selinexor, SN-38, Solamargine, Solanine, Talirine, Temozolomide, Tesetaxel, SG3199 (Tesirine), Thapsigargin, Tomatine, Topotecan, Tubulysin B, Valrubicin, Vinblastine, Vincristine, Vinorelbine, VIP126, Zorubicin.

Expedient embodiments of the inventive small-molecule-drug-conjugate (SMDC) or prodrug are characterized by one of the following features or a combination of two or more of the following features insofar the combined features are not mutually exclusive or contradictory and according to which:
Ct is a radical of Dinaciclib;
Ct is a radical of NVP-2;
Ct is a radical of Erlotinib;
Ct is a radical of Imatinib;
Ct is a radical of Sorafenib;
Ct is a radical of Bufalin;
Ct is a radical of Acetylbufalin;
initiators (F1, F2, F3, F4) independently of each other comprise or have a structure selected from the group of structures comprising

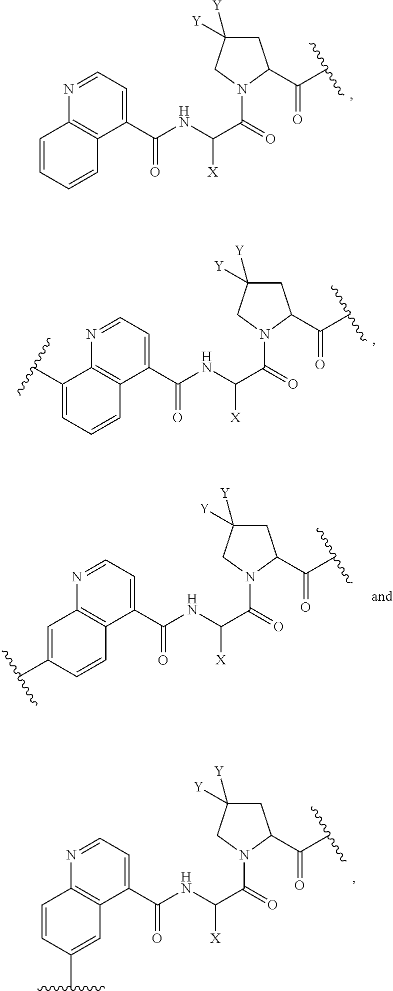

where the pyrrolidine ring is oriented towards the self-immolative linker L, Y=—H or —F and X=—H or —CH₃;
initiators (F1, F2, F3, F4) independently of each other comprise or have a structure selected from the group of structures comprising

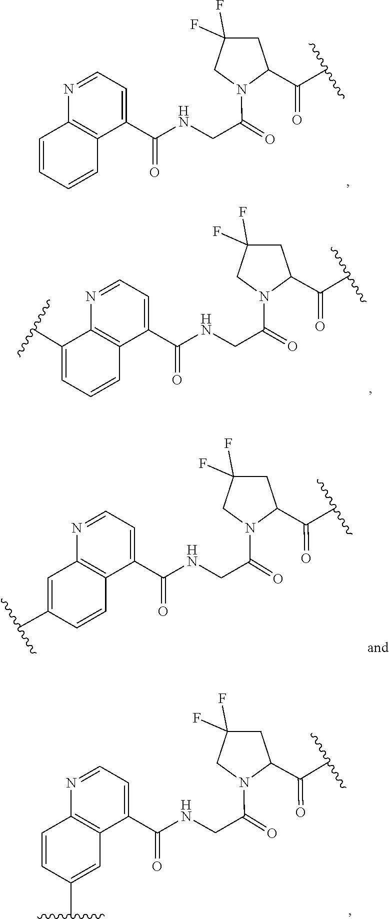 [SMILES: *C(=O)C1CC(F)(F)CN1C(=O)CNC(=O)c1ccnc2ccccc12 ; *C(=O)C1CC(F)(F)CN1C(=O)CNC(=O)c1ccnc2c(*)cccc12 ; *C(=O)C1CC(F)(F)CN1C(=O)CNC(=O)c1ccnc2cc(*)ccc12 ; *C(=O)C1CC(F)(F)CN1C(=O)CNC(=O)c1ccnc2ccc(*)cc12]

where the pyrrolidine ring is oriented towards the self-immolative linker L;
two, three, four or more of initiators (F1, F2, F3, F4) are different from one another;
two, three, four or more of initiators (F1, F2, F3, F4) are equal;
the SMDC comprises one initiator F1;
the SMDC comprises two initiators (F1, F2);
the SMDC comprises four initiators (F1, F2, F3, F4);
L comprises a coupling moiety for Ct, said coupling moiety having a structure selected from the group of structures comprising

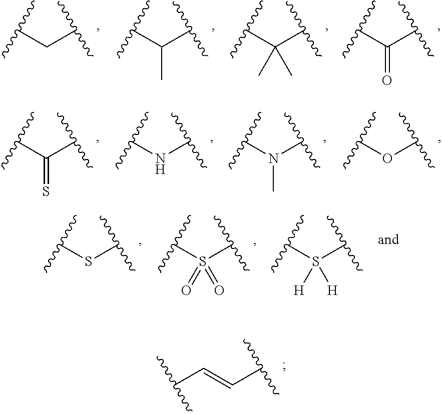

L comprises a coupling moiety for Ct, said coupling moiety having the structure

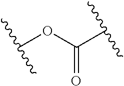 [SMILES: *OC(*)=O]

where the terminal carbonyl is covalently bound to a nitrogen radical of Ct;
L comprises a coupling moiety for Ct, said coupling moiety having the structure

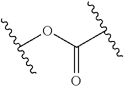 [SMILES: *OC(*)=O]

where the terminal carbonyl is covalently bound to an amine radical of Ct;
L comprises a coupling moiety for Ct, said coupling moiety having the structure

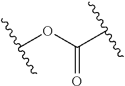 [SMILES: *OC(*)=O]

where the terminal carbonyl is covalently bound to an oxygen radical of Ct;
L comprises a coupling moiety for Ct, said coupling moiety having the structure

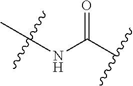 [SMILES: *NC(*)=O]

where the terminal carbonyl is covalently bound to a nitrogen radical of Ct;
   L comprises a coupling moiety for Ct, said coupling moiety having the structure

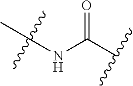 [SMILES: *NC(*)=O]

where the terminal carbonyl is covalently bound to an amine radical of Ct;
L comprises a coupling moiety for Ct, said coupling moiety having the structure

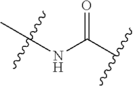 [SMILES: *NC(*)=O]

where the terminal carbonyl is covalently bound to an oxygen radical of Ct;
L comprises a coupling moiety for Ct, said coupling moiety having the structure

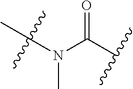 [SMILES: *C(=O)N(*)C]

where the terminal carbonyl is covalently bound to a nitrogen radical of Ct;
L comprises a coupling moiety for Ct, said coupling moiety having the structure

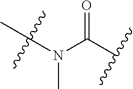 [SMILES: *C(=O)N(*)C]

where the terminal carbonyl is covalently bound to an amine radical of Ct;
L comprises a coupling moiety for Ct, said coupling moiety having the structure

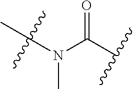 [SMILES: *C(=O)N(*)C]

where the terminal carbonyl is covalently bound to an oxygen radical of Ct;
L comprises a coupling moiety for Ct, said coupling moiety having the structure

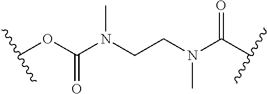 [SMILES: *OC(=O)N(C)CCN(C)C(*)=O]

where the terminal carbonyl is covalently bound to a nitrogen radical of Ct;
L comprises a coupling moiety for Ct, said coupling moiety having the structure

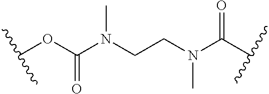 [SMILES: *OC(=O)N(C)CCN(C)C(*)=O]

where the terminal carbonyl is covalently bound to an amine radical of Ct;
L comprises a coupling moiety for Ct, said coupling moiety having the structure

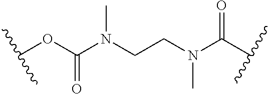 [SMILES: *OC(=O)N(C)CCN(C)C(*)=O]

where the terminal carbonyl is covalently bound to an oxygen radical of Ct;
L comprises one, two or more branching moieties having the structure

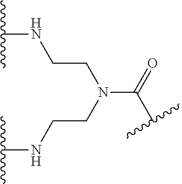 [SMILES: *NCCN(CCN*)C(*)=O]

where the terminal carbonyl is oriented towards Ct or covalently bound to Ct;
L comprises one or more branching moieties having the structure

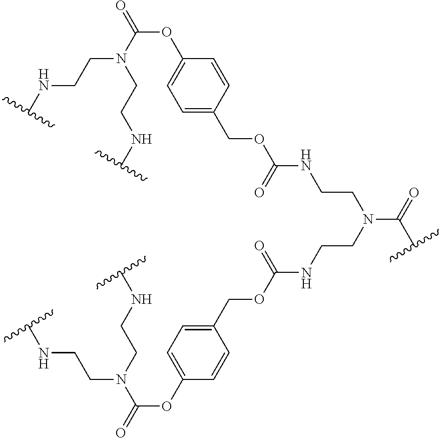 [SMILES: *NCCN(CCN*)C(=O)Oc1ccc(COC(=O)NCCN(CCNC(=O)OCc2ccc(OC(=O)N(CCN*)CCN*)cc2)C(*)=O)cc1]

where the terminal carbonyl is oriented towards Ct or covalently bound to Ct;
L comprises one, two, three, four or more coupling moieties for initiators (F1, F2, F3, F4), said one, two, three, four or more coupling moieties independently of each other having the structure

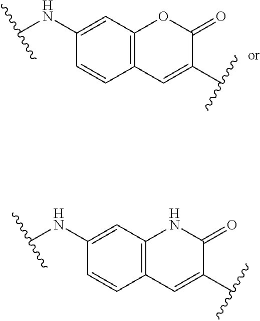 [SMILES: *Nc1ccc2cc(*)c(=O)oc2c1 ; *Nc1ccc2cc(*)c(=O)[nH]c2c1]

where the terminal amine is covalently bound to an initiator (F1, F2, F3, F4);
L comprises one, two, three, four or more coupling moieties for initiators (F1, F2, F3, F4), said one, two, three, four or more coupling moieties independently of each other having the structure

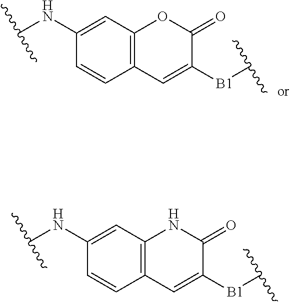

where the terminal amine is covalently bound to an initiator (F1, F2, F3, F4) and B1 independently of each other is selected from the group of moieties comprising

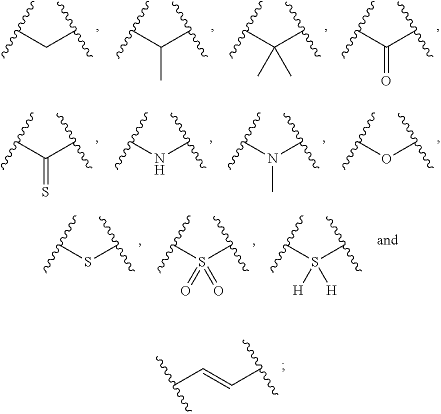

L comprises one, two, three, four or more coupling moieties for initiators (F1, F2, F3, F4), said one, two, three, four or more coupling moieties independently of each other having a structure selected from the group comprising

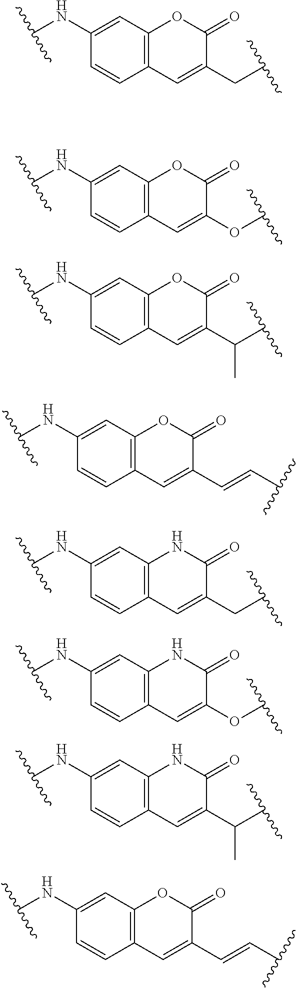

where the terminal amine is covalently bound to an initiator (F1, F2, F3, F4);
L comprises one, two, three, four or more coupling moieties for initiators (F1, F2, F3, F4), said one, two, three, four or more coupling moieties independently of each other having the structure

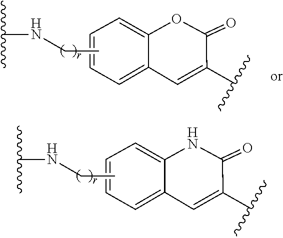

where
   the terminal amine is covalently bound to an initiator (F1, F2, F3, F4); and
   r=0, 1, 2, 3, 4, 5, 6, 7, 8, 9 or 10;
L comprises one, two, three, four or more coupling moieties for initiators (F1, F2, F3, F4), said one, two, three, four or more coupling moieties independently of each other having the structure

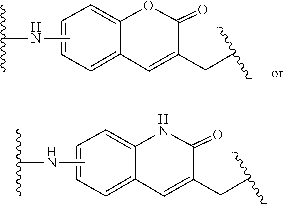

where the terminal amine is covalently bound to an initiator (F1, F2, F3, F4);
L comprises one, two, three, four or more coupling moieties for initiators (F1, F2, F3, F4), said one, two, three, four or more coupling moieties independently of each other having the structure

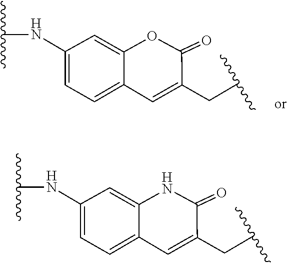 [SMILES: *Cc1cc2ccc(N*)cc2oc1=O ; *Cc1cc2ccc(N*)cc2[nH]c1=O]

where the terminal amine is covalently bound to an initiator (F1, F2, F3, F4);
L comprises a second pharmacokinetic moiety R²;
L comprises a second pharmacokinetic moiety R² and 1, 2, 3, 4, 5, 6, 7 or 8 further pharmacokinetic moieties (R³, R⁴, R⁵, R⁶, R⁷, R⁸, R⁹, R¹⁰);
L comprises one, two, three, four or more coupling moieties for initiators (F1, F2, F3, F4), said one, two, three, four or more coupling moieties independently of each other having a structure selected from the group comprising structures (a), (b), (c), (d), (e), (f), (g), (h), (i), (j), (k), (l), (m), (n) and (o) with

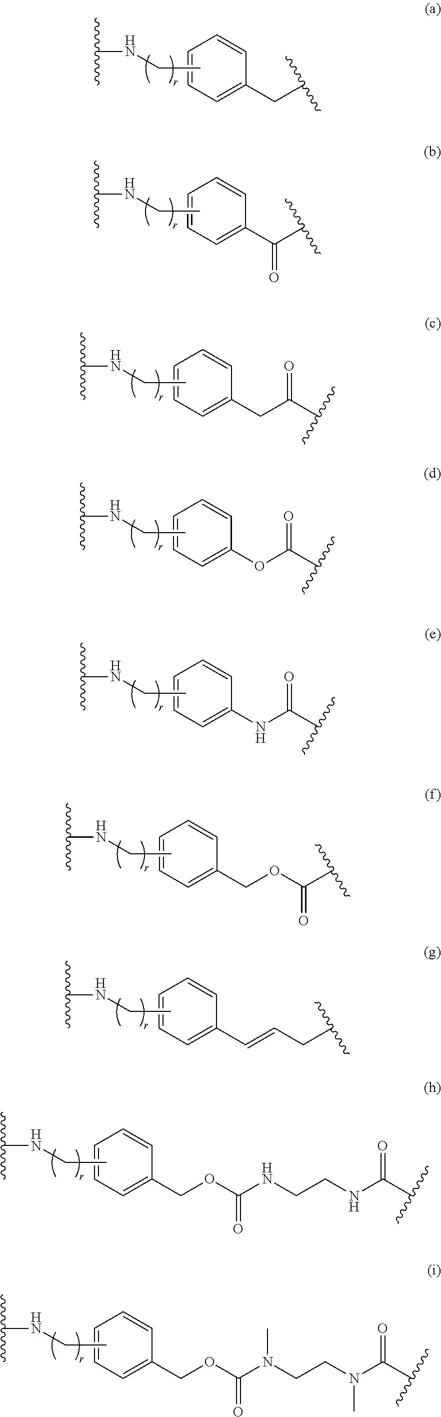

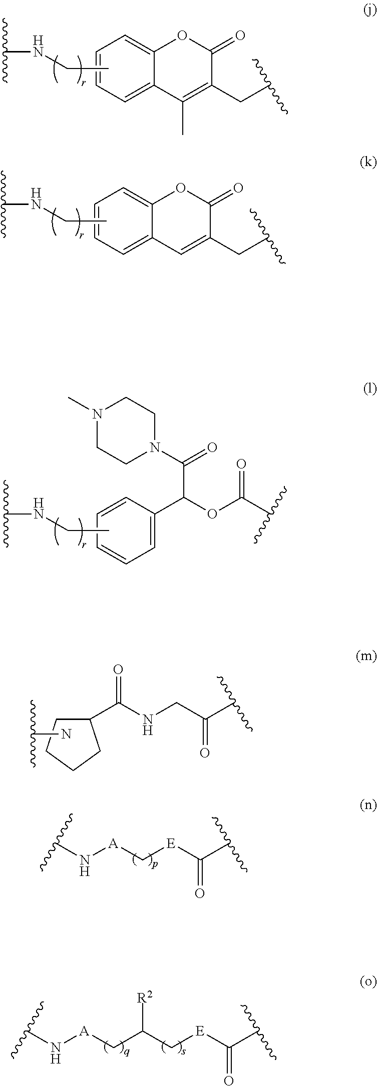

where
   the terminal amine is covalently bound to an initiator (F1, F2, F3, F4);
   r=0, 1, 2, 3, 4, 5, 6, 7, 8, 9 or 10;
   -A- is absent or selected from the group comprising —O—, —NH—, —CH(OH)—, —CO—, —N(CH₃)—, —S— and —SH₂—;
   -E- is selected from the group comprising —CH₂—, —O—, —NH—, —N(CH₃)—, —S— and —SH₂—;
   p=0, 1, 2, 3, 4, 5, 6, 7, 8, 9 or 10;
   q=0, 1, 2, 3, 4, 5, 6, 7, 8, 9 or 10 and s=0, 1, 2, 3, 4, 5, 6, 7, 8, 9 or 10 and q+s=0, 1, 2, 3, 4, 5, 6, 7, 8, 9 or 10;
   —R² is —H, —CH₃ or a second pharmacokinetic modulating moiety;
L comprises one initiator F1 and one moiety having the structure

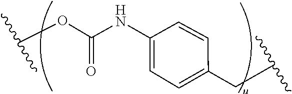

where the terminal carbamate is oriented towards the initiator F1 and u=1, 2, 3, 4, 5, 6, 7, 8, 9, 10, 11, 12, 13, 14, 15, 16, 17, 18, 19 or 20;
L comprises two, three, four or more branches which independently of each other comprise an initiator (F1, F2, F3, F4) and a moiety having a structure of type

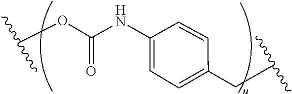

where the terminal carbamate is oriented towards the initiator (F1, F2, F3, F4), u=1, 2, 3, 4, 5, 6, 7, 8, 9, 10, 11, 12, 13, 14, 15, 16, 17, 18, 19 or 20 and the number u in one branch may differ from that in other branches;
self-immolative linker L comprises 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40 or more moieties which independently of each other have a structure selected from the group of structures comprising

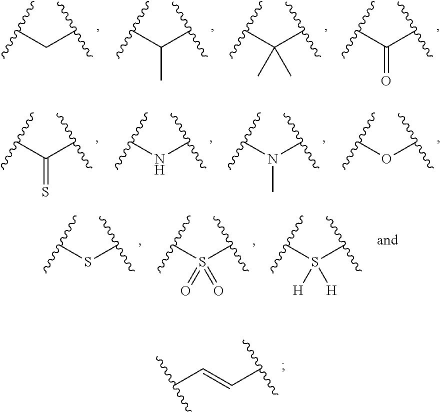

each of pharmacokinetic moieties (R¹, R², R³, R⁴, R⁵, R⁶, R⁷, R⁸, R⁹, R¹⁰) independently of each other has a structure selected from the group of structures comprising

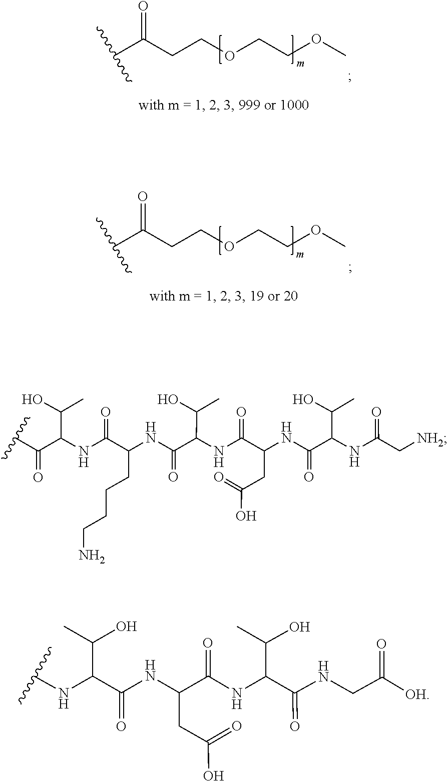

each of pharmacokinetic moieties (R¹, R², R³, R⁴, R⁵, R⁶, R⁷, R⁸, R⁹, R¹⁰) independently of one another is a residue of a peptide comprising 1, 2, 3, 4, 5, 6, 7, 8, 9, 10, 11, 12, 13, 14, 15, 16, 17, 18, 19 or 20 amino acids independently selected from the group comprising Ala, Arg, Asn, Asp, Cys, Gln, Glu, Gly, His, Ile, Leu, Lys, Met, Phe, Pro, Ser, Thr, Trp, Tyr, Val, Pyl, Sec, GABA or γ-Aminobutyric acid, Homoserine, DOPA or 3,4-Dihydroxyphenyl-alanine, Citrulline, β-Alanine and Thyroxine;
each of pharmacokinetic moieties (R¹, R², R³, R⁴, R⁵, R⁶, R⁷, R⁸, R⁹, R¹⁰) independently of one another is a residue of a lactide oligomer comprising 4, 5, . . . , 39 or 40 mer units;
each of pharmacokinetic moieties (R¹, R², R³, R⁴, R⁵, R⁶, R⁷, R⁸, R⁹, R¹⁰) independently of one another is a residue of a lactide-co-glycolide oligomer comprising 4, 5, . . . , 39 or 40 mer units;
each of pharmacokinetic moieties (R¹, R², R³, R⁴, R⁵, R⁶, R⁷, R⁸, R⁹, R¹⁰) independently of one another is a residue of an acrylate oligomer comprising 4, 5, . . . , 39 or 40 mer units;
each of pharmacokinetic moieties (R¹, R², R³, R⁴, R⁵, R⁶, R⁷, R⁸, R⁹, R¹⁰) independently of one another is a residue of a methacrylate oligomer comprising 4, 5, . . . , 39 or 40 mer units.

In preferred embodiments the inventive SMDC or prodrugs comprise one or more moieties that encompass at least part of one or more FAP-activatable initiators and at least part of the self-immolative linker and have a structure of type

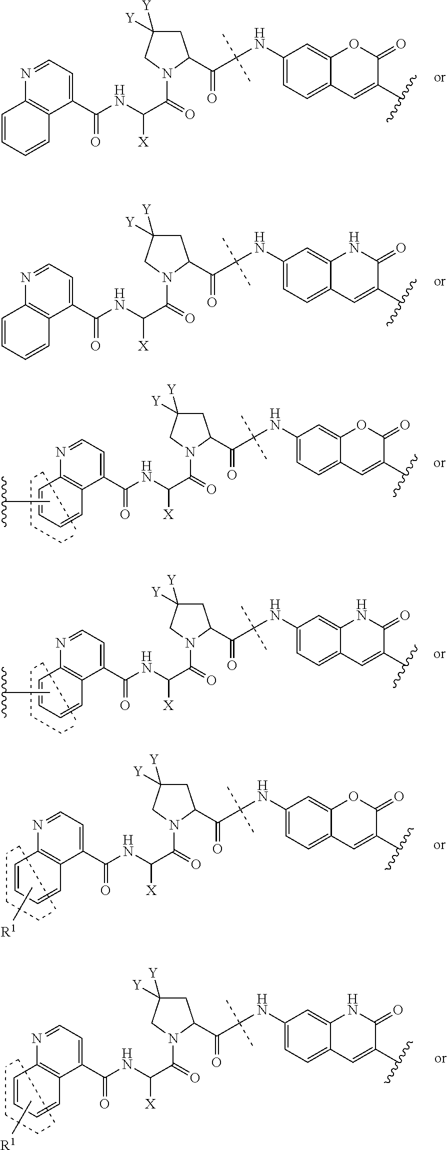

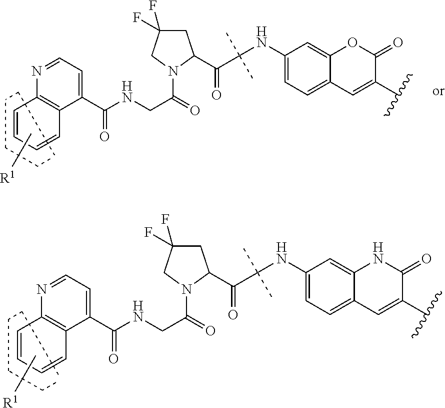

where X=—H or —CH₃, Y=—H or —F, R¹ is H or a pharmacokinetic modulating moiety and the dotted line indicates the enzymatic cleavage site.

Preferred embodiments of the inventive SMDC or prodrugs comprise one or more moieties that independently of one another have the general structure

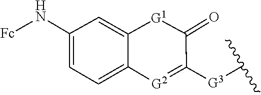

wherein Fc designates one of the FAP-cleavable moieties described in the entire preceding text, G¹=—O— or —NH—; G²=—CH— or —C(CH₃)—; preferably G²=—CH—; and G³ is selected from the group comprising moieties

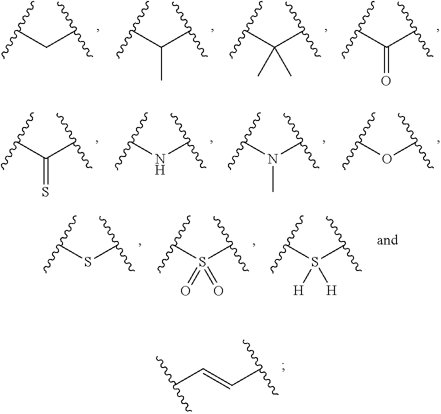

preferably G³=—CH₂— or —CH(CH₃)— or —O— or —CH═CH—; and in particular G³=CH₂— or —CH(CH₃)—.

Preferred embodiments of the inventive SMDC or prodrugs comprise one or more moieties that independently of one another have a structure selected from the group of structures comprising

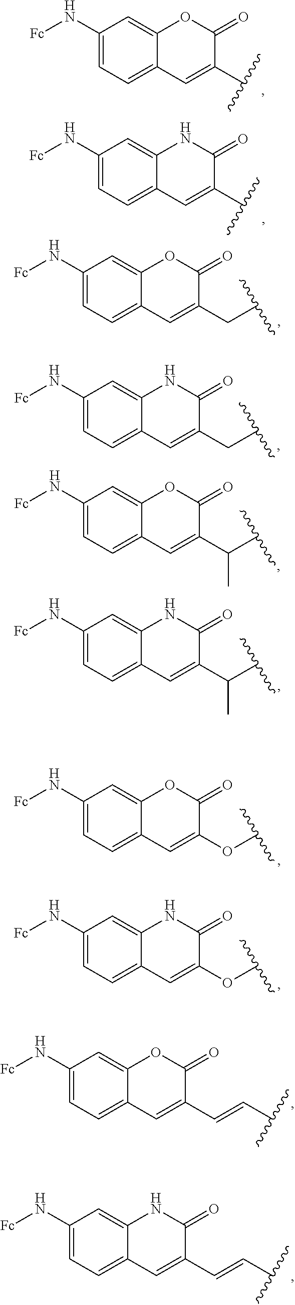

wherein Fc designates one of the FAP-cleavable moieties described in the entire preceding text.

Moieties of the above depicted type are cleaved by FAP in a highly effective manner (k_{cat}/K_{M}>10⁶ s⁻¹·M⁻¹) and with excellent selectivity for FAP versus prolyl oligopeptidase (PREP). PREP is-unlike FAP-ubiquitously expressed in healthy tissue. High selectivity for FAP over PREP limits off-target prodrug activation in healthy tissue and concomitant systemic toxicity.

In order to increase the steric probability and efficiency of intratumoral FAP-activation and parent drug delivery the present invention further proposes prodrugs that comprise two, three, four or more FAP-activatable initiators.

A large variety of heterobifunctional linkers are commercially available either as ready-made compound, crosslinking kit or service (e.g. from https://www.carbolution.de/, https://bezwadabiomedical.com/, https://broadpharm.com, https://p3bio.com/amino-acids/fmoc-amino-acids/, https://www.thermofisher.com, https://www.profacgen.com). Some vendors offer comprehensive libraries of Fmoc- and tBu-protected amino acids. The Crosslinking Technical Handbook, ThermoFisher® Scientific (2022; https://assets.thermo-fisher.com/TFS-Assets/BID/Handbooks/bioconjugation-technical-handbook.pdf) describes numerous linker chemistries and bioconjugation strategies. Commercially available linker compounds afford practically unlimited possibilities for trivial modification of self-immolative linkers without affecting key pharmacologic properties of the inventive prodrugs to a clinically meaningful extent. Hence, it is noted that a partly generic description of self-immolative linker structures in the present invention does not diminish the technical feasibility and medicinal effect of the inventive prodrugs.

US 2017/0119901 A1 in paragraphs 401-431 (Examples 1 and 2) describes the synthesis of FAP-activatable prodrugs. The synthetic schemata of US 2017/0119901 A1, which are incorporated by reference in the present patent application, enable the skilled person to prepare a large variety of FAP-activatable prodrugs of type Fc-L-Ct in an analogous manner

The beneath cited articles further disclose various self-immolative linkers and methods for their preparation:
A. Alouane, R. Labruère, T. Le Saux, F. Schmidt, L. Jullien; Self-Immolative Spacers: Kinetic Aspects, Structure-Property Relationships, and Applications; Angew. Chem. Int. Ed. 2015, 54, 7492-7509; doi: 10.1002/anie.201500088;
A. G. Gavriel, M. R. Sambrook, A. T. Russell, W. Hayes; Recent advances in self-immolative linkers and their applications in polymeric reporting systems; Polym. Chem., 2022, 13, 3188; doi: 10.1039/d2py00414c;
D. Xiao, L. Zhao, F. Xie, S. Fan, L. Liu, W. Li, R. Cao, S. Li, W. Zhong, X. Zhou; A bifunctional molecule-based strategy for the development of theranostic antibody-drug conjugate; Theranostics 2021, 11(6): 2550-2563; doi: 10.7150/thno.51232.

The technical disclosure of the above articles by Alouane et al., Gavriel et al. and Xiao et al. is incorporated by reference in the present patent application.

In the present invention the terms “small-molecule-drug-conjugate”, “SMDC” and “prodrug” are synonymous and refer to a chemical compound that comprises one or more initiator or trigger moieties that are activatable by fibroblast activation protein (FAP), a linear or branched self-immolative linker moiety and a radical or residue of a chemotherapeutic compound, wherein the self-immolative linker is arranged between the radical or residue of the chemotherapeutic compound and each of the one or more initiator or trigger moieties.

The term “activatable by fibroblast activation protein (FAP)” paraphrases catalytic (i.e. rapid and highly efficient) cleavage of the initiator or trigger moiety from the self-immolative linker. Cleavage efficiency is typically expressed in units of [s⁻¹·M⁻¹] as the ratio k_{cat}/K_{M} of the catalytic rate constant k_{cat} and the Michaelis-Menten constant K_{M} (cf. https://en.wikipedia.org/wiki/Michaelis-Menten_kinetics). K_{cat} and K_{M} are readily determined via commonly known enzymatic assay techniques.

In the present invention the term “residue of . . . ” or “radical of . . . ” refers to a chemical compound having at least one unpaired valence electron (cf. https://en.wikipedia.org/wiki/Radical_(chemistry)).

The term “residue of a chemotherapeutic compound” or “radical of a chemotherapeutic compound” designates a chemotherapeutic compound less a positively charged hydrogen ion. The term “protonation” refers to the addition of a positively charged hydrogen ion to a radical or residue of a chemotherapeutic compound.

The entire content of all prior art documents cited in this patent application is incorporated by reference. In particular, the chemical synthesis methods described in the cited prior art documents are used directly or in an analogous, suitably adapted manner to prepare the prodrug.

### EXAMPLES

### Example 1: Synthesis of FAP-Cleavable Moiety and Self-Immolative Linker Conjugate

The general synthetic pathway outlined beneath in Scheme 1 rests on:
A. De Decker, G. Vliegen, D. Van Rompaey, A. Peeraer, A. Bracke, L. Verckist, K. Jansen, R. Geiss-Friedlander, K. Augustyns, H. De Winter, I. De Meester, A.-M. Lambeir, P. Van der Veken, Novel Small Molecule-Derived, Highly Selective Substrates for Fibroblast Activation Protein (FAP), ACS Med. Chem. Lett. 2019, 10, 8, 1173-1179).

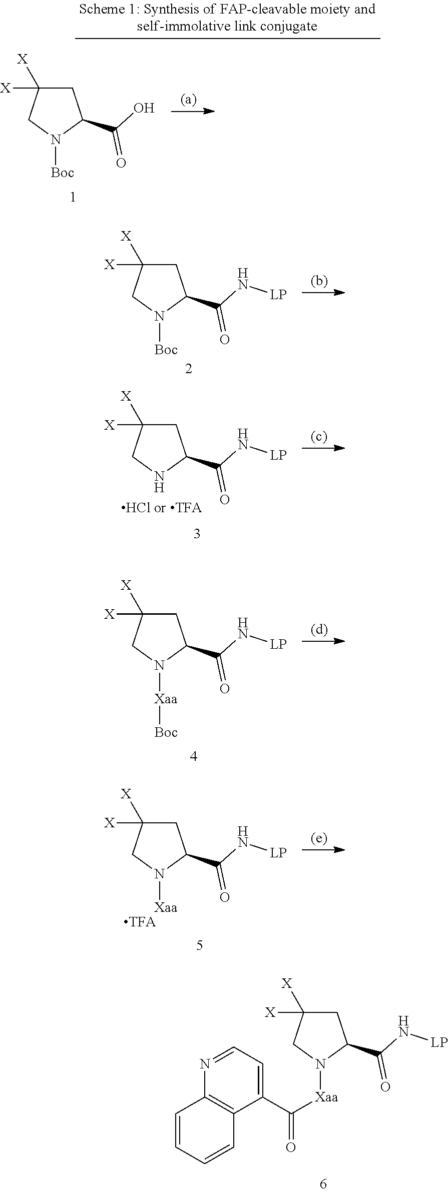

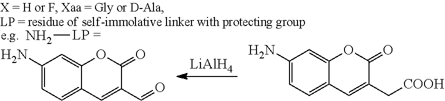

Commercially available compound 1 (i.e. Boc-L-proline or Boc-4,4-difluoro-L-proline) is coupled with protected self-immolative linker precursor NH₂-LP (see also Example 2). Resulting compound 2 is deprotected to obtain intermediate 3. The latter is coupled to Boc-protected D-alanine or glycine, thus, yielding protected conjugate 4. Acidolytic deprotection of compound 4 yields intermediate 5, from which conjugate 6 is synthesized by acylating the free amine group with quinoline-4-carboxylic acid.

### Reagents and Conditions:

(a) 1-chloro-N,N,2-trimethyl-1-propenylamine TEA, DCM: THF (1:1), rt;
(b) HCl or TFA, DCM, rt;
(c) Boc-Xaa, T₃P, DIPEA, DCM, rt;
(d) TFA, DCM, rt;
(e) quinoline-4-carboxylic acid, T₃P, DIPEA, DCM.

As illustrated in Scheme 1 the self-immolative linker precursor NH₂-LP can be readily prepared from commercially available 6-Amino-2-oxochromene-3-carboxylic acid (CAS no. 91587-88-1) through reduction with LiAlH₄.

### Example 2: Synthesis of Coumarin-Based Self-Immolative Linker and Conjugation with FAP-Cleavable Moiety

The synthesis outlined beneath in Scheme 2 is based on:
R. Weinstain, E. Segal, R. Satchi-Fainarob, D. Shabat; Real-time monitoring of drug release; Chem. Commun., 2010, 46, 553-555; and
N. C. Lim, J. V. Schuster, M. C. Porto, M. A. Tanudra, L. Yao, H. C. Freake, C. Brückner; Coumarin-Based Chemosensors for Zinc (II): Toward the Determination of the Design Algorithm for CHEF-Type and Ratiometric Probes; Inorganic Chemistry, 2005, Vol. 44, No. 6, 2018-2030

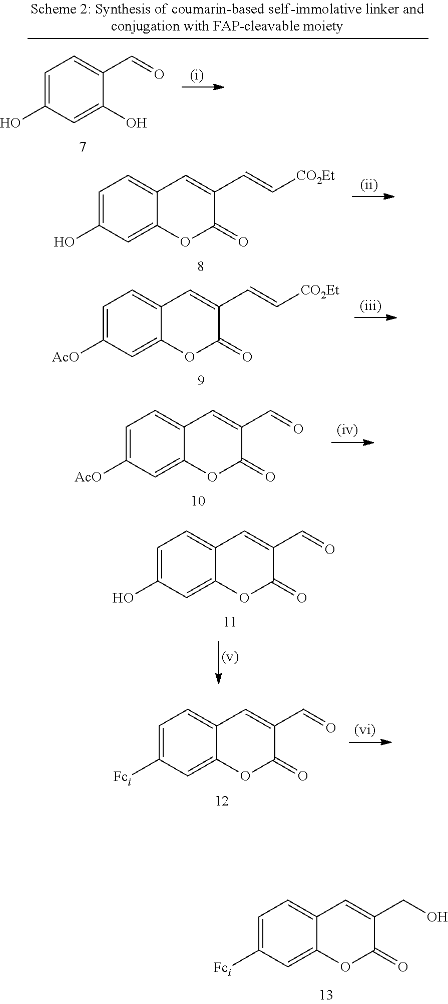

(i) 2,4-Dihydroxybenzaldehyde 7 (0.74 g, 5.36 mmol) is dissolved in EtOH (15 mL). Diethyl glutaconate (1.0 mL, 5.65 mmol) is added, followed by 3 drops of piperidine (dried over KOH pellets). The obtained solution is refluxed for 24 h. The reaction mixture is allowed to slowly cool to room temperature and then chilled to −20° C. The yellow crystals formed are filtered off and dried to yield 3-(7-Hydroxy-2-oxo-2H-chromen-3-yl) acrylic acid ethyl ester 8 (1.24 g, 89% yield).

(ii) Acrylic acid ethyl ester 8 (0.200 g, 0.77 mmol) is dissolved in dry pyridine (4 mL), and acetic anhydride (4 mL) is added. The reaction mixture is stirred at ambient temperature for 0.5 h, subsequently poured onto ice and stirred for additional 10 min. The resulting white precipitate is filtered and dried yielding 3-(7-Acetoxy-2-oxo-2H-chromen-3-yl) acrylic acid ethyl ester 9 (0.210 g, 90%).

(iii) Acrylic acid ethyl ester 9 (2.20 g, 7.28 mmol) is dissolved in THF (200 mL). OsO₄ (2 mL of 4% w/w in water) is added to the mixture and stirred for 0.5 h. NaIO₄ (3.42 g, 16 mmol) is added, and the suspension is stirred at ambient temperature. Once the starting material is consumed (ca. 5 d), the solution is dried by rotary evaporation. The resulting solid is partitioned between water and CH₂Cl₂. The organic layer was taken to dryness by rotary evaporation. Intermediate acetic acid 3-formyl-2-oxo-2H-chromen-7-yl ester 10 is isolated as a white solid (1.40 g, 83%) using column chromatography (silica-solvent gradient from CH₂Cl₂ to CH₂Cl₂/5% CH₃CN).

(iv) Ester 10 (620 mg, 2.66 mmol) is dissolved in 32% NH₄OH solution in water and acetonitrile (MeCN) is slowly added until the reaction mixture becomes homogenous. The reaction is followed to completion (20 minutes) by TLC (EtOAc: Hex 1:1). EtOAc is added and the solution is washed twice with HCl [1M]. The organic phase is dried over MgSO₄, then filtered and the solvent removed under reduced pressure to give compound 11 (456 mg, 90%).

(v) Compound 11 is conjugated with FAP-cleavable moiety Fcᵢ via common amide (peptide) bond formation to obtain compound 12.

(vi) Conjugate 12 (100 mg, 0.53 mmol, 1 eq) is dissolved in MeOH (4 mL) and sodium borohydride (30 mg, 0.79 mmol, 1.5 eq) is added. The reaction is monitored to completion (10 minutes) by TLC (EtOAc: Hex 1:1). The reaction mixture is diluted with EtOAc, washed once with saturated NH₄Cl solution, dried over MgSO₄, then filtered and the solvent is removed under reduced pressure. The crude product is purified by column chromatography on silica gel (EtOAc: Hex 1:1) to give compound 13 (70-86% yield.

### Example 3: Synthesis of 7-amino-3-(1-hydroxyethyl)-2H-chromen-2-one

The synthetic route depicted beneath in Scheme 3 is based on: D. Xiao, L. Zhao, F. Xie, S. Fan, L. Liu, W. Lᵢ, R. Cao, S. Lᵢ, W. Zhong, X. Zhou; A bifunctional molecule-based strategy for the development of theranostic antibody-drug conjugate; Theranostics 2021, 11 (6): 2550-2563; doi: 10.7150/thno.51232.

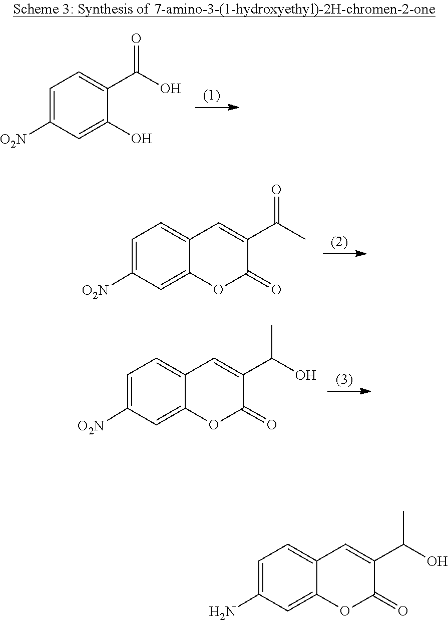

Synthesis of 3-acetyl-7-nitro-2H-chromen-2-one (1): To a stirring mixture of 2-hydroxy-4-nitrobenzaldehyde (5.00 g, 30 mmol) and ethyl acetoacetate (4.6 mL, 36 mmol), 349 μL of piperidine are added. After reflux for 1.5 h, the yellowish solid precipitate is filtered off, subsequently washed with ethanol to afford intermediate 1 (4.00 g, 57.1% yield).

Synthesis of 3-(1-hydroxyethyl)-7-nitro-2H-chromen-2-one (2): To a solution of intermediate 1 (2.40 g, 10.30 mmol) in methanol and tetrahydrofuran (1:1, 200 mL total) is added sodium borohydride (390 mg, 10.30 mmol) and cerium chloride (2.54 g, 10.3 mmol) at 0° C. After completion of the reaction within 1.5 h, the solvent is concentrated in vacuo and the crude product purified by column chromatography (1:1.5 EtOAc/hexanes) to give intermediate 2 as yellow solid (1.80 g, 74.3% yield).

Synthesis of 7-amino-3-(1-hydroxyethyl)-2H-chromen-2-one (3): Intermediate 2 (500 mg, 2.13 mmol), Iron (III) chloride hexahydrate (115 mg, 0.45 mmol), hydrazine hydrate (1.50 g, 25.6 mmol) and active carbon (305 mg, 25.6 mmol) are mixed in absolute ethanol (30 mL) and refluxed for 2 h. The solution is filtered and the filtrate concentrated in vacuo and the crude product purified by column chromatography (1:1 EtOAc/hexanes) to yield 3 as white solid (300 mg, 68.8% yield).

### Example 4: Ether Bond Formation Between Different Alcohols

The synthetic strategy outlined beneath in Scheme 4 follows:
P. K. Sahoo, S. S. Gawali, C. Gunanathan; Iron-Catalyzed Selective Etherification and Transetherification Reactions Using Alcohols; ACS Omega 2018, 3, 124-136.

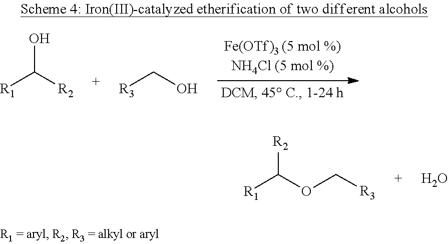

Secondary alcohol (0.5 mmol), primary alcohol (0.5 mmol), Fe(OTf)₃ (0.025 mmol, 5 mol %) and NH₄Cl (0.025 mmol, 5 mol %) in DCM (2 mL) are heated at 45° C. for 1 to 24 h. Fe(NO₃)₃·9H₂O (0.025 mmol, 5 mol %) is used as catalyst. Reaction is carried out at 70° C. Product is isolated via column chromatographic purification with typical yield between 40 and 93%.

### Example 5: Conjugation of Alcohol and Amine Through N—O Bond Formation

The reaction strategy outlined beneath in Scheme 5a-5e bears on:
J. Hill, A. A. Hettikankanamalage, D. Crich; Diversity-Oriented Synthesis of N,N,O-Trisubstituted Hydroxylamines from Alcohols and Amines by N—O Bond Formation; J. Am. Chem. Soc. 2020, 142, 14820-14825.

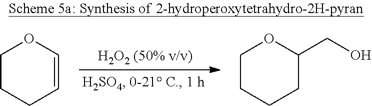 [SMILES: C1=COCCC1 ; OCC1CCCCO1]

H₂SO₄ (18.4 M, 0.05 mL, 0.92 mmol, 0.01 eq) is added to a stirred solution of H₂O₂ (50% v/v) (3.8 mL, 58.8 mmol, 2 eq.) at 0° C. The solution is stirred for 10 min, after which, 3,4-dihydropyran (2.68 mL, 29.4 mmol, 1 eq.) is added dropwise at 0° C. and the solution is stirred for 1 h. Following, the reaction mixture is diluted with Et₂O (15 mL) and quenched by addition of saturated NH₄Cl (30 mL) solution. The resulting biphasic mixture is transferred to a separatory funnel and the layers separated. The aqueous layer is extracted with ethyl acetate (5× 40 mL) and the organic layers are combined, dried over Na₂SO₄, filtered, and concentrated in vacuo. The obtained residue is purified by flash column chromatography on silica (eluent: 5:95 EtOAc: Hexanes) to obtain the compound 2-hydroperoxytetrahydro-2H-pyran as a colorless oil (2.04 g, 17.3 mmol, 59%).

aper, or wood. I.e. the substrate may comprise or consist of a separate barrier coating formed on a film.

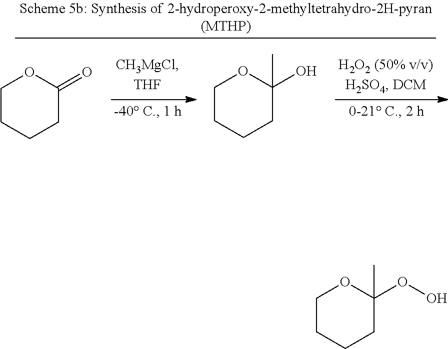

CH₃MgCl 3.0 M solution in THF (20 mmol, 6.67 mL, 1.0 eq) is added dropwise over 10 minutes in to a solution of δ-valerolactone (20 mmol, 1.86 mL, 1.0 equiv.) in 40 mL of anhydrous THF at −40° C. under an argon atmosphere. The reaction is stirred for 1 h at −40° C. After consumption of starting material as indicated by TLC and MS, the reaction mixture is brought to −20° C. and quenched with a saturated solution of NH₄Cl (40 mL) followed by diluting with DI water (20 mL) at room temperature. The resulting biphasic mixture is separated and the aqueous layer extracted with EtOAc (5× 40 mL). The organic layers are combined and dried over Na₂SO₄, filtered, and concentrated in vacuo. The crude reaction mixture is used subsequently without further purification.

Sulfuric acid (18.4 M, 109 μL, 2.0 mmol, 0.10 eq.) is added to a stirred solution of 2-methyltetrahydro-2H-pyran-2-ol (2.3 g, 20 mmol, 1 eq.) obtained in the previous step in 100 mL of DCM at 0° C. Aqueous hydrogen peroxide solution (50% w/w) (6.8 mL, 100 mmol, 5.00 eq.) is added dropwise over 5 min and stirred another 10 min at 0° C. The reaction is brought to room temperature and stirred for 2 h. The reaction is quenched with a saturated NH₄Cl solution (40 mL) and the resulting biphasic mixture is separated and the aqueous layer extracted with EtOAc (5×40 mL). The combined organic layers are dried over Na₂SO₄, filtered and concentrated in vacuo. The obtained residue is purified by flash column chromatography on silica (eluent: 0:100 DCM-8:92 Et₂O:DCM) to obtain 2-hydroperoxy-2 methyltetrahydro-2H-pyran as a clear, colorless oil (1.72 g, 13.0 mmol, 65% total yield).

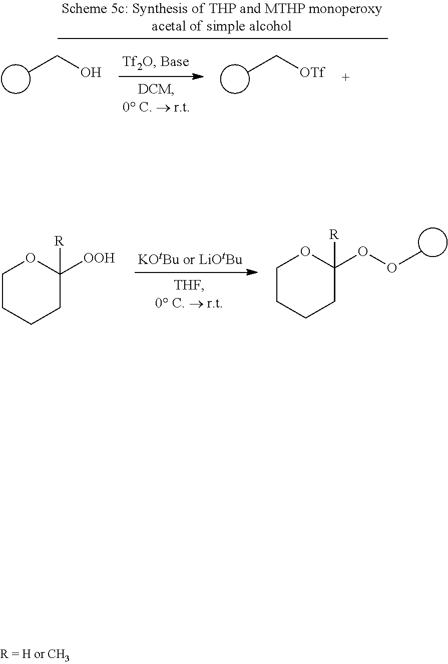

Anhydrous DCM (0.17-0.60 M), alcohol (1.0 eq.) and base (1.5 eq.) are added to an oven dried flask under argon atmosphere at 0° C. The solution is stirred for 10 min, after which, Tf₂O (1.2-1.5 eq.) is added dropwise. The solution is stirred for 30 to 60 min at 0° C. Following, HCl (10%, 10 mL) is added and the layers are separated. The organic layer is washed with saturated NaHCO₃ (1×10 mL). The aqueous layer is extracted with EtOAc (3×5 mL) and the organic layers are combined and washed with saturated NaCl solution (1×10 mL), dried over MgSO₄, filtered, and concentrated in vacuo. The triflate is extracted via flash silica column chromatography (eluent: EtOAc: Hexanes) and used in the following step.

Lithium tert-butoxide or potassium tert-butoxide (1.2-1.5 eq.) is added in a single portion under argon atmosphere (balloon) to a stirred solution of THP or MTHP (1.0-2.0 eq.) in anhydrous THF (0.2-0.5 M). The solution is stirred for 10 min at 0° C., after which, a portion of the triflate (1.0-2.0 eq.) obtained in the previous step is added dropwise via syringe. The solution is stirred for 1 h at 0° C., after which, the mixture is allowed to reach room temperature and stirred for an additional 1-24 h. The reaction mixture is quenched with NaHCO₃ (20 mL) and diluted with EtOAc (10 mL). The layers are separated and the aqueous layer extracted with EtOAc (3×5 mL). The combined organic layers are dried over MgSO₄/Na₂SO₄, filtered, and concentrated in vacuo. Flash column chromatography on silica (eluent: EtOAc: Hexanes) yields the monoperoxy acetal.

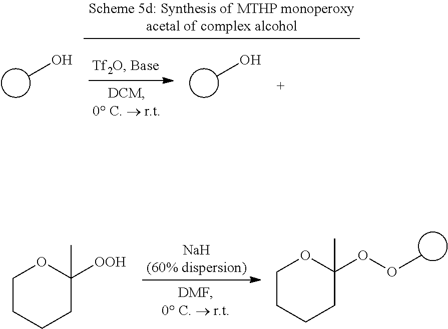

Anhydrous DCM (0.13-0.50 M), alcohol (1.0 eq.) and pyridine (2.0 eq.) are added to an oven dried flask under argon atmosphere at 0° C. The solution is stirred for 10 min, thereafter, Tf₂O (1.2-1.5 eq.) is added dropwise. The solution is stirred for 30 to 60 min at 0° C. and subsequently diluted with a few drops of MeOH and 10% HCl (1×10 mL). The layers are separated and the organic layer washed with saturated NaHCO₃ (1×10 mL). The aqueous layer is extracted with EtOAc (3×5 mL) and the organic layers are combined and washed with saturated NaCl solution (1×10 mL), dried over MgSO₄, filtered, and concentrated in vacuo. The triflate is extracted via flash silica column chromatography (eluent: EtOAc: Hexanes) and used in the following step.

NaH (60% dispersion in mineral oil, 1.2-1.5 eq.) is added in a single portion under argon atmosphere to a stirred solution of MTHP (1.0 eq.) in anhydrous DMF. The solution is stirred for 10 min at 0° C., thereafter, the triflate (1.3 eq.) obtained in the previous step is added dropwise via syringe. The solution is stirred for 1 h. The mixture is allowed to settle at room temperature and stirred for an additional 1-16 h. The reaction mixture is then diluted with EtOAc (10 mL) and quenched with saturated NaHCO₃ (10 mL). The layers are separated and the aqueous layer extracted with EtOAc (3×5 mL). The organic layers are dried over Na₂SO₄/MgSO₄, filtered, and concentrated in vacuo. The monoperoxy acetal is extracted via flash column chromatography on silica (eluent: EtOAc: Hexanes).

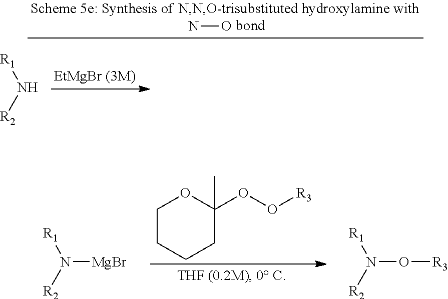

Amine (0.25-11.0 mmol, 2.5 eq.) and 0.25-11.0 mL anhydrous THF are added to an oven/flame dried flask at 0° C. under an argon atmosphere. To this solution EtMgBr (3M in diethyl ether) (0.2-8.7 mmol, 2.0 eq.) is added dropwise and the reaction mixture is stirred for 10-30 min at 0° C. (magnesium amide formation produces substantial amount of gas, hence, at larger scale caution is mandated). The magnesium amide is subsequently transferred via syringe to a stirred solution of THP or MTHP monoperoxyacetal (0.10-4.36 mmol, 1.0 eq.) stirred in additional 0.25-11.0 mL anhydrous THF (0.2 M total) under argon atmosphere at 0° C. The solution is stirred until the starting material is consumed as indicated by TLC and MS, after which, the mixture is quenched by addition of ice water and the layers are separated. The aqueous layer is extracted with EtOAc and the combined organics are dried over MgSO₄/Na₂SO₄, filtered, and concentrated in vacuo. Flash column chromatography on silica or neutral alumina (eluent: EtOAc: Hexanes or DCM: EtOAc) yield the N,N,O-trisubstituted hydroxylamines.

### Example 6: Amide Bond Formation

A generic example of an amide coupling reaction is shown in scheme 6.

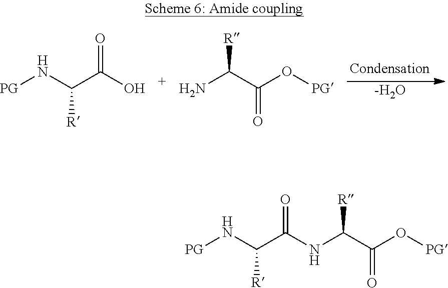

Owing to a virtually unlimited set of readily available carboxylic acid and amine derivatives, amide coupling strategies open up a simple route for the synthesis of novel compounds. The person skilled in the art is aware of numerous reagents and protocols for amide coupling. The most commonly used amide coupling strategy is based on the condensation of a carboxylic acid with an amine. For this purpose, the carboxylic acid is generally activated. Prior to the activation, remaining functional groups are protected. The reaction is carried out in two steps, either in one reaction medium (single pot) with direct conversion of the activated carboxylic acid, or in two steps with isolation of activated “trapped” carboxylic acid and reaction with an amine.

The carboxylic reacts here with a coupling agent to form a reactive intermediate which can be reacted in isolated form or directly with an amine. Numerous reagents are available for carboxylic acid activation, such as acid halide (chloride, fluoride), azides, anhydrides or carbodiimides. In addition, reactive intermediates formed may be esters such as pentafluorophenyl or hydroxysuccinimido esters. Intermediates formed from acyl chlorides or azides are highly reactive. However, harsh reaction conditions and high reactivity are frequently a barrier to use for sensitive substrates or amino acids. By contrast, amide coupling strategies that utilize carbodiimides such as DCC (dicyclohexylcarbodiimide) or DIC (diisopropylcarbodiimide) open up a broad spectrum of application. Frequently, especially in the case of solid-phase synthesis, additives are used to improve reaction efficiency. Aminium salts are highly efficient peptide coupling reagents having short reaction times and minimal racemization. With some additives, for example HOBt, it is impossible to completely prevent racemization. Aminium reagents are used in an equimolar amount with the carboxylic acid in order to prevent excess reaction with the free amine of the peptide. Phosphonium salts react with carboxylate, which generally requires two equivalents of a base, for example DIEA. A significant advantage of phosphonium salts over iminium reagents is that phosphonium does not react with the free amino group of the amine component. This enables couplings in a molar ratio of acid and amine and helps to prevent the intramolecular cyclization of linear peptides and excessive use of costly amine components.

An extensive summary of reaction strategies and reagents for amide couplings can be found in the following review articles:
Analysis of Past and Present Synthetic Methodologies on Medicinal Chemistry: Where Have All the New Reactions Gone?; D. G. Brown, J. Boström; J. Med. Chem. 2016, 59, 4443-4458;
Peptide Coupling Reagents, More than a Letter Soup; A. El-Faham, F. Albericio; Chem. Rev. 2011, 111, 6557-6602;
Rethinking amide bond synthesis; V. R. Pattabiraman, J. W. Bode; Nature, Vol. 480 (2011) 22/29;
Amide bond formation: beyond the myth of coupling reagents; E. Valeur, M. Bradley; Chem. Soc. Rev., 2009, 38, 606-631

### Example 7: Inventive Prodrugs with Single FAP-Activatable Initiator

Schemes 7a-7m show exemplary prodrugs with one FAP-activatable initiator. Dotted lines indicate FAP enzymatic cleavage and drug release from the self-immolative linker.

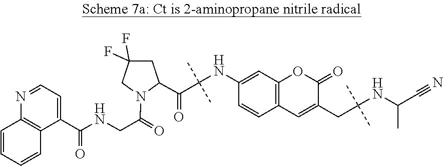

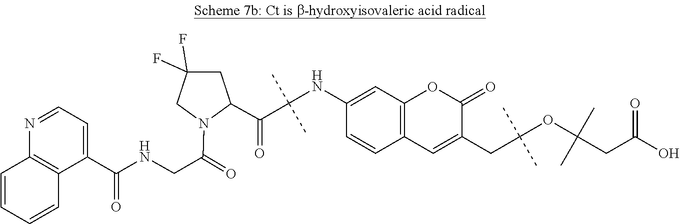

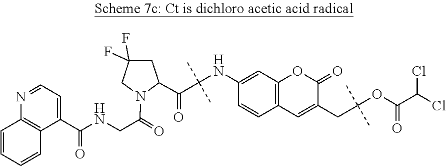

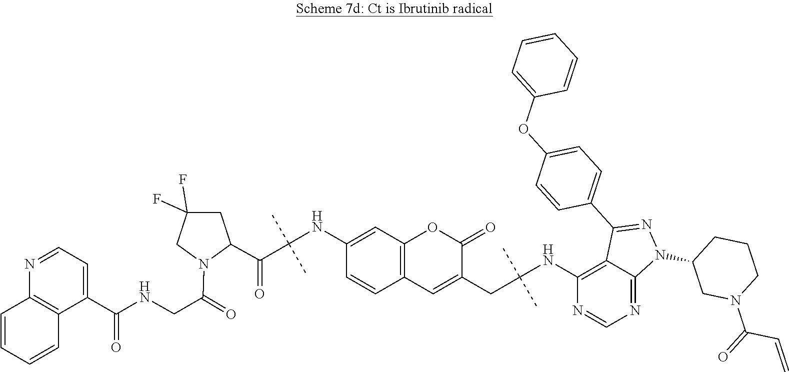

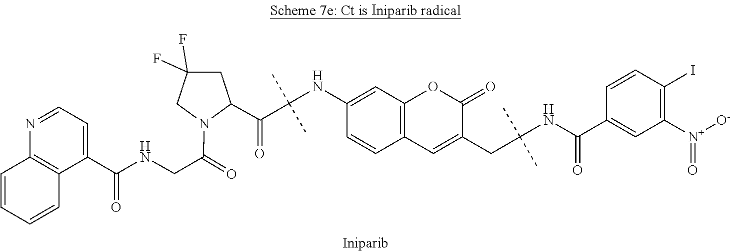

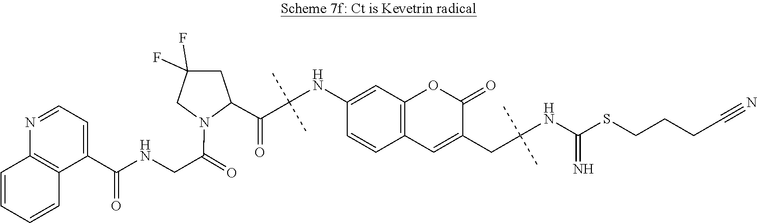

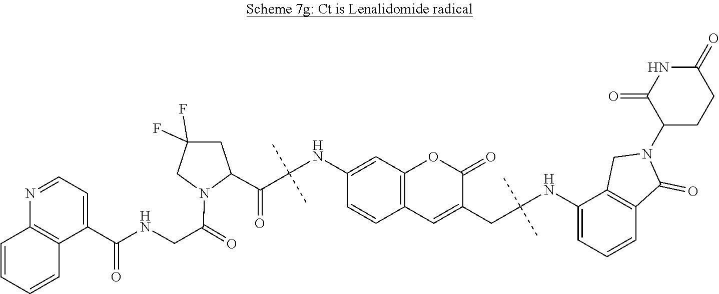

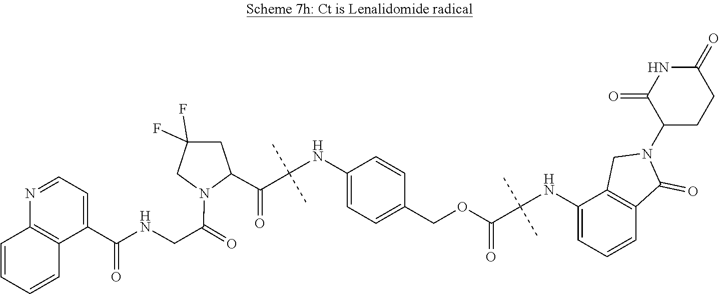

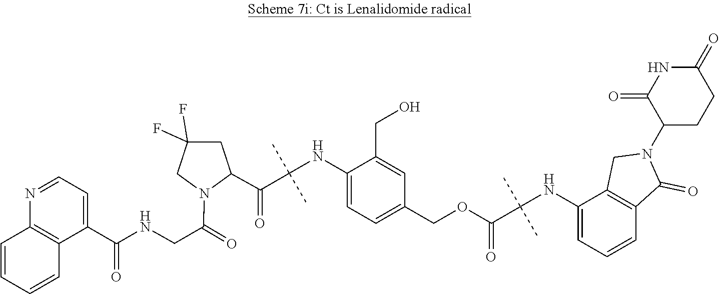

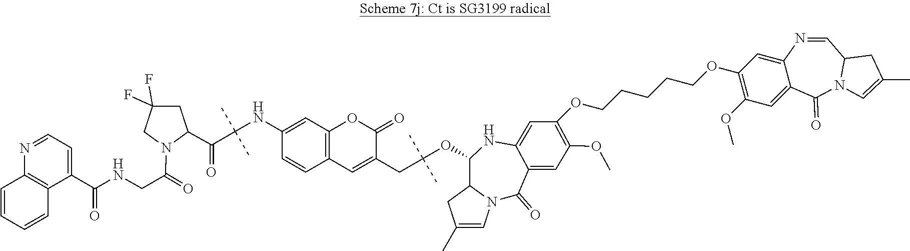

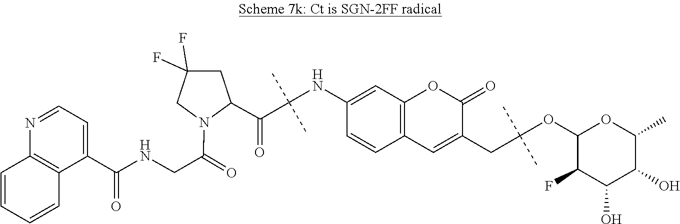

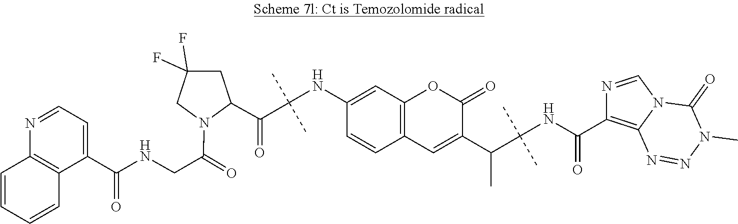

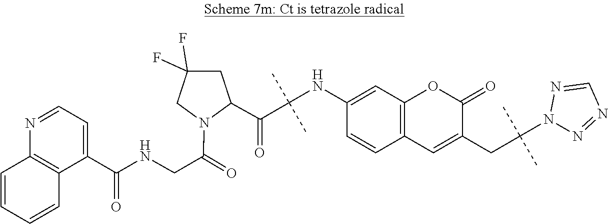

### Example 8: Inventive Prodrugs with Two FAP-Activatable Initiators

Schemes 8a-8c show exemplary prodrugs with two FAP-activatable initiators. Dotted lines indicate FAP enzymatic cleavage and drug release from the self-immolative linker.

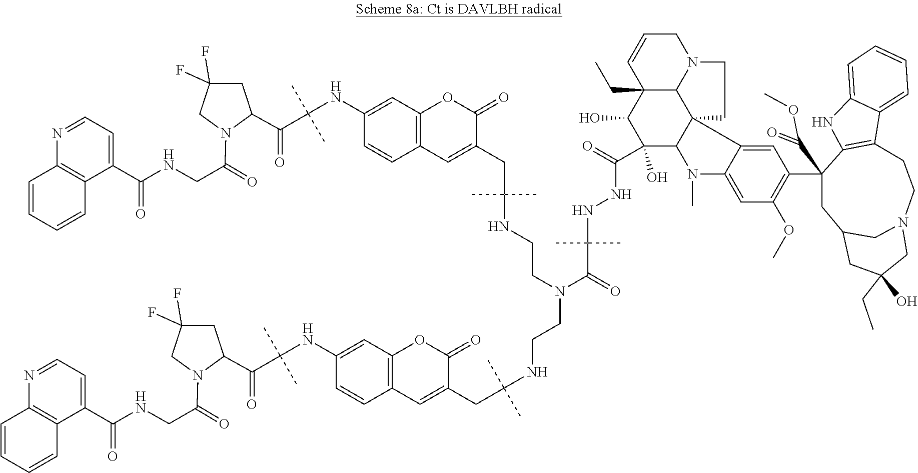

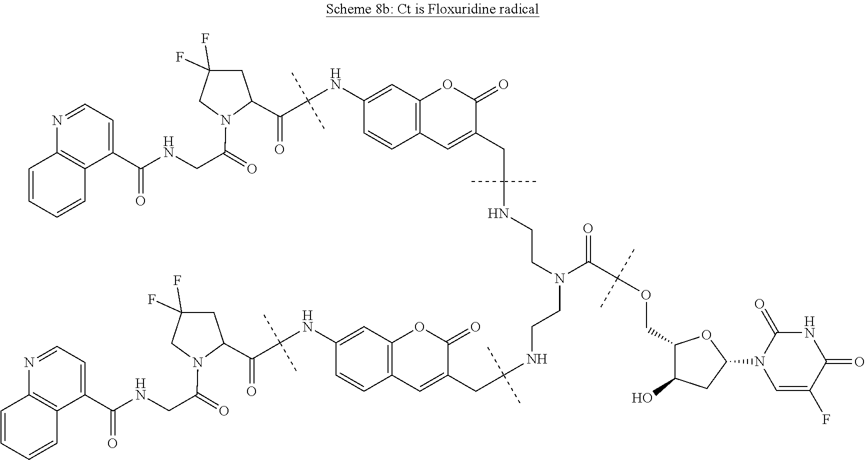

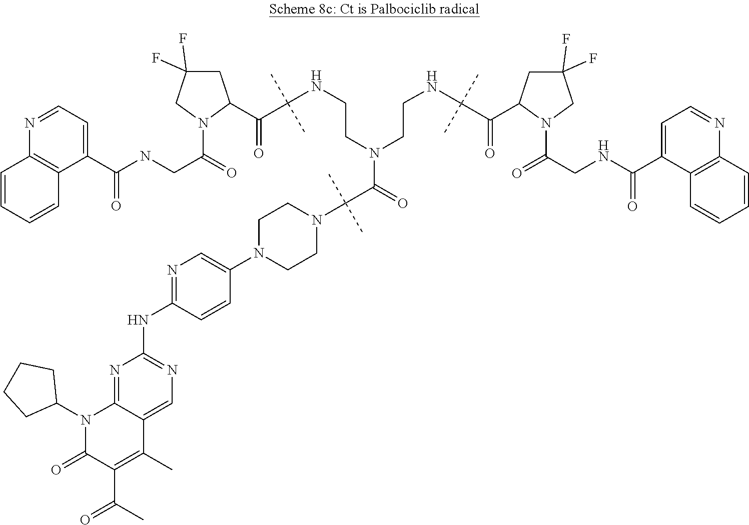

1. Prodrug comprising a chemotherapeutic compound radical Ct, a linear or branched self-immolative linker L and one, two, three, four or more initiators (F1, F2, F3, F4), wherein
L is covalently bound to a nitrogen, amine or oxygen radical of Ct;
L comprises one, two, three, four or more amine radicals;
each of initiators (F1, F2, F3, F4) is covalently bound to an amine radical of L;
each of initiators (F1, F2, F3, F4) is configured for enzymatic cleavage from L by fibroblast activation protein (FAP);
L is configured for release of Ct upon cleavage of any one of initiators (F1, F2, F3, F4);
initiators (F1, F2, F3, F4) independently of one another comprise or have a structure selected from the group of structures comprising 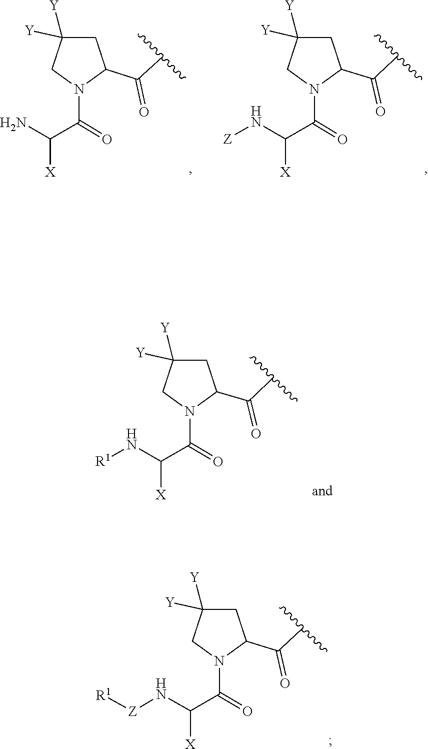
wherein the terminal carbonyl is covalently bound to an amine radical of the self-immolative linker L, X=—H or —CH₃, Y=—H or —F, —R¹ is a radical of a first pharmaco-kinetic modulating moiety and Z is a moiety having a structure selected from the group comprising structures (1), (2), (3), (4), (5), (6), (7), (8), (9), (10), (11), (12), (13), (14), (15), (16), (17), (18), (19), (20), (21), (22), (23), (24) and (25) with 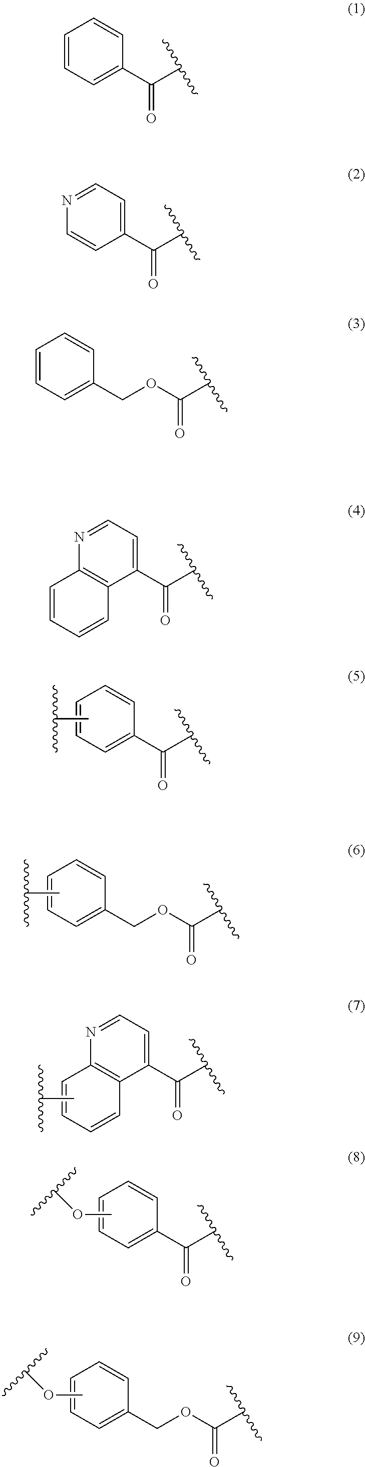 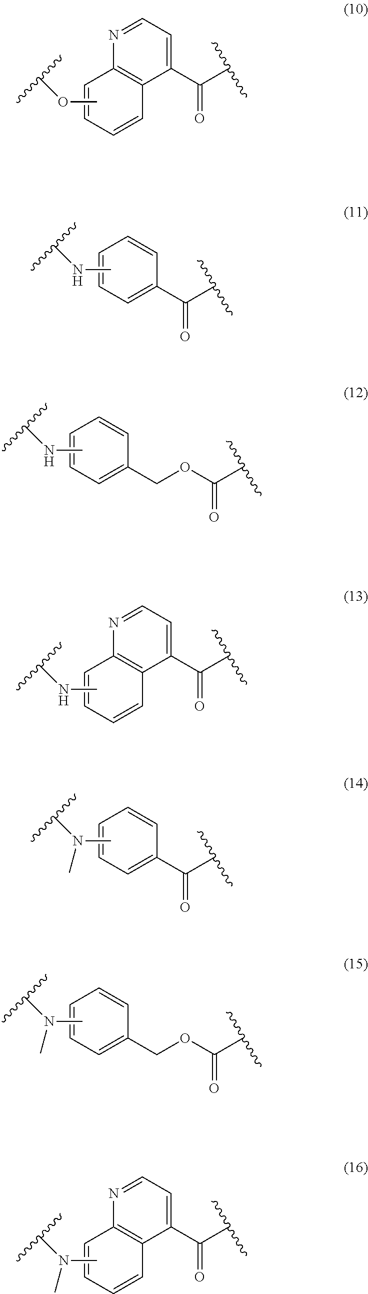 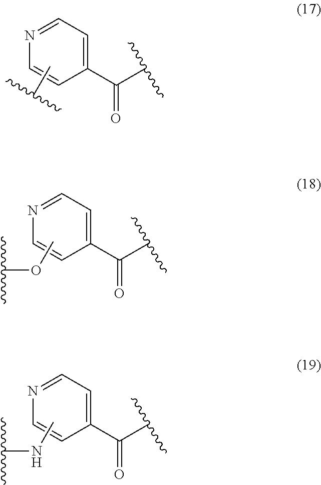 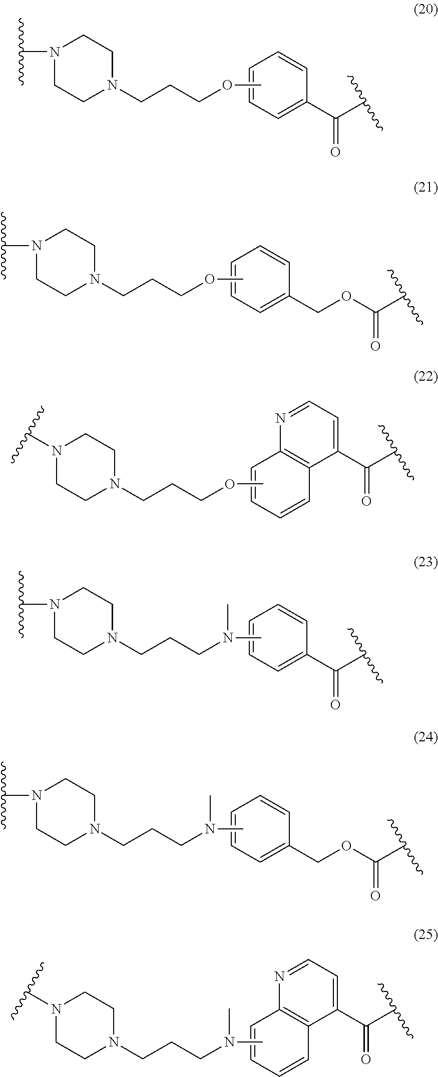
and
Ct is a radical of a chemotherapeutic compound selected from the group comprising 1,2,3,4-Tetrahydrogen-staurosporine, 17-Dmag, 2-Aminopropanenitrile, 4SC202, ABBV-CLS-484, Abemaciclib, Abexinostat, Acalabrutinib, Acetylbufalin, Aderbasib, Afatinib, Afuresertib, Alectinib, Alisertib, Alpelisib, Alvocidib, AMD3465, Anlotinib, Apalutamide, AR-42, Asciminib, Atuveciclib, Avapritinib, Axitinib, AZD7762, BAY1125976, Belinostat, β-Hydroxyisovaleric acid, BF211, Bicalutamide, Binimetinib, Bortezomib, Bosutinib, Brigatinib, Bufalin, Buparlisib, Buthionine sulfoximine, Cabozantinib, Capivasertib, Capmatinib, Carfilzomib, CEP-9722, Ceralasertib, Ceritinib, Chidamide, CHR-3996, Citarinostat, Cobimetinib, CompK, Copanlisib, Crenolanib, Crizotinib, CUDC-101, Dabrafenib, Daclatasvir, Dacomitinib, Darolutamide, Dasatinib, Dasatinib D1, Dasatinib D2, Dasatinib D3, Dasatinib D4, Decitabine, Defactinib, Degarelix, Diethylstilbestrol, Dinaciclib, Dp44mT, DpC, DUPA, Duvelisib, E7016, Ebvaciclib, Eganelisib, Elimusertib, Emavusertib, Enasidenib, Encorafenib, Enitociclib, Entinostat, Entrectinib, Enzalutamide, Epacadostat, Epigallocatechin gallate, Epoxomicin, Erdafitinib, Erismodegib, Erlotinib, Everolimus, Fasudil, Fedratinib, Filgotinib, Foslinanib, Fostamatinib, Fruquintinib, Galunisertib, Ganetespib, Gedatolisib, Gefitinib, GFH018, Gilteritinib, Givinostat, Glasdegib, Goserelin, GSK2256098, GSK269962A, GSK690693, GUL, Halofuginone, Hymecromone, Ibrutinib, Icotinib, Idelalisib, Imatinib, Imiquimod, Infigratinib, Iniparib, Ipatasertib, Itacitinib, Ivaltinostat, Ivosidenib, Ixazomib, Kevetrin, Lapatinib, Larotrectinib, Lenalidomide, Leniolisib, Lenvatinib, Leuprolide, Linsitinib, Lonafarnib, Lorlatinib, Losartan, Lucitanib, Luminespib, M1096, Marizomib, ME-344, Merestinib, Metformin, MG132, Midostaurin, Miransertib, Mivavotinib, MK2206, MMP-9 Inhibitor I, Mobocertinib, Mocetinostat, Motesanib, MRTX1133, Navitoclax, Nazartinib, Nedisertib, Neratinib, Nilotinib, Nilutamide, Nintedanib, Niraparib, NMS-P118, NMS-P515, NSC668394, NSC95397, Numidargistat, NVP-2, Olaparib, Olmutinib, Omipalisib, Oprozomib, Osimertinib, OTS-964, Palbociclib, Pamiparib, Panobinostat, Paricalcitol, Parsaclisib, Pazopanib, Pemetrexed, Pemigatinib, Pevonedistat, Pexidartinib, Pifusertib, Plerixafor, PMPA, Ponatinib, Practinostat, Pralsetinib, Prednisone, Prexasertib, Prinomastat, Propranolol, Quisinostat, Quizartinib, Ralimetinib, Ravoxertinib, Regorafenib, Relugolix, Resminostat, Resveratrol, Retaspimycin, Retinoic acid, Ribociclib, Ricolinostat, Rigosertib, Ripretinib, RO-3306, Rocilinostat, Rogaratinib, Romidepsin, Rucaparib, Ruxolitinib, S2, S5, Saridegib, SBI-0654454, SCH772984, Seliciclib, Selitrectinib, Selpercatinib, Selumetinib, SGN-2FF, SGX393, Shikonin, Silibinin, Sitravatinib, Sonidegib, Sorafenib, Sotorasib, Staurosporine, SU11274, Sunitinib, Surufatinib, Tacedinaline, Tadalafil, Talazoparib, Taletrectinib, Tarloxotinib, Taselisib, Tazemetostat, Tefinostat, Temsirolimus, Tetrazole, Tivozanib, Tofacitinib, Tozasertib, Trametinib, Tranilast, Tretinoin, Trichostatin, Tucatinib, Tucidinostat, Tuvusertib, Ubenimex, Umbralisib, Uprosertib, USL311, Vactosertib, Valproic acid, Valsartan, Vandetanib, Veliparib, Vemurafenib, Venetoclax, Verteporfin, Vismodegib, Vorinostat, WRG-28, WZ811, Xevinapant, Zandelisib, Zanubrutinib, ZM447439, Abiraterone, Aclarubicin, Adozelesin, Alrestatin, Amanitin, Amrubicin, Anthramycin, Arenastatin, Bizelesin, Bleomycin, Camptothecin, Capecitabine, Carzelesin, CC-1065, Chaconine, Chlorambucil, Cryptophycin-24, Cyclophosphamide, Cytarabine, Dacarbazine, Dactinomycin, Daunorubicin, DAVLBH, Deruxtecan, Dexamethasone, Dichloro acetic acid, Dimethyl-SGD-1882, Docetaxel, Dolastatin-10, Doxorubicin, Duocarmycin A, Duocarmycin B1, Duocarmycin B2, Duocarmycin C1, Duocarmycin C2, Duocarmycin D, Duocarmycin GA, Duocarmycin SA, Emetine, Epirubicin, Eribulin, Etoposide, Floxuridine, Fludarabine, Fluorouracil, Flutamide, Fulvestrant, Gemcitabine, Idarubicin, Ifosfamide, Irinotecan, L-Asparaginase, Lomustine, Melphalan, Mertansine, Methotrexate, Milataxel, Mitoxantrone, Monomethyl Auristatin E, Maytansine, Maytansinoid, Ozogamicin, Paclitaxel, Pirarubicin, Pixantrone, Podophyllotoxin, Procarbazine, Rapamycin, Rachelmycin, Salinomycin, SB-T-1214, Selinexor, SN-38, Solamargine, Solanine, Talirine, Temozolomide, Tesetaxel, SG3199 (Tesirine), Thapsigargin, Tomatine, Topotecan, Tubulysin B, Valrubicin, Vinblastine, Vincristine, Vinorelbine, VIP126, Zorubicin.

2. The prodrug of claim 1, characterized in that Ct is a radical of Acetylbufalin, Bufalin, Dinaciclib, Erlotinib, Ibrutinib, Imatinib, Lenalidomide, NVP-2, Osimertinib, Palbociclib or Sorafenib.

3. The prodrug of claim 1, characterized in that initiators (F1, F2, F3, F4) independently of one another comprise or have a structure selected from the group of structures comprising 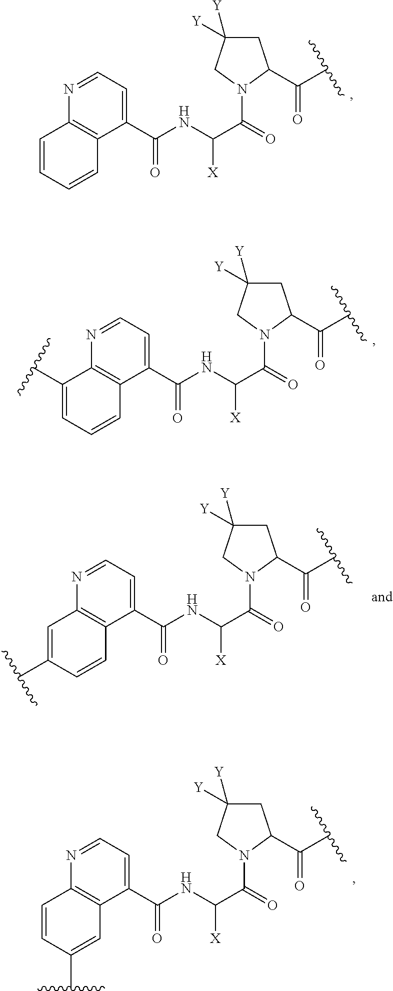
where the terminal carbonyl is covalently bound to an amine radical of the self-immolative linker L, Y=—H or —F and X=—H or —CH₃.

4. The prodrug of claim 1, characterized in that initiators (F1, F2, F3, F4) independently of one another comprise or have a structure selected from the group of structures comprising 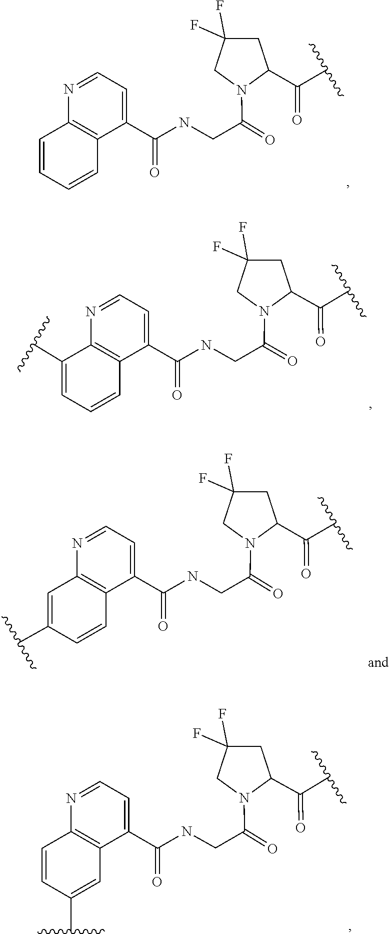
where the terminal carbonyl is covalently bound to an amine radical of the self-immolative linker L.

5. The prodrug of claim 1, characterized in that two, three, four or more of initiators (F1, F2, F3, F4) are different from one another.

6. The prodrug of claim 1, characterized in that two, three, four or more of initiators (F1, F2, F3, F4) are equal.

7. The prodrug of claim 1, characterized in that it comprises one initiator F1.

8. The prodrug of claim 1, characterized in that it comprises two initiators (F1, F2).

9. The prodrug of claim 1, characterized in that it comprises four initiators (F1, F2, F3, F4).

10. The prodrug of claim 1, characterized in that L comprises a coupling moiety for Ct, said coupling moiety having a structure selected from the group of structures comprising

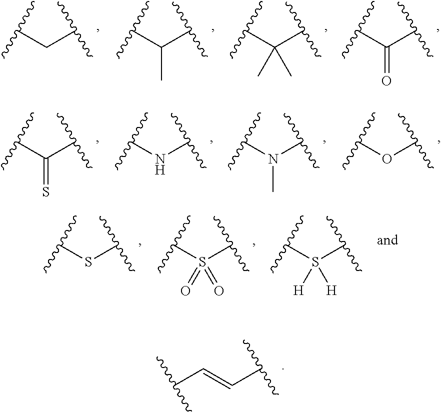

11. The prodrug of claim 1, characterized in that L comprises a coupling moiety for Ct, said coupling moiety having the structure 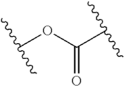 [SMILES: *OC(*)=O]
where the terminal carbonyl is covalently bound to a nitrogen radical of Ct.

12. The prodrug of claim 1, characterized in that L comprises a coupling moiety for Ct, said coupling moiety having the structure 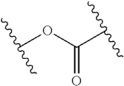 [SMILES: *OC(*)=O]
where the terminal carbonyl is covalently bound to an amine radical of Ct.

13. The prodrug of claim 1, characterized in that L comprises a coupling moiety for Ct, said coupling moiety having the structure 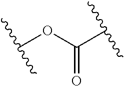 [SMILES: *OC(*)=O]
where the terminal carbonyl is covalently bound to an oxygen radical of Ct.

14. The prodrug of claim 1, characterized in that L comprises a coupling moiety for Ct, said coupling moiety having the structure 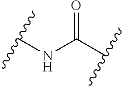 [SMILES: *NC(*)=O]
where the terminal carbonyl is covalently bound to a nitrogen radical of Ct.

15. The prodrug of claim 1, characterized in that L comprises a coupling moiety for Ct, said coupling moiety having the structure 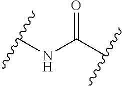 [SMILES: *NC(*)=O]
where the terminal carbonyl is covalently bound to an amine radical of Ct.

16. The prodrug of claim 1, characterized in that L comprises a coupling moiety for Ct, said coupling moiety having the structure 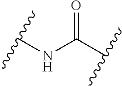 [SMILES: *NC(*)=O]
where the terminal carbonyl is covalently bound to an oxygen radical of Ct.

17. The prodrug of claim 1, characterized in that L comprises a coupling moiety for Ct, said coupling moiety having the structure 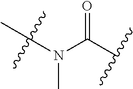 [SMILES: *C(=O)N(*)C]
where the terminal carbonyl is covalently bound to a nitrogen radical of Ct.

18. The prodrug of claim 1, characterized in that L comprises a coupling moiety for Ct, said coupling moiety having the structure 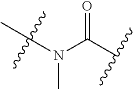 [SMILES: *C(=O)N(*)C]
where the terminal carbonyl is covalently bound to an amine radical of Ct.

19. The prodrug of claim 1, characterized in that L comprises a coupling moiety for Ct, said coupling moiety having the structure 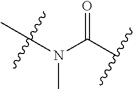 [SMILES: *C(=O)N(*)C]
where the terminal carbonyl is covalently bound to an oxygen radical of Ct.

20. The prodrug of claim 1, characterized in that L comprises a coupling moiety for Ct, said coupling moiety having the structure 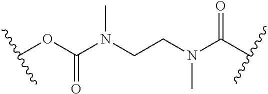 [SMILES: *OC(=O)N(C)CCN(C)C(*)=O]
where the terminal carbonyl is covalently bound to a nitrogen radical of Ct.

21. The prodrug of claim 1, characterized in that L comprises a coupling moiety for Ct, said coupling moiety having the structure 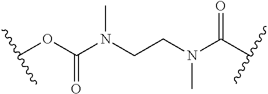 [SMILES: *OC(=O)N(C)CCN(C)C(*)=O]
where the terminal carbonyl is covalently bound to an amine radical of Ct.

22. The prodrug of claim 1, characterized in that L comprises a coupling moiety for Ct, said coupling moiety having the structure 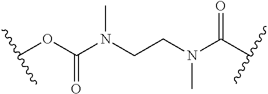 [SMILES: *OC(=O)N(C)CCN(C)C(*)=O]
where the terminal carbonyl is covalently bound to an oxygen radical of Ct.

23. The prodrug of claim 1, characterized in that L comprises a moiety having structure 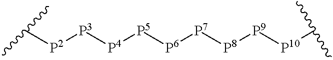
where P¹⁰ is covalently bound to Ct, moieties P^{j} with 2≤j≤h and 2≤h≤10 independently from one another are selected from the group comprising 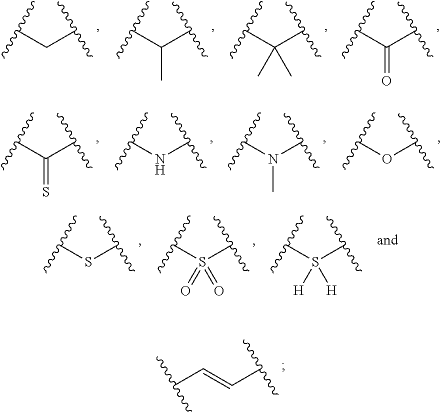
and P^{j} with h<j≤10 are absent.

24. The prodrug of claim 1, characterized in that L comprises one, two or more branching moieties having the structure 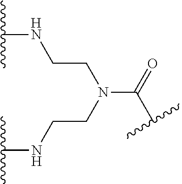 [SMILES: *NCCN(CCN*)C(*)=O]
where the terminal carbonyl is oriented towards Ct or covalently bound to Ct.

25. The prodrug of claim 1, characterized in that L comprises one or more branching moieties having the structure 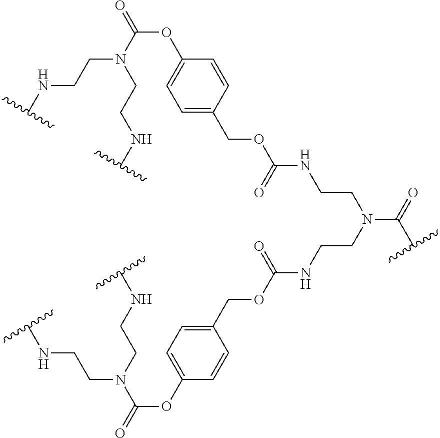 [SMILES: *NCCN(CCN*)C(=O)Oc1ccc(COC(=O)NCCN(CCNC(=O)OCc2ccc(OC(=O)N(CCN*)CCN*)cc2)C(*)=O)cc1]
where the terminal carbonyl is oriented towards Ct or covalently bound to Ct.

26. The prodrug of claim 1, characterized in that L comprises one, two, three, four or more coupling moieties for initiators (F1, F2, F3, F4), said one, two, three, four or more coupling moieties independently of one another having the structure 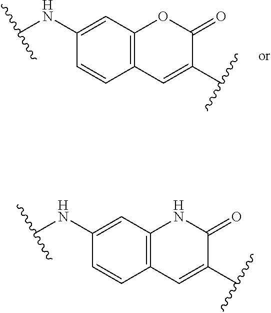 [SMILES: *Nc1ccc2cc(*)c(=O)oc2c1 ; *Nc1ccc2cc(*)c(=O)[nH]c2c1]
where the terminal amine is covalently bound to an initiator (F1, F2, F3, F4).

27. The prodrug of claim 26, characterized in that L comprises one, two, three, four or more coupling moieties for initiators (F1, F2, F3, F4), said one, two, three, four or more coupling moieties independently of one another having the structure 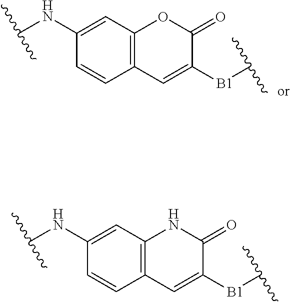
where the terminal amine is covalently bound to an initiator (F1, F2, F3, F4) and B1 independently of one another is selected from the group of moieties comprising

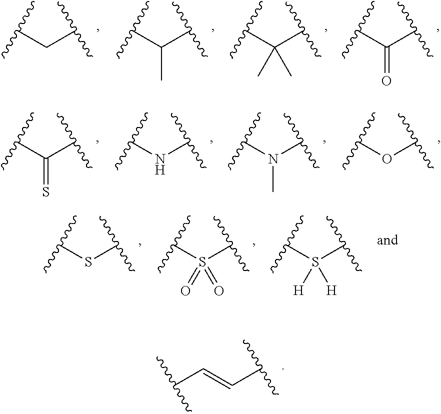

28. The prodrug of claim 26, characterized in that L comprises one, two, three, four or more coupling moieties for initiators (F1, F2, F3, F4), said one, two, three, four or more coupling moieties independently of one another having a structure selected from the group comprising 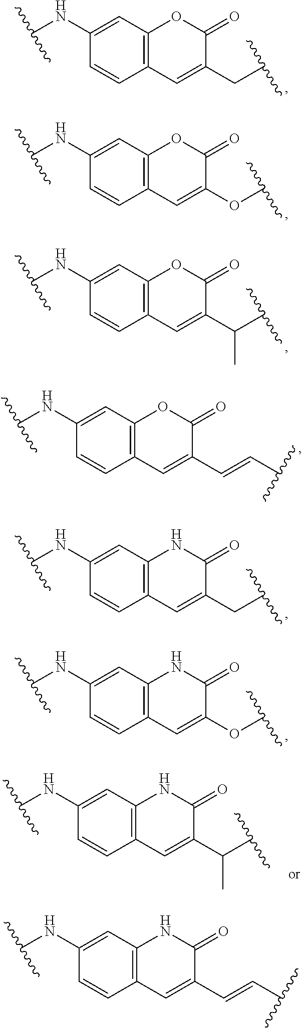
where the terminal amine is covalently bound to an initiator (F1, F2, F3, F4).

29. The prodrug of claim 26, characterized in that it comprises one initiator F1.

30. The prodrug of claim 29, characterized in that L has a structure according to claim 26.

31. The prodrug of claim 1, characterized in that L comprises one, two, three, four or more coupling moieties for initiators (F1, F2, F3, F4), said one, two, three, four or more coupling moieties independently of one another having the structure 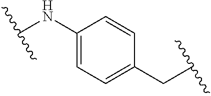 [SMILES: *Cc1ccc(N*)cc1]
where the terminal amine is covalently bound to an initiator (F1, F2, F3, F4).

32. The prodrug of claim 31, characterized in that L comprises one, two, three, four or more coupling moieties for initiators (F1, F2, F3, F4), said one, two, three, four or more coupling moieties independently of one another having the structure 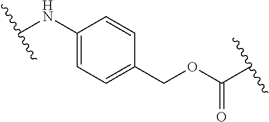 [SMILES: *Nc1ccc(COC(*)=O)cc1]
where the terminal amine is covalently bound to an initiator (F1, F2, F3, F4).

33. The prodrug of claim 1, characterized in that L comprises one, two, three, four or more coupling moieties for initiators (F1, F2, F3, F4), said one, two, three, four or more coupling moieties independently of one another having the structure 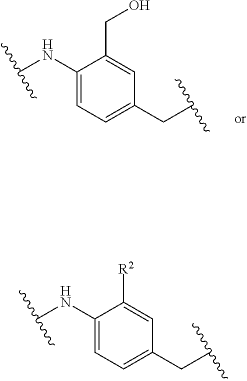
where the terminal amine is covalently bound to an initiator (F1, F2, F3, F4) and R² is a radical of a second pharmacokinetic modulating moiety.

34. The prodrug of claim 32, characterized in that L comprises one, two, three, four or more coupling moieties for initiators (F1, F2, F3, F4), said one, two, three, four or more coupling moieties independently of one another having the structure 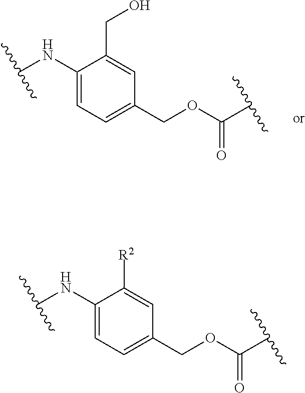
where the terminal amine is covalently bound to an initiator (F1, F2, F3, F4) and R² is a radical of a second pharmacokinetic modulating moiety.
